(19) United States
(12) Patent Application Publication
(10) Pub. No.: US 2023/0422610 A1
(43) Pub. Date: Dec. 28, 2023

(54) SULFUROUS COMPOUNDS FOR ORGANIC ELECTROLUMINESCENT DEVICES

(71) Applicant: Merck Patent GmbH, Darmstadt (DE)

(72) Inventors: Amir Hossain PARHAM, Darmstadt (DE); Christian EHRENREICH, Darmstadt (DE)

(21) Appl. No.: 18/035,313

(22) PCT Filed: Nov. 9, 2021
(86) PCT No.: PCT/EP2021/081045

§ 371 Date: May 4, 2023

(30) Foreign Application Priority Data
Nov. 10, 2020 (EP) 20206660.1

(51) Int. Cl. H10K 85/60; C07D 495/16; C09K 11/06; C07D 519/00

(52) U.S. Cl. CPC H10K 85/657; C07D 495/16; C09K 11/06; H10K 85/6572; H10K 85/654; H10K 85/6574; C07D 519/00; H10K 85/633; H10K 85/636; H10K 50/12

(57) ABSTRACT

The present invention relates to sulfurous compounds which are suitable for use in electronic devices, and to electronic devices, in particular organic electroluminescent devices containing said compounds.

The present invention relates to sulfurous compounds for use in electronic devices, especially in organic electroluminescent devices, and to electronic devices, especially organic electroluminescent devices comprising these materials.

Emitting materials used in organic electroluminescent devices are frequently phosphorescent organometallic complexes. For quantum-mechanical reasons, up to four times the energy efficiency and power efficiency is possible using organometallic compounds as phosphorescent emitters. In electroluminescent devices, especially also in electroluminescent devices that exhibit triplet emission (phosphorescence), there is generally still a need for improvement. The properties of phosphorescent electroluminescent devices are not just determined by the triplet emitters used. More particularly, the other materials used, such as matrix materials, are also of particular significance here. Improvements in these materials can thus also lead to distinct improvements in the properties of the electroluminescent devices.

WO 2019/022435 discloses thionylcarbazole derivatives as matrix materials for phosphorescent emitters. Moreover, U.S. Ser. No. 10/312,455 B2 discloses compounds suitable as TADF (thermally activated delayed fluorescence) emitters. In addition, US 2019/100543 A1 discloses complexes comprising thionylcarbazole structural elements.

In general terms, in the case of these materials, for example for use as matrix materials, there is still a need for improvement, particularly in relation to the lifetime, but also in relation to the efficiency and operating voltage of the device.

It is therefore an object of the present invention to provide compounds which are suitable for use in an organic electronic device, especially in an organic electroluminescent device, and which lead to good device properties when used in this device, and to provide the corresponding electronic device.

More particularly, the problem addressed by the present invention is that of providing compounds which lead to a high lifetime, good efficiency and low operating voltage. Particularly the properties of the matrix materials too have a major influence on the lifetime and efficiency of the organic electroluminescent device.

A further problem addressed by the present invention can be considered that of providing compounds suitable for use in phosphorescent or fluorescent electroluminescent devices, especially as a matrix material. A particular problem addressed by the present invention is that of providing matrix materials that are suitable for red- and yellow-phosphorescing electroluminescent devices, especially for red-phosphorescing electroluminescent devices, and if appropriate also for blue-phosphorescing electroluminescent devices.

In addition, the compounds, especially when they are used as matrix materials, as hole transport materials or as electron blocker materials in organic electroluminescent devices, should lead to devices having excellent color purity.

A further problem can be considered that of providing electronic devices having excellent performance very inexpensively and in constant quality.

Furthermore, it should be possible to use or adapt the electronic devices for many purposes. More particularly, the performance of the electronic devices should be maintained over a broad temperature range.

It has been found that, surprisingly, particular compounds described in detail below solve this problem, are of good suitability for use in electroluminescent devices and lead to improvements in the organic electroluminescent device, especially in relation to lifetime, color purity, efficiency and operating voltage. The present invention therefore provides these compounds and electronic devices, especially organic electroluminescent devices, comprising such compounds.

The present invention provides a compound comprising at least one structure of the formula (1), preferably a compound of the formula (1):

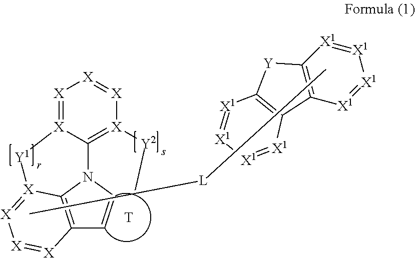

where the symbols and indices used are as follows:
T is a heteroaromatic 5-membered ring which has a sulfur atom and is fused to the azole ring via two adjacent and mutually bonded carbon atoms and which may be substituted by one or more R³ groups;
L is a connecting group which is preferably selected from a bond or an aromatic or heteroaromatic ring system which has 5 to 40 aromatic ring atoms and may be substituted by one or more R² radicals, more preferably a bond;
X is N, CR, or C if an L, Y¹ or Y² group binds thereto, with the proviso that not more than two of the X groups in one cycle are N, and X is preferably C or CR;
X¹ is N, CR¹, or C if the L group binds thereto, with the proviso that not more than two of the X groups in one cycle are N, and X¹ is preferably CR¹,
Y is NAr, NL, O, S, C(R²)₂, C(L)(R²), where NL means that the L group binds to the nitrogen atom of the NL group, and C(L)(R²) means that the L group binds to the carbon atom of the C(L)(R²) group;
Y¹ is a bond, NL, NR², NAr′, O, S, C(R²)₂, where NL means that the L group binds to the nitrogen atom of the NL group;
r is 0 or 1, where r=0 means that the Y¹ group is absent;
Y² is a bond, NL, NR², NAr, O, S, C(R²)₂, where NL means that the L group binds to the nitrogen atom of the NL group;
s is 0 or 1, where s=0 means that the Y² group is absent;
Ar is the same or different at each instance and is an aromatic or heteroaromatic ring system which has 5 to 40 aromatic ring atoms and may be substituted by one or more R² radicals;
R is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R radicals together, or one R radical together with an R², R³ radical, may also form an aliphatic or heteroaliphatic ring system; preferably, the R radicals do not form any such ring system;

R¹ is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R¹ radicals together or one R¹ radical together with one R² radical may also form an aromatic, heteroaromatic, aliphatic or heteroaliphatic ring system; preferably, the R¹ radicals do not form any such ring system;

R² is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R² radicals together or one R² radical together with one R, R¹, R³ radical may also form an aromatic, heteroaromatic, aliphatic or heteroaliphatic ring system; preferably, the R² radicals do not form any such ring system;

R³ is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, preferably 5 to 40 aromatic ring atoms, and may be substituted in each case by one or more R⁴ radicals; at the same time, two R³ radicals together or one R³ radical together with one R, R² radical may also form an aromatic, heteroaromatic, aliphatic or heteroaliphatic ring system; preferably, the R³ radicals do not form any such ring system;
Ar′ is the same or different at each instance and is an aromatic or heteroaromatic ring system which has 5 to 40 aromatic ring atoms and may be substituted by one or more R⁴ radicals;
R⁴ is the same or different at each instance and is H, D, F, Cl, Br, I, N(R⁵)₂, CN, NO₂, OR⁵, SR⁵, Si(R⁵)₃, B(OR⁵)₂, C(═O)R⁵, P(═O)(R⁵)₂, S(═O)R⁵, S(═O)₂R⁵, OSO₂R⁵, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁵ radicals and where one or more nonadjacent CH₂ groups may be replaced by Si(R⁵)₂, C═O, NR⁵, O, S or CONR⁵, or an aromatic or heteroaromatic ring system which has 5 to 40 aromatic ring atoms and may be substituted in each case by one or more R² radicals; at the same time, two or more R⁴ radicals together may form an aromatic, heteroaromatic, aliphatic or heteroaliphatic ring system; preferably, the R⁴ radicals do not form any such ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F; where the sum of r and s is 1 or 2, preferably 1.

An aryl group in the context of this invention contains 6 to 40 carbon atoms; a heteroaryl group in the context of this invention contains 2 to 40 carbon atoms and at least one heteroatom, with the proviso that the sum total of carbon atoms and heteroatoms is at least 5. The heteroatoms are preferably selected from N, O and/or S. An aryl group or heteroaryl group is understood here to mean either a simple aromatic cycle, i.e. benzene, or a simple heteroaromatic cycle, for example pyridine, pyrimidine, thiophene, etc., or a fused (annelated) aryl or heteroaryl group, for example naphthalene, anthracene, phenanthrene, quinoline, isoquinoline, etc. Aromatics joined to one another by a single bond, for example biphenyl, by contrast, are not referred to as an aryl or heteroaryl group but as an aromatic ring system.

An electron-deficient heteroaryl group in the context of the present invention is a heteroaryl group having at least one heteroaromatic six-membered ring having at least one nitrogen atom. Further aromatic or heteroaromatic five-membered or six-membered rings may be fused onto this six-membered ring. Examples of electron-deficient heteroaryl groups are pyridine, pyrimidine, pyrazine, pyridazine, triazine, quinoline, quinazoline or quinoxaline.

An aromatic ring system in the context of this invention contains 6 to 60 carbon atoms in the ring system. A heteroaromatic ring system in the context of this invention contains 2 to 60 carbon atoms and at least one heteroatom in the ring system, with the proviso that the sum total of carbon atoms and heteroatoms is at least 5. The heteroatoms are preferably selected from N, O and/or S. An aromatic or heteroaromatic ring system in the context of this invention shall be understood to mean a system which does not necessarily contain only aryl or heteroaryl groups, but in which it is also possible for two or more aryl or heteroaryl groups to be joined by a non-aromatic unit, for example a carbon, nitrogen or oxygen atom. For example, systems such as fluorene, 9,9′-spirobifluorene, 9,9-diarylfluorene, triarylamine, diaryl ethers, stilbene, etc. shall also be regarded as aromatic ring systems in the context of this invention, and likewise systems in which two or more aryl groups are joined, for example, by a short alkyl group. Preferably, the aromatic ring system is selected from fluorene, 9,9′-spirobifluorene, 9,9-diarylamine or groups in which two or more aryl and/or heteroaryl groups are joined to one another by single bonds.

In the context of the present invention, an aliphatic hydrocarbyl radical or an alkyl group or an alkenyl or alkynyl group which may contain 1 to 20 carbon atoms and in which individual hydrogen atoms or CH₂ groups may also be substituted by the abovementioned groups is preferably understood to mean the methyl, ethyl, n-propyl, i-propyl, n-butyl, i-butyl, s-butyl, t-butyl, 2-methylbutyl, n-pentyl, s-pentyl, neopentyl, cyclopentyl, n-hexyl, neohexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, heptynyl or octynyl radicals. An alkoxy group having 1 to carbon atoms is preferably understood to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy and 2,2,2-trifluoroethoxy. A thioalkyl group having 1 to 40 carbon atoms is understood to mean especially methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio. In general, alkyl, alkoxy or thioalkyl groups according to the present invention may be straight-chain, branched or cyclic, where one or more nonadjacent CH₂ groups may be replaced by the abovementioned groups; in addition, it is also possible for one or more hydrogen atoms to be replaced by D, F, Cl, Br, I, CN or NO₂, preferably F, CI or CN, further preferably F or CN, especially preferably CN.

An aromatic or heteroaromatic ring system which has 5-60 or 5-40 aromatic ring atoms and may also be substituted in each case by the abovementioned radicals and which may be joined to the aromatic or heteroaromatic system via any desired positions is understood to mean especially groups derived from benzene, naphthalene, anthracene, benzanthracene, phenanthrene, pyrene, chrysene, perylene, fluoranthene, naphthacene, pentacene, benzopyrene, biphenyl, biphenylene, terphenyl, triphenylene, fluorene, spirobifluorene, dihydrophenanthrene, dihydropyrene, tetrahydropyrene, cis- or trans-indenofluorene, cis- or trans-indenocarbazole, cis- or trans-indolocarbazole, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, hexaazatriphenylene, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole, or groups derived from combinations of these systems.

The wording that two or more radicals together may form a ring, in the context of the present description, should be understood to mean, inter alia, that the two radicals are joined to one another by a chemical bond with formal elimination of two hydrogen atoms. This is illustrated by the following scheme:

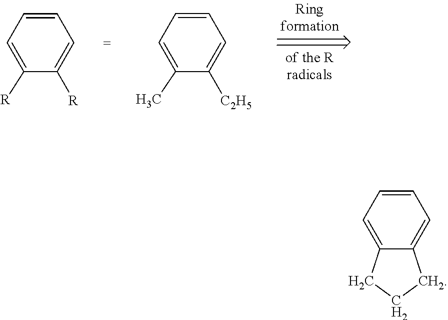

In addition, however, the abovementioned wording shall also be understood to mean that, if one of the two radicals is hydrogen, the second radical binds to the position to which the hydrogen atom was bonded, forming a ring. This will be illustrated by the following scheme:

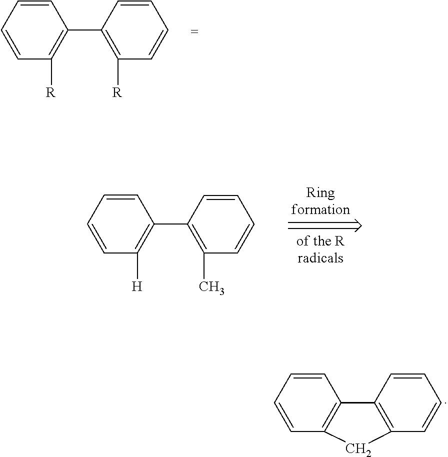

In a preferred configuration, the compounds of the invention may preferably comprise at least one structure of the formulae (1a), (1b), (1c), (1d), (1e), (1f), (1g), (1h), (1i), (1j), (1k), (1l), (1m), (1n), (1o), (1p), (1q), (1r), (1s), (1t), (1u), (1v), (1w), (1x), (1y), (1z) and (1za) and are more preferably selected from the compounds of the formulae (1a), (1b), (1c), (1d), (1e), (1f), (1g), (1h), (1i), (1j), (1k), (1l), (1m), (1n), (1o), (1p), (1q), (1r), (1s), (1t), (1u), (1v), (1w), (1x), (1y), (1z) and (1za):

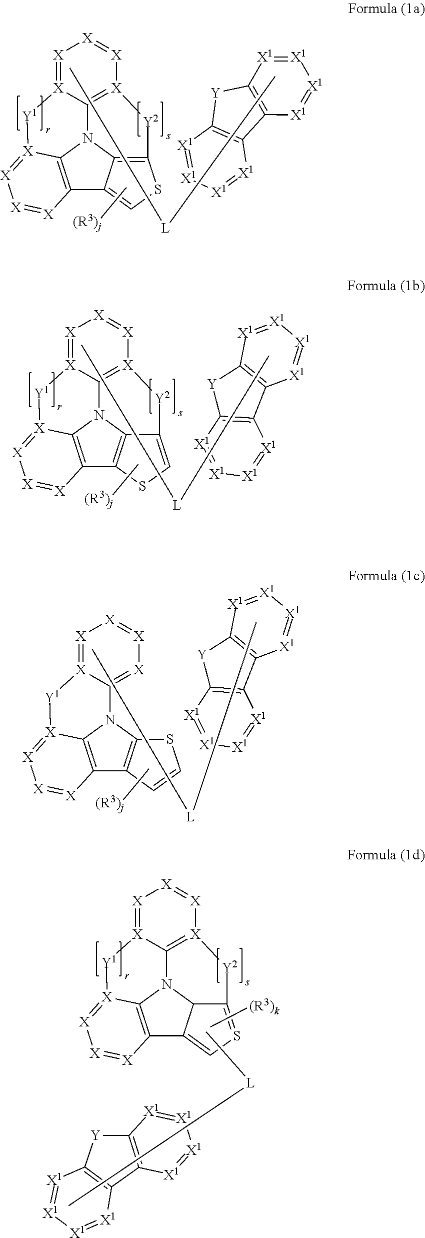

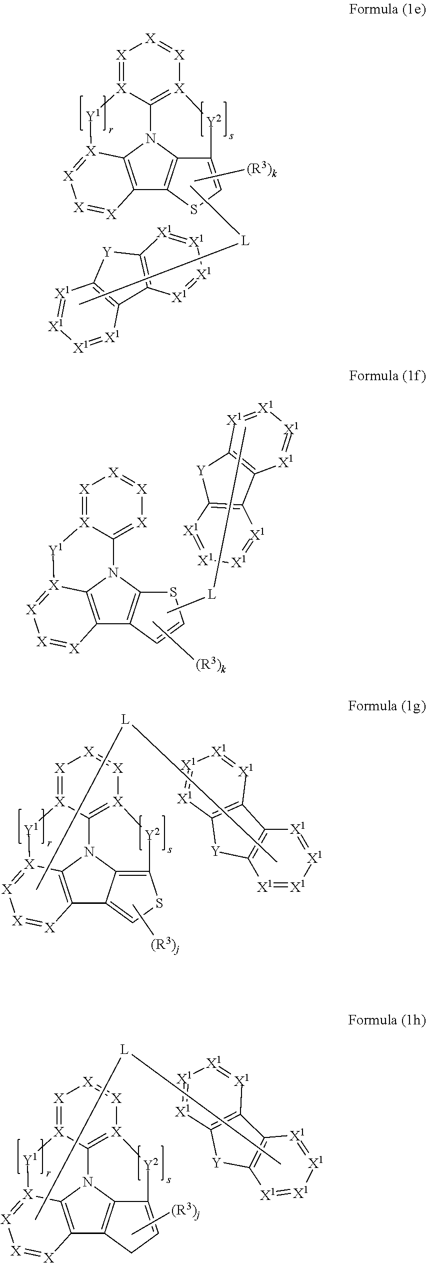

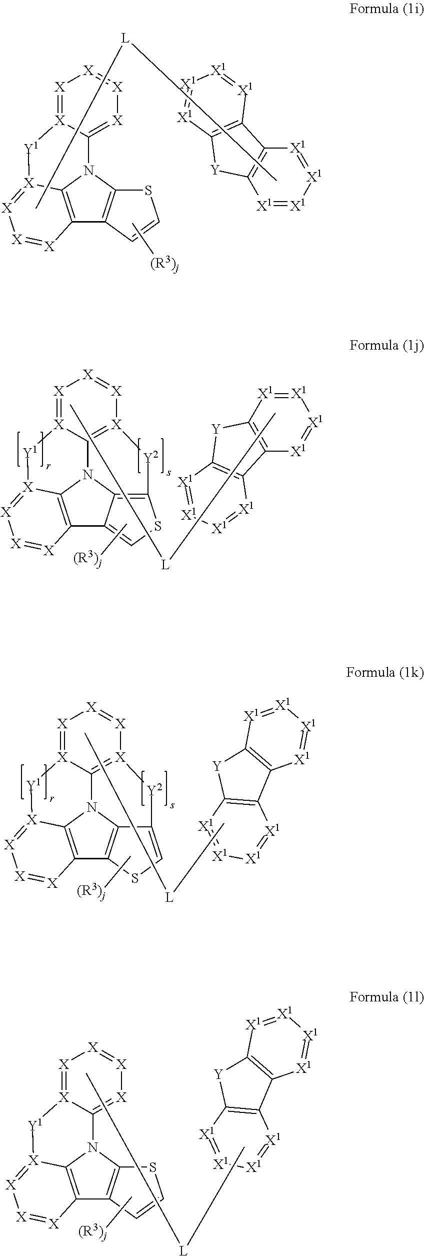

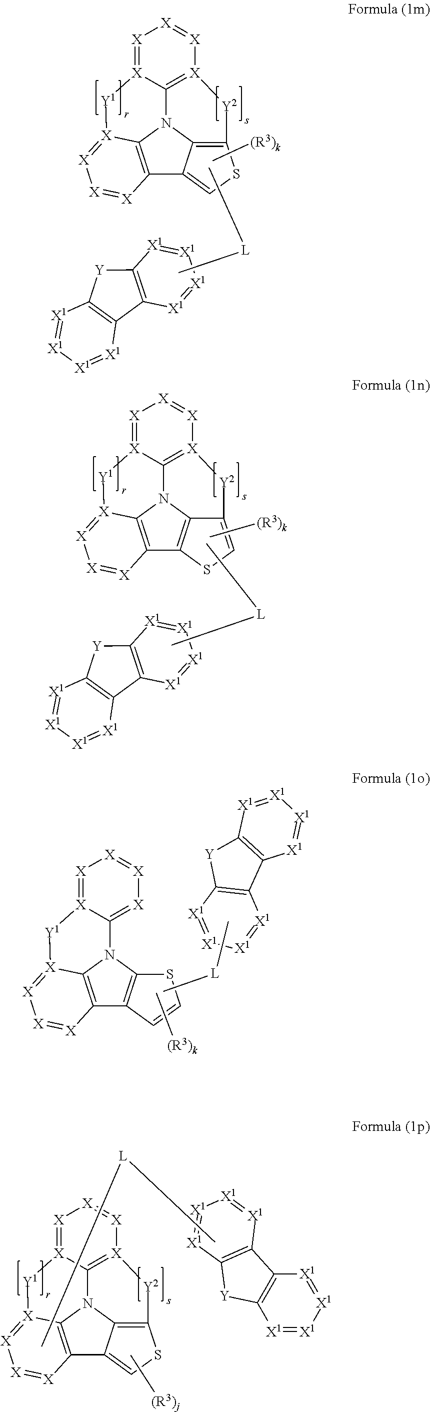

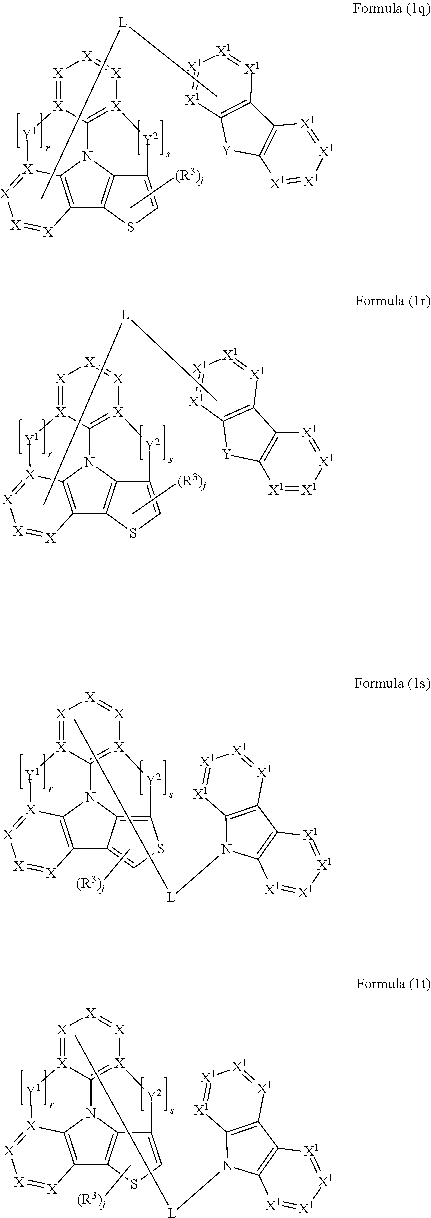

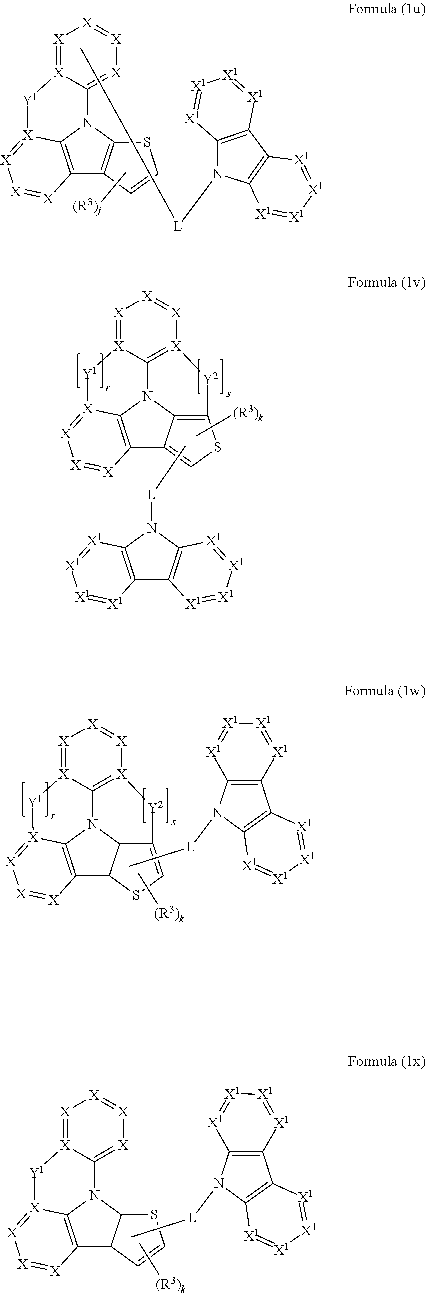

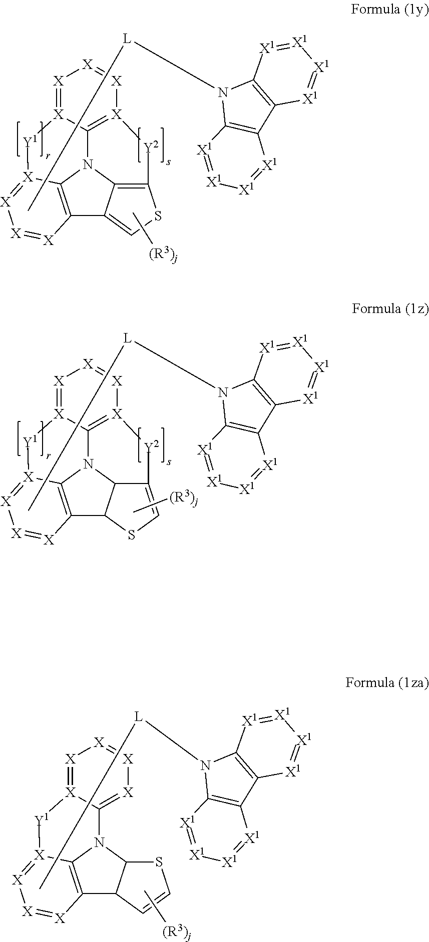

where the symbols Y, Y¹, Y², X, X¹, r, s, and R³ have the definitions given above, especially for formula (1), j is 0, 1 or 2, preferably 0 or 1, and k is 0 or 1, where s+k=0 or 1 and j+s=0, 1 or 2.

With regard to the above-detailed formulae (1a), (1 b), (1c), (1d), (1e), (1f), (1g), (1h), (1i), (1j), (1k), (1l), (1m), (1n), (1o), (1p), (1q), (1r), (1s), (1t), (1u), (1v), (1w), (1x), (1y), (1z) and (1za), it should be noted that these are similar in part, but differ at least in part in the points of attachment of the connecting group L. For instance, the connecting group L in the formulae (1a), (1b), (1c) may bind to any suitable point of attachment on the two structure groups connected by the connecting group L. In the formulae (1d), (1e), (1f), the connecting group L binds to the thiofuran ring, and in the formulae (1g), (1h), (1i) does not bind to the thiofuran ring. In the formulae (1j), (1k), (1l), the connecting group L binds to an aromatic or heteroaromatic structural element of the fluorene, dibenzofuran, dibenzothiofuran or carbazole group, where the connecting group L may bind to any suitable point of attachment in the bridged thionylcarbazole radical. The further structures result correspondingly from these differences.

It may preferably be the case that, in compounds of the formulae ((1), (1a), (1b), (1c), (1d), (1e), (1f), (1g), (1h), (1i), (1j), (1k), (1l), (1m), (1n), (1o), (1p), (1q), (1r), (1s), (1t), (1u), (1v), (1w), (1x), (1y), (1z) and (1za), not more than four and preferably not more than two X groups are N; more preferably, all X groups are CR or C, where preferably not more than 4, more preferably not more than 3 and especially preferably not more than 2 of the CR groups that X represents are not the CH group.

It may further be the case that, in compounds of the formulae (1), (1a), (1b), (1c), (1d), (1e), (1f), (1g), (1h), (1i), (1j), (1k), (1l), (1m), (1n), (1o), (1p), (1q), (1r), (1s), (1t), (1u), (1v), (1w), (1x), (1y), (1z) and (1za), not more than four X¹ groups are N and preferably not more than one X¹ group is N; more preferably, all X¹ groups are CR¹ or C, where preferably not more than 3 and more preferably not more than 2 of the CR groups that X¹ represents are not the CH group.

In a further embodiment, it may be the case that, in compounds of the formulae (1), (1a), (1b), (1c), (1d), (1e), (1f), (1g), (1h), (1i), (1j), (1k), (1l), (1m), (1n), (1o), (1p), (1q), (1r), (1s), (1 t), (1u), (1v), (1w), (1x), (1y), (1z) and (1za), the index r=1 and the index s=0, such that the Y¹ group is present and the Y² group is absent.

In a further-preferred embodiment, it may be the case that the compounds of the invention comprise a structure of the formulae (2a), (2b), (2c), (2d), (2e), (2f), (2g), (2h), (2i), (2j), (2k), (21), (2m), (2n), (20), (2p), (2q), (2r), (2s), (2t), (2u), (2v), (2w), (2x), (2y), (2z) and (2za), where the compounds of the invention may more preferably be selected from the compounds of the formulae(2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z) and (2za):

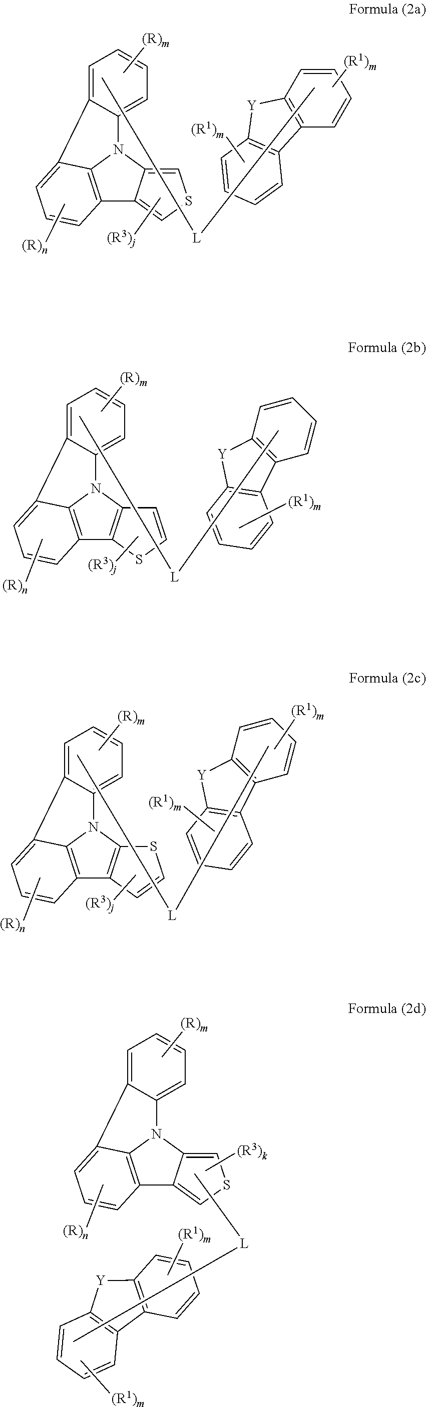

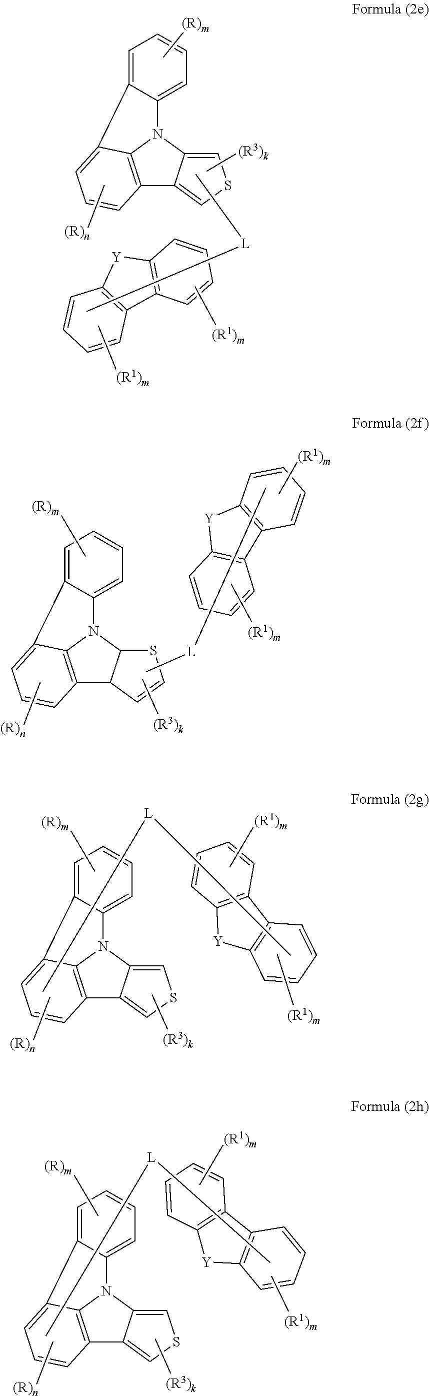

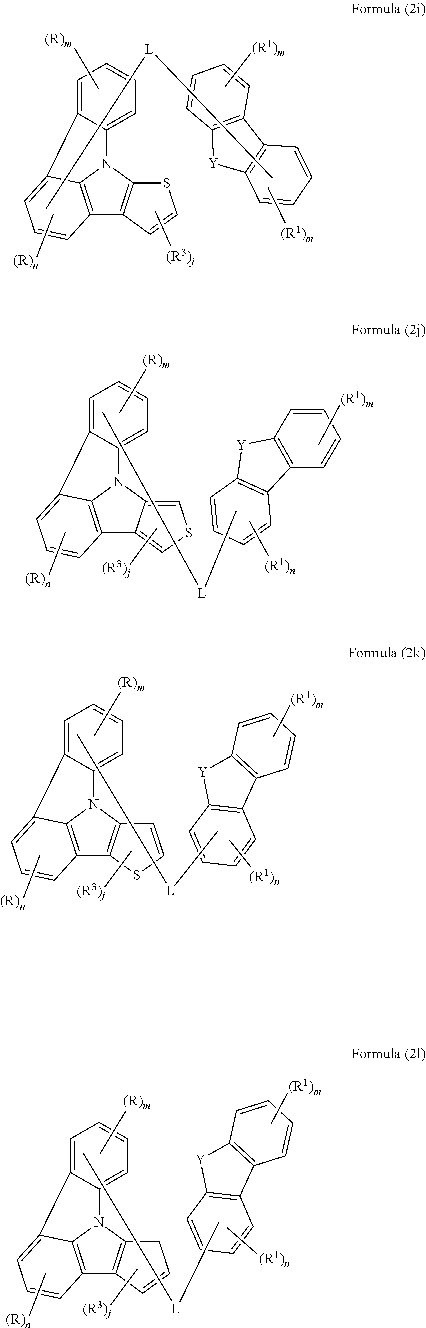

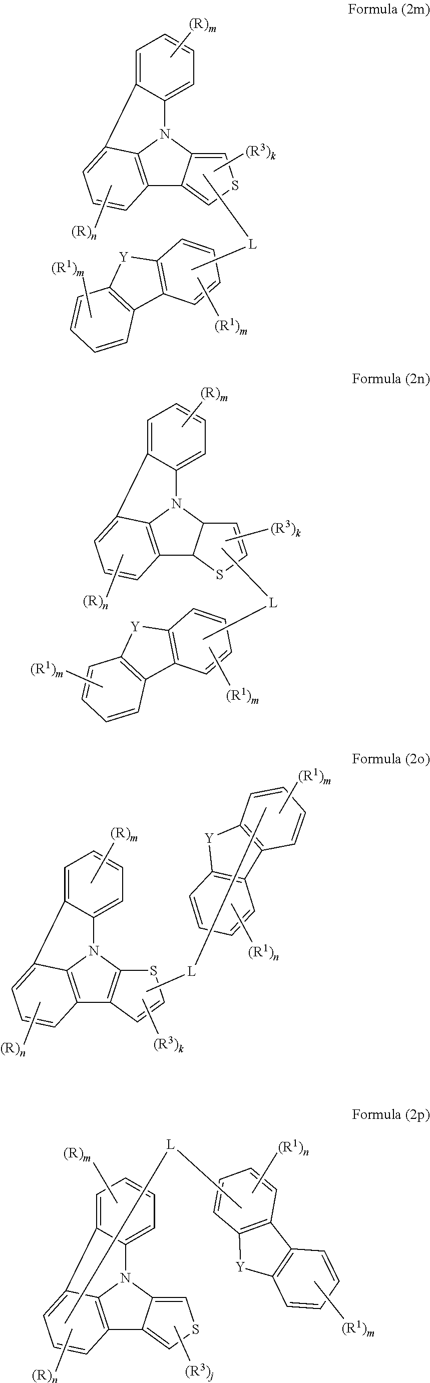

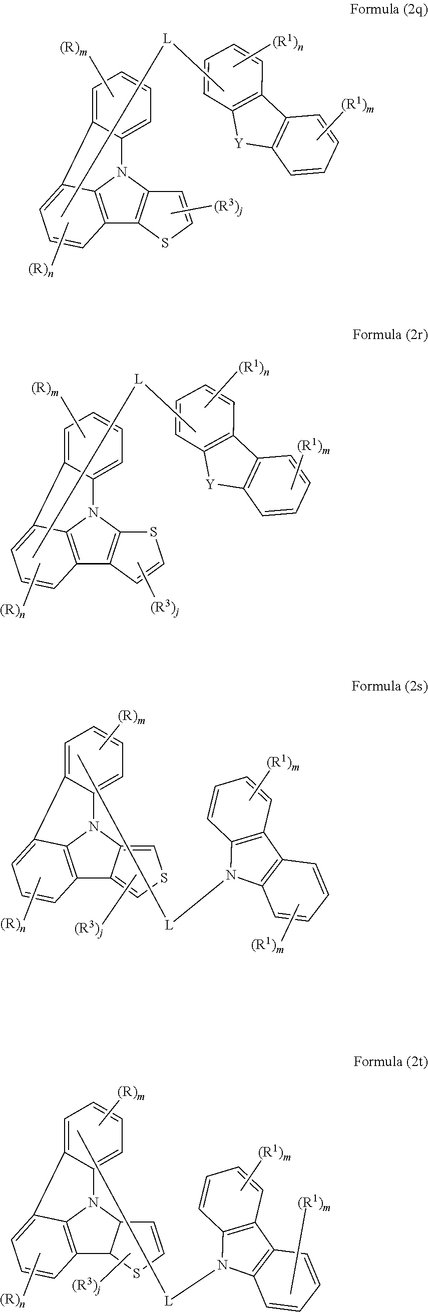

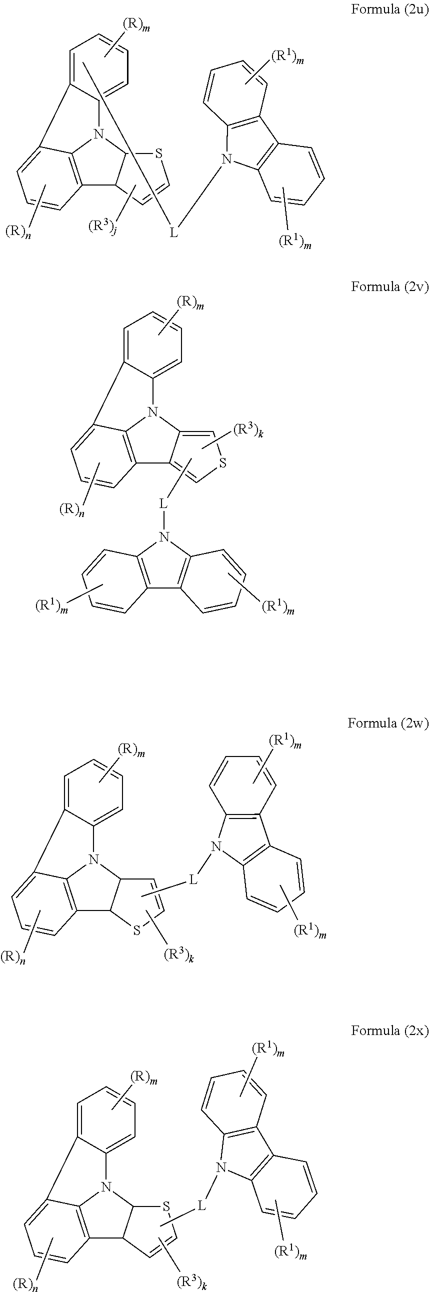

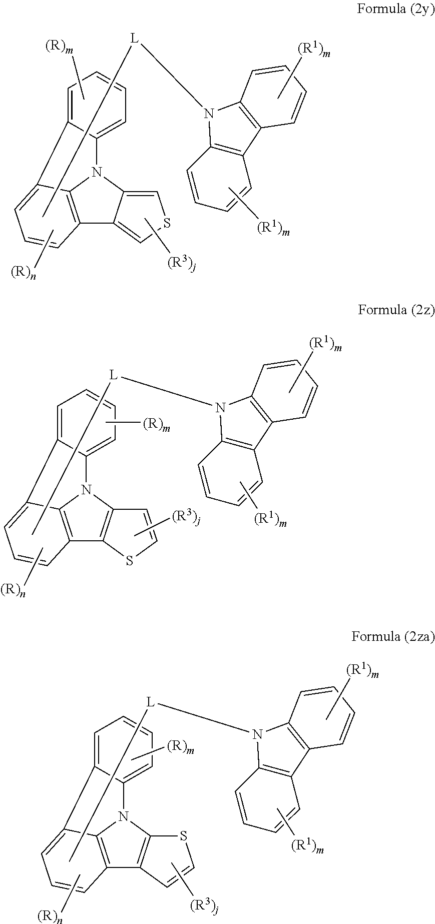

where L, Y, R, R¹ and R³ have the definitions given above, especially for formula (1), the index k is 0 or 1, the index j is 0, 1 or 2, preferably 0 or 1, the index n is 0, 1, 2 or 3, preferably 0, 1 or 2 and most preferably 0 or 1, and the index m is 0, 1, 2, 3 or 4, preferably 0, 1 or 2 and very preferably 0 or 1.

With regard to the above-detailed formulae (2a), (2b), (2c), (2d), (2e), (2f), (2g), (2h), (2i), (2j), (2k), (21), (2m), (2n), (20), (2p), (2q), (2r), (2s), (2t), (2u), (2v), (2w), (2x), (2y), (2z) and (2za), it should be noted that these are similar in part, but differ at least in the points of attachment of the connecting group L. For instance, the connecting group L in the formulae (2a), (2b), (2c) may bind to any suitable point of attachment on the two structure groups connected by the connecting group L. In the formulae (2d), (2e), (2f), the connecting group L binds to the thiofuran ring, and in the formulae (2g), (2h), (2i) does not bind to the thiofuran ring. In the formulae (2j), (2k), (2l), the connecting group L binds to an aromatic structural element of the fluorene, dibenzofuran, dibenzothiofuran or carbazole group, where the connecting group L may bind to any suitable point of attachment in the bridged thionylcarbazole radical. The further structures result correspondingly from these differences.

In a preferred embodiment, it may be the case that, in compounds of the formulae (1), (1a), (1b), (1c), (1d), (1e), (1f), (1g), (1h), (1i), (1j), (1k), (1l), (1m), (1n), (1o), (1p), (1q), (1r), (1s), (1 t), (1u), (1v), (1w), (1x), (1y), (1z) and (1za), the index s=1 and the index r=0, such that the Y² group is present and the Y¹ group is absent.

In a further-preferred embodiment, it may be the case that the compounds of the invention comprise a structure of the formulae (3a), (3b), (3c), (3d), (3e), (3f), (3g), (3h), (3i), (3j), (3k), (3l), (3m), (3n), (3o), (3p), (3q) and (3r), where the compounds of the invention may more preferably be selected from the compounds of the formulae(3a), (3b), (3c), (3d), (3e), (3f), (3g), (3h), (3i), (3j), (3k), (3l), (3m), (3n), (3o), (3p), (3q) and (3r):

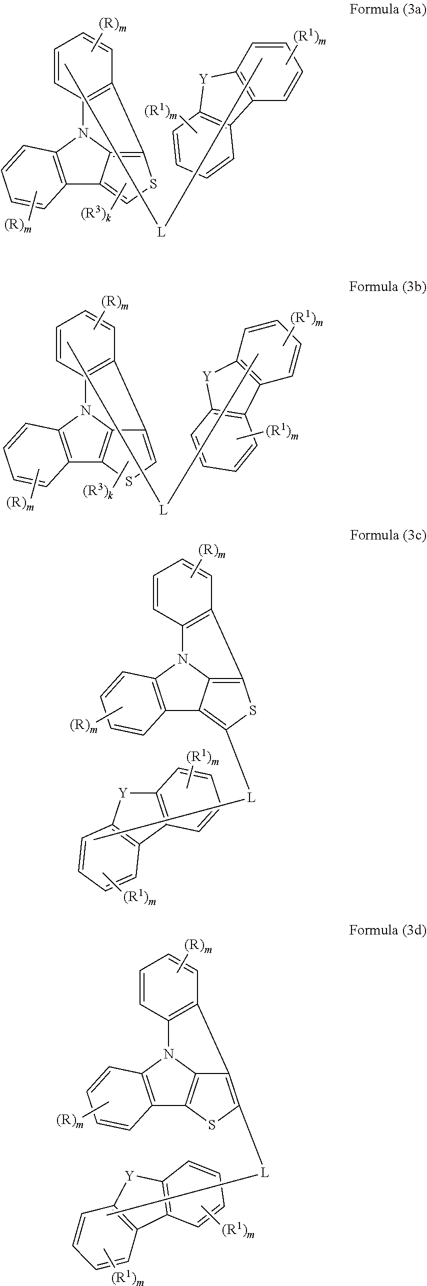

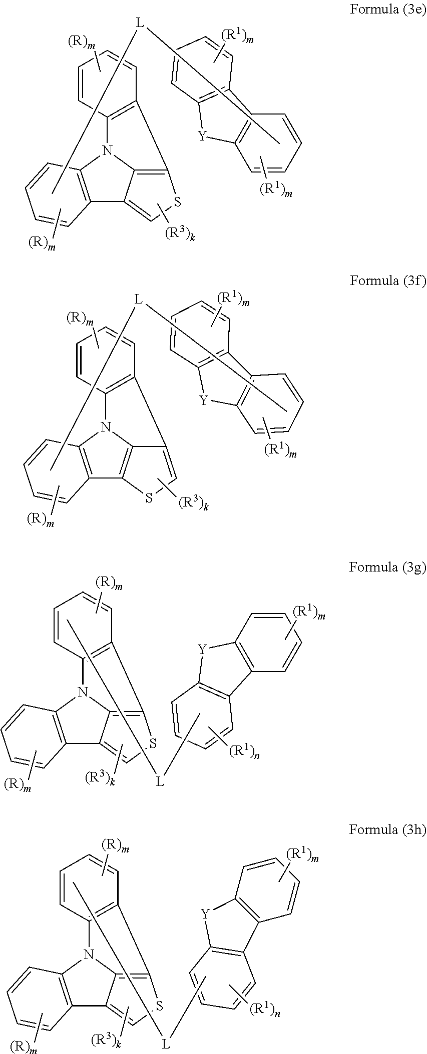

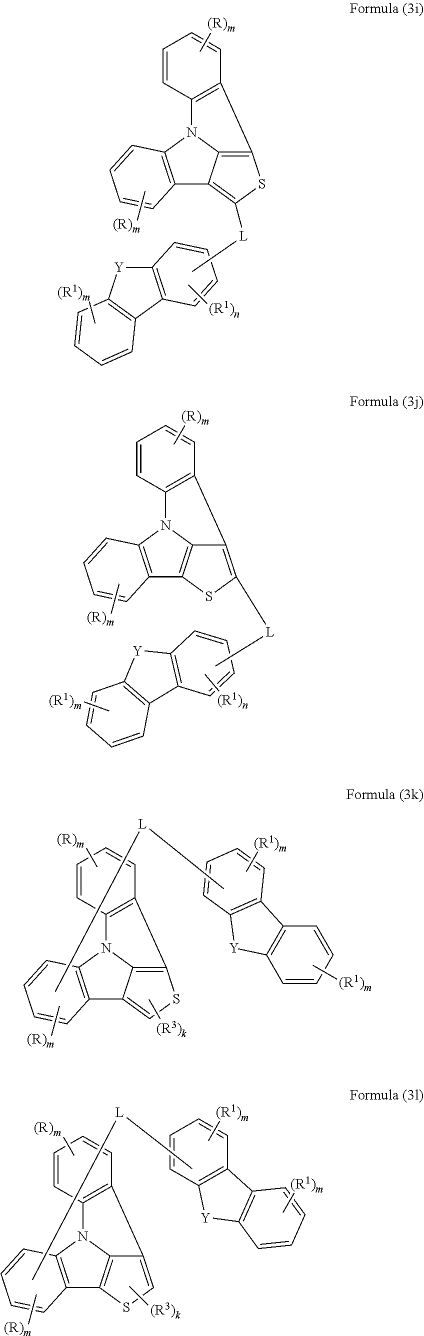

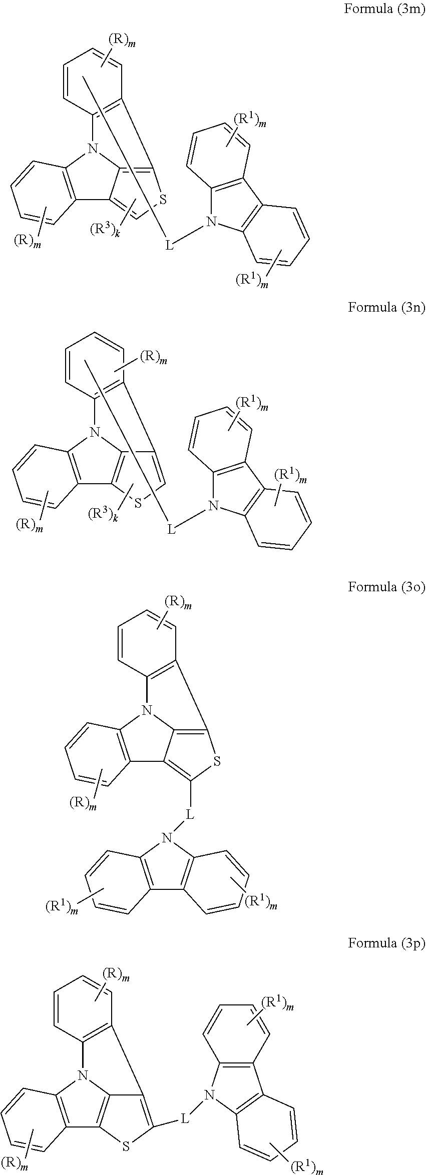

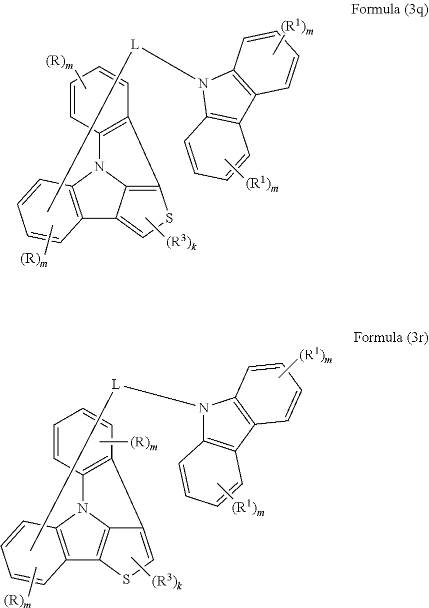

where L, Y, R, R¹ and R³ have the definitions given above, especially for formula (1), the index k is 0 or 1, the index n is 0, 1, 2 or 3, preferably 0, 1 or 2 and very preferably 0 or 1, and the index m is 0, 1, 2, 3 or 4, preferably 0, 1 or 2 and very preferably 0 or 1.

With regard to the above-detailed formulae (3a), (3b), (3c), (3d), (3e), (3f), (3g), (3h), (3i), (3j), (3k), (3l), (3m), (3n), (3o), (3p), (3q) and (3r), it should be noted that these are similar in part, but differ at least in the points of attachment of the connecting group L. For instance, the connecting group L in the formulae (3a), (3b) may bind to any suitable point of attachment on the two structure groups connected by the connecting group L. In the formulae (3c), (3d), the connecting group L binds to the thiofuran ring, and in the formulae (3e), (3f) does not bind to the thiofuran ring. In the formulae (3g), (3h), the connecting group L binds to an aromatic structural element of the fluorene, dibenzofuran, dibenzothiofuran or carbazole group, where the connecting group L may bind to any suitable point of attachment in the bridged thionylcarbazole radical. The further structures result correspondingly from these differences.

In a further-preferred embodiment, it may be the case that the compounds of the invention comprise a structure of the formulae (4a), (4b), (4c), (4d), (4e), (4f), (4g), (4h), (4i), (4j), (4k), (4l), (4m), (4n), (4o), (4p), (4q) and (4r), where the compounds of the invention may more preferably be selected from the compounds of the formulae (4a), (4b), (4c), (4d), (4e), (4f), (4g), (4h), (4i), (4j), (4k), (4l), (4m), (4n), (4o), (4p), (4q) and (4r):

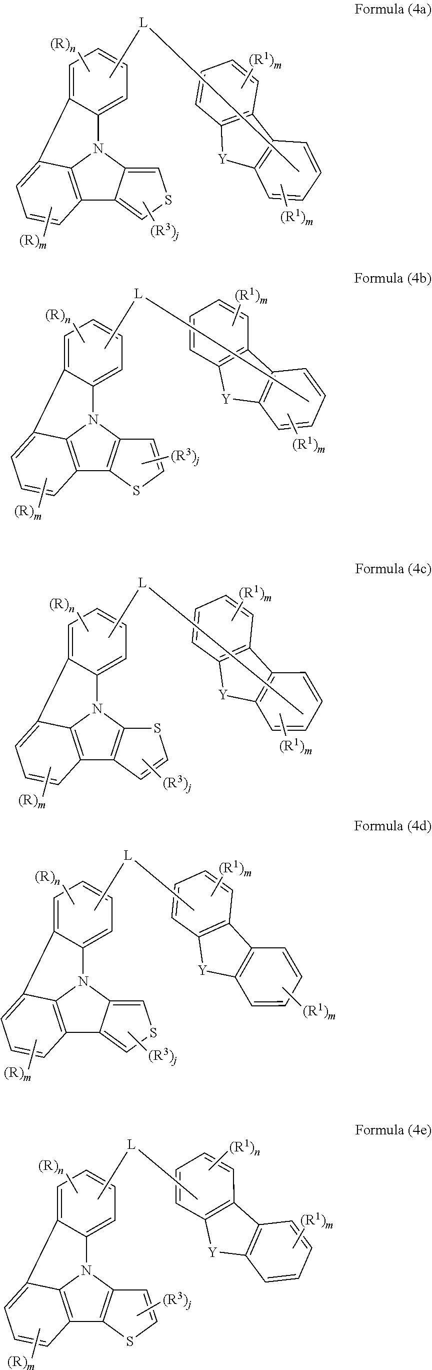

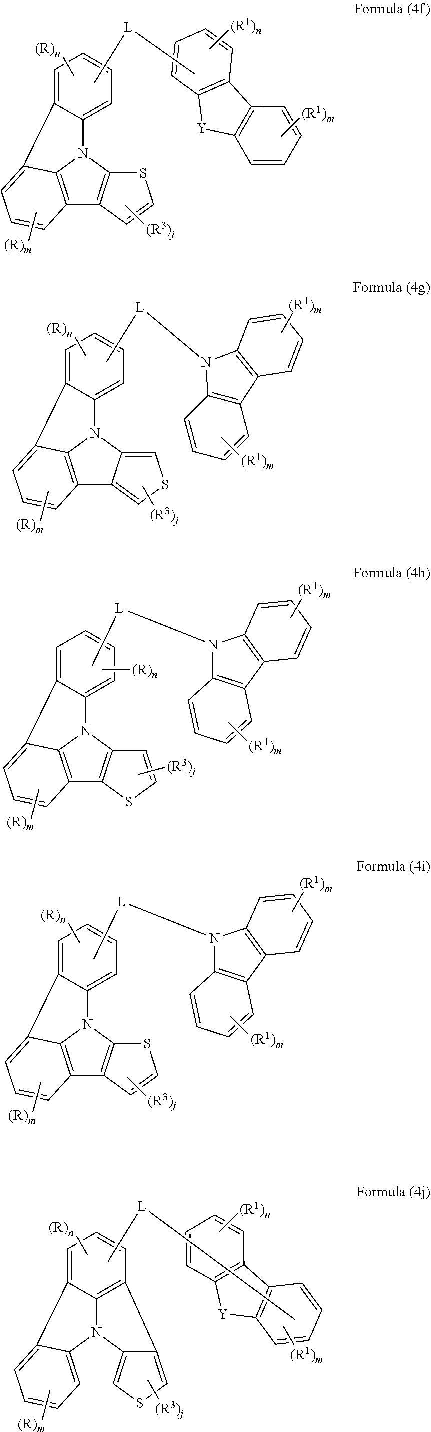

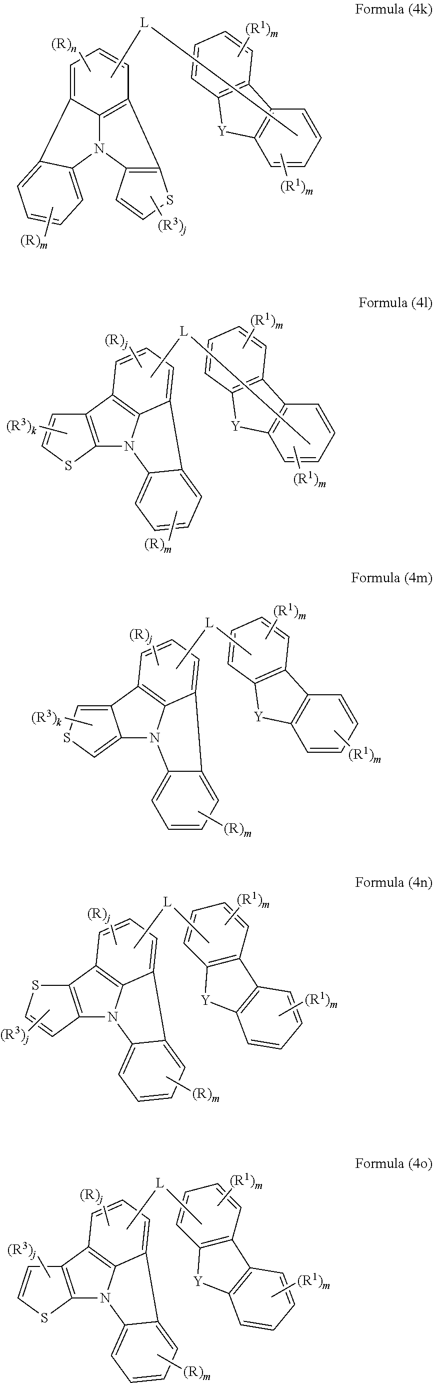

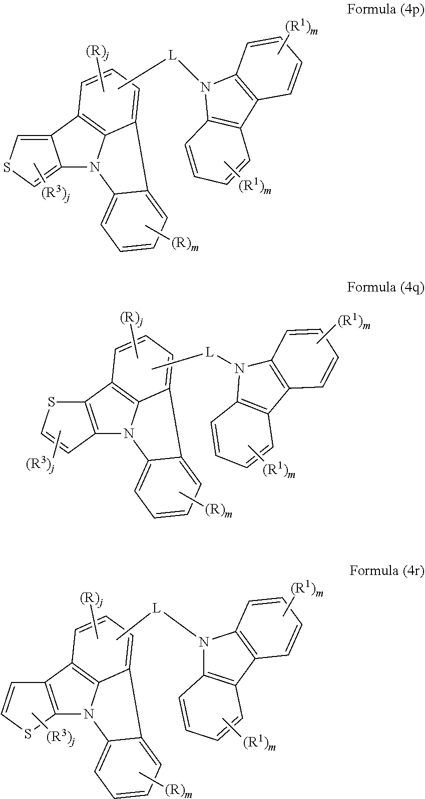

where L, Y, R, R¹ and R³ have the definitions given above, especially for formula (1), the index k is 0 or 1, the index j is 0, 1 or 2, preferably 0 or 1, the index n is 0, 1, 2 or 3, preferably 0, 1 or 2 and most preferably 0 or 1, and the index m is 0, 1, 2, 3 or 4, preferably 0, 1 or 2 and very preferably 0 or 1.

In a preferred embodiment of the present invention, it may be the case that the compound comprises at least one structure of the formulae (5a), (5b), (5c), (5d), (5e), (5f), (5g), (5h) and (5i), particular preference being given to a compound selected from the compounds of the formulae (5a), (5b), (5c), (5d), (5e), (5f), (5g), (5h) and (5i):

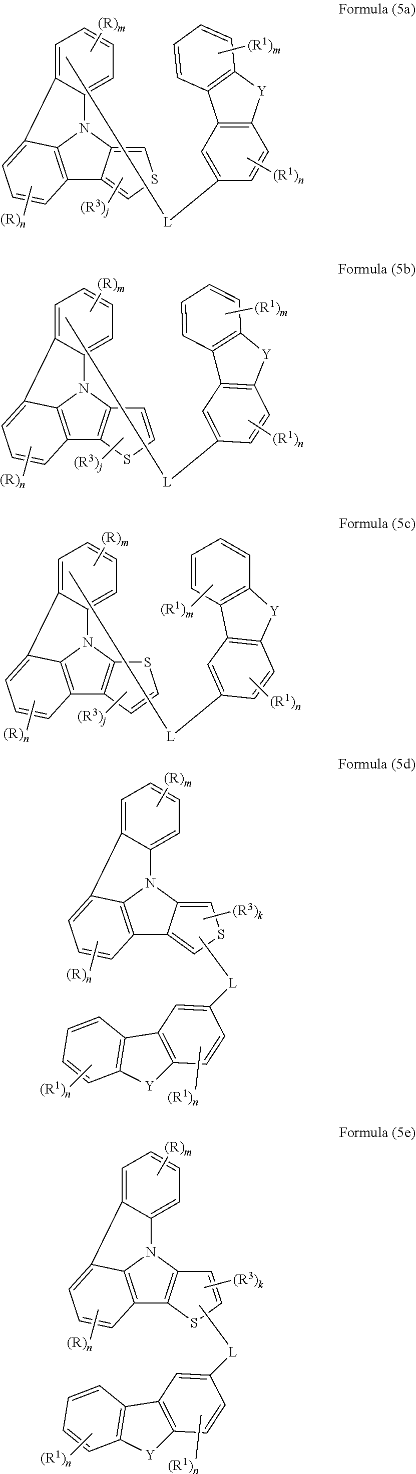

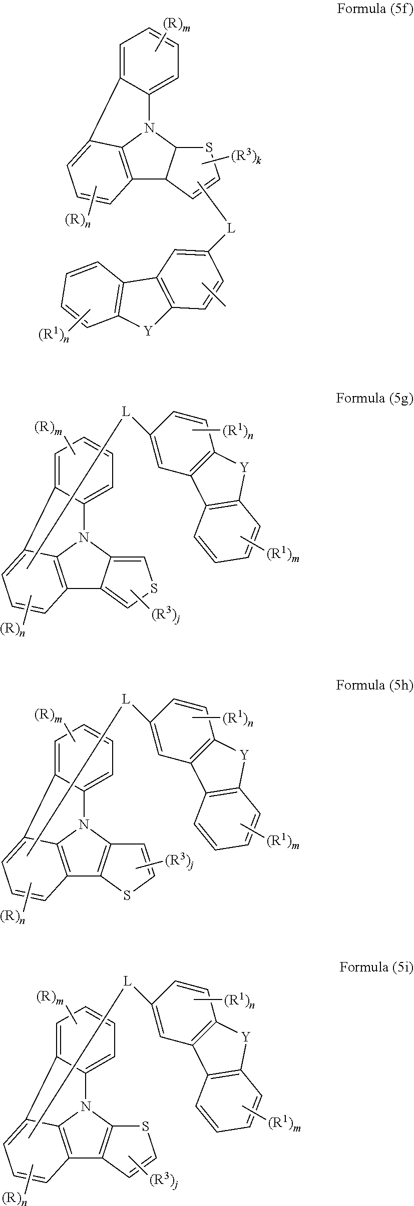

where L, Y, R, R¹ and R³ have the definitions given above, especially for formula (1), the index k is 0 or 1, the index j is 0, 1 or 2, preferably 0 or 1, the index n is 0, 1, 2 or 3, preferably 0, 1 or 2 and most preferably 0 or 1, and the index m is 0, 1, 2, 3 or 4, preferably 0, 1 or 2 and very preferably 0 or 1.

With regard to the above-detailed formulae (5a), (5b), (5c), (5d), (5e), (5f), (5g), (5h) and (5i), it should be noted that these are similar in part, but differ at least in part in the points of attachment of the connecting group L. For instance, the connecting group L in the formulae (5a), (5b), (5c) may bind to any suitable point of attachment on the thionylcarbazole radical. In the formulae (5d), (5e), (5f), the connecting group L binds to the thiofuran ring, and in the formulae (5g), (5h) and (5i) does not bind to the thiofuran ring.

In a preferred embodiment of the invention, the compounds preferably comprise at least one structure of the formulae (6a), (6b), (6c), (6d), (6e), (6f), (6g) and (6h), and the compounds are more preferably selected from the compounds of the formulae (6a), (6b), (6c), (6d), (6e), (6f), (6g) and (6h):

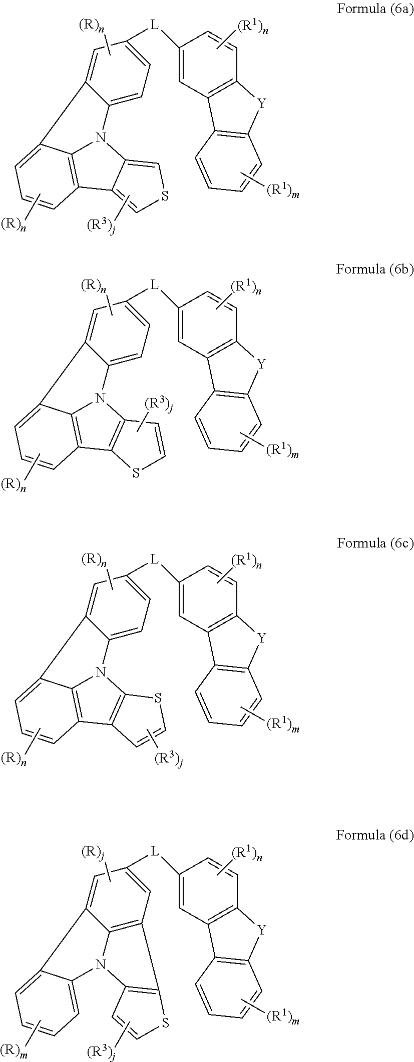

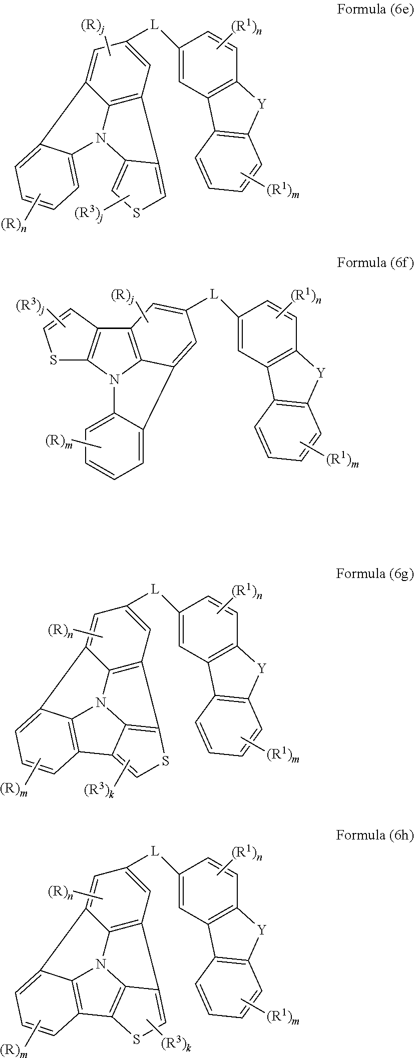

where L, Y, R, R¹ and R³ have the definitions given above, especially for formula (1), the index k is 0 or 1, the index j is 0, 1 or 2, preferably 0 or 1, the index n is 0, 1, 2 or 3, preferably 0, 1 or 2 and most preferably 0 or 1, and the index m is 0, 1, 2, 3 or 4, preferably 0, 1 or 2 and very preferably 0 or 1.

The sum total of the indices k, j, m and n in structures/compounds of the formulae (2a) to (2za), (3a) to (3r), (4a) to (4r), (5a) to (5i) and (6a) to (6h) is preferably not more than 6, especially preferably not more than 4 and more preferably not more than 2.

The L group is a connecting group which is preferably selected from a bond, an aromatic or heteroaromatic ring system which has 5 to 40 aromatic ring atoms and may be substituted by one or more R² radicals, and an N-containing group, preferably a mono-, di- or triarylamine group.

In a preferred embodiment, the connecting group L is more preferably selected from a bond or an aromatic or heteroaromatic ring system which has 5 to 40 aromatic ring atoms and may be substituted by one or more R² radicals; more preferably, L is a bond.

In a further embodiment, the L group is a radical that joins two structural elements via a nitrogen-containing group, especially via a mono-, di- or triarylamine group. For example, the L group may be a group of the formulae —N(Ar^{a})—,
—N(Ar^{a})—Ar^{b}— or —Ar^{c}—N(Ar^{a})—Ar^{b}—, where Ar^{a}, Ar^{b} and Ar^{c} are the same or different at each instance and are an aromatic or heteroaromatic ring system which has 5 to 24 aromatic ring atoms and may be substituted in each case by one or more R² radicals. The total number of aromatic ring atoms in Ar^{a}, Ar^{b} and Ar^{c} here is not more than 60 and preferably not more than 40.

In this case, Ar^{c} and Ar^{a} may also be bonded to one another and/or Ar^{a} and Ar^{b} to one another via a group selected from C(R²)₂, NR², O and S. Preferably, Ar^{c} and Ar^{a} are joined to one another or Ar^{a} and Ar^{b} to one another in the respective ortho position to the bond to the nitrogen atom. In a further embodiment of the invention, none of the Ar^{a}, Ar^{b} or Ar^{c} groups are bonded to one another.

Preferably, Ar^{c} is an aromatic or heteroaromatic ring system which has 6 to 24 aromatic ring atoms, preferably 6 to 12 aromatic ring atoms, and may be substituted in each case by one or more R² radicals. More preferably, Ar^{c} is selected from the group consisting of ortho-, meta- or para-phenylene or ortho-, meta- or para-biphenyl, each of which may be substituted by one or more R⁴ radicals, but are preferably unsubstituted. Most preferably, Ar^{c} is an unsubstituted phenylene group.

Preferably, Ar^{a} and Ar^{b} are the same or different at each instance and are an aromatic or heteroaromatic ring system which has 6 to 24 aromatic ring atoms and may be substituted in each case by one or more R² radicals. Particularly preferred Ar^{a} and Ar^{b} groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R² radicals. Most preferably, Ar^{a} and Ar^{b} are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

Preferably, the L group may form through-conjugation with the groups to which the L group of formula (1) or the preferred embodiments of this formula is bonded. Through-conjugation of the aromatic or heteroaromatic systems is formed as soon as direct bonds are formed between adjacent aromatic or heteroaromatic rings. A further bond between the aforementioned conjugated groups, for example via a sulfur, nitrogen or oxygen atom or a carbonyl group, is not detrimental to conjugation. In the case of a fluorene system, the two aromatic rings are bonded directly, where the sp³-hybridized carbon atom in position 9 does prevent fusion of these rings, but conjugation is possible, since this sp³-hybridized carbon atom in position 9 does not necessarily lie between the groups connected via the connecting group L. In contrast, in the case of a second spirobifluorene structure, through-conjugation can be formed if the bond between the groups connected via the connecting group L is via the same phenyl group in the spirobifluorene structure or via phenyl groups in the spirobifluorene structure that are bonded directly to one another and are in one plane. If the bond between the groups connected via the connecting group L is via different phenyl groups in the second spirobifluorene structure bonded via the spa-hybridized carbon atom in position 9, the conjugation is interrupted.

In a further preferred embodiment of the invention, L is a bond or an aromatic or heteroaromatic ring system which has 5 to 14 aromatic or heteroaromatic ring atoms, preferably an aromatic ring system which has 6 to 12 carbon atoms, and which may be substituted by one or more R² radicals, but is preferably unsubstituted, where R² may have the definition given above, especially for formula (1). More preferably, L is a bond or an aromatic ring system having 6 to 10 aromatic ring atoms or a heteroaromatic ring system having 6 to 13 heteroaromatic ring atoms, each of which may be substituted by one or more R² radicals, but is preferably unsubstituted, where R² may have the definition given above, especially for formula (1).

Further preferably, the symbol L shown in formula (1) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L group shown in formula (1) comprises an aromatic ring system having not more than two fused aromatic and/or heteroaromatic 6-membered rings, preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R² radicals, but are preferably unsubstituted.

It may further be the case that the L group shown in formula (1) inter alia has not more than 1 nitrogen atom, preferably not more than 2 heteroatoms, especially preferably not more than one heteroatom and more preferably no heteroatom.

It may additionally be the case that the L group does not form a fused aromatic or heteroaromatic ring system with the groups to which the L group binds, where this includes the R, R¹, R² or R³ radicals by which the L group or any of the groups to which the L group binds may be substituted.

Preference is given to compounds comprising structures of the formula (1), preferably representable by structures of the formula (1) in which the L group is a bond or a group selected from the formulae (L¹-1) to (L¹-16):

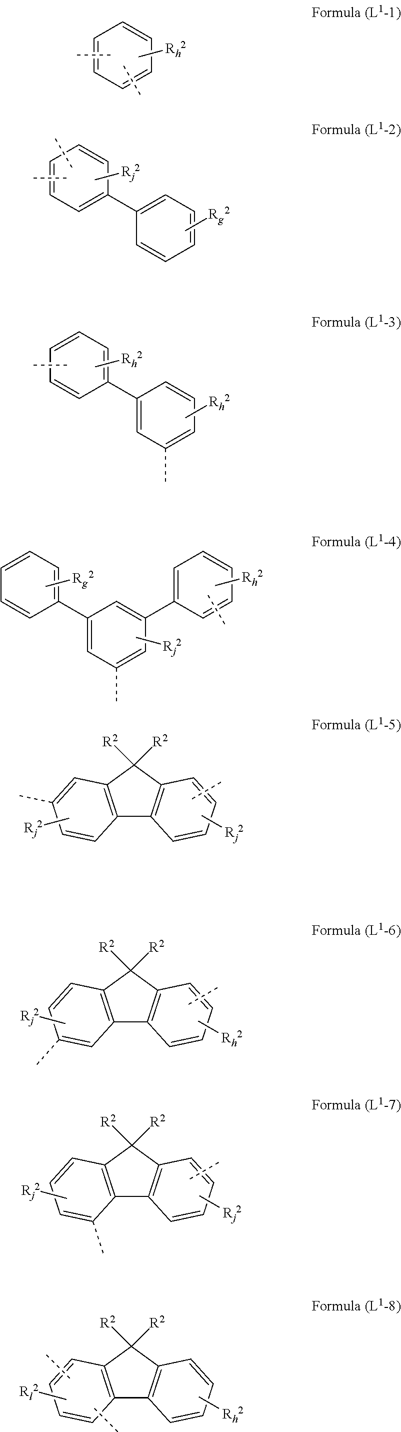

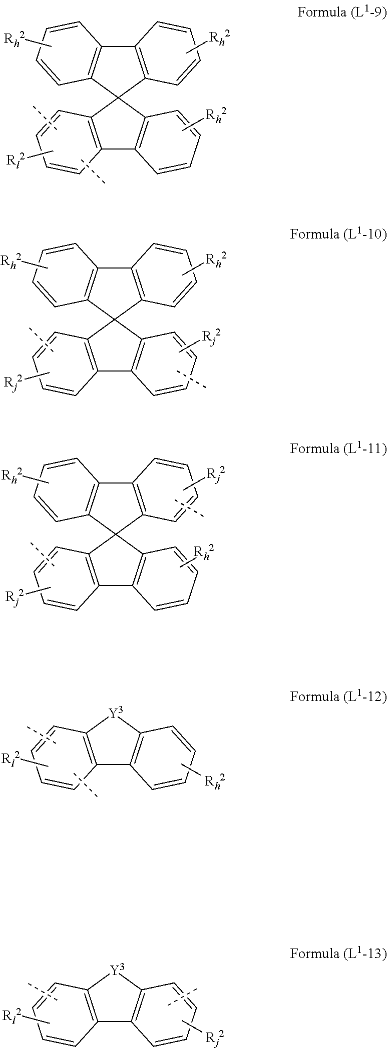

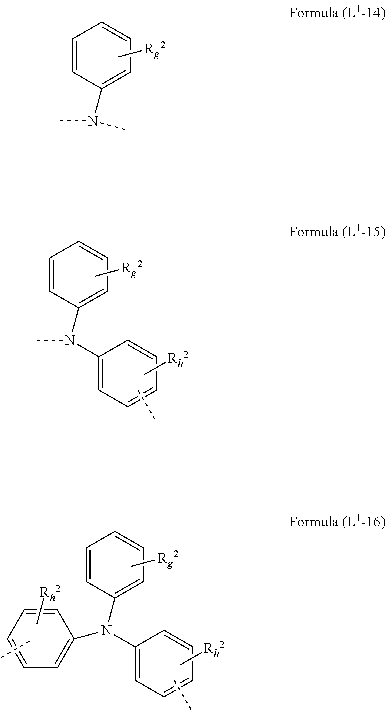

where the dotted bonds each mark the positions of attachment, Y³ is the same or different at each instance and is preferably O, S, NAr′, NR², preferably 0 or S, the index k is 0 or 1, the index I is 0, 1 or 2, the index j is independently at each instance 0, 1, 2 or 3; the index h is independently at each instance 0, 1, 2, 3 or 4, the index g is 0, 1, 2, 3, 4 or 5; and the symbol R² has the definition given above, especially for formula (1), where L is preferably a bond or is an aromatic ring system which has 5 to 40 aromatic ring atoms and does not comprise any heteroatoms.

It may further be the case that the L group in formula (1) is a bond and Y is selected from NR², NAr, O, S, and Y is preferably NAr.

For preferred embodiments of the structures/compounds of formula (1) detailed above and hereinafter, the remarks made in relation to the L radical are correspondingly applicable.

Preferred aromatic or heteroaromatic ring systems Ar are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3, 4 or 9 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R² radicals.

It may further be the case that the substituents R, R¹, R² and R³ according to the above formulae do not form a fused aromatic or heteroaromatic ring system, preferably any fused ring system, with the ring atoms of the ring system. This includes the formation of a fused ring system with possible substituents R⁴, R⁵ which may be bonded to the R, R¹, R², R³ radicals.

When two radicals that may especially be selected from R¹, R², R³, R⁴, R⁵, R⁶, R⁷, R⁸, R⁹, R¹⁹ and/or R¹¹ form a ring system with one another, this ring system may be mono- or polycyclic, aliphatic, heteroaliphatic, aromatic or heteroaromatic. In this case, the radicals which together form a ring system may be adjacent, meaning that these radicals are bonded to the same carbon atom or to carbon atoms directly bonded to one another, or they may be further removed from one another. In addition, the ring systems provided with the substituents R¹, R², R³, R⁴, R⁵, R⁶, R⁷, R⁸, R⁹, R¹⁰ and/or R¹¹ may also be joined to one another via a bond, such that this can bring about a ring closure. In this case, each of the corresponding bonding sites has preferably been provided with a substituent R¹, R², R³, R⁴, R⁵, R⁶, R⁷, R⁸, R⁹, R¹⁹ and/or R¹¹.

It may further be the case that R, R¹, R² and/or R³ are the same or different at each instance and are selected from the group consisting of H, D or an aromatic or heteroaromatic ring system selected from the groups of the following formulae Ar-1 to Ar-75, and/or the Ar and/or Ar′ group is the same or different at each instance and is selected from the groups of the following formulae Ar-1 to Ar-75:

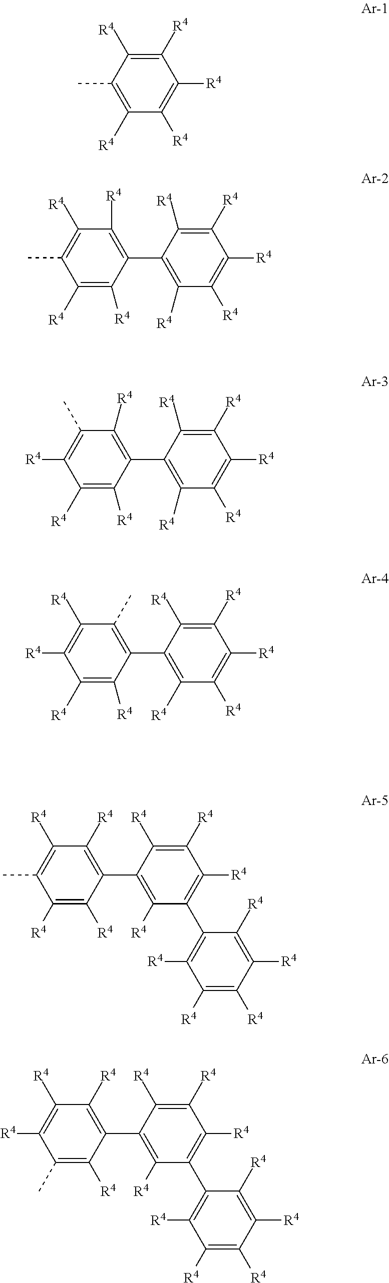

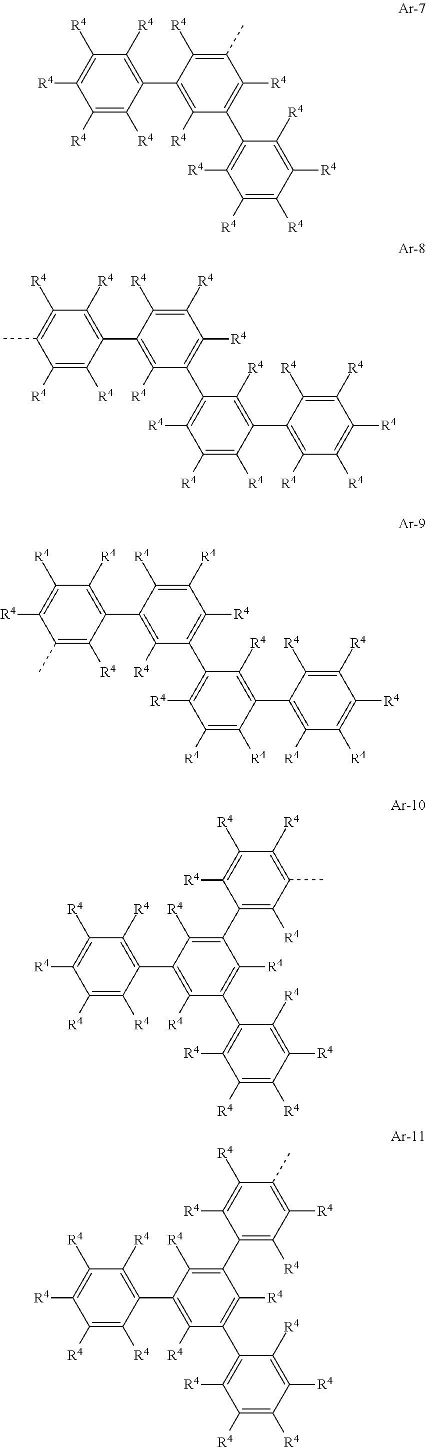

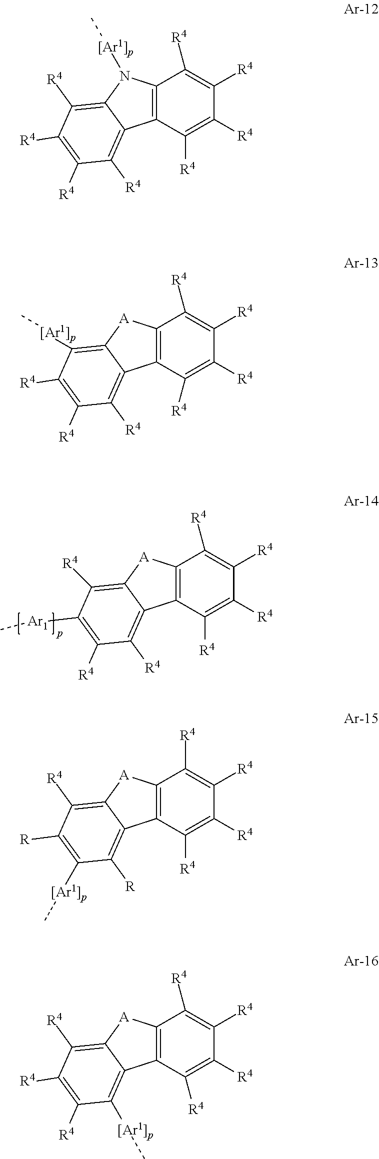

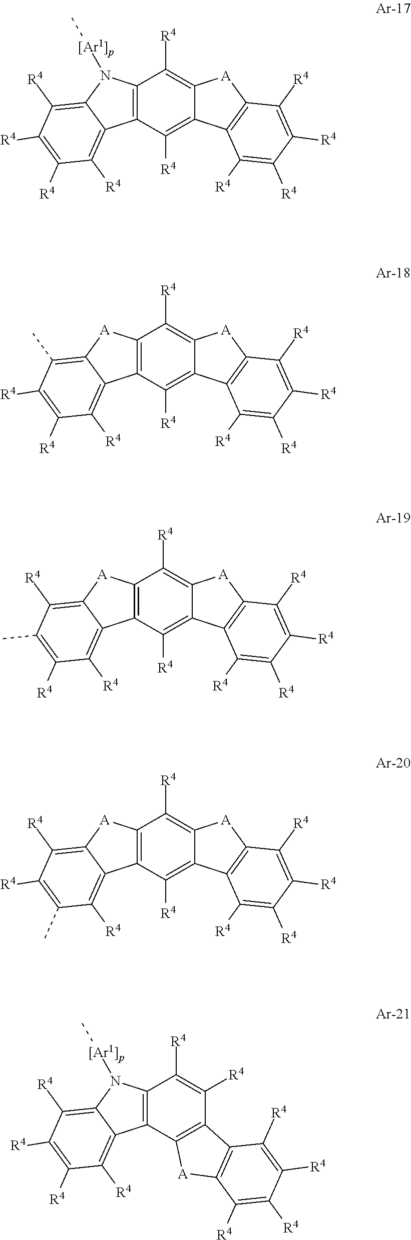

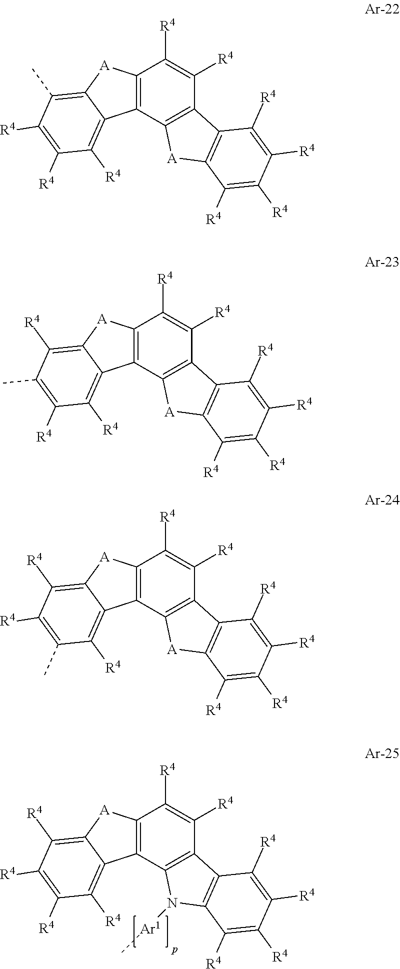

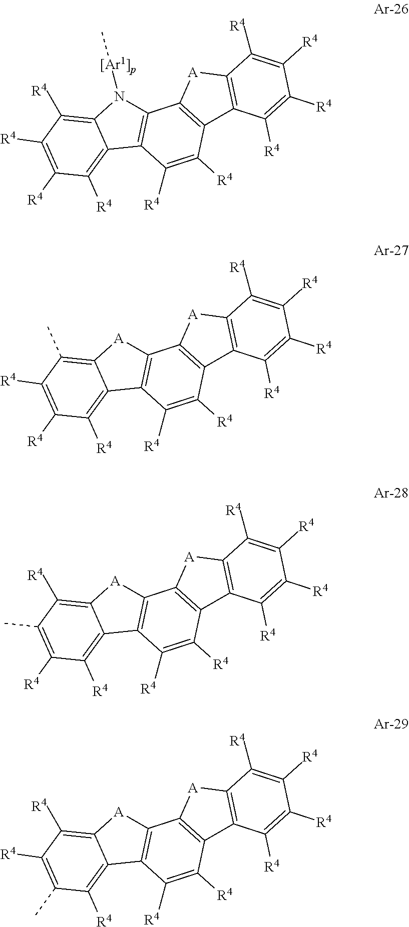

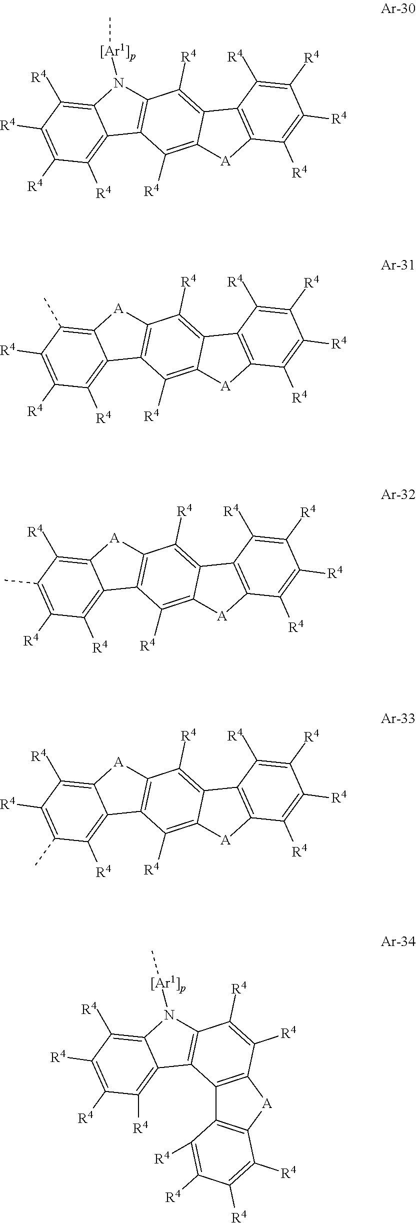

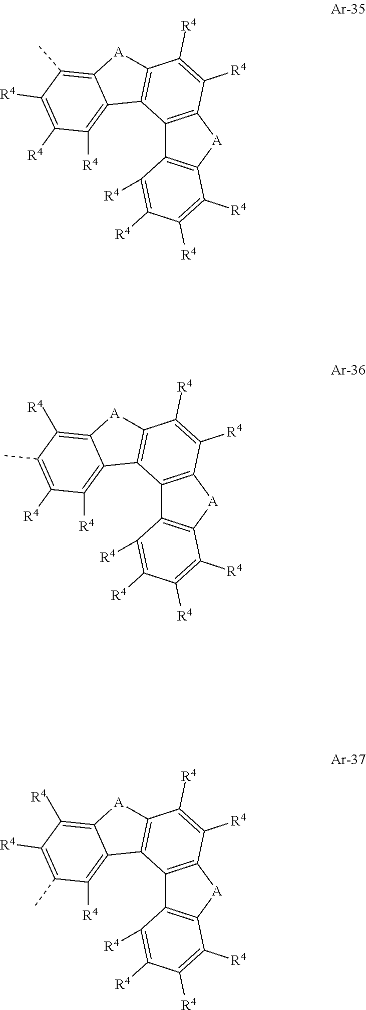

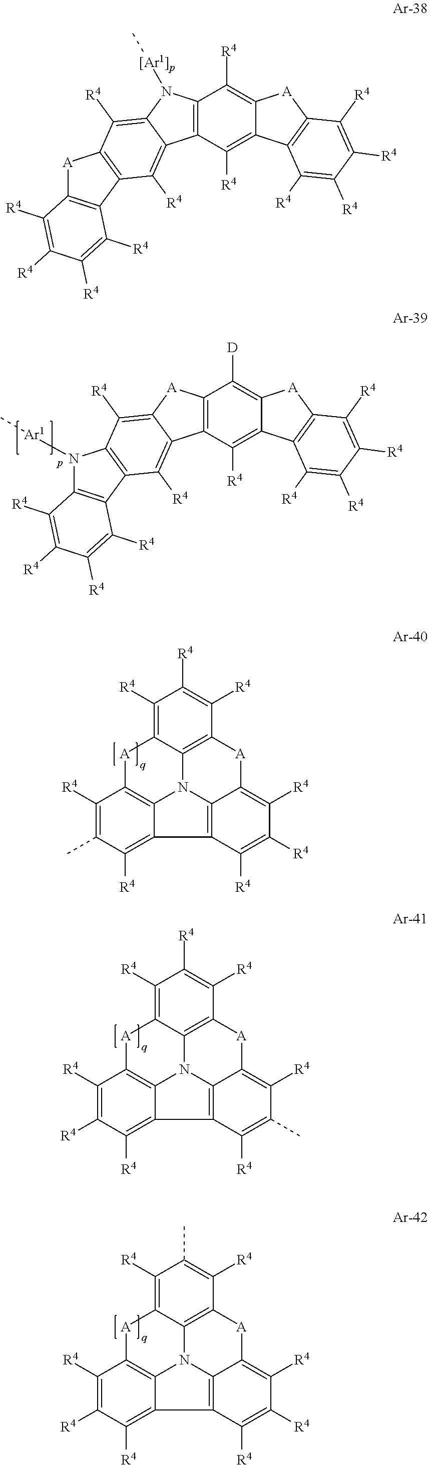

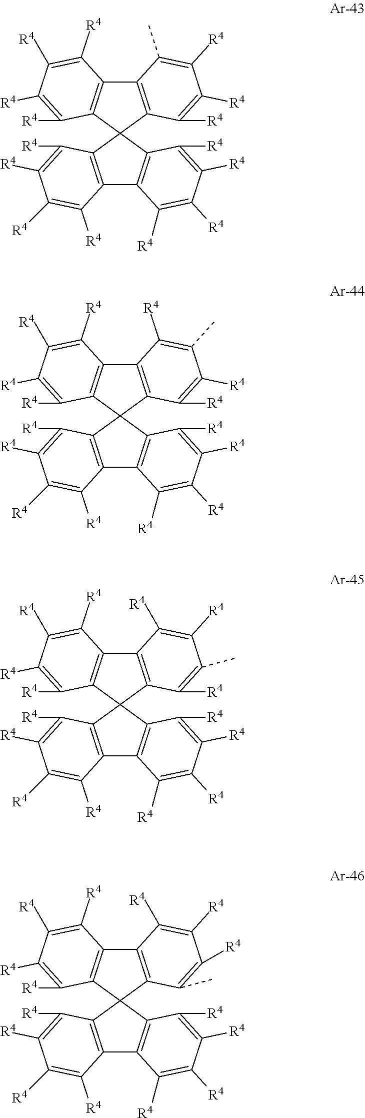

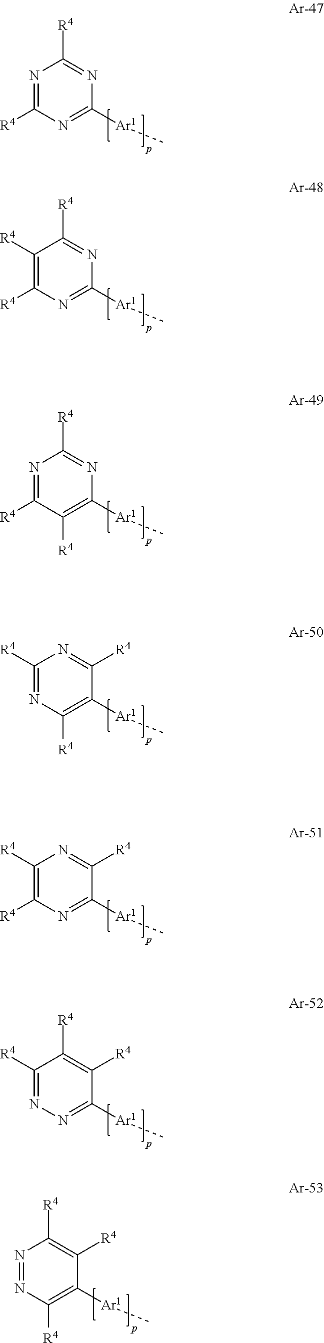

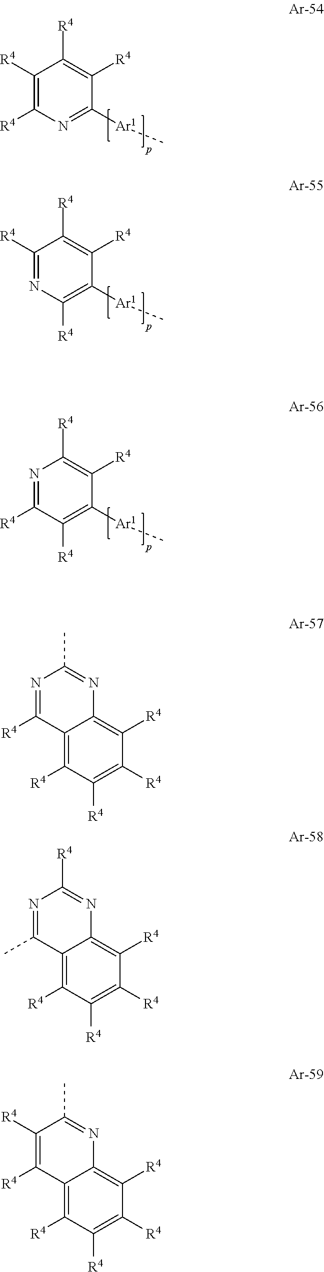

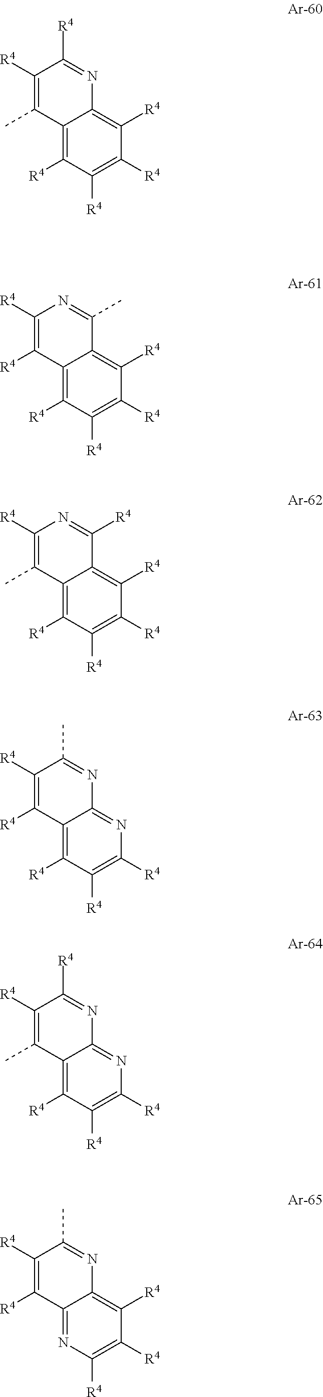

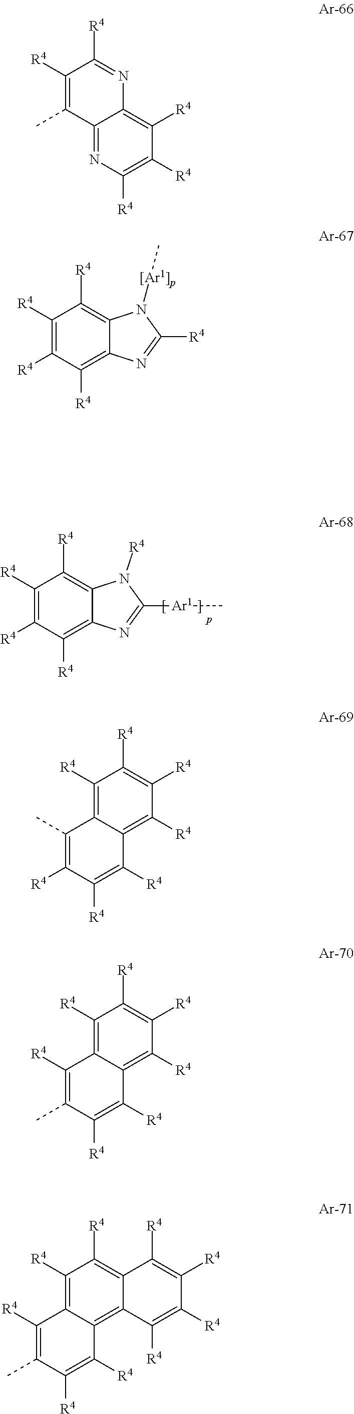

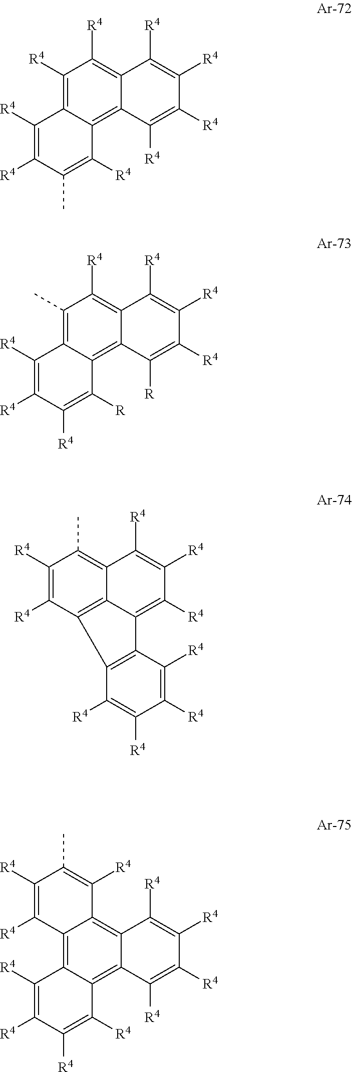

where R⁴ is as defined above, the dotted bond represents the site of attachment, and in addition:
Ar¹ is the same or different at each instance and is a bivalent aromatic or heteroaromatic ring system which has 6 to 18 aromatic ring atoms and may be substituted in each case by one or more R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

The above-detailed structures of the formulae (Ar-1) to (Ar-75) are preferred configurations of the Ar radical as defined, for example, in structures of the formula (1), in which case the substituents R⁴ in formulae (Ar-1) to (Ar-75) should be replaced by R², where R² has the definition set out above, especially for formula (1).

The above-detailed structures of the formulae (Ar-1) to (Ar-75) are preferred configurations of the Ar^{a}, Ar^{b} and Ar^{c} radicals as defined, for example, for preferred connecting groups L, in which case the substituents R⁴ in formulae (Ar-1) to (Ar-75) should be replaced by R², where R² has the definition set out above, especially for formula (1). In addition, the Ar^{b} and Ar^{c} radicals include a further site of attachment.

Preference is given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16).

When the abovementioned groups for Ar have two or more A groups, possible options for these include all combinations from the definition of A. Preferred embodiments in that case are those in which one A group is NR⁴ and the other A group is C(R⁴)₂ or in which both A groups are NR⁴ or in which both A groups are O.

When A is NR⁴, the substituent R⁴ bonded to the nitrogen atom is preferably an aromatic or heteroaromatic ring system which has 5 to 24 aromatic ring atoms and may also be substituted by one or more R⁵ radicals. In a particularly preferred embodiment, this R⁴ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁵ radicals. Preference is given to phenyl, biphenyl, terphenyl and quaterphenyl having bonding patterns as listed above for Ar-1 to Ar-11, where these structures, rather than by R⁴, may be substituted by one or more R⁵ radicals, but are preferably unsubstituted. Preference is further given to triazine, pyrimidine and quinazoline as listed above for Ar-47 to Ar-50, Ar-57 and Ar-58, where these structures, rather than by R⁴, may be substituted by one or more R⁵ radicals.

When A is C(R⁴)₂, the substituents R⁴ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁵ radicals. Most preferably, R⁴ is a methyl group or a phenyl group. In this case, the R⁴ radicals together may also form a ring system, which leads to a spiro system.

There follows a description of preferred substituents R, R¹, R² and R³.

In a preferred embodiment of the invention, R, R¹, R² and R³ are the same or different at each instance and are selected from the group consisting of H, D, F, CN, NO₂, Si(R⁴)₃, B(OR⁴)₂, a straight-chain alkyl group having 1 to 20 carbon atoms or a branched or cyclic alkyl group having 3 to 20 carbon atoms, where the alkyl group may be substituted in each case by one or more R⁴ radicals, or an aromatic or heteroaromatic ring system which has 5 to 60 aromatic ring atoms, preferably 5 to 40 aromatic ring atoms, and may be substituted in each case by one or more R⁴ radicals.

In a further-preferred embodiment of the invention, R, R¹, R² and R³ are the same or different at each instance and are selected from the group consisting of H, D, F, a straight-chain alkyl group having 1 to 20 carbon atoms or a branched or cyclic alkyl group having 3 to 20 carbon atoms, where the alkyl group may be substituted in each case by one or more R⁴ radicals, or an aromatic or heteroaromatic ring system which has 5 to 60 aromatic ring atoms, preferably 5 to 40 aromatic ring atoms, and may be substituted in each case by one or more R⁴ radicals.

In a further-preferred embodiment of the invention, R, R¹, R² and R³ are the same or different at each instance and are selected from the group consisting of H, D, an aromatic or heteroaromatic ring system which has 6 to 30 aromatic ring atoms and may be substituted by one or more R⁴ radicals, and an N(Ar′)₂ group. More preferably, R, R¹, R² are the same or different at each instance and are selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁴ radicals.

Preferred aromatic or heteroaromatic ring systems R, R¹, R², R³ and Ar′ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁴ radicals. The structures Ar-1 to Ar-75 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16).

Further suitable R, R¹, R² and R³ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40.

Ar⁴ and Ar² here may also be bonded to one another and/or Ar² and Ar³ to one another by a group selected from C(R⁴)₂, NR⁴, O and S. Preferably, Ar⁴ and Ar² are joined to one another and Ar² and Ar³ to one another in the respective ortho position to the bond to the nitrogen atom. In a further embodiment of the invention, none of the Ar², Ar³ and Ar⁴ groups are bonded to one another.

Preferably, Ar⁴ is an aromatic or heteroaromatic ring system which has 6 to 24 aromatic ring atoms, preferably 6 to 12 aromatic ring atoms, and may be substituted in each case by one or more R⁴ radicals. More preferably, Ar⁴ is selected from the group consisting of ortho-, meta- or para-phenylene or ortho-, meta- or para-biphenyl, each of which may be substituted by one or more R⁴ radicals, but are preferably unsubstituted. Most preferably, Ar⁴ is an unsubstituted phenylene group.

Preferably, Ar² and Ar³ are the same or different at each instance and are an aromatic or heteroaromatic ring system which has 6 to 24 aromatic ring atoms and may be substituted in each case by one or more R⁴ radicals. Particularly preferred Ar² and Ar³ groups are the same or different at each instance and are selected from the group consisting of benzene, ortho-, meta- or para-biphenyl, ortho-, meta- or para-terphenyl or branched terphenyl, ortho-, meta- or para-quaterphenyl or branched quaterphenyl, 1-, 2-, 3- or 4-fluorenyl, 1-, 2-, 3- or 4-spirobifluorenyl, 1- or 2-naphthyl, indole, benzofuran, benzothiophene, 1-, 2-, 3- or 4-carbazole, 1-, 2-, 3- or 4-dibenzofuran, 1-, 2-, 3- or 4-dibenzothiophene, indenocarbazole, indolocarbazole, 2-, 3- or 4-pyridine, 2-, 4- or 5-pyrimidine, pyrazine, pyridazine, triazine, phenanthrene or triphenylene, each of which may be substituted by one or more R¹ radicals. Most preferably, Ar² and Ar³ are the same or different at each instance and are selected from the group consisting of benzene, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene, especially 1-, 2-, 3- or 4-fluorene, or spirobifluorene, especially 1-, 2-, 3- or 4-spirobifluorene.

In a further preferred embodiment of the invention, R⁴ is the same or different at each instance and is selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R² radicals, or an aromatic or heteroaromatic ring system which has 6 to 24 aromatic ring atoms and may be substituted in each case by one or more R⁵ radicals. In a particularly preferred embodiment of the invention, R⁴ is the same or different at each instance and is selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁵ radicals, but is preferably unsubstituted, or an aromatic or heteroaromatic ring system which has 6 to 13 aromatic ring atoms and may be substituted in each case by one or more R⁵ radicals, but is preferably unsubstituted.

In a further preferred embodiment of the invention, R⁵ is the same or different at each instance and is H, an alkyl group having 1 to 4 carbon atoms or an aryl group having 6 to 10 carbon atoms, which may be substituted by an alkyl group having 1 to 4 carbon atoms, but is preferably unsubstituted.

At the same time, in compounds of the invention that are processed by vacuum evaporation, the alkyl groups preferably have not more than five carbon atoms, more preferably not more than 4 carbon atoms, most preferably not more than 1 carbon atom. For compounds that are processed from solution, suitable compounds are also those substituted by alkyl groups, especially branched alkyl groups, having up to 10 carbon atoms or those substituted by oligoarylene groups, for example ortho-, meta- or para-terphenyl or branched terphenyl or quaterphenyl groups.

When the compounds of the formula (1) or the preferred embodiments are used as matrix material for a phosphorescent emitter or in a layer directly adjoining a phosphorescent layer, it is further preferable when the compound does not contain any fused aryl or heteroaryl groups in which more than two six-membered rings are fused directly to one another. An exception to this is formed by phenanthrene and triphenylene which, because of their high triplet energy, may be preferable in spite of the presence of fused aromatic six-membered rings.

In addition, it is a feature of preferred compounds of the invention that they are sublimable. These compounds generally have a molar mass of less than about 1200 g/mol.

It may further be the case that the compound comprising structures of formula (1), preferably the compound of formula (1) or a preferred embodiment of that structure/compound is not in direct contact with a metal atom, and is preferably not a ligand for a metal complex.

The abovementioned preferred embodiments may be combined with one another as desired within the restrictions defined in claim 1. In a particularly preferred embodiment of the invention, the abovementioned preferences occur simultaneously.

Examples of preferred compounds according to the embodiments detailed above are the compounds detailed in the following table:

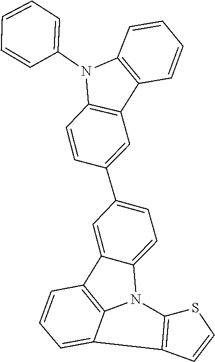 [SMILES: c1ccc(-n2c3ccccc3c3cc(-c4ccc5c(c4)c4cccc6c7ccsc7n5c46)ccc32)cc1]

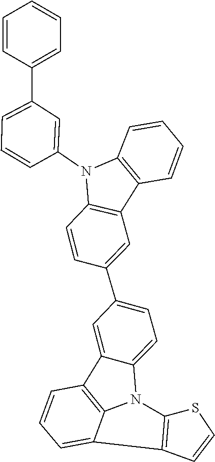 [SMILES: c1ccc(-c2cccc(-n3c4ccccc4c4cc(-c5ccc6c(c5)c5cccc7c8ccsc8n6c57)ccc43)c2)cc1]

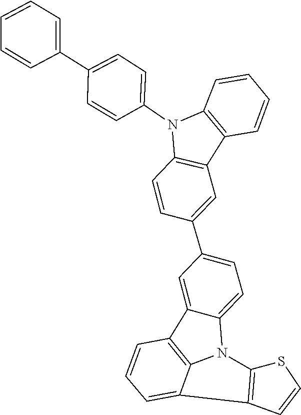 [SMILES: c1ccc(-c2ccc(-n3c4ccccc4c4cc(-c5ccc6c(c5)c5cccc7c8ccsc8n6c57)ccc43)cc2)cc1]

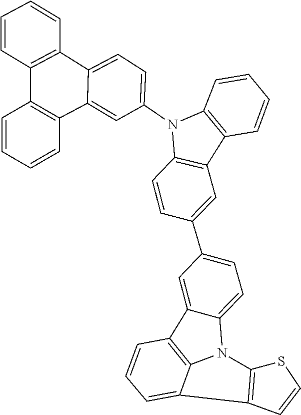 [SMILES: c1ccc2c(c1)c1ccccc1c1cc(-n3c4ccccc4c4cc(-c5ccc6c(c5)c5cccc7c8ccsc8n6c57)ccc43)ccc21]

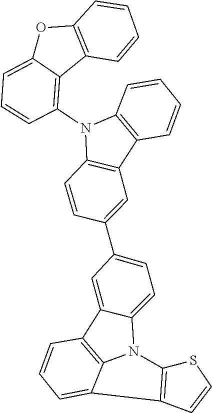 [SMILES: c1ccc2c(c1)oc1cccc(-n3c4ccccc4c4cc(-c5ccc6c(c5)c5cccc7c8ccsc8n6c57)ccc43)c12]

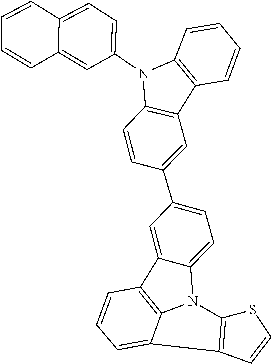 [SMILES: c1ccc2cc(-n3c4ccccc4c4cc(-c5ccc6c(c5)c5cccc7c8ccsc8n6c57)ccc43)ccc2c1]

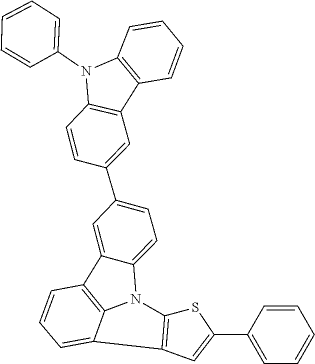 [SMILES: c1ccc(-c2cc3c4cccc5c6cc(-c7ccc8c(c7)c7ccccc7n8-c7ccccc7)ccc6n(c3s2)c54)cc1]

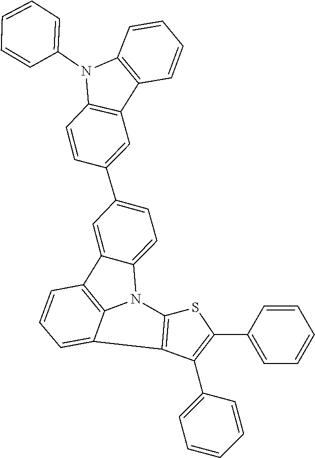 [SMILES: c1ccc(-c2sc3c(c2-c2ccccc2)c2cccc4c5cc(-c6ccc7c(c6)c6ccccc6n7-c6ccccc6)ccc5n3c42)cc1]

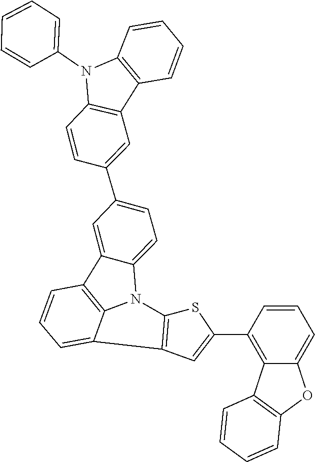 [SMILES: c1ccc(-n2c3ccccc3c3cc(-c4ccc5c(c4)c4cccc6c7cc(-c8cccc9oc%10ccccc%10c89)sc7n5c46)ccc32)cc1]

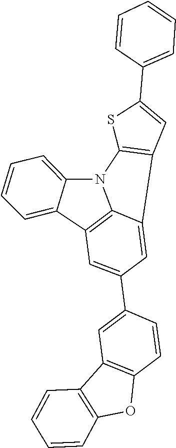 [SMILES: c1ccc(-c2cc3c4cc(-c5ccc6oc7ccccc7c6c5)cc5c6ccccc6n(c3s2)c54)cc1]

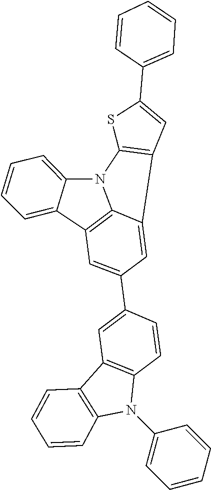 [SMILES: c1ccc(-c2cc3c4cc(-c5ccc6c(c5)c5ccccc5n6-c5ccccc5)cc5c6ccccc6n(c3s2)c54)cc1]

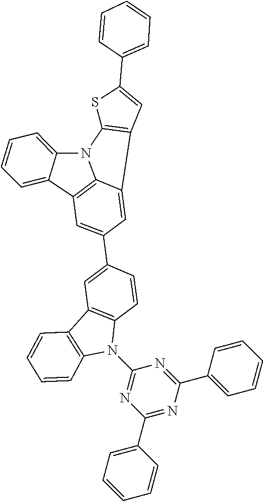 [SMILES: c1ccc(-c2nc(-c3ccccc3)nc(-n3c4ccccc4c4cc(-c5cc6c7ccccc7n7c8sc(-c9ccccc9)cc8c(c5)c67)ccc43)n2)cc1]

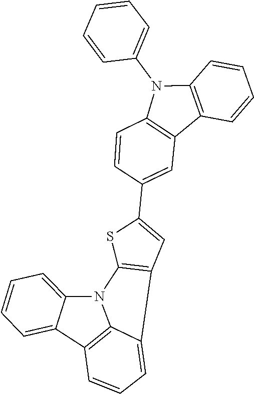 [SMILES: c1ccc(-n2c3ccccc3c3cc(-c4cc5c6cccc7c8ccccc8n(c5s4)c76)ccc32)cc1]

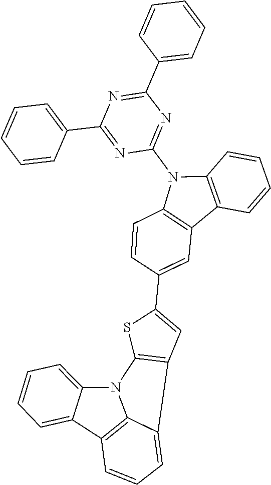 [SMILES: c1ccc(-c2nc(-c3ccccc3)nc(-n3c4ccccc4c4cc(-c5cc6c7cccc8c9ccccc9n(c6s5)c87)ccc43)n2)cc1]

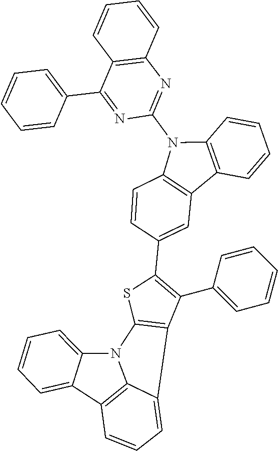 [SMILES: c1ccc(-c2nc(-n3c4ccccc4c4cc(-c5sc6c(c5-c5ccccc5)c5cccc7c8ccccc8n6c75)ccc43)nc3ccccc23)cc1]

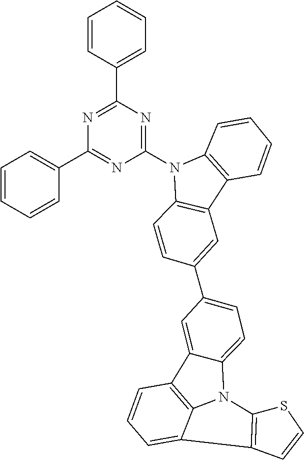 [SMILES: c1ccc(-c2nc(-c3ccccc3)nc(-n3c4ccccc4c4cc(-c5ccc6c(c5)c5cccc7c8ccsc8n6c57)ccc43)n2)cc1]

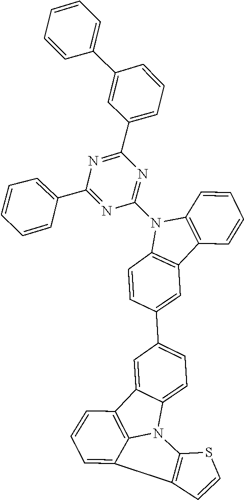 [SMILES: c1ccc(-c2cccc(-c3nc(-c4ccccc4)nc(-n4c5ccccc5c5cc(-c6ccc7c(c6)c6cccc8c9ccsc9n7c68)ccc54)n3)c2)cc1]

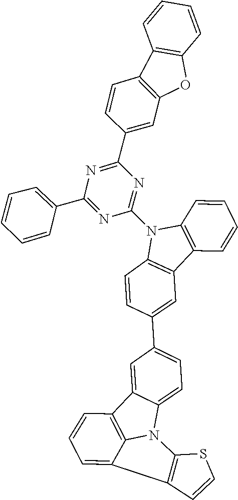 [SMILES: c1ccc(-c2nc(-c3ccc4c(c3)oc3ccccc34)nc(-n3c4ccccc4c4cc(-c5ccc6c(c5)c5cccc7c8ccsc8n6c57)ccc43)n2)cc1]

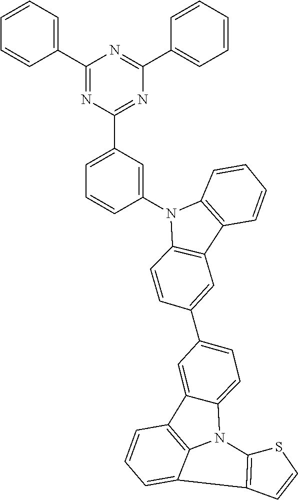 [SMILES: c1ccc(-c2nc(-c3ccccc3)nc(-c3cccc(-n4c5ccccc5c5cc(-c6ccc7c(c6)c6cccc8c9ccsc9n7c68)ccc54)c3)n2)cc1]

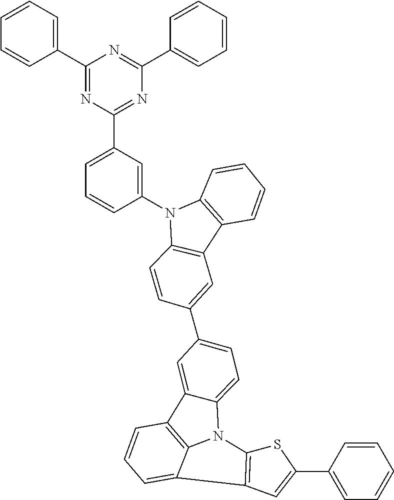 [SMILES: c1ccc(-c2nc(-c3ccccc3)nc(-c3cccc(-n4c5ccccc5c5cc(-c6ccc7c(c6)c6cccc8c9cc(-c%10ccccc%10)sc9n7c68)ccc54)c3)n2)cc1]

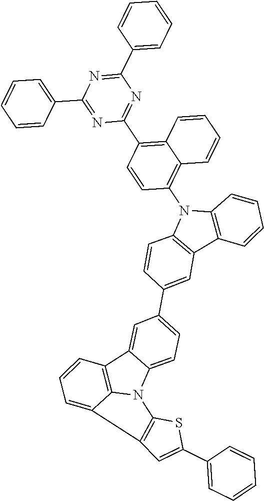 [SMILES: c1ccc(-c2nc(-c3ccccc3)nc(-c3ccc(-n4c5ccccc5c5cc(-c6ccc7c(c6)c6cccc8c9cc(-c%10ccccc%10)sc9n7c68)ccc54)c4ccccc34)n2)cc1]

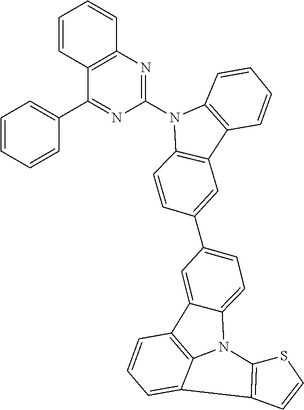 [SMILES: c1ccc(-c2nc(-n3c4ccccc4c4cc(-c5ccc6c(c5)c5cccc7c8ccsc8n6c57)ccc43)nc3ccccc23)cc1]

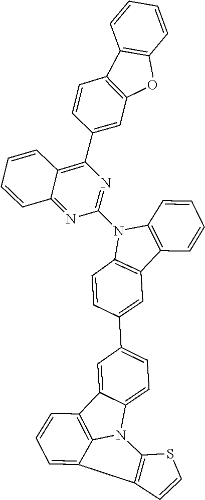 [SMILES: c1ccc2c(-c3ccc4c(c3)oc3ccccc34)nc(-n3c4ccccc4c4cc(-c5ccc6c(c5)c5cccc7c8ccsc8n6c57)ccc43)nc2c1]

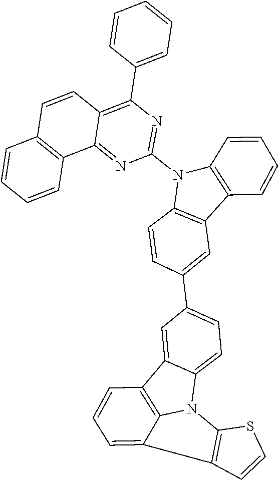 [SMILES: c1ccc(-c2nc(-n3c4ccccc4c4cc(-c5ccc6c(c5)c5cccc7c8ccsc8n6c57)ccc43)nc3c2ccc2ccccc23)cc1]

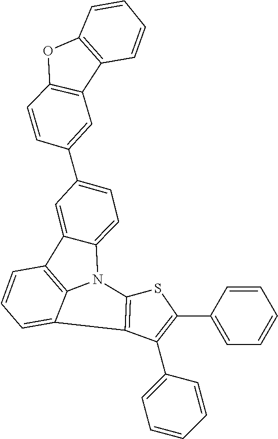 [SMILES: c1ccc(-c2sc3c(c2-c2ccccc2)c2cccc4c5cc(-c6ccc7oc8ccccc8c7c6)ccc5n3c42)cc1]

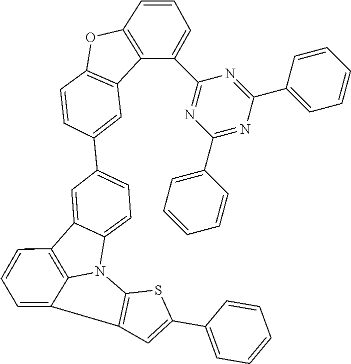 [SMILES: c1ccc(-c2nc(-c3ccccc3)nc(-c3cccc4oc5ccc(-c6ccc7c(c6)c6cccc8c9cc(-c%10ccccc%10)sc9n7c68)cc5c34)n2)cc1]

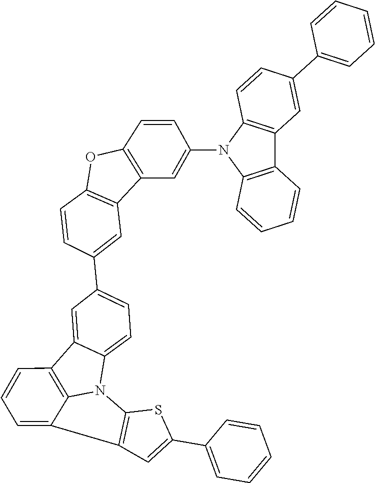 [SMILES: c1ccc(-c2ccc3c(c2)c2ccccc2n3-c2ccc3oc4ccc(-c5ccc6c(c5)c5cccc7c8cc(-c9ccccc9)sc8n6c57)cc4c3c2)cc1]

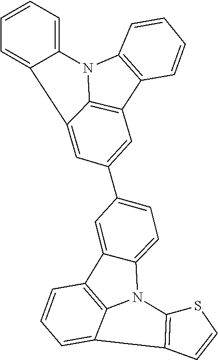 [SMILES: c1ccc2c(c1)c1cc(-c3ccc4c(c3)c3cccc5c6ccsc6n4c35)cc3c4ccccc4n2c13]

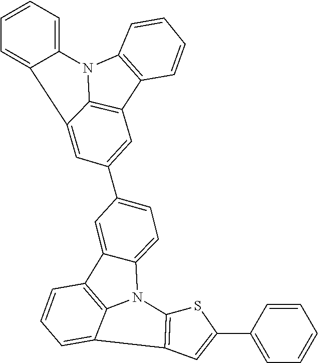 [SMILES: c1ccc(-c2cc3c4cccc5c6cc(-c7cc8c9ccccc9n9c%10ccccc%10c(c7)c89)ccc6n(c3s2)c54)cc1]

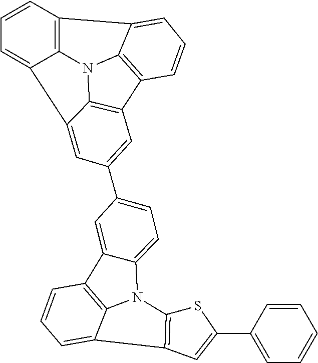 [SMILES: c1ccc(-c2cc3c4cccc5c6cc(-c7cc8c9cccc%10c%11cccc%12c(c7)c8n(c%109)c%11%12)ccc6n(c3s2)c54)cc1]

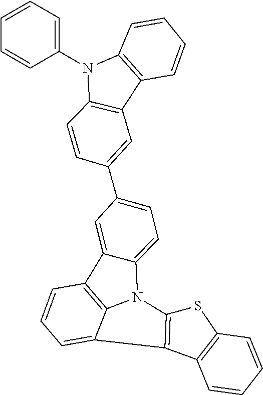 [SMILES: c1ccc(-n2c3ccccc3c3cc(-c4ccc5c(c4)c4cccc6c7c8ccccc8sc7n5c46)ccc32)cc1]

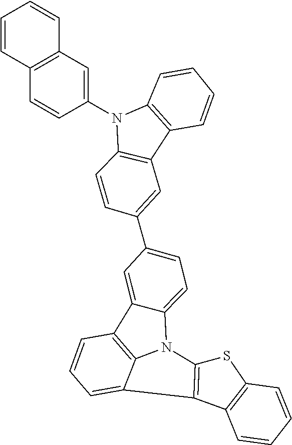 [SMILES: c1ccc2cc(-n3c4ccccc4c4cc(-c5ccc6c(c5)c5cccc7c8c9ccccc9sc8n6c57)ccc43)ccc2c1]

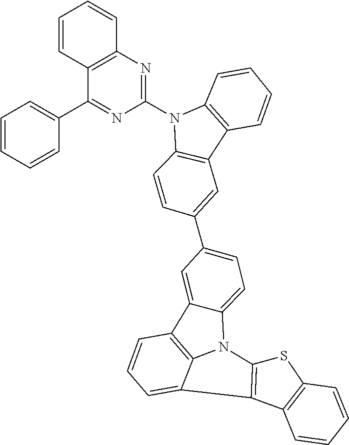 [SMILES: c1ccc(-c2nc(-n3c4ccccc4c4cc(-c5ccc6c(c5)c5cccc7c8c9ccccc9sc8n6c57)ccc43)nc3ccccc23)cc1]

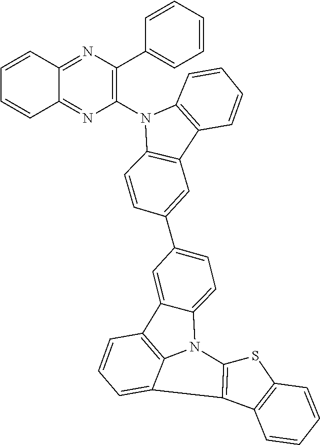 [SMILES: c1ccc(-c2nc3ccccc3nc2-n2c3ccccc3c3cc(-c4ccc5c(c4)c4cccc6c7c8ccccc8sc7n5c46)ccc32)cc1]

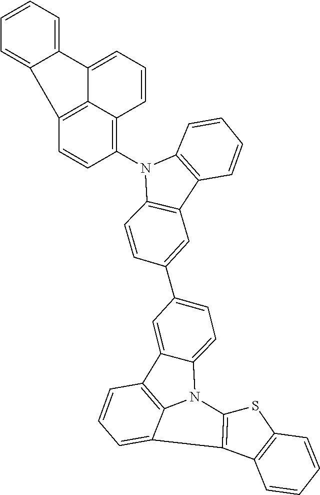 [SMILES: c1ccc2c(c1)-c1cccc3c(-n4c5ccccc5c5cc(-c6ccc7c(c6)c6cccc8c9c%10ccccc%10sc9n7c68)ccc54)ccc-2c13]

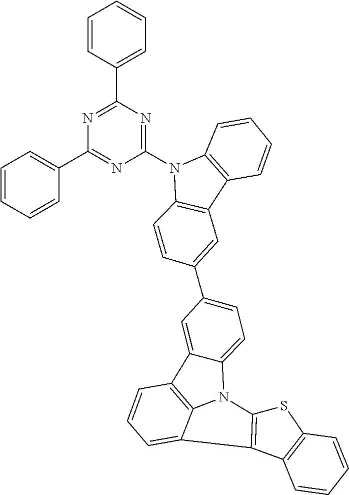 [SMILES: c1ccc(-c2nc(-c3ccccc3)nc(-n3c4ccccc4c4cc(-c5ccc6c(c5)c5cccc7c8c9ccccc9sc8n6c57)ccc43)n2)cc1]

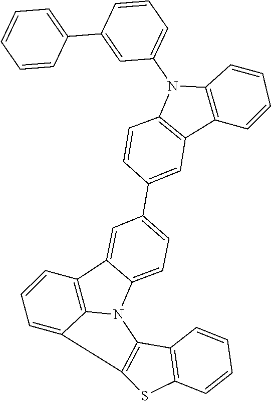 [SMILES: c1ccc(-c2cccc(-n3c4ccccc4c4cc(-c5ccc6c(c5)c5cccc7c8sc9ccccc9c8n6c57)ccc43)c2)cc1]

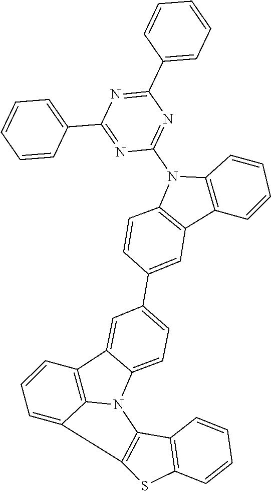 [SMILES: c1ccc(-c2nc(-c3ccccc3)nc(-n3c4ccccc4c4cc(-c5ccc6c(c5)c5cccc7c8sc9ccccc9c8n6c57)ccc43)n2)cc1]

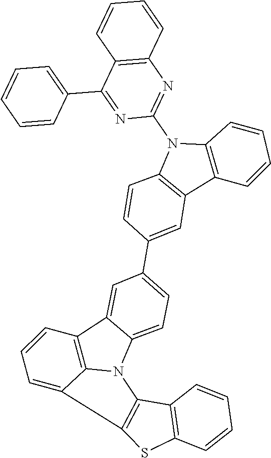 [SMILES: c1ccc(-c2nc(-n3c4ccccc4c4cc(-c5ccc6c(c5)c5cccc7c8sc9ccccc9c8n6c57)ccc43)nc3ccccc23)cc1]

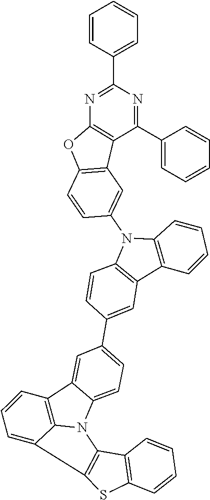 [SMILES: c1ccc(-c2nc(-c3ccccc3)c3c(n2)oc2ccc(-n4c5ccccc5c5cc(-c6ccc7c(c6)c6cccc8c9sc%10ccccc%10c9n7c68)ccc54)cc23)cc1]

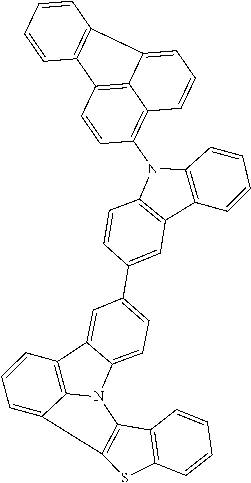 [SMILES: c1ccc2c(c1)-c1cccc3c(-n4c5ccccc5c5cc(-c6ccc7c(c6)c6cccc8c9sc%10ccccc%10c9n7c68)ccc54)ccc-2c13]

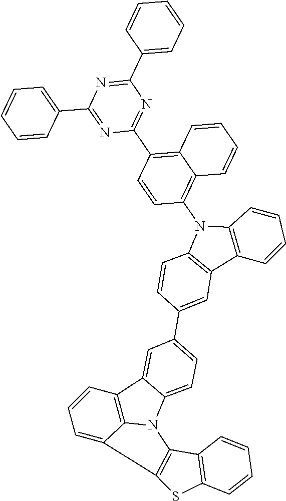 [SMILES: c1ccc(-c2nc(-c3ccccc3)nc(-c3ccc(-n4c5ccccc5c5cc(-c6ccc7c(c6)c6cccc8c9sc%10ccccc%10c9n7c68)ccc54)c4ccccc34)n2)cc1]

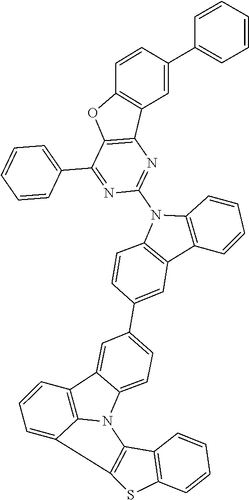 [SMILES: c1ccc(-c2ccc3oc4c(-c5ccccc5)nc(-n5c6ccccc6c6cc(-c7ccc8c(c7)c7cccc9c%10sc%11ccccc%11c%10n8c79)ccc65)nc4c3c2)cc1]

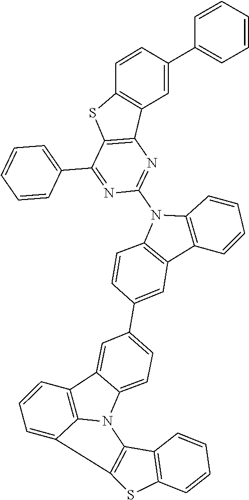 [SMILES: c1ccc(-c2ccc3sc4c(-c5ccccc5)nc(-n5c6ccccc6c6cc(-c7ccc8c(c7)c7cccc9c%10sc%11ccccc%11c%10n8c79)ccc65)nc4c3c2)cc1]

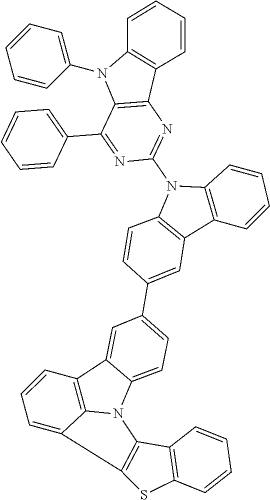 [SMILES: c1ccc(-c2nc(-n3c4ccccc4c4cc(-c5ccc6c(c5)c5cccc7c8sc9ccccc9c8n6c57)ccc43)nc3c4ccccc4n(-c4ccccc4)c23)cc1]

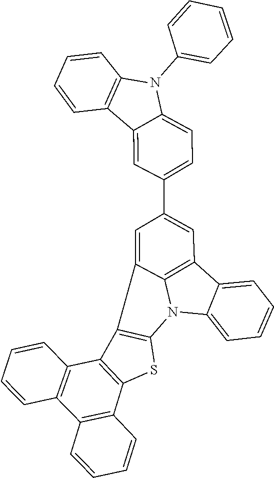 [SMILES: c1ccc(-n2c3ccccc3c3cc(-c4cc5c6ccccc6n6c7sc8c9ccccc9c9ccccc9c8c7c(c4)c56)ccc32)cc1]

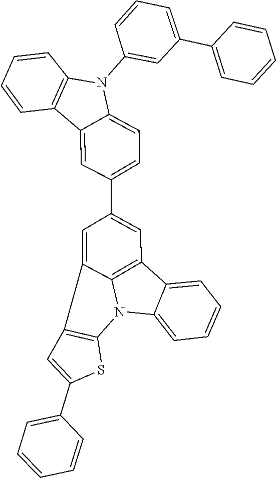 [SMILES: c1ccc(-c2cccc(-n3c4ccccc4c4cc(-c5cc6c7ccccc7n7c8sc(-c9ccccc9)cc8c(c5)c67)ccc43)c2)cc1]

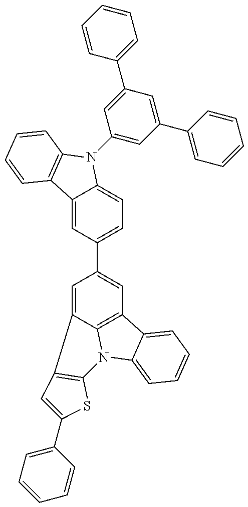 [SMILES: c1ccc(-c2cc(-c3ccccc3)cc(-n3c4ccccc4c4cc(-c5cc6c7ccccc7n7c8sc(-c9ccccc9)cc8c(c5)c67)ccc43)c2)cc1]

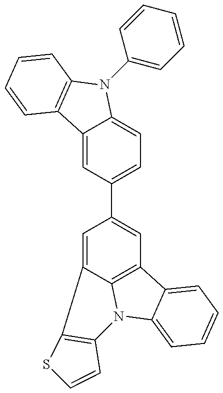 [SMILES: c1ccc(-n2c3ccccc3c3cc(-c4cc5c6ccccc6n6c7ccsc7c(c4)c56)ccc32)cc1]

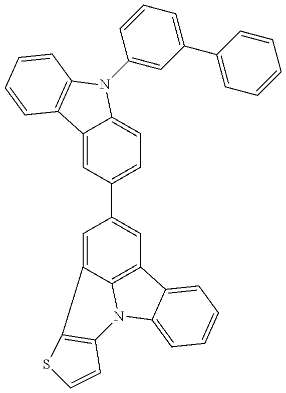 [SMILES: c1ccc(-c2cccc(-n3c4ccccc4c4cc(-c5cc6c7ccccc7n7c8ccsc8c(c5)c67)ccc43)c2)cc1]

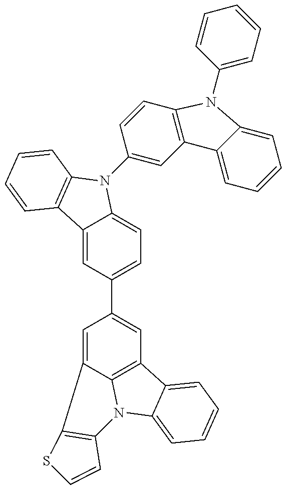 [SMILES: c1ccc(-n2c3ccccc3c3cc(-n4c5ccccc5c5cc(-c6cc7c8ccccc8n8c9ccsc9c(c6)c78)ccc54)ccc32)cc1]

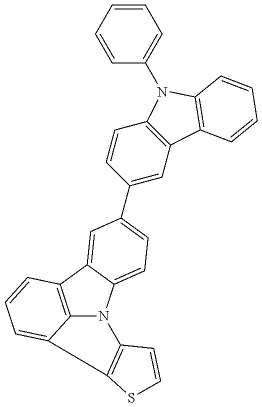 [SMILES: c1ccc(-n2c3ccccc3c3cc(-c4ccc5c(c4)c4cccc6c7sccc7n5c46)ccc32)cc1]

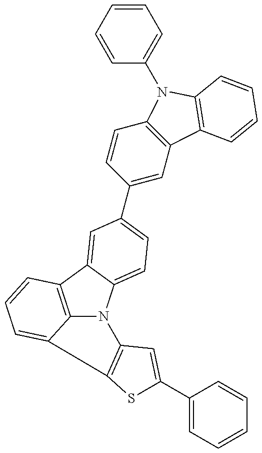 [SMILES: c1ccc(-c2cc3c(s2)c2cccc4c5cc(-c6ccc7c(c6)c6ccccc6n7-c6ccccc6)ccc5n3c42)cc1]

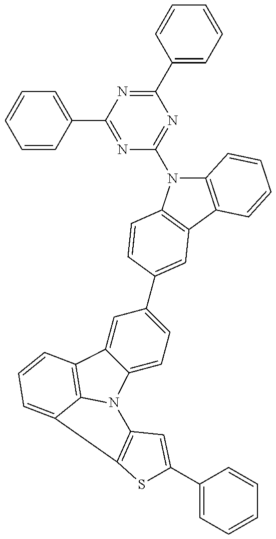 [SMILES: c1ccc(-c2nc(-c3ccccc3)nc(-n3c4ccccc4c4cc(-c5ccc6c(c5)c5cccc7c8sc(-c9ccccc9)cc8n6c57)ccc43)n2)cc1]

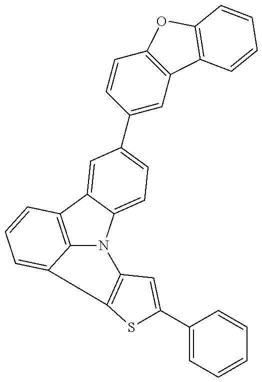 [SMILES: c1ccc(-c2cc3c(s2)c2cccc4c5cc(-c6ccc7oc8ccccc8c7c6)ccc5n3c42)cc1]

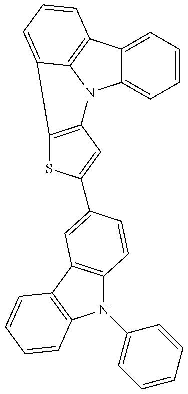 [SMILES: c1ccc(-n2c3ccccc3c3cc(-c4cc5c(s4)c4cccc6c7ccccc7n5c64)ccc32)cc1]

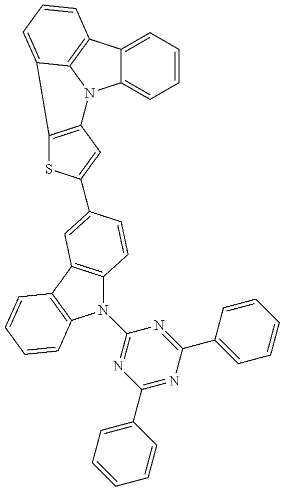 [SMILES: c1ccc(-c2nc(-c3ccccc3)nc(-n3c4ccccc4c4cc(-c5cc6c(s5)c5cccc7c8ccccc8n6c75)ccc43)n2)cc1]

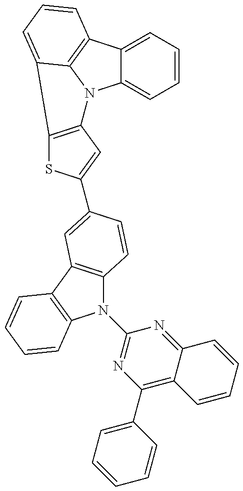 [SMILES: c1ccc(-c2nc(-n3c4ccccc4c4cc(-c5cc6c(s5)c5cccc7c8ccccc8n6c75)ccc43)nc3ccccc23)cc1]

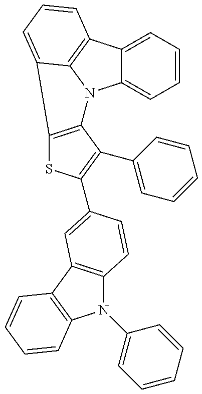 [SMILES: c1ccc(-c2c(-c3ccc4c(c3)c3ccccc3n4-c3ccccc3)sc3c4cccc5c6ccccc6n(c23)c54)cc1]

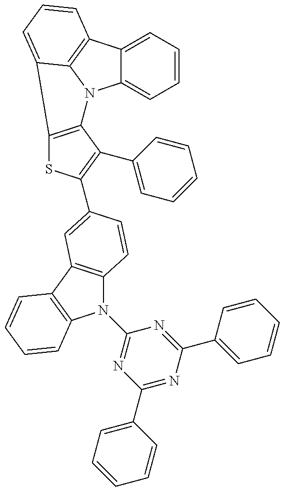 [SMILES: c1ccc(-c2nc(-c3ccccc3)nc(-n3c4ccccc4c4cc(-c5sc6c7cccc8c9ccccc9n(c6c5-c5ccccc5)c87)ccc43)n2)cc1]

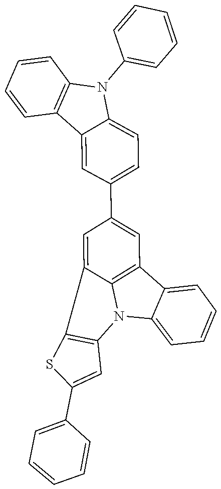 [SMILES: c1ccc(-c2cc3c(s2)c2cc(-c4ccc5c(c4)c4ccccc4n5-c4ccccc4)cc4c5ccccc5n3c42)cc1]

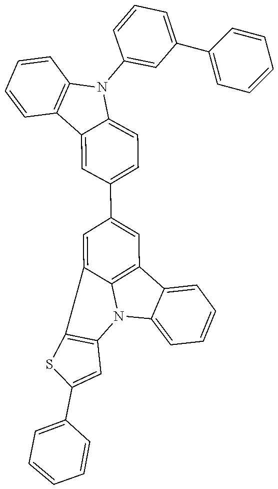 [SMILES: c1ccc(-c2cccc(-n3c4ccccc4c4cc(-c5cc6c7ccccc7n7c8cc(-c9ccccc9)sc8c(c5)c67)ccc43)c2)cc1]

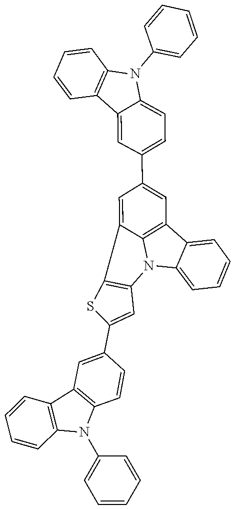 [SMILES: c1ccc(-n2c3ccccc3c3cc(-c4cc5c6ccccc6n6c7cc(-c8ccc9c(c8)c8ccccc8n9-c8ccccc8)sc7c(c4)c56)ccc32)cc1]

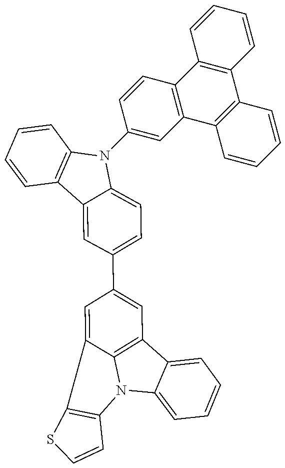 [SMILES: c1ccc2c(c1)c1ccccc1c1cc(-n3c4ccccc4c4cc(-c5cc6c7ccccc7n7c8ccsc8c(c5)c67)ccc43)ccc21]

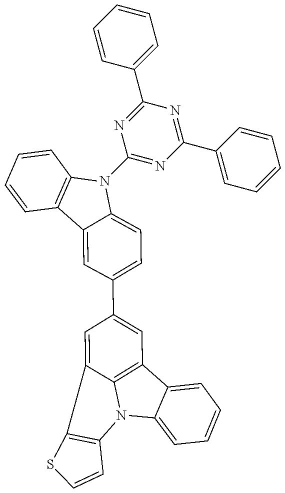 [SMILES: c1ccc(-c2nc(-c3ccccc3)nc(-n3c4ccccc4c4cc(-c5cc6c7ccccc7n7c8ccsc8c(c5)c67)ccc43)n2)cc1]

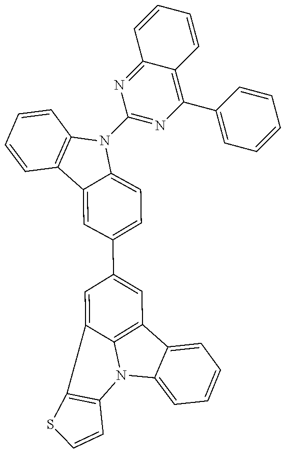 [SMILES: c1ccc(-c2nc(-n3c4ccccc4c4cc(-c5cc6c7ccccc7n7c8ccsc8c(c5)c67)ccc43)nc3ccccc23)cc1]

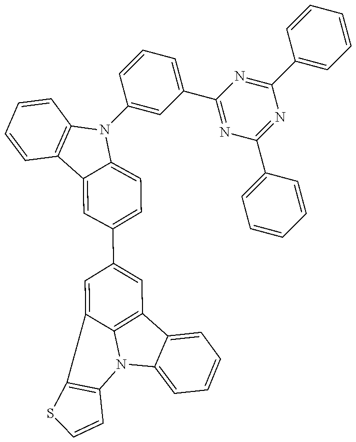 [SMILES: c1ccc(-c2nc(-c3ccccc3)nc(-c3cccc(-n4c5ccccc5c5cc(-c6cc7c8ccccc8n8c9ccsc9c(c6)c78)ccc54)c3)n2)cc1]

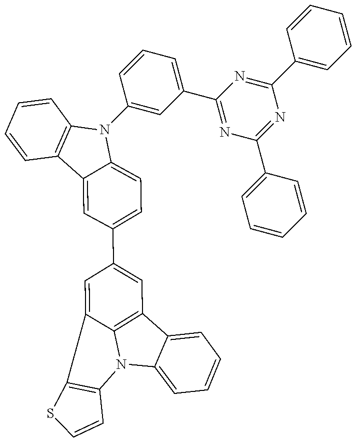 [SMILES: c1ccc(-c2nc(-c3ccccc3)nc(-c3cccc(-n4c5ccccc5c5cc(-c6cc7c8ccccc8n8c9ccsc9c(c6)c78)ccc54)c3)n2)cc1]

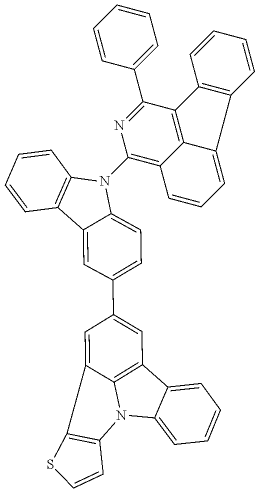 [SMILES: c1ccc(-c2nc(-n3c4ccccc4c4cc(-c5cc6c7ccccc7n7c8ccsc8c(c5)c67)ccc43)c3cccc4c3c2-c2ccccc2-4)cc1]

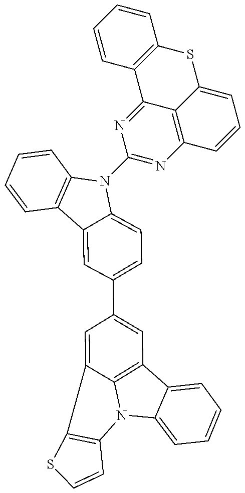 [SMILES: c1ccc2c(c1)Sc1cccc3nc(-n4c5ccccc5c5cc(-c6cc7c8ccccc8n8c9ccsc9c(c6)c78)ccc54)nc-2c13]

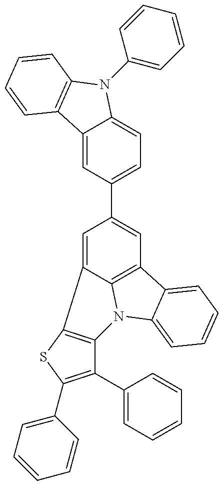 [SMILES: c1ccc(-c2sc3c4cc(-c5ccc6c(c5)c5ccccc5n6-c5ccccc5)cc5c6ccccc6n(c3c2-c2ccccc2)c54)cc1]

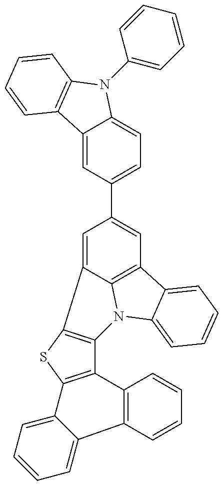 [SMILES: c1ccc(-n2c3ccccc3c3cc(-c4cc5c6ccccc6n6c5c(c4)c4sc5c7ccccc7c7ccccc7c5c46)ccc32)cc1]

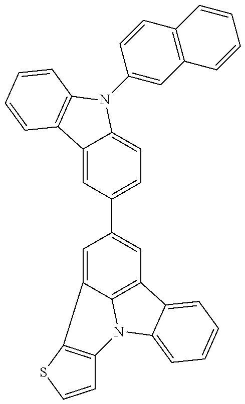 [SMILES: c1ccc2cc(-n3c4ccccc4c4cc(-c5cc6c7ccccc7n7c8ccsc8c(c5)c67)ccc43)ccc2c1]

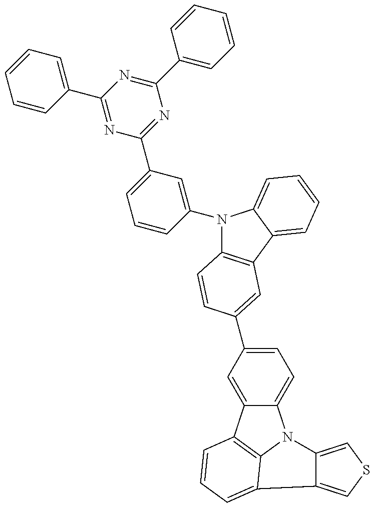 [SMILES: c1ccc(-c2nc(-c3ccccc3)nc(-c3cccc(-n4c5ccccc5c5cc(-c6ccc7c(c6)c6cccc8c9cscc9n7c68)ccc54)c3)n2)cc1]

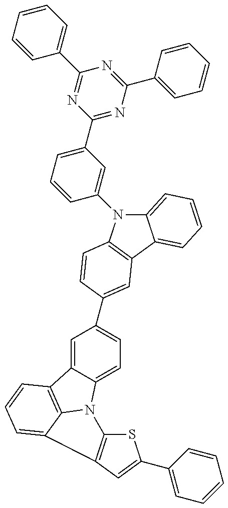 [SMILES: c1ccc(-c2nc(-c3ccccc3)nc(-c3cccc(-n4c5ccccc5c5cc(-c6ccc7c(c6)c6cccc8c9cc(-c%10ccccc%10)sc9n7c68)ccc54)c3)n2)cc1]

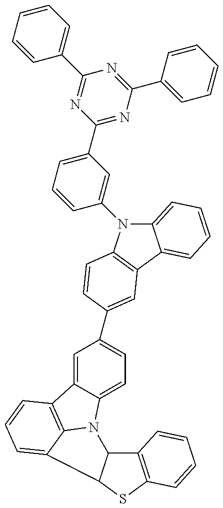 [SMILES: c1ccc(-c2nc(-c3ccccc3)nc(-c3cccc(-n4c5ccccc5c5cc(-c6ccc7c(c6)c6cccc8c6n7C6c7ccccc7SC86)ccc54)c3)n2)cc1]

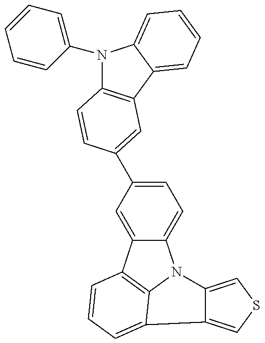 [SMILES: c1ccc(-n2c3ccccc3c3cc(-c4ccc5c(c4)c4cccc6c7cscc7n5c46)ccc32)cc1]

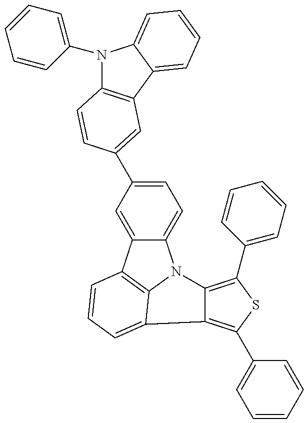 [SMILES: c1ccc(-c2sc(-c3ccccc3)c3c2c2cccc4c5cc(-c6ccc7c(c6)c6ccccc6n7-c6ccccc6)ccc5n3c42)cc1]

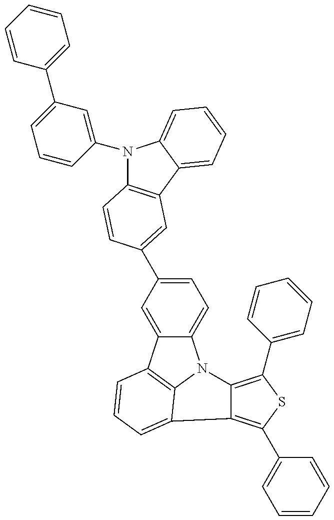 [SMILES: c1ccc(-c2cccc(-n3c4ccccc4c4cc(-c5ccc6c(c5)c5cccc7c8c(-c9ccccc9)sc(-c9ccccc9)c8n6c57)ccc43)c2)cc1]

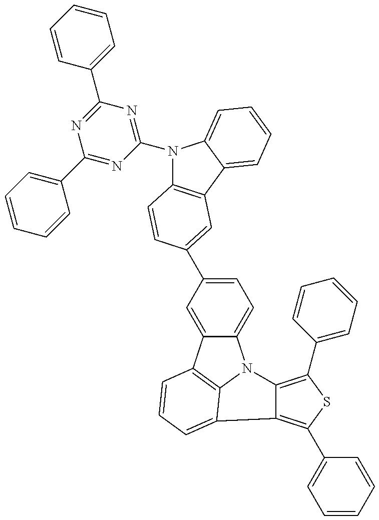 [SMILES: c1ccc(-c2nc(-c3ccccc3)nc(-n3c4ccccc4c4cc(-c5ccc6c(c5)c5cccc7c8c(-c9ccccc9)sc(-c9ccccc9)c8n6c57)ccc43)n2)cc1]

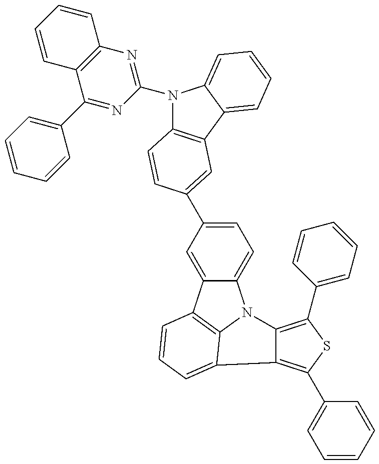 [SMILES: c1ccc(-c2nc(-n3c4ccccc4c4cc(-c5ccc6c(c5)c5cccc7c8c(-c9ccccc9)sc(-c9ccccc9)c8n6c57)ccc43)nc3ccccc23)cc1]

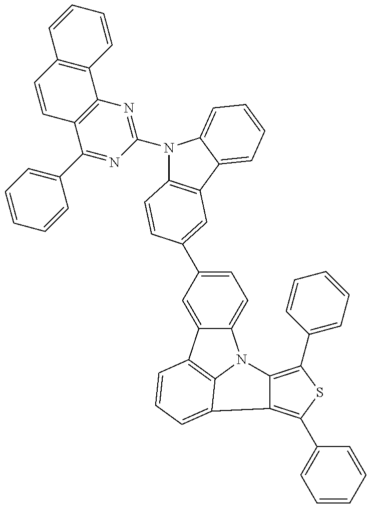 [SMILES: c1ccc(-c2nc(-n3c4ccccc4c4cc(-c5ccc6c(c5)c5cccc7c8c(-c9ccccc9)sc(-c9ccccc9)c8n6c57)ccc43)nc3c2ccc2ccccc23)cc1]

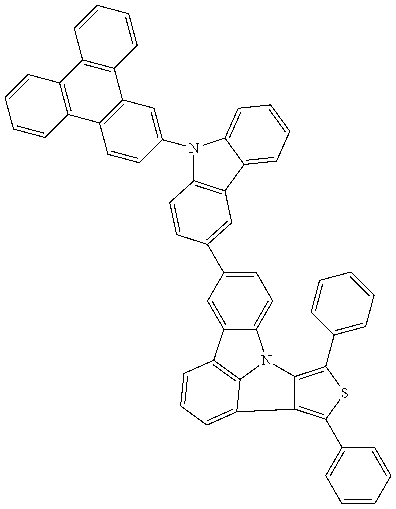 [SMILES: c1ccc(-c2sc(-c3ccccc3)c3c2c2cccc4c5cc(-c6ccc7c(c6)c6ccccc6n7-c6ccc7c8ccccc8c8ccccc8c7c6)ccc5n3c42)cc1]

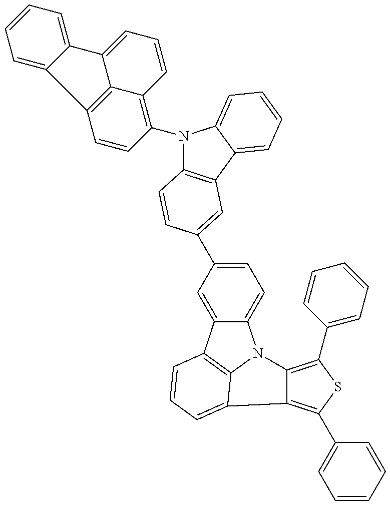 [SMILES: c1ccc(-c2sc(-c3ccccc3)c3c2c2cccc4c5cc(-c6ccc7c(c6)c6ccccc6n7-c6ccc7c8c(cccc68)-c6ccccc6-7)ccc5n3c42)cc1]

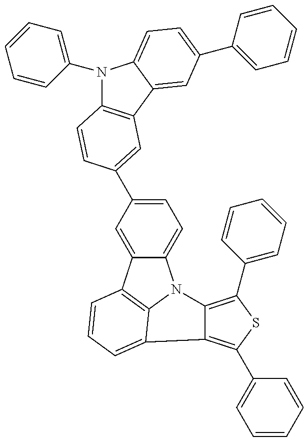 [SMILES: c1ccc(-c2ccc3c(c2)c2cc(-c4ccc5c(c4)c4cccc6c7c(-c8ccccc8)sc(-c8ccccc8)c7n5c46)ccc2n3-c2ccccc2)cc1]

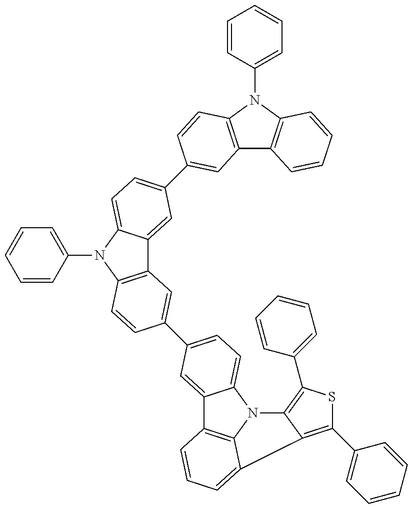 [SMILES: c1ccc(-c2sc(-c3ccccc3)c3c2c2cccc4c5cc(-c6ccc7c(c6)c6cc(-c8ccc9c(c8)c8ccccc8n9-c8ccccc8)ccc6n7-c6ccccc6)ccc5n3c42)cc1]

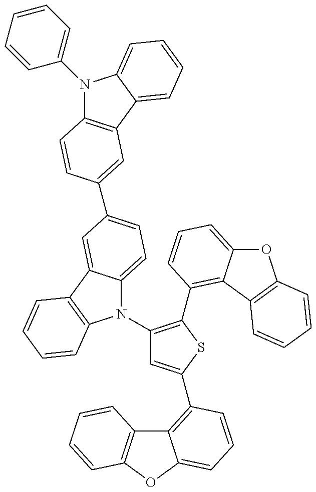 [SMILES: c1ccc(-n2c3ccccc3c3cc(-c4ccc5c(c4)c4ccccc4n5-c4cc(-c5cccc6oc7ccccc7c56)sc4-c4cccc5oc6ccccc6c45)ccc32)cc1]

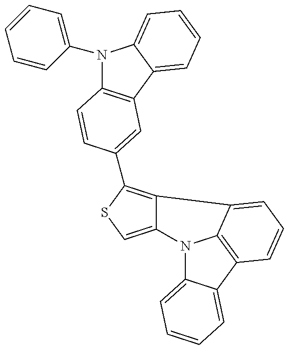 [SMILES: c1ccc(-n2c3ccccc3c3cc(-c4scc5c4c4cccc6c7ccccc7n5c64)ccc32)cc1]

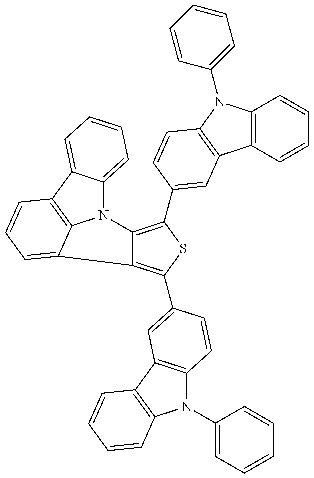 [SMILES: c1ccc(-n2c3ccccc3c3cc(-c4sc(-c5ccc6c(c5)c5ccccc5n6-c5ccccc5)c5c4c4cccc6c7ccccc7n5c64)ccc32)cc1]

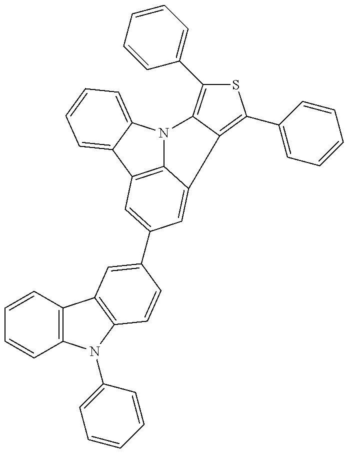 [SMILES: c1ccc(-c2sc(-c3ccccc3)c3c2c2cc(-c4ccc5c(c4)c4ccccc4n5-c4ccccc4)cc4c5ccccc5n3c42)cc1]

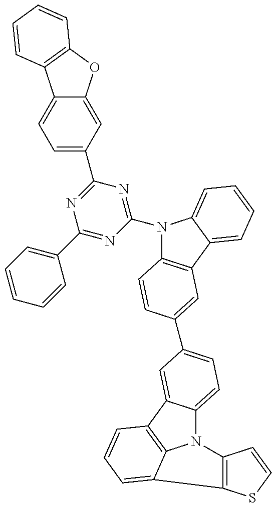 [SMILES: c1ccc(-c2nc(-c3ccc4c(c3)oc3ccccc34)nc(-n3c4ccccc4c4cc(-c5ccc6c(c5)c5cccc7c8sccc8n6c57)ccc43)n2)cc1]

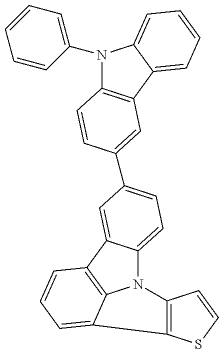 [SMILES: c1ccc(-n2c3ccccc3c3cc(-c4ccc5c(c4)c4cccc6c7sccc7n5c46)ccc32)cc1]

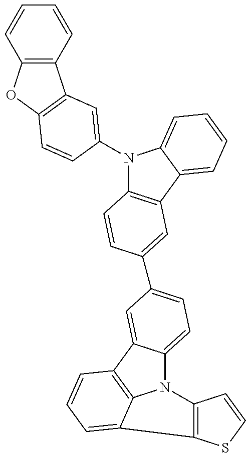 [SMILES: c1ccc2c(c1)oc1ccc(-n3c4ccccc4c4cc(-c5ccc6c(c5)c5cccc7c8sccc8n6c57)ccc43)cc12]

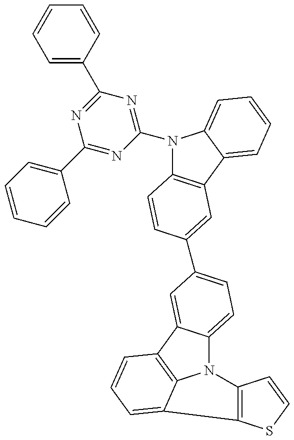 [SMILES: c1ccc(-c2nc(-c3ccccc3)nc(-n3c4ccccc4c4cc(-c5ccc6c(c5)c5cccc7c8sccc8n6c57)ccc43)n2)cc1]

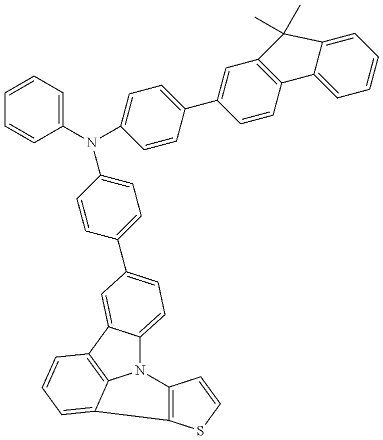 [SMILES: CC1(C)c2ccccc2-c2ccc(-c3ccc(N(c4ccccc4)c4ccc(-c5ccc6c(c5)c5cccc7c8sccc8n6c57)cc4)cc3)cc21]

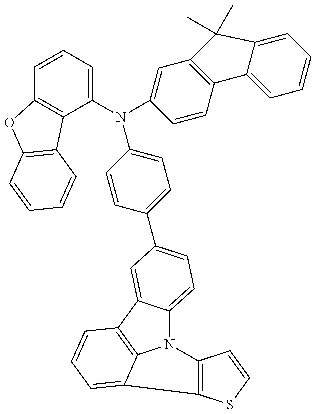 [SMILES: CC1(C)c2ccccc2-c2ccc(N(c3ccc(-c4ccc5c(c4)c4cccc6c7sccc7n5c46)cc3)c3cccc4oc5ccccc5c34)cc21]

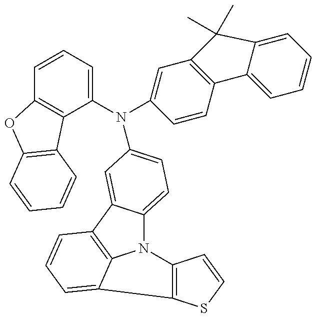 [SMILES: CC1(C)c2ccccc2-c2ccc(N(c3ccc4c(c3)c3cccc5c6sccc6n4c35)c3cccc4oc5ccccc5c34)cc21]

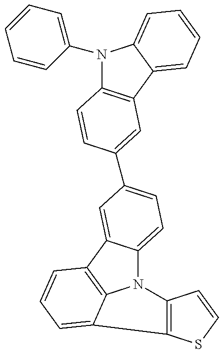 [SMILES: c1ccc(-n2c3ccccc3c3cc(-c4ccc5c(c4)c4cccc6c7sccc7n5c46)ccc32)cc1]

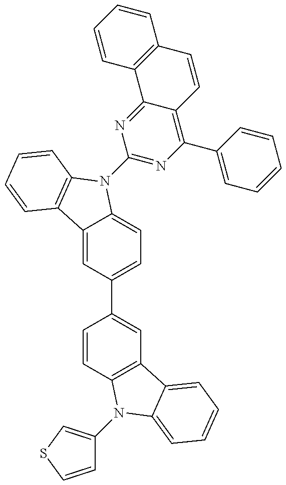 [SMILES: c1ccc(-c2nc(-n3c4ccccc4c4cc(-c5ccc6c(c5)c5ccccc5n6-c5ccsc5)ccc43)nc3c2ccc2ccccc23)cc1]

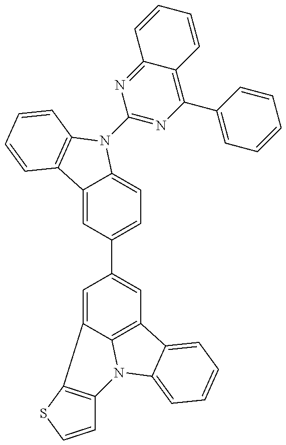 [SMILES: c1ccc(-c2nc(-n3c4ccccc4c4cc(-c5cc6c7ccccc7n7c8ccsc8c(c5)c67)ccc43)nc3ccccc23)cc1]

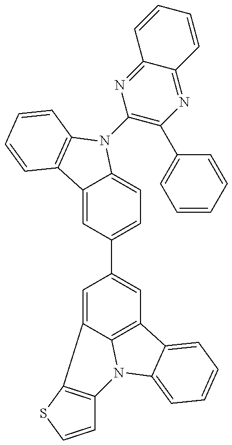 [SMILES: c1ccc(-c2nc3ccccc3nc2-n2c3ccccc3c3cc(-c4cc5c6ccccc6n6c7ccsc7c(c4)c56)ccc32)cc1]

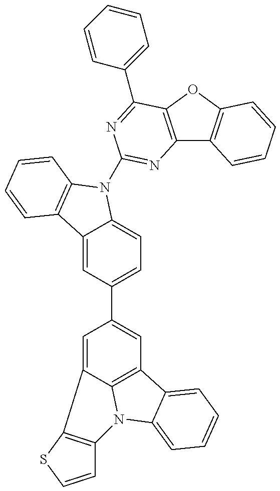 [SMILES: c1ccc(-c2nc(-n3c4ccccc4c4cc(-c5cc6c7ccccc7n7c8ccsc8c(c5)c67)ccc43)nc3c2oc2ccccc23)cc1]

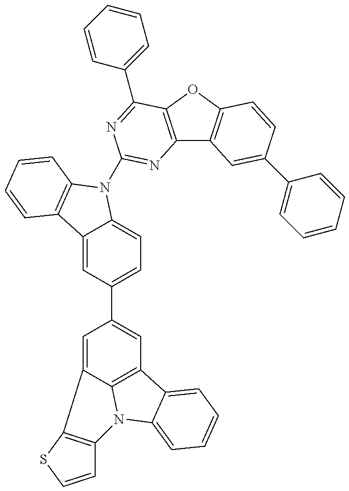 [SMILES: c1ccc(-c2ccc3oc4c(-c5ccccc5)nc(-n5c6ccccc6c6cc(-c7cc8c9ccccc9n9c%10ccsc%10c(c7)c89)ccc65)nc4c3c2)cc1]

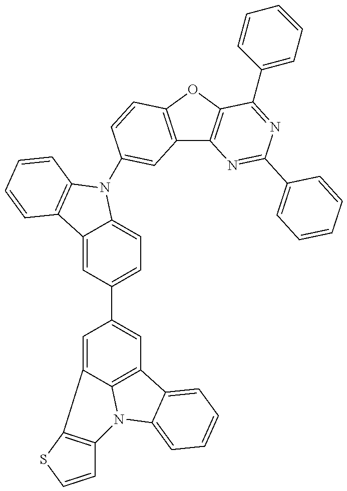 [SMILES: c1ccc(-c2nc(-c3ccccc3)c3oc4ccc(-n5c6ccccc6c6cc(-c7cc8c9ccccc9n9c%10ccsc%10c(c7)c89)ccc65)cc4c3n2)cc1]

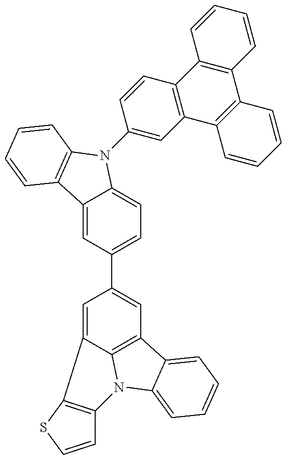 [SMILES: c1ccc2c(c1)c1ccccc1c1cc(-n3c4ccccc4c4cc(-c5cc6c7ccccc7n7c8ccsc8c(c5)c67)ccc43)ccc21]

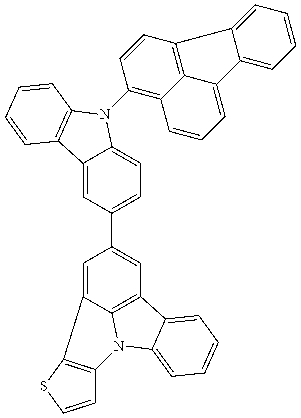 [SMILES: c1ccc2c(c1)-c1cccc3c(-n4c5ccccc5c5cc(-c6cc7c8ccccc8n8c9ccsc9c(c6)c78)ccc54)ccc-2c13]

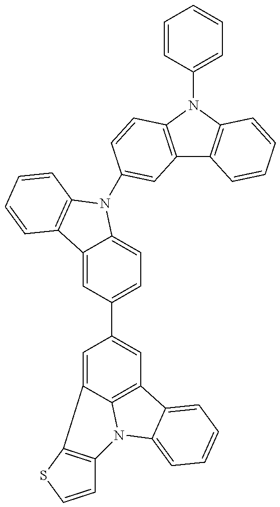 [SMILES: c1ccc(-n2c3ccccc3c3cc(-n4c5ccccc5c5cc(-c6cc7c8ccccc8n8c9ccsc9c(c6)c78)ccc54)ccc32)cc1]

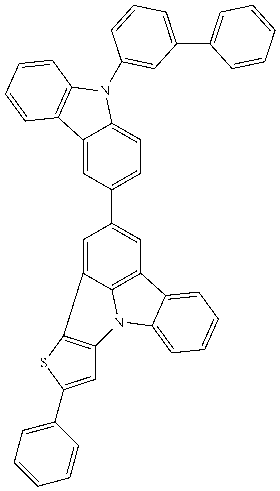 [SMILES: c1ccc(-c2cccc(-n3c4ccccc4c4cc(-c5cc6c7ccccc7n7c8cc(-c9ccccc9)sc8c(c5)c67)ccc43)c2)cc1]

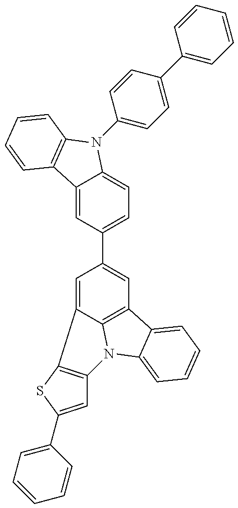 [SMILES: c1ccc(-c2ccc(-n3c4ccccc4c4cc(-c5cc6c7ccccc7n7c8cc(-c9ccccc9)sc8c(c5)c67)ccc43)cc2)cc1]

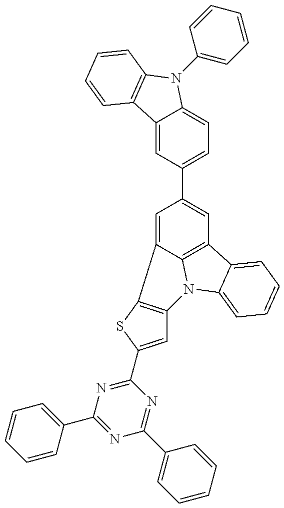 [SMILES: c1ccc(-c2nc(-c3ccccc3)nc(-c3cc4c(s3)c3cc(-c5ccc6c(c5)c5ccccc5n6-c5ccccc5)cc5c6ccccc6n4c53)n2)cc1]

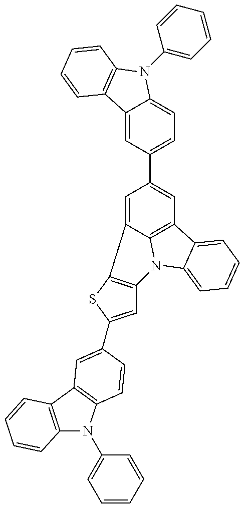 [SMILES: c1ccc(-n2c3ccccc3c3cc(-c4cc5c6ccccc6n6c7cc(-c8ccc9c(c8)c8ccccc8n9-c8ccccc8)sc7c(c4)c56)ccc32)cc1]

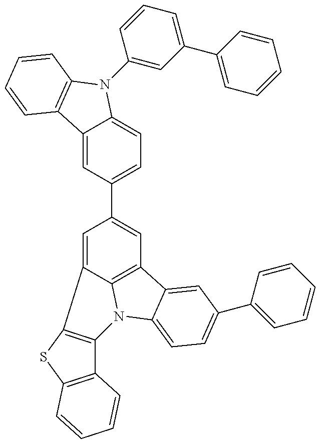 [SMILES: c1ccc(-c2cccc(-n3c4ccccc4c4cc(-c5cc6c7cc(-c8ccccc8)ccc7n7c6c(c5)c5sc6ccccc6c57)ccc43)c2)cc1]

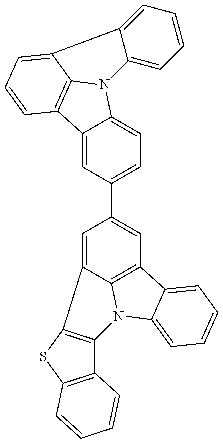 [SMILES: c1ccc2c(c1)sc1c3cc(-c4ccc5c(c4)c4cccc6c7ccccc7n5c64)cc4c5ccccc5n(c43)c21]

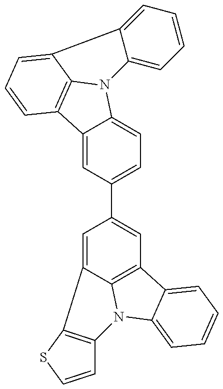 [SMILES: c1ccc2c(c1)c1cccc3c4cc(-c5cc6c7ccccc7n7c8ccsc8c(c5)c67)ccc4n2c13]

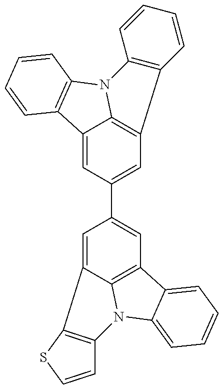 [SMILES: c1ccc2c(c1)c1cc(-c3cc4c5ccccc5n5c6ccsc6c(c3)c45)cc3c4ccccc4n2c13]

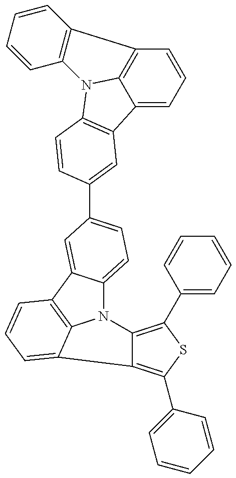 [SMILES: c1ccc(-c2sc(-c3ccccc3)c3c2c2cccc4c5cc(-c6ccc7c(c6)c6cccc8c9ccccc9n7c86)ccc5n3c42)cc1]

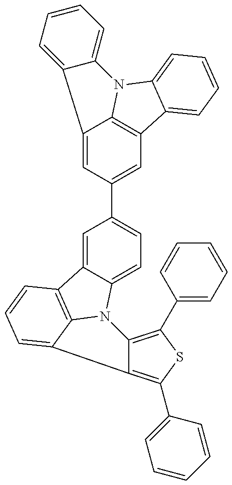 [SMILES: c1ccc(-c2sc(-c3ccccc3)c3c2c2cccc4c5cc(-c6cc7c8ccccc8n8c9ccccc9c(c6)c78)ccc5n3c42)cc1]

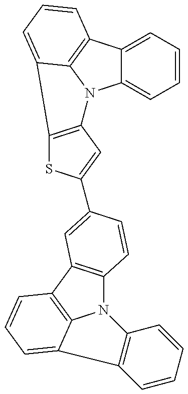 [SMILES: c1ccc2c(c1)c1cccc3c4cc(-c5cc6c(s5)c5cccc7c8ccccc8n6c75)ccc4n2c13]

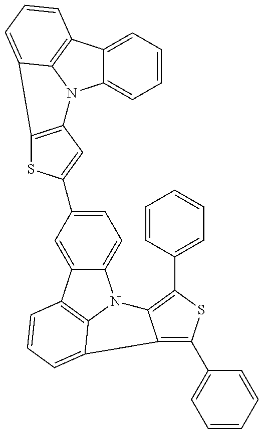 [SMILES: c1ccc(-c2sc(-c3ccccc3)c3c2c2cccc4c5cc(-c6cc7c(s6)c6cccc8c9ccccc9n7c86)ccc5n3c42)cc1]

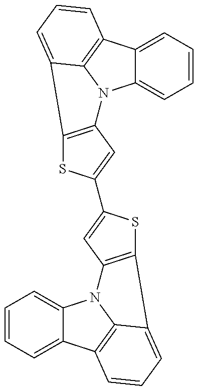 [SMILES: c1ccc2c(c1)c1cccc3c4sc(-c5cc6c(s5)c5cccc7c8ccccc8n6c75)cc4n2c13]

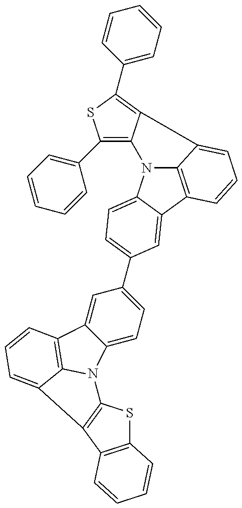 [SMILES: c1ccc(-c2sc(-c3ccccc3)c3c2c2cccc4c5cc(-c6ccc7c(c6)c6cccc8c9c%10ccccc%10sc9n7c68)ccc5n3c42)cc1]

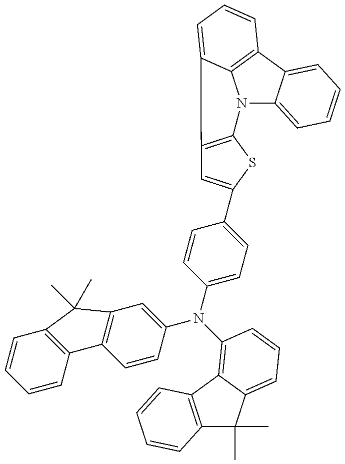 [SMILES: CC1(C)c2ccccc2-c2ccc(N(c3ccc(-c4cc5c6cccc7c8ccccc8n(c5s4)c76)cc3)c3cccc4c3-c3ccccc3C4(C)C)cc21]

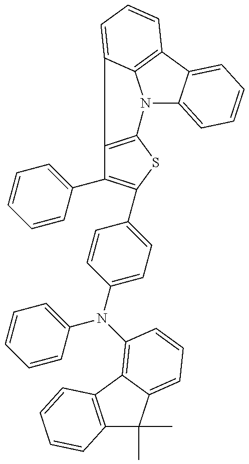 [SMILES: CC1(C)c2ccccc2-c2c(N(c3ccccc3)c3ccc(-c4sc5c(c4-c4ccccc4)c4cccc6c7ccccc7n5c64)cc3)cccc21]

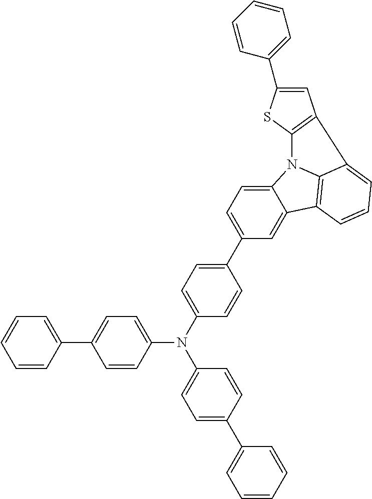 [SMILES: c1ccc(-c2ccc(N(c3ccc(-c4ccccc4)cc3)c3ccc(-c4ccc5c(c4)c4cccc6c7cc(-c8ccccc8)sc7n5c46)cc3)cc2)cc1]

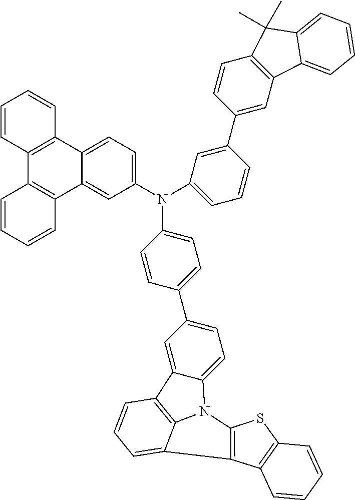 [SMILES: CC1(C)c2ccccc2-c2cc(-c3cccc(N(c4ccc(-c5ccc6c(c5)c5cccc7c8c9ccccc9sc8n6c57)cc4)c4ccc5c6ccccc6c6ccccc6c5c4)c3)ccc21]

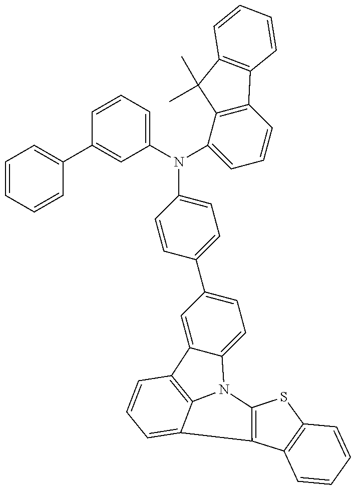 [SMILES: CC1(C)c2ccccc2-c2cccc(N(c3ccc(-c4ccc5c(c4)c4cccc6c7c8ccccc8sc7n5c46)cc3)c3cccc(-c4ccccc4)c3)c21]

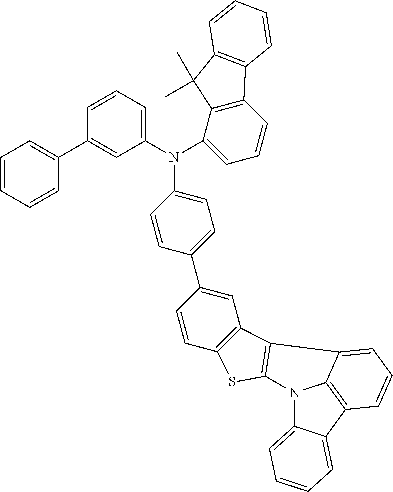 [SMILES: CC1(C)c2ccccc2-c2cccc(N(c3ccc(-c4ccc5sc6c(c5c4)c4cccc5c7ccccc7n6c54)cc3)c3cccc(-c4ccccc4)c3)c21]

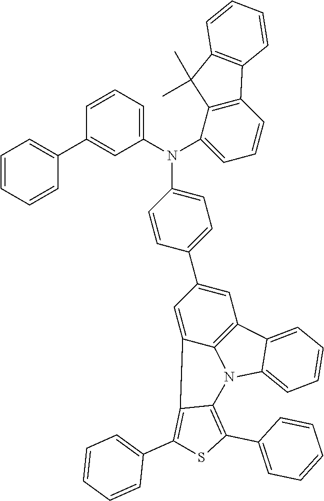 [SMILES: CC1(C)c2ccccc2-c2cccc(N(c3ccc(-c4cc5c6ccccc6n6c7c(-c8ccccc8)sc(-c8ccccc8)c7c(c4)c56)cc3)c3cccc(-c4ccccc4)c3)c21]

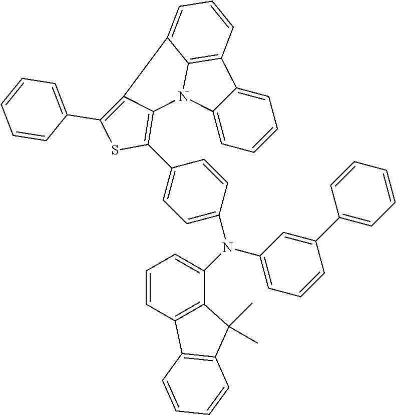 [SMILES: CC1(C)c2ccccc2-c2cccc(N(c3ccc(-c4sc(-c5ccccc5)c5c6cccc7c8ccccc8n(c45)c76)cc3)c3cccc(-c4ccccc4)c3)c21]

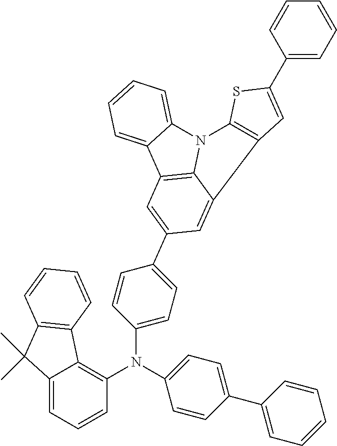 [SMILES: CC1(C)c2ccccc2-c2c(N(c3ccc(-c4ccccc4)cc3)c3ccc(-c4cc5c6ccccc6n6c7sc(-c8ccccc8)cc7c(c4)c56)cc3)cccc21]

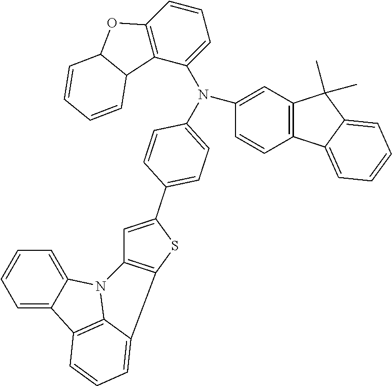 [SMILES: CC1(C)c2ccccc2-c2ccc(N(c3ccc(-c4cc5c(s4)c4cccc6c7ccccc7n5c64)cc3)c3cccc4c3C3C=CC=CC3O4)cc21]

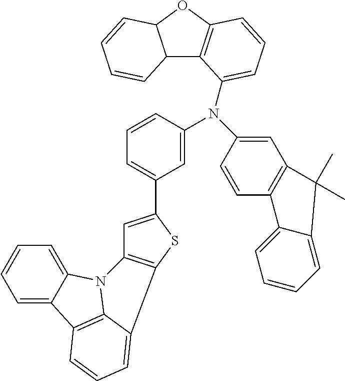 [SMILES: CC1(C)c2ccccc2-c2ccc(N(c3cccc(-c4cc5c(s4)c4cccc6c7ccccc7n5c64)c3)c3cccc4c3C3C=CC=CC3O4)cc21]

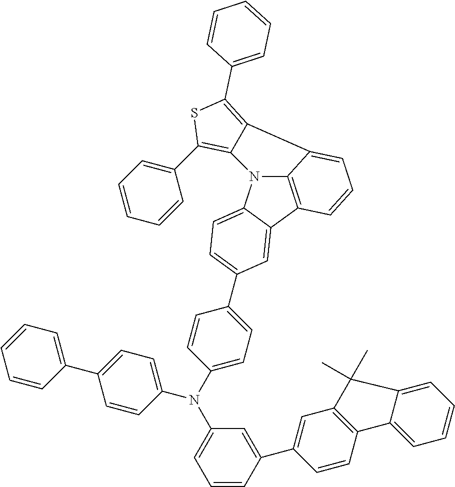 [SMILES: CC1(C)c2ccccc2-c2ccc(-c3cccc(N(c4ccc(-c5ccccc5)cc4)c4ccc(-c5ccc6c(c5)c5cccc7c8c(-c9ccccc9)sc(-c9ccccc9)c8n6c57)cc4)c3)cc21]

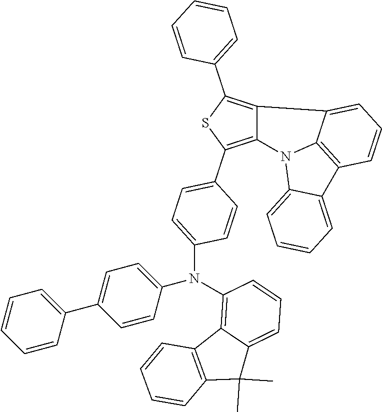 [SMILES: CC1(C)c2ccccc2-c2c(N(c3ccc(-c4ccccc4)cc3)c3ccc(-c4sc(-c5ccccc5)c5c6cccc7c8ccccc8n(c45)c76)cc3)cccc21]

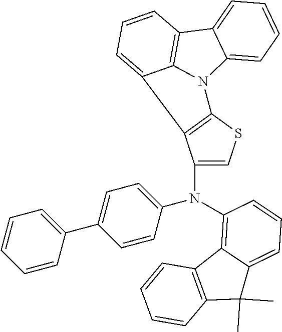 [SMILES: CC1(C)c2ccccc2-c2c(N(c3ccc(-c4ccccc4)cc3)c3csc4c3c3cccc5c6ccccc6n4c53)cccc21]

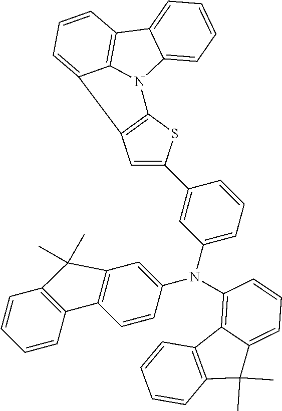 [SMILES: CC1(C)c2ccccc2-c2ccc(N(c3cccc(-c4cc5c6cccc7c8ccccc8n(c5s4)c76)c3)c3cccc4c3-c3ccccc3C4(C)C)cc21]

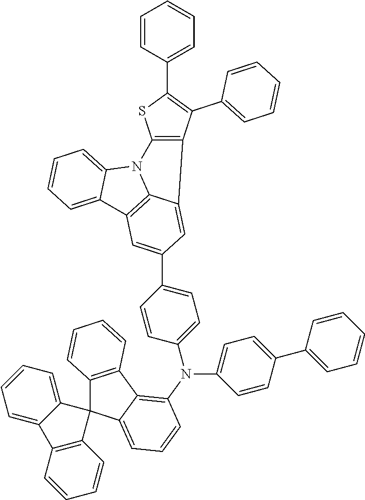 [SMILES: c1ccc(-c2ccc(N(c3ccc(-c4cc5c6ccccc6n6c7sc(-c8ccccc8)c(-c8ccccc8)c7c(c4)c56)cc3)c3cccc4c3-c3ccccc3C43c4ccccc4-c4ccccc43)cc2)cc1]

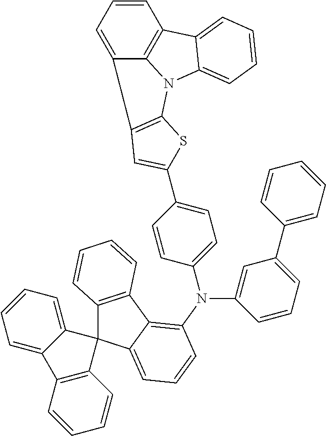 [SMILES: c1ccc(-c2cccc(N(c3ccc(-c4cc5c6cccc7c8ccccc8n(c5s4)c76)cc3)c3cccc4c3-c3ccccc3C43c4ccccc4-c4ccccc43)c2)cc1]

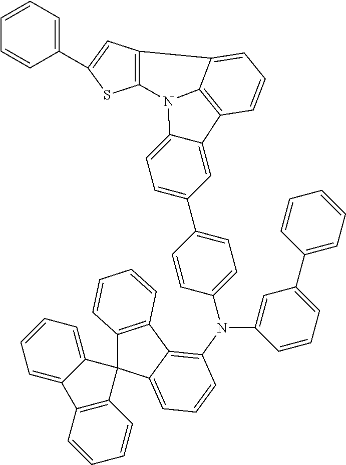 [SMILES: c1ccc(-c2cccc(N(c3ccc(-c4ccc5c(c4)c4cccc6c7cc(-c8ccccc8)sc7n5c46)cc3)c3cccc4c3-c3ccccc3C43c4ccccc4-c4ccccc43)c2)cc1]

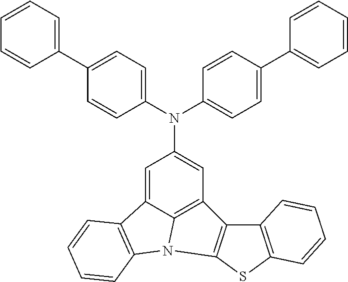 [SMILES: c1ccc(-c2ccc(N(c3ccc(-c4ccccc4)cc3)c3cc4c5ccccc5n5c6sc7ccccc7c6c(c3)c45)cc2)cc1]

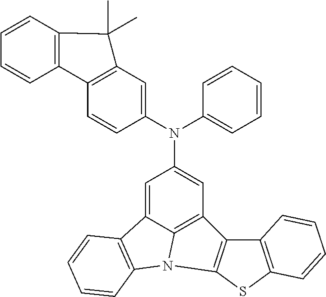 [SMILES: CC1(C)c2ccccc2-c2ccc(N(c3ccccc3)c3cc4c5ccccc5n5c6sc7ccccc7c6c(c3)c45)cc21]

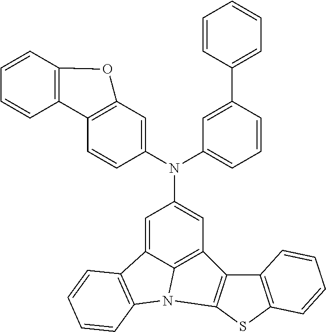 [SMILES: c1ccc(-c2cccc(N(c3ccc4c(c3)oc3ccccc34)c3cc4c5ccccc5n5c6sc7ccccc7c6c(c3)c45)c2)cc1]

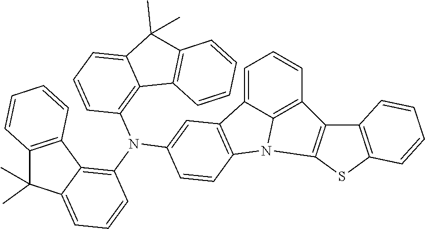 [SMILES: CC1(C)c2ccccc2-c2c(N(c3ccc4c(c3)c3cccc5c6c7ccccc7sc6n4c35)c3cccc4c3-c3ccccc3C4(C)C)cccc21]

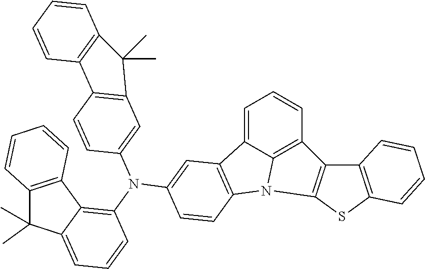 [SMILES: CC1(C)c2ccccc2-c2ccc(N(c3ccc4c(c3)c3cccc5c6c7ccccc7sc6n4c35)c3cccc4c3-c3ccccc3C4(C)C)cc21]

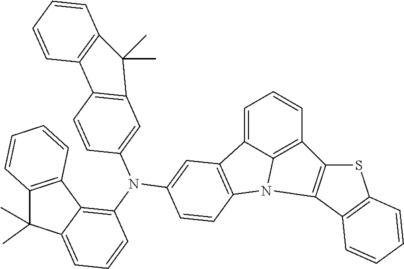 [SMILES: CC1(C)c2ccccc2-c2ccc(N(c3ccc4c(c3)c3cccc5c6sc7ccccc7c6n4c35)c3cccc4c3-c3ccccc3C4(C)C)cc21]

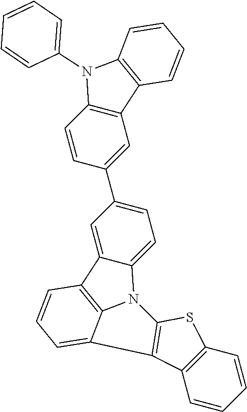 [SMILES: c1ccc(-n2c3ccccc3c3cc(-c4ccc5c(c4)c4cccc6c7c8ccccc8sc7n5c46)ccc32)cc1]

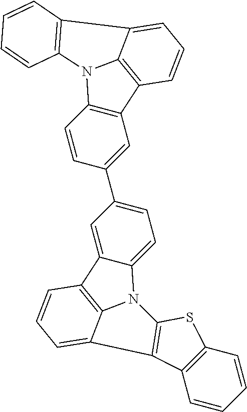 [SMILES: c1ccc2c(c1)sc1c2c2cccc3c4cc(-c5ccc6c(c5)c5cccc7c8ccccc8n6c75)ccc4n1c32]

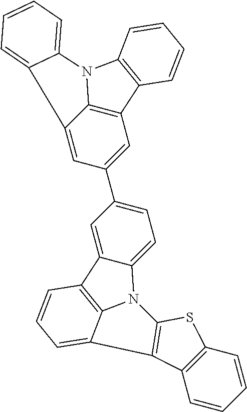 [SMILES: c1ccc2c(c1)sc1c2c2cccc3c4cc(-c5cc6c7ccccc7n7c8ccccc8c(c5)c67)ccc4n1c32]

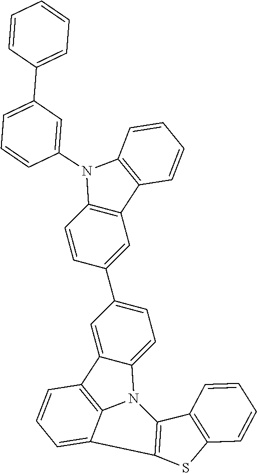 [SMILES: c1ccc(-c2cccc(-n3c4ccccc4c4cc(-c5ccc6c(c5)c5cccc7c8sc9ccccc9c8n6c57)ccc43)c2)cc1]

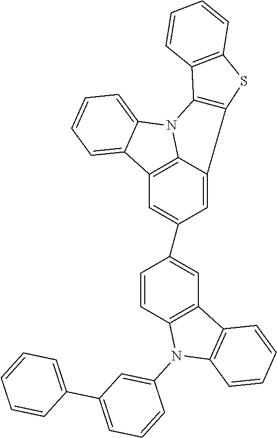 [SMILES: c1ccc(-c2cccc(-n3c4ccccc4c4cc(-c5cc6c7ccccc7n7c6c(c5)c5sc6ccccc6c57)ccc43)c2)cc1]

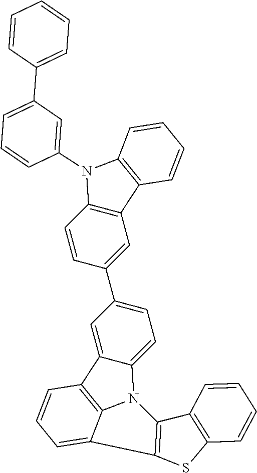 [SMILES: c1ccc(-c2cccc(-n3c4ccccc4c4cc(-c5ccc6c(c5)c5cccc7c8sc9ccccc9c8n6c57)ccc43)c2)cc1]

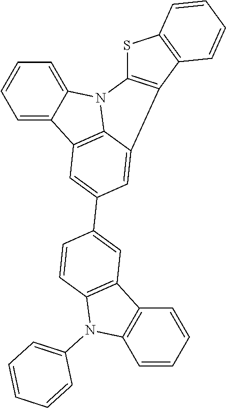 [SMILES: c1ccc(-n2c3ccccc3c3cc(-c4cc5c6ccccc6n6c7sc8ccccc8c7c(c4)c56)ccc32)cc1]

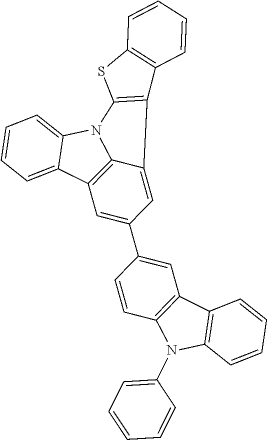 [SMILES: c1ccc(-n2c3ccccc3c3cc(-c4cc5c6ccccc6n6c7sc8ccccc8c7c(c4)c56)ccc32)cc1]

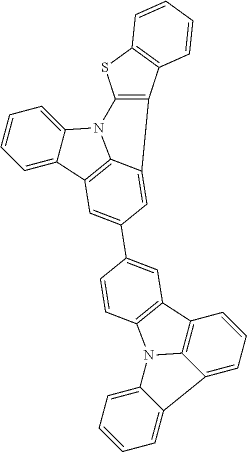 [SMILES: c1ccc2c(c1)sc1c2c2cc(-c3ccc4c(c3)c3cccc5c6ccccc6n4c53)cc3c4ccccc4n1c32]

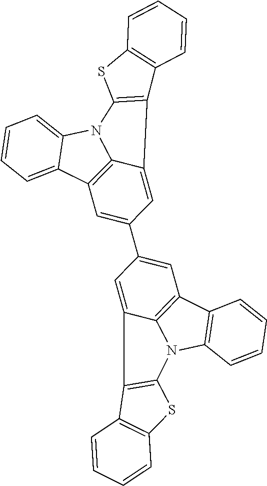 [SMILES: c1ccc2c(c1)sc1c2c2cc(-c3cc4c5ccccc5n5c6sc7ccccc7c6c(c3)c45)cc3c4ccccc4n1c32]

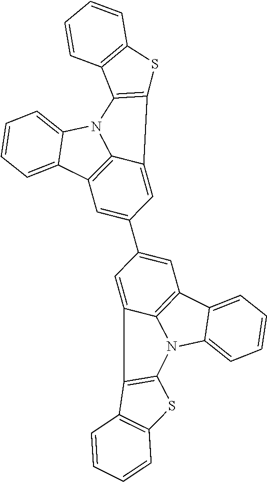 [SMILES: c1ccc2c(c1)sc1c2c2cc(-c3cc4c5ccccc5n5c4c(c3)c3sc4ccccc4c35)cc3c4ccccc4n1c32]

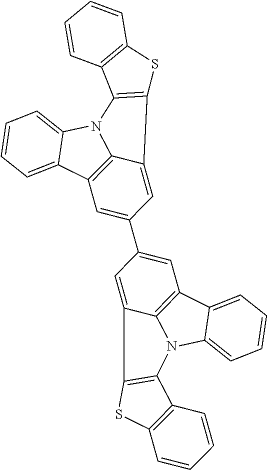 [SMILES: c1ccc2c(c1)sc1c3cc(-c4cc5c6ccccc6n6c5c(c4)c4sc5ccccc5c46)cc4c5ccccc5n(c43)c21]

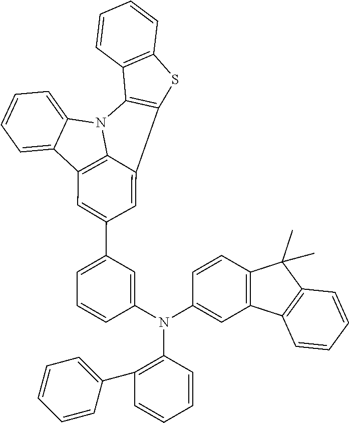 [SMILES: CC1(C)c2ccccc2-c2cc(N(c3cccc(-c4cc5c6ccccc6n6c5c(c4)c4sc5ccccc5c46)c3)c3ccccc3-c3ccccc3)ccc21]

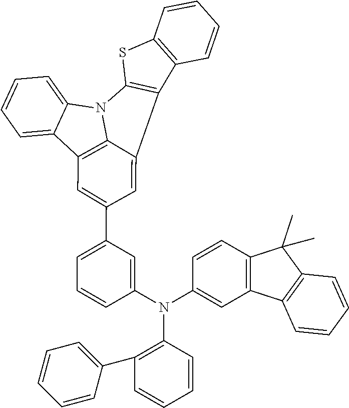 [SMILES: CC1(C)c2ccccc2-c2cc(N(c3cccc(-c4cc5c6ccccc6n6c7sc8ccccc8c7c(c4)c56)c3)c3ccccc3-c3ccccc3)ccc21]

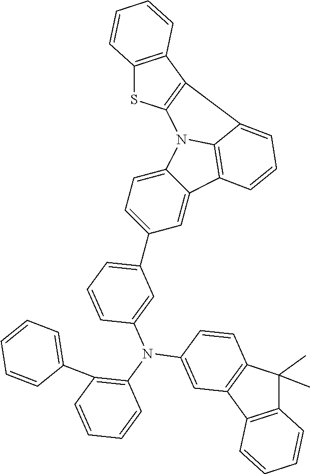 [SMILES: CC1(C)c2ccccc2-c2cc(N(c3cccc(-c4ccc5c(c4)c4cccc6c7c8ccccc8sc7n5c46)c3)c3ccccc3-c3ccccc3)ccc21]

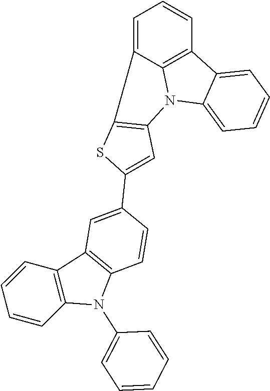 [SMILES: c1ccc(-n2c3ccccc3c3cc(-c4cc5c(s4)c4cccc6c7ccccc7n5c64)ccc32)cc1]

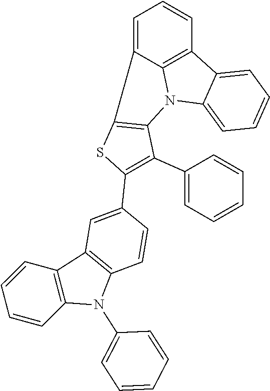 [SMILES: c1ccc(-c2c(-c3ccc4c(c3)c3ccccc3n4-c3ccccc3)sc3c4cccc5c6ccccc6n(c23)c54)cc1]

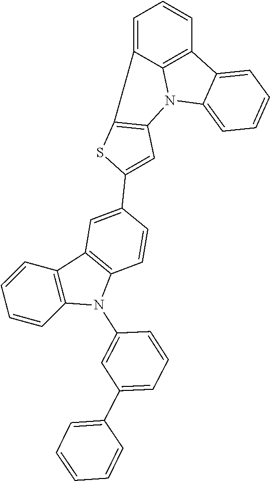 [SMILES: c1ccc(-c2cccc(-n3c4ccccc4c4cc(-c5cc6c(s5)c5cccc7c8ccccc8n6c75)ccc43)c2)cc1]

The base structure of the compounds of the invention can be prepared by the routes outlined in the schemes which follow. The individual synthesis steps, for example C—C coupling reactions according to Suzuki, C—N coupling reactions according to Hartwig-Buchwald or cyclization reactions, are known in principle to those skilled in the art. Further information relating to the synthesis of the compounds of the invention can be found in the synthesis examples. One possible synthesis of the base structure is shown in scheme 1. This can be effected by the reactions set out in U.S. Ser. No. 10/312,455 B2. Alternatively, the coupling can be effected with the amino group of an optionally substituted carbazole, followed by a ring closure reaction. Schemes 3 to 5 show various options for the introduction of the fluorene, dibenzofuran, dibenzothiophene or carbazole group. It is possible here to introduce a fluorene, dibenzofuran, dibenzothiophene or carbazole compound substituted by a suitable reactive group, for example a boron-containing group, in a Suzuki coupling reaction, as shown in schemes 3 to 5:

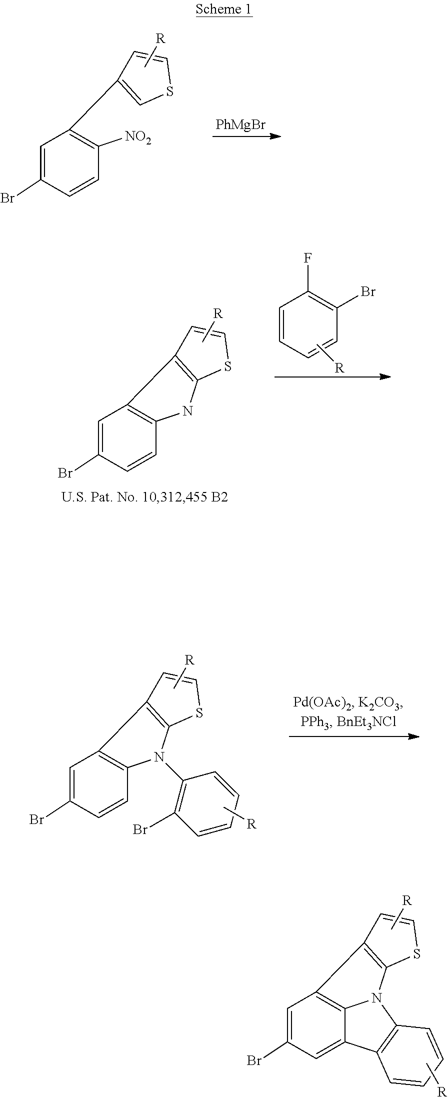

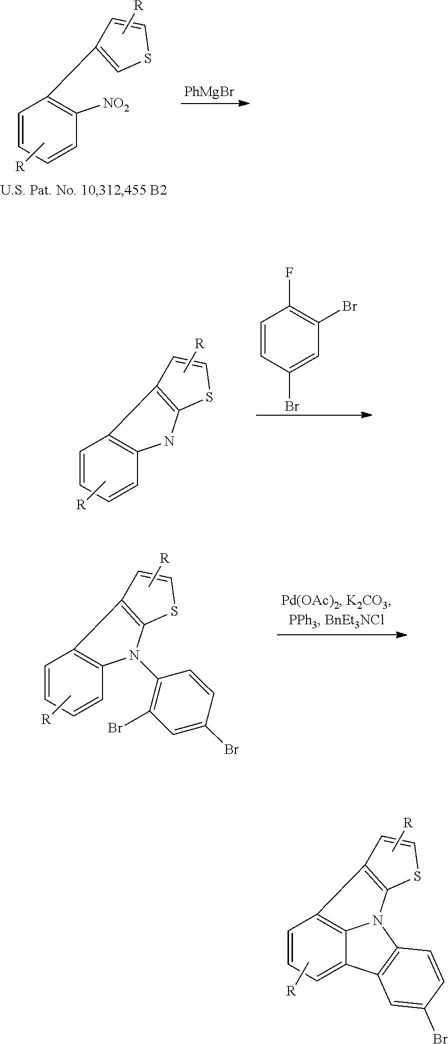

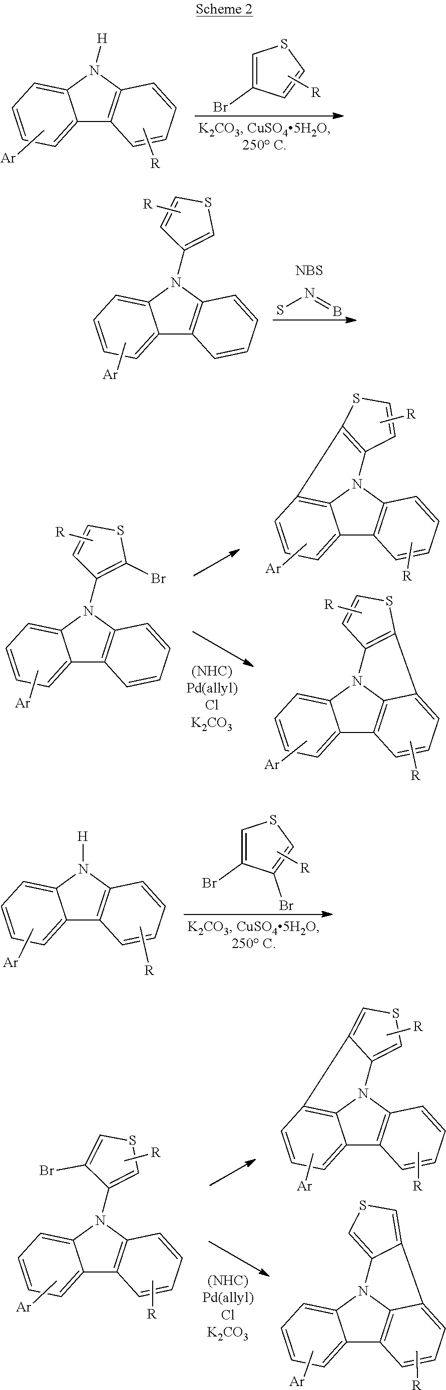

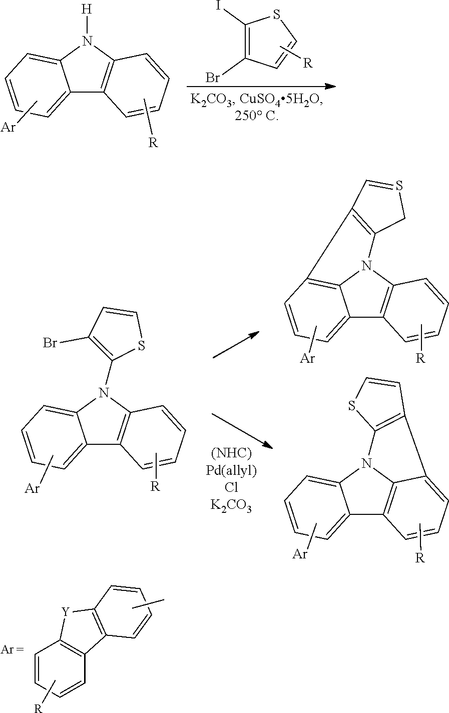

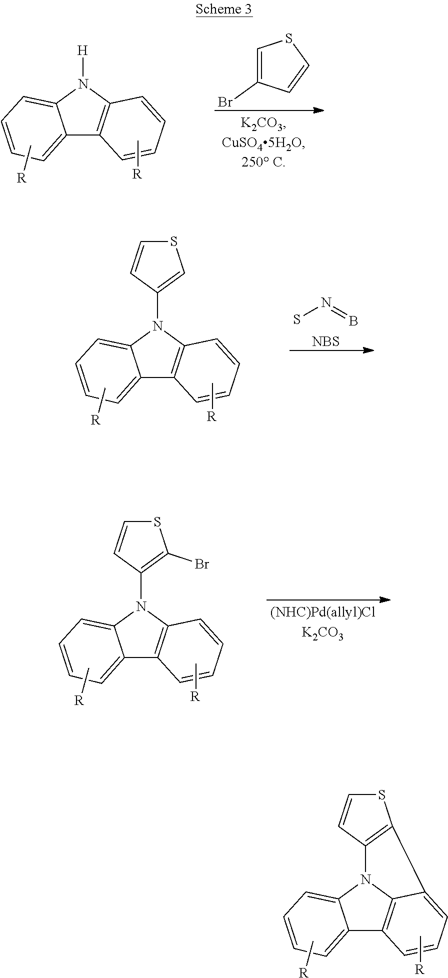

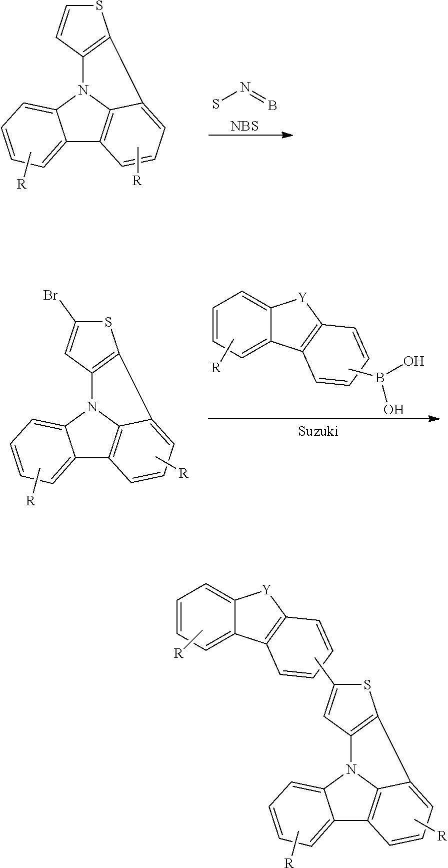

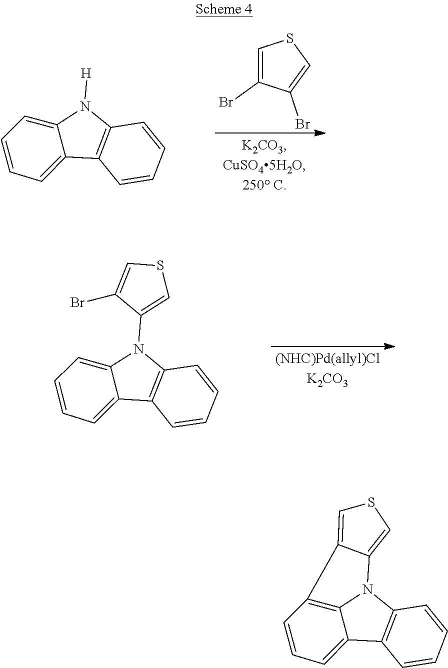 [SMILES: c1ccc2c(c1)[nH]c1ccccc12 ; Brc1cscc1Br ; Brc1cscc1-n1c2ccccc2c2ccccc21 ; c1ccc2c(c1)c1cccc3c4cscc4n2c13]

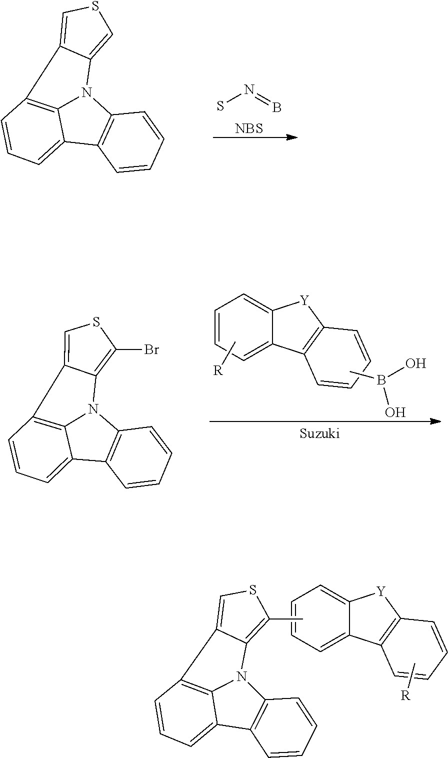

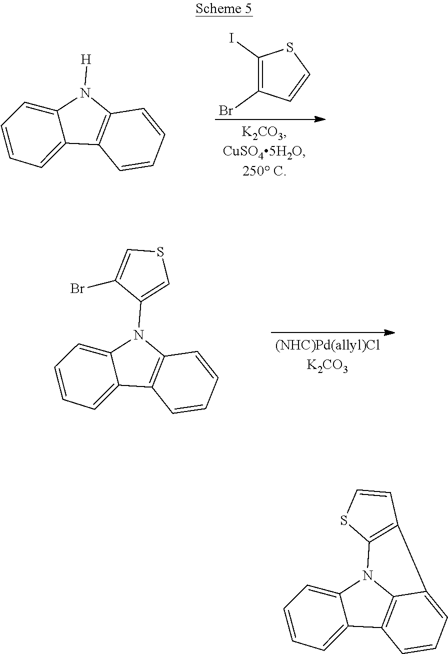 [SMILES: c1ccc2c(c1)[nH]c1ccccc12 ; Brc1ccsc1I ; Brc1cscc1-n1c2ccccc2c2ccccc21 ; c1ccc2c(c1)c1cccc3c4ccsc4n2c13]

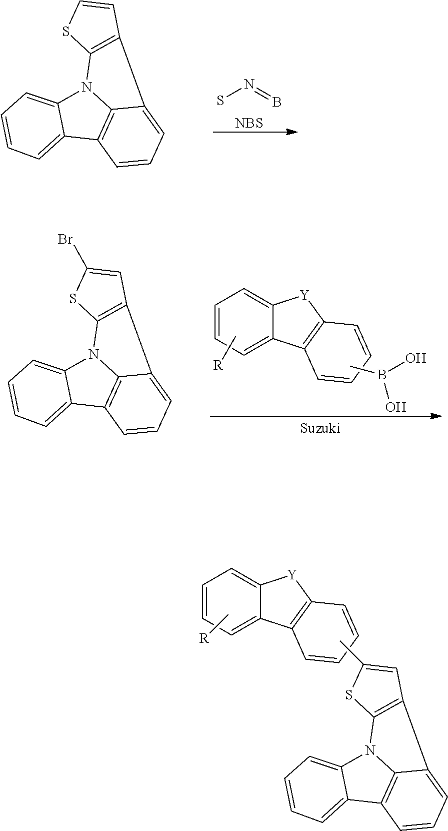

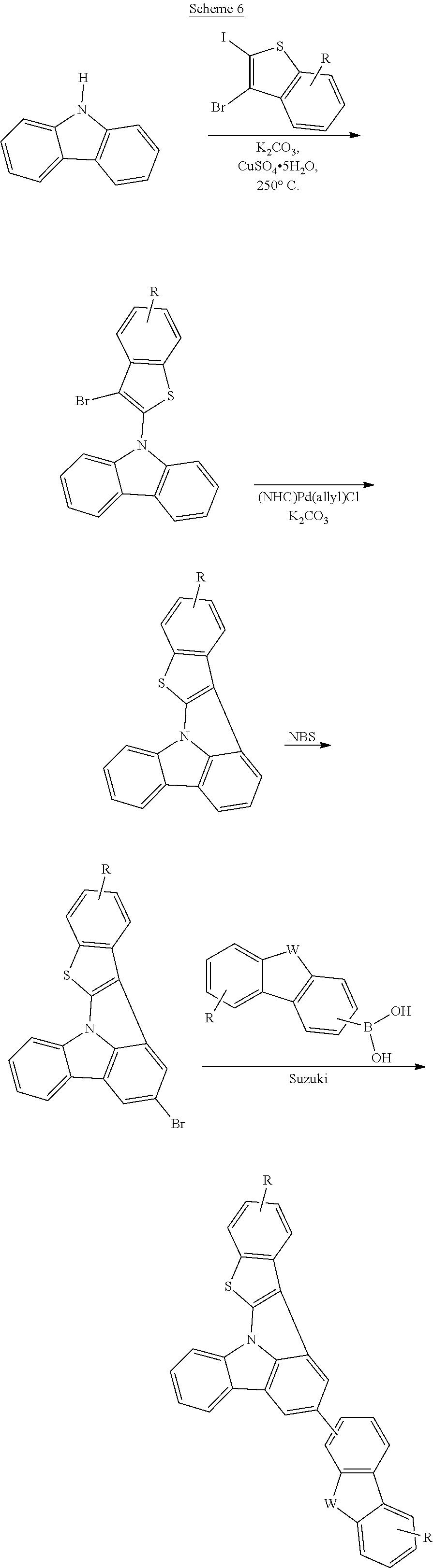

The definition of the symbols used in schemes 1 to 6 corresponds essentially to that which was defined for formula (1), dispensing with numbering and complete representation of all symbols for reasons of clarity.

The present invention therefore further provides a process for preparing a compound of the invention, wherein a thiofuran compound is reacted with an aromatic or heteroaromatic nitrogen compound by means of a coupling reaction.

For the processing of the compounds of the invention from a liquid phase, for example by spin-coating or by printing methods, formulations of the compounds of the invention are required. These formulations may, for example, be solutions, dispersions or emulsions. For this purpose, it may be preferable to use mixtures of two or more solvents. Suitable and preferred solvents are, for example, toluene, anisole, o-, m- or p-xylene, methyl benzoate, mesitylene, tetralin, veratrole, THF, methyl-THF, THP, chlorobenzene, dioxane, phenoxytoluene, especially 3-phenoxytoluene, (−)-fenchone, 1,2,3,5-tetramethylbenzene, 1,2,4,5-tetramethylbenzene, 1-methylnaphthalene, 2-methylbenzothiazole, 2-phenoxyethanol, 2-pyrrolidinone, 3-methylanisole, 4-methylanisole, 3,4-dimethylanisole, 3,5-dimethylanisole, acetophenone, α-terpineol, benzothiazole, butyl benzoate, cumene, cyclohexanol, cyclohexanone, cyclohexylbenzene, decalin, dodecylbenzene, ethyl benzoate, indane, NMP, p-cymene, phenetole, 1,4-diisopropylbenzene, dibenzyl ether, diethylene glycol butyl methyl ether, triethylene glycol butyl methyl ether, diethylene glycol dibutyl ether, triethylene glycol dimethyl ether, diethylene glycol monobutyl ether, tripropylene glycol dimethyl ether, tetraethylene glycol dimethyl ether, 2-isopropylnaphthalene, pentylbenzene, hexylbenzene, heptylbenzene, octylbenzene, 1,1-bis(3,4-dimethylphenyl)ethane, 2-methylbiphenyl, 3-methylbiphenyl, 1-methylnaphthalene, 1-ethylnaphthalene, ethyl octanoate, diethyl sebacate, octyl octanoate, heptylbenzene, menthyl isovalerate, cyclohexyl hexanoate or mixtures of these solvents.

The present invention therefore further provides a formulation or a composition comprising at least one compound of the invention and at least one further compound. The further compound may, for example, be a solvent, especially one of the abovementioned solvents or a mixture of these solvents. If the further compound comprises a solvent, this mixture is referred to herein as formulation. The further compound may alternatively be at least one further organic or inorganic compound which is likewise used in the electronic device, for example an emitting compound and/or a further matrix material. Suitable emitting compounds and further matrix materials are listed at the back in connection with the organic electroluminescent device. The further compound may also be polymeric.

The present invention further provides for the use of a compound of the invention in an electronic device, especially in an organic electroluminescent device.

The present invention still further provides an electronic device comprising at least one compound of the invention. An electronic device in the context of the present invention is a device comprising at least one layer comprising at least one organic compound. This component may also comprise inorganic materials or else layers formed entirely from inorganic materials.

The electronic device is more preferably selected from the group consisting of organic electroluminescent devices (OLEDs, sOLED, PLEDs, LECs, etc.), preferably organic light-emitting diodes (OLEDs), organic light-emitting diodes based on small molecules (sOLEDs), organic light-emitting diodes based on polymers (PLEDs), light-emitting electrochemical cells (LECs), organic laser diodes (O-laser), organic plasmon-emitting devices (D. M. Koller et al., Nature Photonics 2008, 1-4), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs) and organic electrical sensors, preferably organic electroluminescent devices (OLEDs, sOLED, PLEDs, LECs, etc.), more preferably organic light-emitting diodes (OLEDs), organic light-emitting diodes based on small molecules (sOLEDs), organic light-emitting diodes based on polymers (PLEDs), especially phosphorescent OLEDs.

The organic electroluminescent device comprises cathode, anode and at least one emitting layer. Apart from these layers, it may also comprise further layers, for example in each case one or more hole injection layers, hole transport layers, hole blocker layers, electron transport layers, electron injection layers, exciton blocker layers, electron blocker layers and/or charge generation layers. It is likewise possible for interlayers having an exciton-blocking function, for example, to be introduced between two emitting layers. However, it should be pointed out that not necessarily every one of these layers need be present. In this case, it is possible for the organic electroluminescent device to contain an emitting layer, or for it to contain a plurality of emitting layers. If a plurality of emission layers are present, these preferably have several emission maxima between 380 nm and 750 nm overall, such that the overall result is white emission; in other words, various emitting compounds which may fluoresce or phosphoresce are used in the emitting layers. Especially preferred are systems having three emitting layers, where the three layers show blue, green and orange or red emission. The organic electroluminescent device of the invention may also be a tandem electroluminescent device, especially for white-emitting OLEDs.

The compound of the invention may be used in different layers, according to the exact structure. Preference is given to an organic electroluminescent device comprising a compound of formula (1) or the above-recited preferred embodiments in an emitting layer as matrix material for phosphorescent emitters or for emitters that exhibit TADF (thermally activated delayed fluorescence), especially for phosphorescent emitters. In addition, the compound of the invention can also be used in an electron transport layer and/or in a hole transport layer and/or in an exciton blocker layer and/or in a hole blocker layer. More preferably, the compound of the invention is used as matrix material for phosphorescent emitters, especially for red-, orange-, green- or yellow-phosphorescing, preferably green-phosphorescing, emitters in an emitting layer or as hole transport or electron blocker material in a hole transport or electron blocker layer, more preferably as matrix material in an emitting layer.

When the compound of the invention is used as matrix material for a phosphorescent compound in an emitting layer, it is preferably used in combination with one or more phosphorescent materials (triplet emitters). Phosphorescence in the context of this invention is understood to mean luminescence from an excited state having higher spin multiplicity, i.e. a spin state >1, especially from an excited triplet state. In the context of this application, all luminescent complexes with transition metals or lanthanides, especially all iridium, platinum and copper complexes, shall be regarded as phosphorescent compounds.

The mixture of the compound of the invention and the emitting compound contains between 99% and 1% by volume, preferably between 98% and 10% by volume, more preferably between 97% and 60% by volume and especially between 95% and 80% by volume of the compound of the invention, based on the overall mixture of emitter and matrix material. Correspondingly, the mixture contains between 1% and 99% by volume, preferably between 2% and 90% by volume, more preferably between 3% and 40% by volume and especially between 5% and 20% by volume of the emitter, based on the overall mixture of emitter and matrix material.

In one embodiment of the invention, the compound of the invention is used here as the sole matrix material (“single host”) for the phosphorescent emitter.

A further embodiment of the present invention is the use of the compound of the invention as matrix material for a phosphorescent emitter in combination with a further matrix material. Suitable matrix materials which can be used in combination with the inventive compounds are aromatic ketones, aromatic phosphine oxides or aromatic sulfoxides or sulfones, for example according to WO 2004/013080, WO 2004/093207, WO 2006/005627 or WO 2010/006680, triarylamines, carbazole derivatives, e.g. CBP (N,N-biscarbazolylbiphenyl) or the carbazole derivatives disclosed in WO 2005/039246, US 2005/0069729, JP 2004/288381, EP 1205527, WO 2008/086851 or WO 2013/041176, indolocarbazole derivatives, for example according to WO 2007/063754 or WO 2008/056746, indenocarbazole derivatives, for example according to WO 2010/136109, WO 2011/000455, WO 2013/041176 or WO 2013/056776, azacarbazole derivatives, for example according to EP 1617710, EP 1617711, EP 1731584, JP 2005/347160, bipolar matrix materials, for example according to WO 2007/137725, silanes, for example according to WO 2005/111172, azaboroles or boronic esters, for example according to WO 2006/117052, triazine derivatives, for example according to WO 2007/063754, WO 2008/056746, WO 2010/015306, WO 2011/057706, WO 2011/060859 or WO 2011/060877, zinc complexes, for example according to EP 652273 or WO 2009/062578, diazasilole or tetraazasilole derivatives, for example according to WO 2010/054729, diazaphosphole derivatives, for example according to WO 2010/054730, bridged carbazole derivatives, for example according to WO 2011/042107, WO 2011/060867, WO 2011/088877 and WO 2012/143080, triphenylene derivatives, for example according to WO 2012/048781, dibenzofuran derivatives, for example according to WO 2015/169412, WO 2016/015810, WO 2016/023608, WO 2017/148564 or WO 2017/148565, or biscarbazoles, for example according to JP 3139321 B2.

It is likewise possible for a further phosphorescent emitter which emits at a shorter wavelength than the actual emitter to be present as co-host in the mixture. Particularly good results are achieved when the emitter used is a red-phosphorescing emitter and the co-host used in combination with the compound of the invention is a yellow-phosphorescing emitter.

In addition, the co-host used may be a compound that does not take part in charge transport to a significant degree, if at all, as described, for example, in WO 2010/108579. Especially suitable in combination with the compound of the invention as co-matrix material are compounds which have a large bandgap and themselves take part at least not to a significant degree, if any at all, in the charge transport of the emitting layer. Such materials are preferably pure hydrocarbons. Examples of such materials can be found, for example, in WO 2009/124627 or in WO 2010/006680.

Particularly preferred co-host materials which can be used in combination with the compounds of the invention are compounds of one of the formulae (7), (8), (9) and (10):

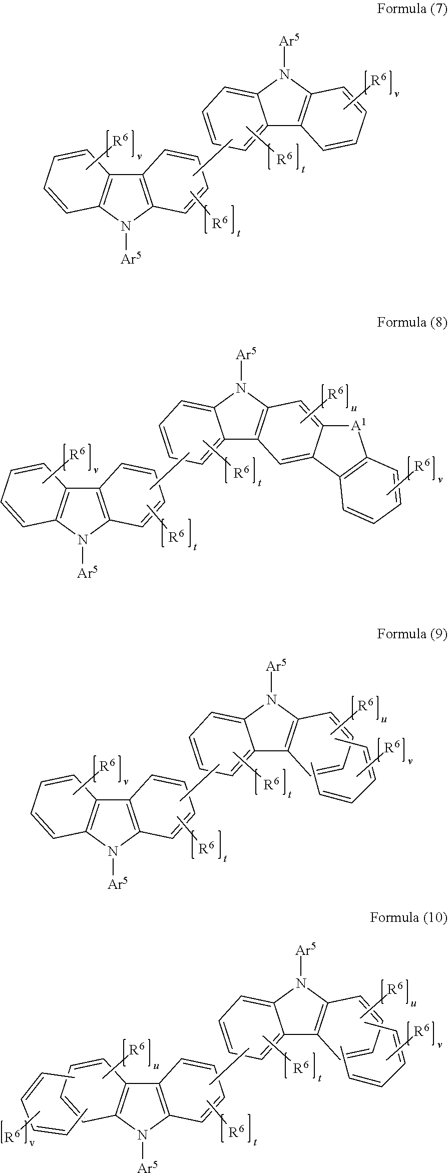

where the symbols and indices used are as follows:
R⁶ is the same or different at each instance and is H, D, F, Cl, Br, I, N(R⁷)₂, N(Ar″)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, preferably 5 to 40 aromatic ring atoms, and may be substituted in each case by one or more R⁷ radicals; at the same time, two R⁶ radicals together may also form an aromatic, heteroaromatic, aliphatic or heteroaliphatic ring system; preferably, the R⁶ radicals do not form any such ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S,
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, ORB, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system; preferably, the R⁷ radicals do not form any such ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
s is the same or different at each instance and is 0, 1, 2, 3 or 4, preferably 0 or 1 and very preferably 0;
t is the same or different at each instance and is 0, 1, 2 or 3, preferably 0 or 1 and very preferably 0;
u is the same or different at each instance and is 0, 1 or 2, preferably 0 or 1 and very preferably 0.

The sum total of the indices s, t and u in compounds of the formulae (7), (8), (9) and (10) is preferably not more than 6, especially preferably not more than 4 and more preferably not more than 2.

In a preferred embodiment of the invention, R⁶ is the same or different at each instance and is selected from the group consisting of H, D, F, CN, NO₂, Si(R⁷)₃, B(OR⁷)₂, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R⁷ radicals, or an aromatic or heteroaromatic ring system which has 5 to 60 aromatic ring atoms, preferably 5 to 40 aromatic ring atoms, and may be substituted in each case by one or more R⁷ radicals.

In a further-preferred embodiment of the invention, R⁶ is the same or different at each instance and is selected from the group consisting of H, D, an aromatic or heteroaromatic ring system which has 6 to 30 aromatic ring atoms and may be substituted by one or more R⁷ radicals, and an N(Ar″)₂ group. More preferably, R⁶ is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R⁷ radicals.

Preferred aromatic or heteroaromatic ring systems R⁶ or Ar″ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ radicals. The structures Ar-1 to Ar-75 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-75 set out above, in relation to the R⁶ and Ar″ radicals, the substituents R⁴ should be replaced by the corresponding R⁷ radicals. The preferences set out above for the R² and R³ groups are correspondingly applicable to the R⁶ group.

Further suitable R⁶ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40. Further preferences for the Ar², Ar³ and Ar⁴ groups have been set out above and are correspondingly applicable.

It may further be the case that the substituents R⁶ according to the above formulae do not form a fused aromatic or heteroaromatic ring system, preferably any fused ring system, with the ring atoms of the ring system. This includes the formation of a fused ring system with possible substituents R⁷, R⁸ which may be bonded to the R⁶ radicals.

When A¹ is NR⁷, the substituent R⁷ bonded to the nitrogen atom is preferably an aromatic or heteroaromatic ring system which has 5 to 24 aromatic ring atoms and may also be substituted by one or more R⁸ radicals. In a particularly preferred embodiment, this R⁷ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R⁸ radicals. Preference is given to phenyl, biphenyl, terphenyl and quaterphenyl having bonding patterns as listed above for Ar-1 to Ar-11, where these structures, rather than by R⁴, may be substituted by one or more R⁸ radicals, but are preferably unsubstituted. Preference is further given to triazine, pyrimidine and quinazoline as listed above for Ar-47 to Ar-Ar-57 and Ar-58, where these structures, rather than by R⁴, may be substituted by one or more R⁸ radicals.

When A¹ is C(R⁷)₂, the substituents R⁷ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R⁸ radicals. Most preferably, R⁷ is a methyl group or a phenyl group. In this case, the R⁷ radicals together may also form a ring system, which leads to a spiro system.

In addition, preferred co-host materials which can be used in combination with the compounds of the invention are compounds of one of the formulae (11), (12), (13), (14), (15), (16), (17) and (18):

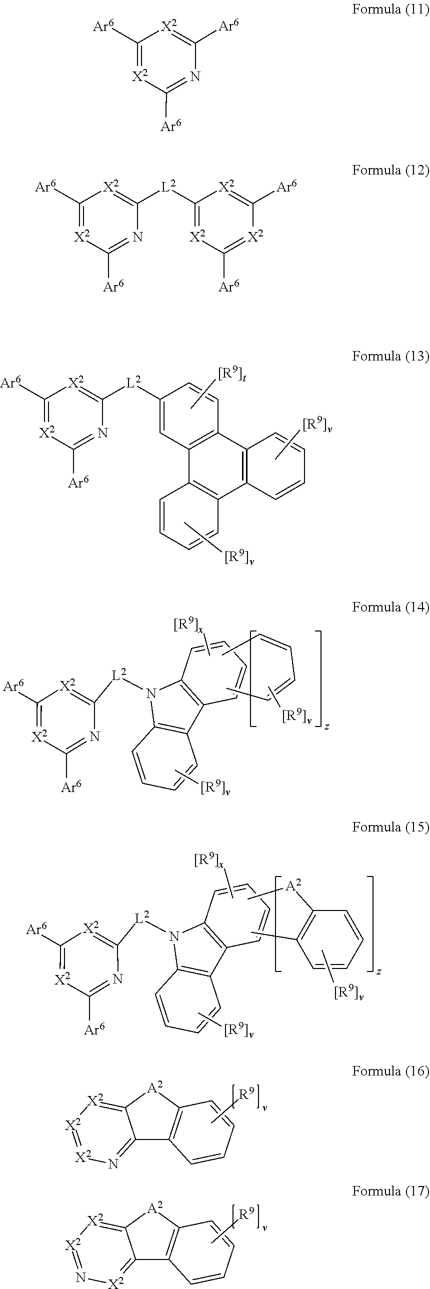

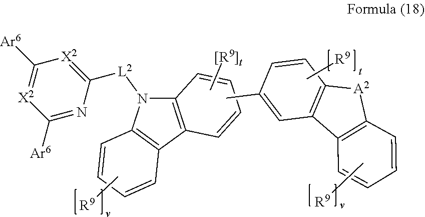

where the symbols and indices used are as follows:
X² is N or CR⁹, with the proviso that not more than two of the X² groups in one cycle are N, and preferably at least one X² is N;
L² is a connecting group which is preferably selected from a bond or an aromatic or heteroaromatic ring system which has 5 to 40 aromatic ring atoms and may be substituted by one or more R⁹ radicals, more preferably a bond;
A² is C(R¹⁰)₂, NR¹⁰, O or S;
Ar⁶ is the same or different at each instance and is an aromatic or heteroaromatic ring system which has 5 to 40 aromatic ring atoms and may be substituted by one or more R¹⁰ radicals;
R⁹ is the same or different at each instance and is H, D, F, Cl, Br, I, N(R¹⁰)₂, N(Ar″)₂, CN, NO₂, OR¹⁰, SR¹⁰, COOR¹⁰, C(═O)N(R¹⁰)₂, Si(R¹⁰)₃, B(OR¹⁰)₂, C(═O)R¹⁰, P(═O)(R¹⁰)₂, S(═O)R¹⁰, S(═O)₂R¹⁰, OSO₂R¹⁰, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals, where one or more nonadjacent CH₂ groups may be replaced by Si(R¹⁰)₂, C═O, NR¹⁰, O, S or CONR¹⁰, or an aromatic or heteroaromatic ring system which has 5 to 60 aromatic ring atoms, preferably 5 to 40 aromatic ring atoms, and may be substituted in each case by one or more R¹⁰ radicals; at the same time, two R⁹ radicals together may also form an aromatic, heteroaromatic, aliphatic or heteroaliphatic ring system; preferably, the R⁹ radicals do not form any such ring system;
Ar′″ is the same or different at each instance and is an aromatic or heteroaromatic ring system which has 5 to 40 aromatic ring atoms and may be substituted by one or more R¹⁰ radicals;
R¹⁰ is the same or different at each instance and is H, D, F, Cl, Br, I, N(R¹¹)₂, CN, NO₂, OR¹¹, SR¹¹, Si(R¹¹)₃, B(OR¹¹)₂, C(═O)R¹¹, P(═O)(R¹¹)₂, S(═O)R¹¹, S(═O)₂R¹¹, OSO₂R¹¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹¹ radicals and where one or more nonadjacent CH₂ groups may be replaced by Si(R¹¹)₂, C═O, NR¹¹, O, S or CONR¹¹, or an aromatic or heteroaromatic ring system which has 5 to 40 aromatic ring atoms and may be substituted in each case by one or more R¹¹ radicals; at the same time, two or more R¹⁰ radicals together may form an aromatic, heteroaromatic, aliphatic or heteroaliphatic ring system; preferably, the R¹⁰ radicals do not form any such ring system;
R¹¹ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4, preferably 0 or 1 and very preferably 0;
t is the same or different at each instance and is 0, 1, 2 or 3, preferably 0 or 1 and very preferably 0;
x is the same or different at each instance and is 0, 1, 2, 3 or 4, preferably 0 or 1 and very preferably 0;
z is the same or different at each instance and is 0, 1 or 2, preferably 0 or 1 and very preferably 0, where the sum total of x and 2z is not more than 4, preferably not more than 2.

The sum total of the indices v, t, x and z in compounds of the formulae (11), (12), (13), (14), (15), (16), (17) and (18) is preferably not more than 6, especially preferably not more than 4 and more preferably not more than 2.

The L² group is a connecting group which is preferably selected from a bond or an aromatic or heteroaromatic ring system which has 5 to 40 aromatic ring atoms and may be substituted by one or more R⁹ radicals, more preferably a bond.

Preferably, the L² group may form through-conjugation with the groups to which the L² group of formula (11), (12), (13), (14), (15), (16), (17) and (18) or the preferred embodiments of this formula is bonded.

In a further preferred embodiment of the invention, L is a bond or an aromatic or heteroaromatic ring system which has 5 to 14 aromatic or heteroaromatic ring atoms, preferably an aromatic ring system which has 6 to 12 carbon atoms, and which may be substituted by one or more R⁹ radicals, but is preferably unsubstituted, where R⁹ may have the definition given above, especially for formulae (11), (12), (13), (14), (15), (16), (17) and (18). More preferably, L² is a bond or an aromatic ring system having 6 to 10 aromatic ring atoms or a heteroaromatic ring system having 6 to 13 heteroaromatic ring atoms, each of which may be substituted by one or more R⁹ radicals, but is preferably unsubstituted, where R⁹ may have the definition given above, especially for formulae (11), (12), (13), (14), (15), (16), (17) and (18).

Further preferably, the symbol L² shown in formulae (11), (12), (13), (14), (15), (16), (17) and (18) inter alia is the same or different at each instance and is a bond or an aryl or heteroaryl radical having 5 to 24 ring atoms, preferably 6 to 13 ring atoms, more preferably 6 to 10 ring atoms, such that an aromatic or heteroaromatic group of an aromatic or heteroaromatic ring system is bonded to the respective atom of the further group directly, i.e. via an atom of the aromatic or heteroaromatic group.

It may additionally be the case that the L² group shown in formulae (11), (12), (13), (14), (15), (16), (17) and (18) comprises an aromatic ring system having not more than two fused aromatic and/or heteroaromatic 6-membered rings, preferably does not comprise any fused aromatic or heteroaromatic ring system. Accordingly, naphthyl structures are preferred over anthracene structures. In addition, fluorenyl, spirobifluorenyl, dibenzofuranyl and/or dibenzothienyl structures are preferred over naphthyl structures.

Particular preference is given to structures having no fusion, for example phenyl, biphenyl, terphenyl and/or quaterphenyl structures.

Examples of suitable aromatic or heteroaromatic ring systems L² are selected from the group consisting of ortho-, meta- or para-phenylene, ortho-, meta- or para-biphenylene, terphenylene, especially branched terphenylene, quaterphenylene, especially branched quaterphenylene, fluorenylene, spirobifluorenylene, dibenzofuranylene, dibenzothienylene and carbazolylene, each of which may be substituted by one or more R⁹ radicals, but are preferably unsubstituted.

It may further be the case that the L² group shown in formulae (11), (12), (13), (14), (15), (16), (17) and (18) inter alia has not more than 1 nitrogen atom, preferably not more than 2 heteroatoms, especially preferably not more than one heteroatom and more preferably no heteroatom.

It may additionally be the case that the L² group does not form a fused aromatic or heteroaromatic ring system with the groups to which the L² group binds, where this includes the R⁹, R¹⁰ or R¹¹ radicals by which the L² group or any of the groups to which the L² group binds may be substituted.

More preferably, the L² group is a bond or a group selected from the formulae (L¹-1) to (L¹-13) as defined above, where the substituents R² in the structures of the formulae (L¹-1) to (L¹-13) should be replaced by R⁹.

In a preferred embodiment of the invention, R⁹ is the same or different at each instance and is selected from the group consisting of H, D, F, CN, NO₂, Si(R¹⁰)₃, B(OR¹⁰)₂, a straight-chain alkyl group having 1 to 20 carbon atoms or a branched or cyclic alkyl group having 3 to 20 carbon atoms, where the alkyl group may be substituted in each case by one or more R¹⁰ radicals, or an aromatic or heteroaromatic ring system which has 5 to 60 aromatic ring atoms, preferably 5 to 40 aromatic ring atoms, and may be substituted in each case by one or more R¹⁰ radicals.

In a further-preferred embodiment of the invention, R⁹ is the same or different at each instance and is selected from the group consisting of H, D, F, a straight-chain alkyl group having 1 to 20 carbon atoms or a branched or cyclic alkyl group having 3 to 20 carbon atoms, where the alkyl group may be substituted in each case by one or more R¹⁰ radicals, or an aromatic or heteroaromatic ring system which has 5 to 60 aromatic ring atoms, preferably 5 to 40 aromatic ring atoms, and may be substituted in each case by one or more R¹⁰ radicals.

In a further-preferred embodiment of the invention, R⁹ is the same or different at each instance and is selected from the group consisting of H, D, an aromatic or heteroaromatic ring system which has 6 to 30 aromatic ring atoms and may be substituted by one or more R¹⁰ radicals, and an N(Ar′″)₂ group. More preferably, R⁹ is the same or different at each instance and is selected from the group consisting of H or an aromatic or heteroaromatic ring system which has 6 to 24 aromatic ring atoms, preferably 6 to 18 aromatic ring atoms, more preferably 6 to 13 aromatic ring atoms, and may be substituted in each case by one or more R¹⁰ radicals.

Preferred aromatic or heteroaromatic ring systems R⁹ or Ar′″ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R¹⁰ radicals. The structures Ar-1 to Ar-75 listed above are particularly preferred, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-75 set out above, in relation to the R⁶ and Ar″ radicals, the substituents R⁴ should be replaced by the corresponding R¹⁰ radicals. The preferences set out above for the R² and R³ groups are correspondingly applicable to the R⁹ group.

Further suitable R⁹ groups are groups of the formula —Ar⁴—N(Ar²)(Ar³) where Ar², Ar³ and Ar⁴ are the same or different at each instance and are an aromatic or heteroaromatic ring system which has 5 to 24 aromatic ring atoms and may be substituted in each case by one or more R⁴ radicals. The total number of aromatic ring atoms in Ar², Ar³ and Ar⁴ here is not more than 60 and preferably not more than 40. Further preferences for the Ar², Ar³ and Ar⁴ groups have been set out above and are correspondingly applicable.

It may further be the case that the substituents R⁹ according to the above formulae do not form a fused aromatic or heteroaromatic ring system, preferably any fused ring system, with the ring atoms of the ring system. This includes the formation of a fused ring system with possible substituents R¹⁰, R¹¹ which may be bonded to the R⁹ radicals.

When A² is NR¹⁰, the substituent R¹⁰ bonded to the nitrogen atom is preferably an aromatic or heteroaromatic ring system which has 5 to 24 aromatic ring atoms and may also be substituted by one or more R¹¹ radicals. In a particularly preferred embodiment, this R¹⁰ substituent is the same or different at each instance and is an aromatic or heteroaromatic ring system which has 6 to 24 aromatic ring atoms, especially 6 to 18 aromatic ring atoms, which does not have any fused aryl groups and which does not have any fused heteroaryl groups in which two or more aromatic or heteroaromatic 6-membered ring groups are fused directly to one another, and which may also be substituted in each case by one or more R¹¹ radicals. Preference is given to phenyl, biphenyl, terphenyl and quaterphenyl having bonding patterns as listed above for Ar-1 to Ar-11, where these structures, rather than by R⁴, may be substituted by one or more R¹¹ radicals, but are preferably unsubstituted. Preference is further given to triazine, pyrimidine and quinazoline as listed above for Ar-47 to Ar-Ar-57 and Ar-58, where these structures, rather than by R⁴, may be substituted by one or more R¹¹ radicals.

When A² is C(R¹⁰)₂, the substituents R¹⁰ bonded to this carbon atom are preferably the same or different at each instance and are a linear alkyl group having 1 to 10 carbon atoms or a branched or cyclic alkyl group having 3 to 10 carbon atoms or an aromatic or heteroaromatic ring system having 5 to 24 aromatic ring atoms, which may also be substituted by one or more R¹¹ radicals. Most preferably, R¹⁰ is a methyl group or a phenyl group. In this case, the R¹⁰ radicals together may also form a ring system, which leads to a spiro system.

Preferred aromatic or heteroaromatic ring systems Ar⁵ and/or Ar⁶ are selected from phenyl, biphenyl, especially ortho-, meta- or para-biphenyl, terphenyl, especially ortho-, meta- or para-terphenyl or branched terphenyl, quaterphenyl, especially ortho-, meta- or para-quaterphenyl or branched quaterphenyl, fluorene which may be joined via the 1, 2, 3 or 4 position, spirobifluorene which may be joined via the 1, 2, 3 or 4 position, naphthalene, especially 1- or 2-bonded naphthalene, indole, benzofuran, benzothiophene, carbazole which may be joined via the 1, 2, 3 or 4 position, dibenzofuran which may be joined via the 1, 2, 3 or 4 position, dibenzothiophene which may be joined via the 1, 2, 3 or 4 position, indenocarbazole, indolocarbazole, pyridine, pyrimidine, pyrazine, pyridazine, triazine, quinoline, isoquinoline, quinazoline, quinoxaline, phenanthrene or triphenylene, each of which may be substituted by one or more R⁷ or R¹⁰ radicals.

The Ar⁵ and/or Ar⁶ groups here are more preferably independently selected from the groups of the formulae Ar-1 to Ar-75 set out above, preference being given to structures of the formulae (Ar-1), (Ar-2), (Ar-3), (Ar-12), (Ar-13), (Ar-14), (Ar-15), (Ar-16), (Ar-69), (Ar-70), (Ar-75), and particular preference to structures of the formulae (Ar-1), (Ar-2), (Ar-3), (Ar-12), (Ar-13), (Ar-14), (Ar-15), (Ar-16). In the structures Ar-1 to Ar-75 set out above, in relation to the Ar⁵ radicals, the substituents R⁴ should be replaced by the corresponding R⁷ or R¹⁰ radicals.

In a further preferred embodiment of the invention, R⁷ and/or R¹⁰ are the same or different at each instance and are selected from the group consisting of H, D, F, CN, a straight-chain alkyl group having 1 to 10 carbon atoms or a branched or cyclic alkyl group having 3 to 10 carbon atoms, where the alkyl group may be substituted in each case by one or more R⁸ or R¹¹ radicals, or an aromatic or heteroaromatic ring system which has 6 to 24 aromatic ring atoms and may be substituted in each case by one or more R⁸ or R¹¹ radicals. In a particularly preferred embodiment of the invention, R⁷ and/or R¹⁰ are the same or different at each instance and are selected from the group consisting of H, a straight-chain alkyl group having 1 to 6 carbon atoms, especially having 1, 2, 3 or 4 carbon atoms, or a branched or cyclic alkyl group having 3 to 6 carbon atoms, where the alkyl group may be substituted by one or more R⁸ or R¹¹ radicals, but is preferably unsubstituted, or an aromatic or heteroaromatic ring system which has 6 to 13 aromatic ring atoms and may be substituted in each case by one or more R⁸ or R¹¹ radicals, but is preferably unsubstituted.

In a further preferred embodiment of the invention, R⁸ and/or R¹¹ are the same or different at each instance and are H, an alkyl group having 1 to 4 carbon atoms or an aryl group having 6 to 10 carbon atoms, which may be substituted by an alkyl group having 1 to 4 carbon atoms, but is preferably unsubstituted.

Preferred embodiments of the compounds of the formulae (7) and (8) are the compounds of the following formulae (7a) and (8a):

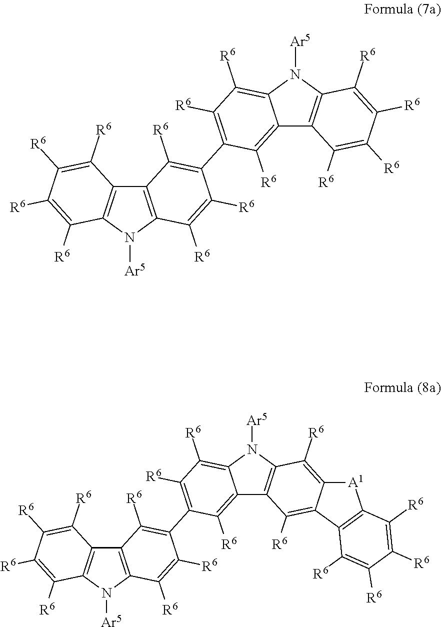

where R⁶, Ar⁵ and A¹ have the definitions given above, especially for formula (7) or (8). In a preferred embodiment of the invention, A in formula (8a) is C(R⁷)₂.

Preferred embodiments of the compounds of the formulae (7a) and (8a) are the compounds of the following formulae (7b) and (8b):

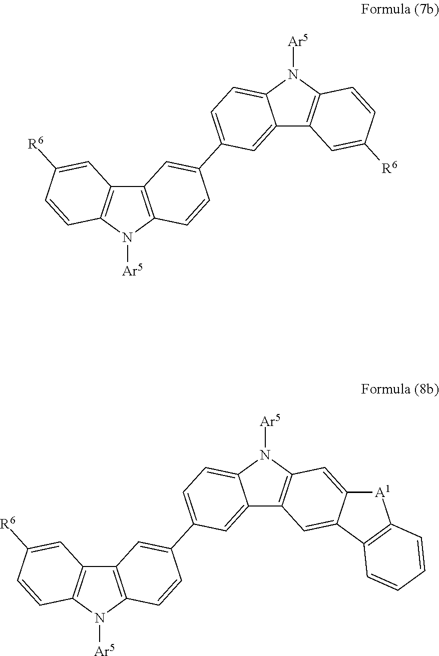

where R⁶, Ar⁵ and A¹ have the definitions given above, especially for formula (7) or (8). In a preferred embodiment of the invention, A in formula (8b) is C(R⁷)₂.

Examples of suitable compounds of formulae (7), (8), (9) and (10) are the compounds depicted below:

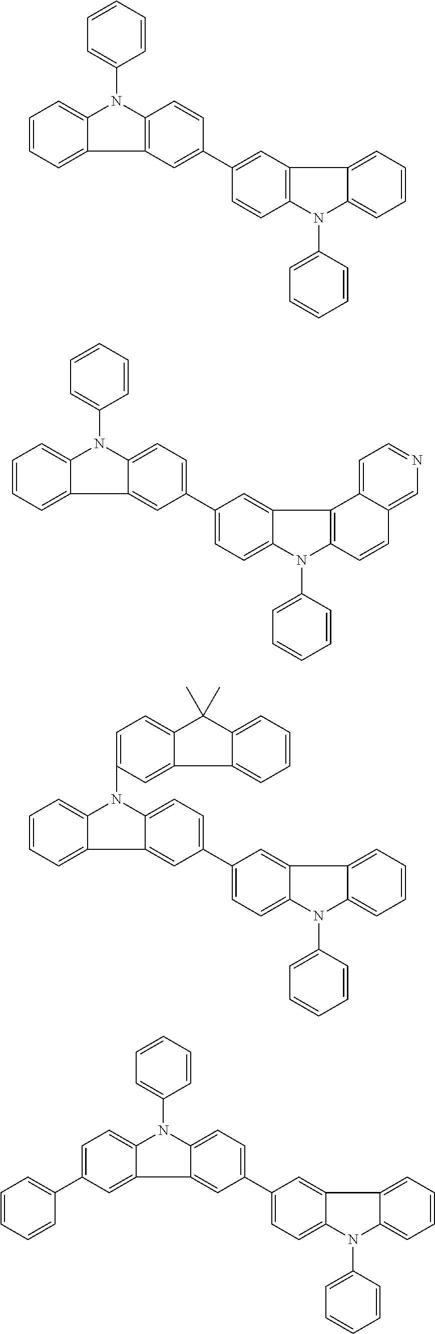 [SMILES: c1ccc(-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4c6ccncc6ccc4n5-c4ccccc4)ccc32)cc1 ; CC1(C)c2ccccc2-c2cc(-n3c4ccccc4c4cc(-c5ccc6c(c5)c5ccccc5n6-c5ccccc5)ccc43)ccc21 ; c1ccc(-c2ccc3c(c2)c2cc(-c4ccc5c(c4)c4ccccc4n5-c4ccccc4)ccc2n3-c2ccccc2)cc1]

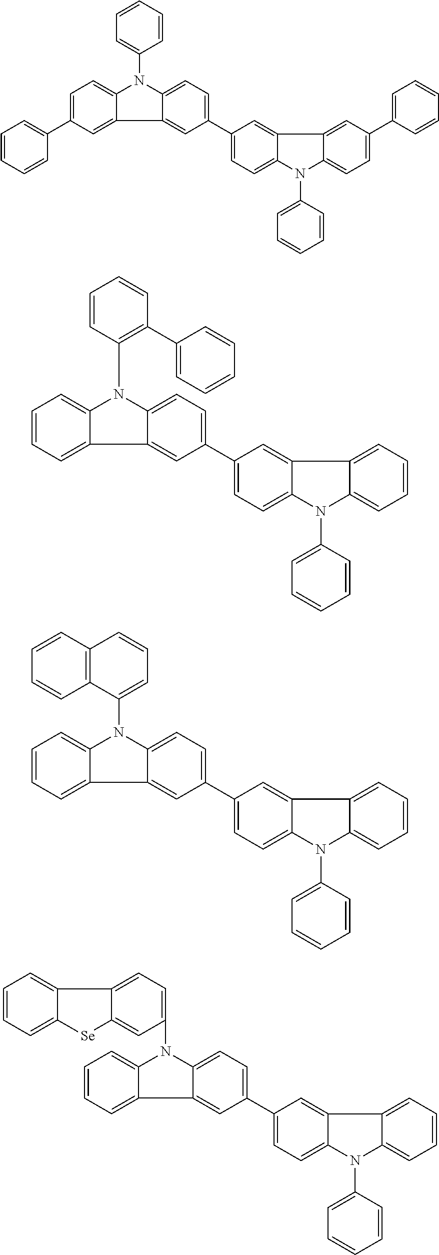 [SMILES: c1ccc(-c2ccc3c(c2)c2cc(-c4ccc5c(c4)c4cc(-c6ccccc6)ccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5c(c4)[se]c4ccccc45)ccc32)cc1]

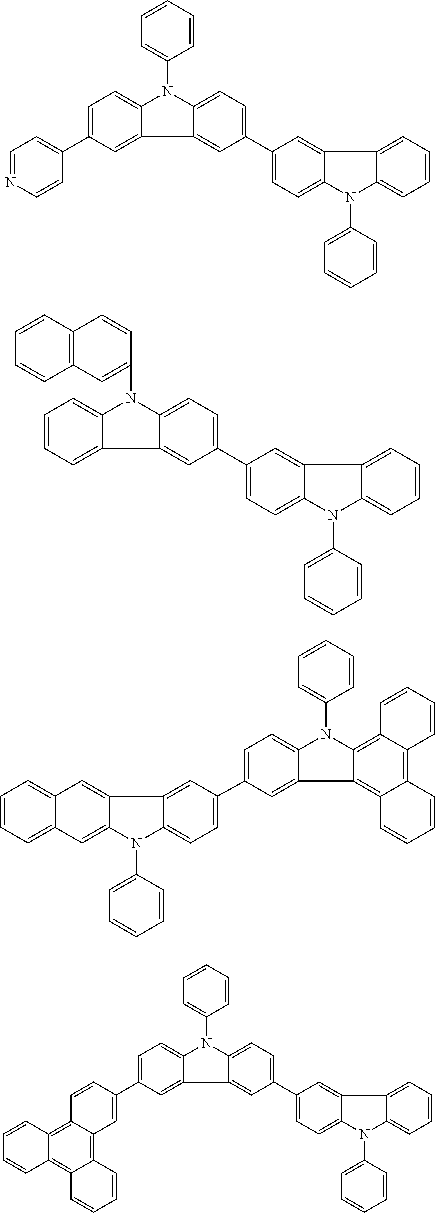

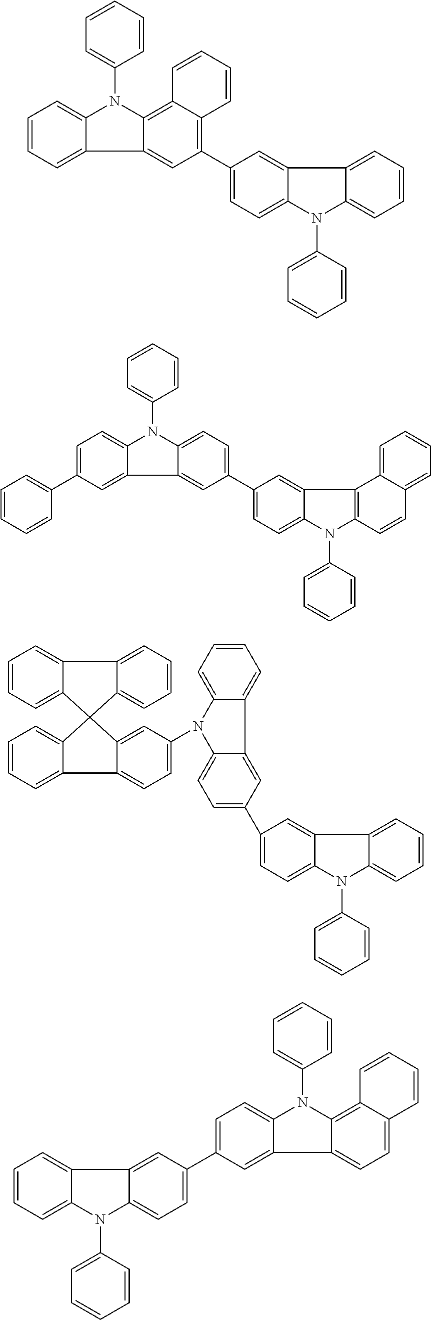 [SMILES: c1ccc(-n2c3ccccc3c3cc(-c4cc5c6ccccc6n(-c6ccccc6)c5c5ccccc45)ccc32)cc1 ; c1ccc(-c2ccc3c(c2)c2cc(-c4ccc5c(c4)c4c6ccccc6ccc4n5-c4ccccc4)ccc2n3-c2ccccc2)cc1 ; c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccc6ccccc6c4n5-c4ccccc4)ccc32)cc1]

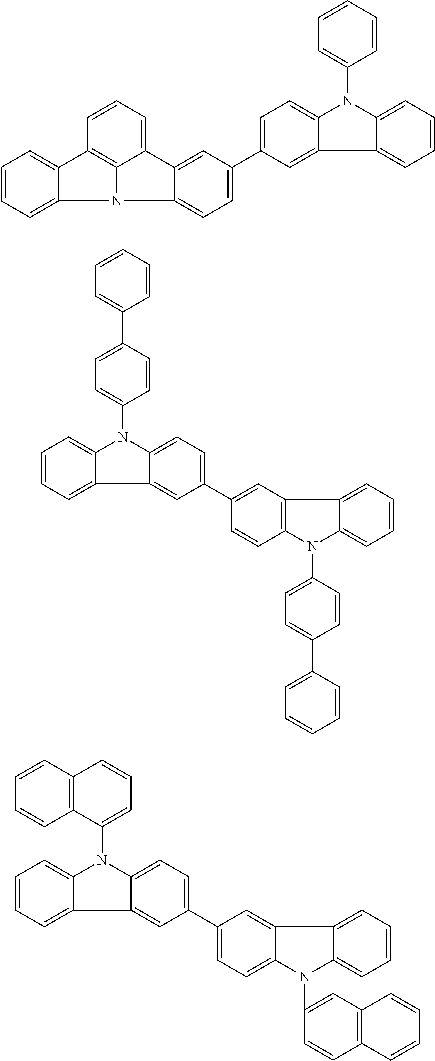 [SMILES: c1ccc(-n2c3ccccc3c3cc(-c4ccc5c(c4)c4cccc6c7ccccc7n5c64)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc2cc(-n3c4ccccc4c4cc(-c5ccc6c(c5)c5ccccc5n6-c5cccc6ccccc56)ccc43)ccc2c1]

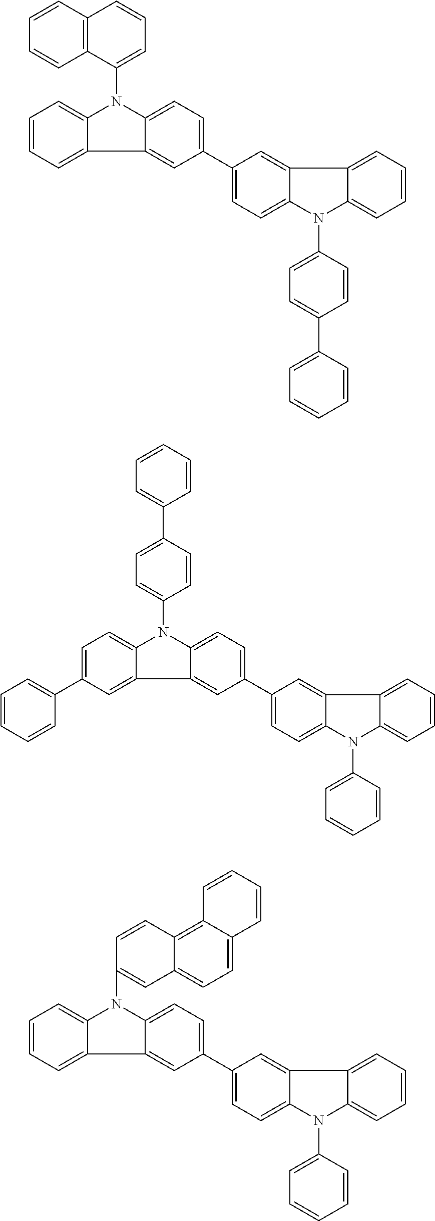 [SMILES: c1ccc(-c2ccc(-n3c4ccccc4c4cc(-c5ccc6c(c5)c5ccccc5n6-c5cccc6ccccc56)ccc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc5c(ccc6ccccc65)c4)ccc32)cc1]

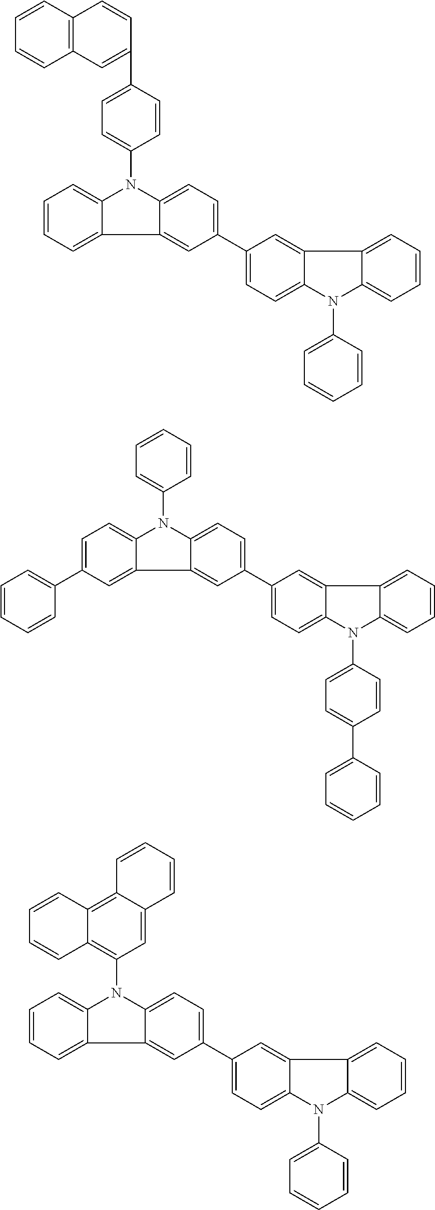 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4cc5ccccc5c5ccccc45)ccc32)cc1]

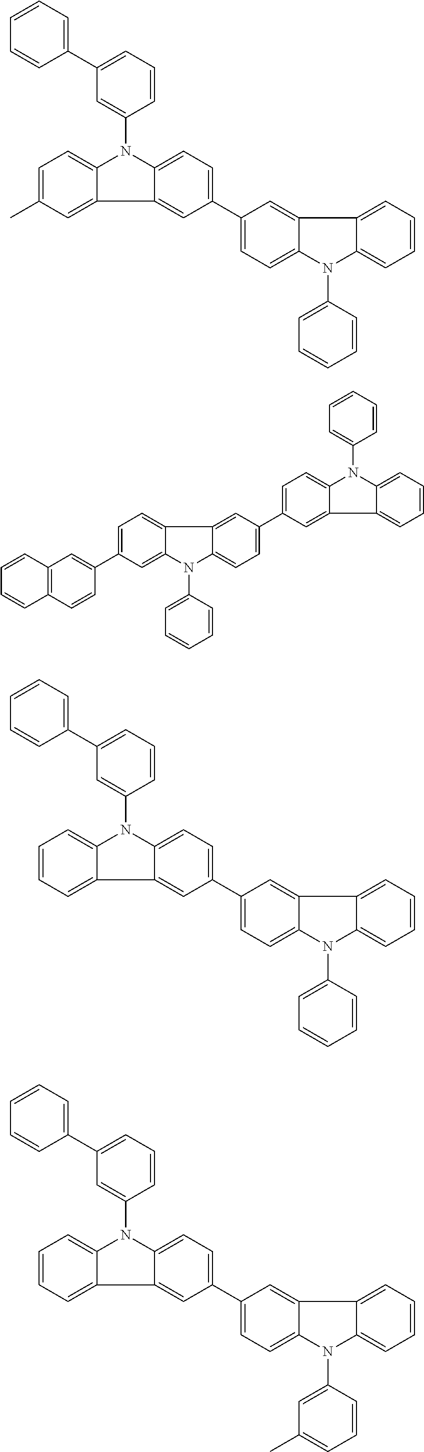

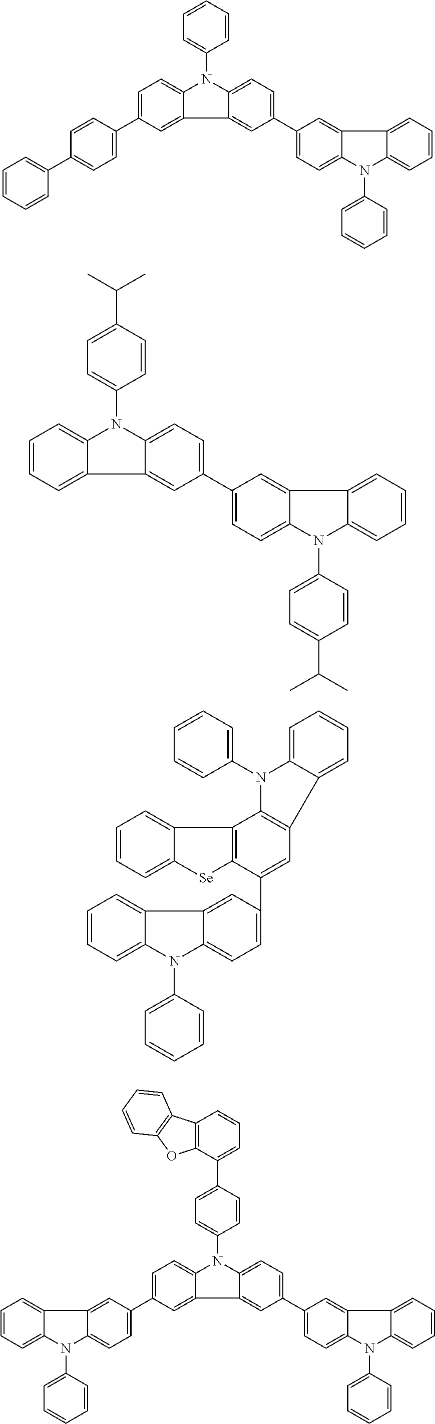

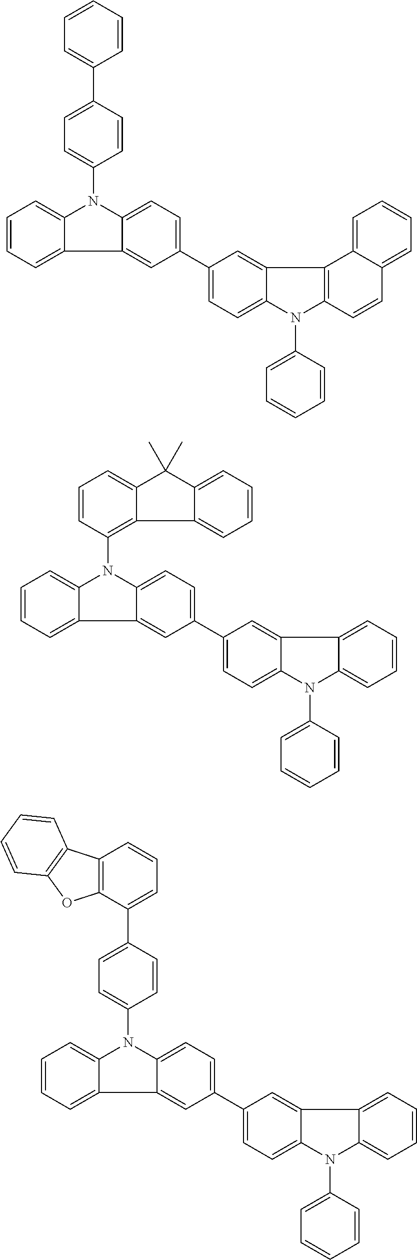 [SMILES: c1ccc(-c2ccc(-n3c4ccccc4c4cc(-c5ccc6c(c5)c5c7ccccc7ccc5n6-c5ccccc5)ccc43)cc2)cc1 ; CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc(-c5cccc6c5oc5ccccc56)cc4)ccc32)cc1]

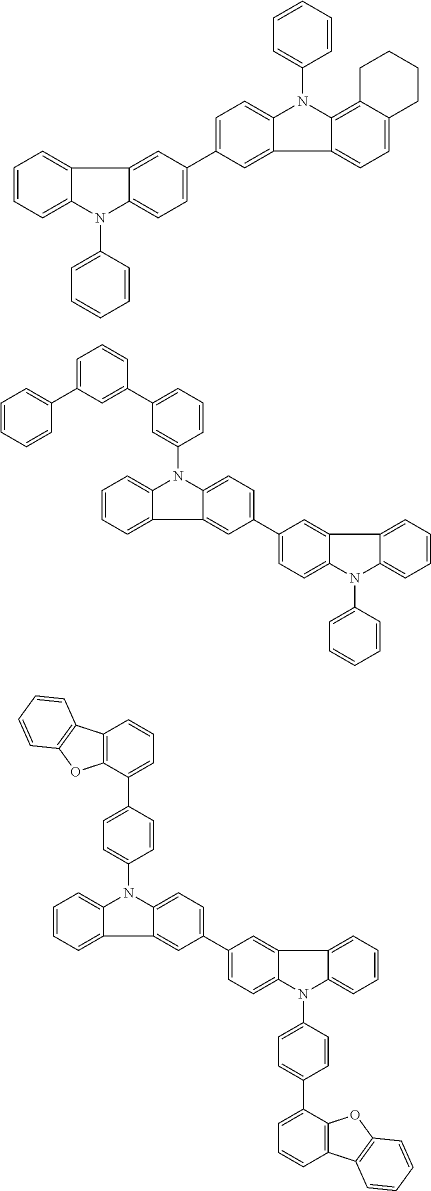

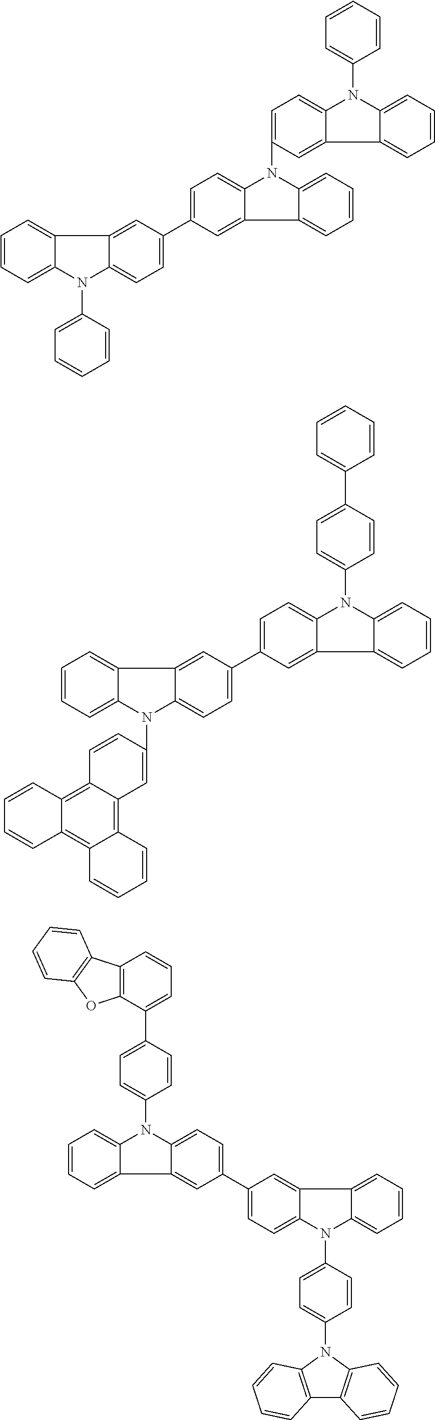 [SMILES: c1ccc(-n2c3ccccc3c3cc(-c4ccc5c(c4)c4ccccc4n5-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; c1ccc2c(c1)oc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

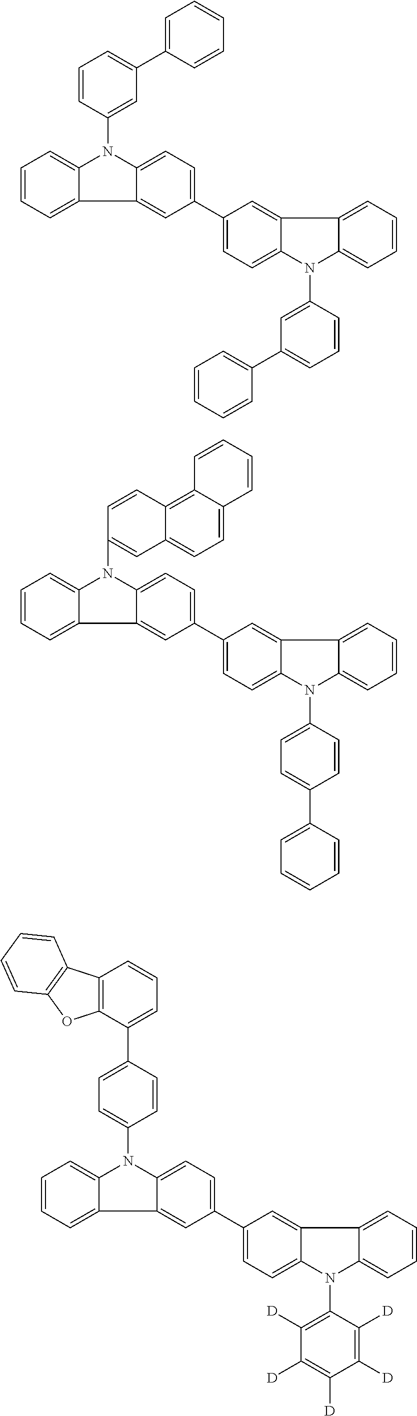

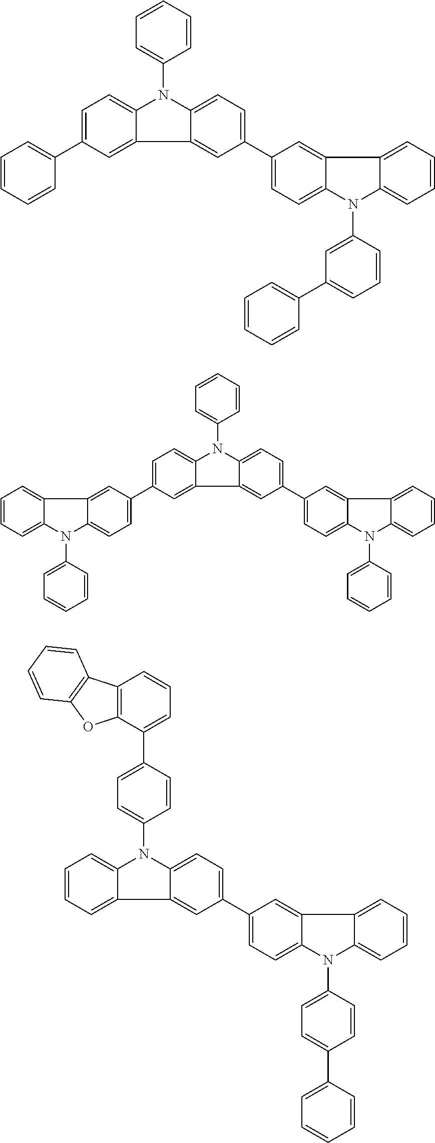

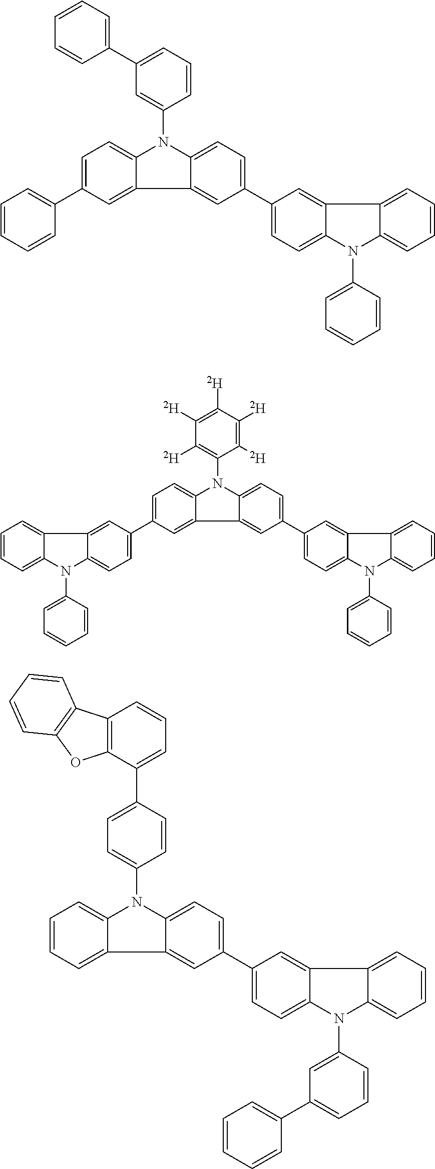

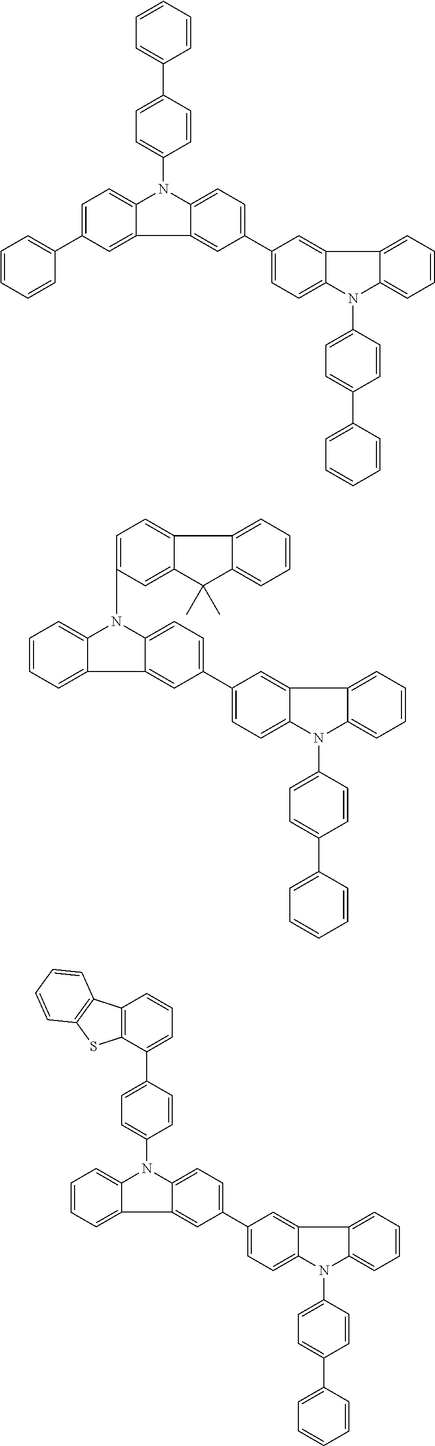 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc(-c6cccc7c6sc6ccccc67)cc5)ccc43)cc2)cc1]

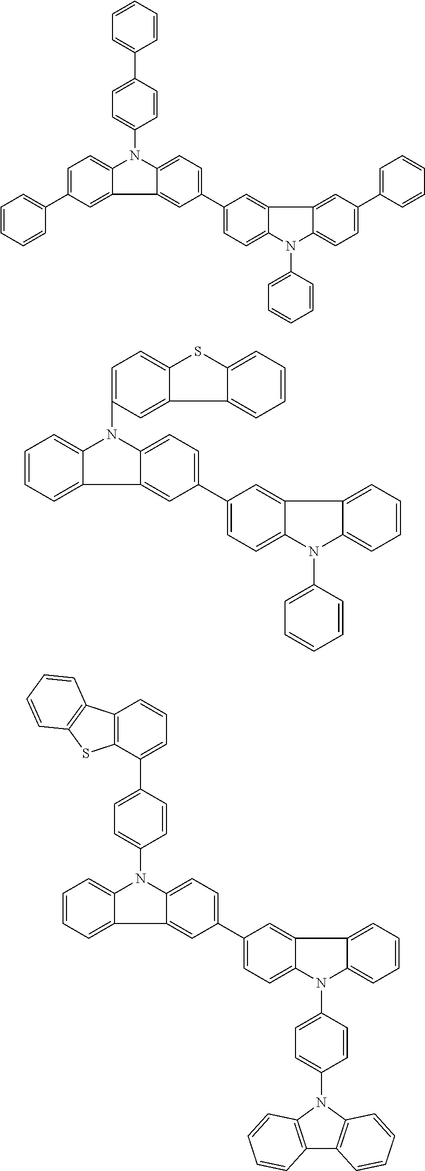 [SMILES: c1ccc(-c2ccc(-n3c4ccc(-c5ccccc5)cc4c4cc(-c5ccc6c(c5)c5cc(-c7ccccc7)ccc5n6-c5ccccc5)ccc43)cc2)cc1 ; c1ccc(-n2c3ccccc3c3cc(-c4ccc5c(c4)c4ccccc4n5-c4ccc5sc6ccccc6c5c4)ccc32)cc1 ; c1ccc2c(c1)sc1c(-c3ccc(-n4c5ccccc5c5cc(-c6ccc7c(c6)c6ccccc6n7-c6ccc(-n7c8ccccc8c8ccccc87)cc6)ccc54)cc3)cccc12]

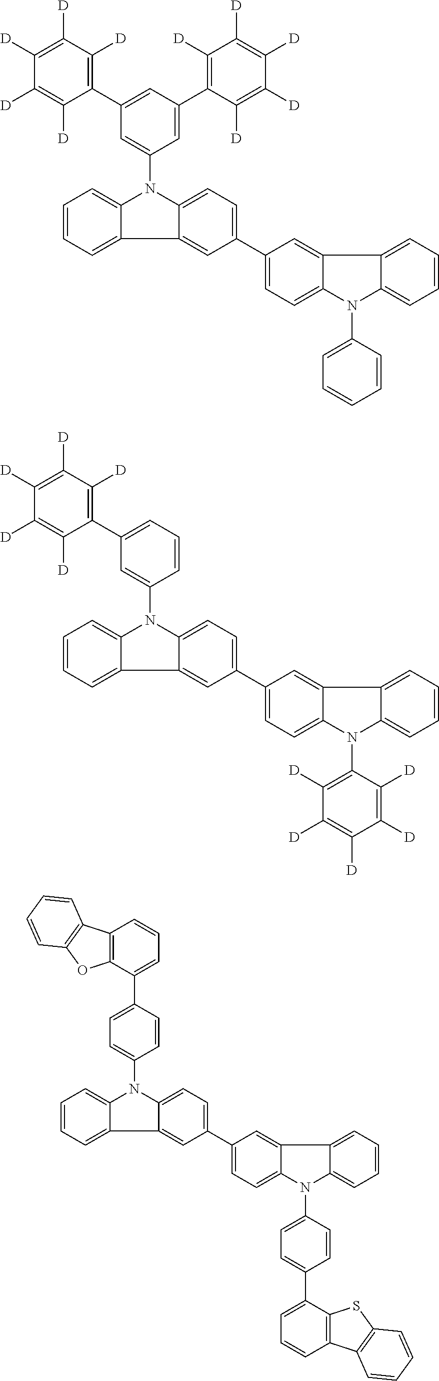 [SMILES: [2H]c1c([2H])c([2H])c(-c2cc(-c3c([2H])c([2H])c([2H])c([2H])c3[2H])cc(-n3c4ccccc4c4cc(-c5ccc6c(c5)c5ccccc5n6-c5ccccc5)ccc43)c2)c([2H])c1[2H] ; [2H]c1c([2H])c([2H])c(-c2cccc(-n3c4ccccc4c4cc(-c5ccc6c(c5)c5ccccc5n6-c5c([2H])c([2H])c([2H])c([2H])c5[2H])ccc43)c2)c([2H])c1[2H] ; c1ccc2c(c1)oc1c(-c3ccc(-n4c5ccccc5c5cc(-c6ccc7c(c6)c6ccccc6n7-c6ccc(-c7cccc8c7sc7ccccc78)cc6)ccc54)cc3)cccc12]

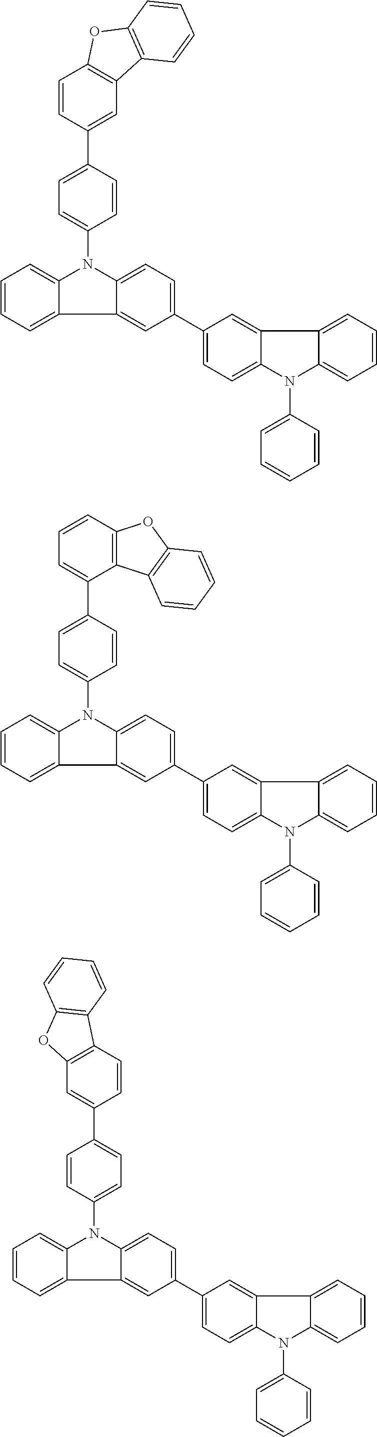 [SMILES: c1ccc(-n2c3ccccc3c3cc(-c4ccc5c(c4)c4ccccc4n5-c4ccc(-c5ccc6oc7ccccc7c6c5)cc4)ccc32)cc1 ; c1ccc(-n2c3ccccc3c3cc(-c4ccc5c(c4)c4ccccc4n5-c4ccc(-c5cccc6oc7ccccc7c56)cc4)ccc32)cc1 ; c1ccc(-n2c3ccccc3c3cc(-c4ccc5c(c4)c4ccccc4n5-c4ccc(-c5ccc6c(c5)oc5ccccc56)cc4)ccc32)cc1]

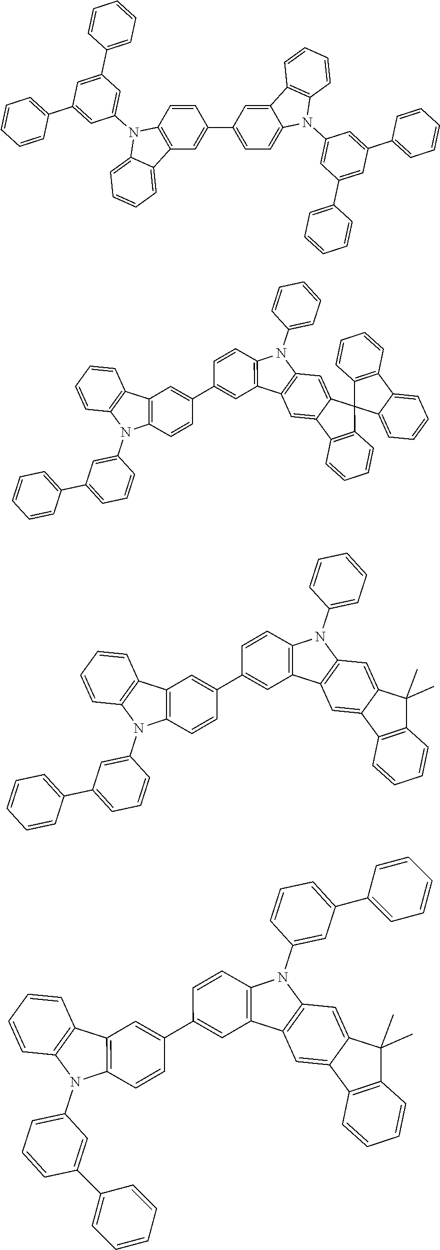

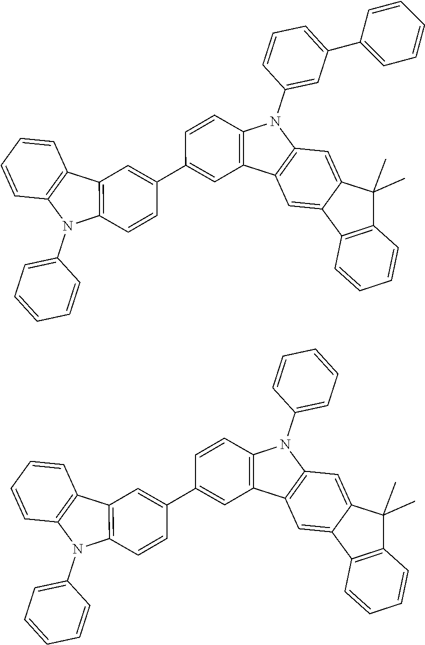 [SMILES: CC1(C)c2ccccc2-c2cc3c4cc(-c5ccc6c(c5)c5ccccc5n6-c5ccccc5)ccc4n(-c4cccc(-c5ccccc5)c4)c3cc21 ; CC1(C)c2ccccc2-c2cc3c4cc(-c5ccc6c(c5)c5ccccc5n6-c5ccccc5)ccc4n(-c4ccccc4)c3cc21]

The combination of at least one compound of formula (1) or the preferred embodiments thereof that are set out above with a compound of one of the formulae (7), (8), (9) and (10) can achieve surprising advantages. The present invention therefore further provides a composition comprising at least one compound of formula (1) or the preferred embodiments thereof that are set out above and at least one further matrix material, wherein the further matrix material is selected from compounds of one of the formulae (7), (8), (9) and (10).

It may preferably be the case that the composition consists of at least one compound of formula (1) or the preferred embodiments thereof that are set out above and at least one compound of one of the formulae (7), (8), (9) and (10). These compositions are especially suitable as what are called pre-mixtures, which can be evaporated together.

The combination of at least one compound of formula (1) or the preferred embodiments thereof that are set out above with a compound of one of the formulae (11), (12), (13), (14), (15), (16), (17) and (18) can achieve surprising advantages. The present invention therefore further provides a composition comprising at least one compound of formula (1) or the preferred embodiments thereof that are set out above and at least one further matrix material, wherein the further matrix material is selected from compounds of one of the formulae (11), (12), (13), (14), (15), (16), (17) and (18).

It may preferably be the case that the composition consists of at least one compound of formula (1) or the preferred embodiments thereof that are set out above and at least one compound of one of the formulae (11), (12), (13), (14), (15), (16), (17) and (18). These compositions are especially suitable as what are called pre-mixtures, which can be evaporated together.

It may preferably further be the case that the composition consists of at least one compound of formula (1) or the preferred embodiments thereof that are set out above and at least one compound of one of the formulae (7), (8), (9), (10), (11), (12), (13), (14), (15), (16), (17) and (18). These compositions are especially suitable as what are called pre-mixtures, which can be evaporated together.

In this context, the compounds of one of the formulae (7), (8), (9), (10), (11), (12), (13), (14), (15), (16), (17) and (18) may each be used individually or as a mixture of two, three or more compounds of the respective structures.

In addition, the compounds of the formulae (7), (8), (9), (10), (11), (12), (13), (14), (15), (16), (17) and (18) may be used individually or as a mixture of two, three or more compounds of different structures.

The compound of formula (1) or the preferred embodiments thereof that are set out above preferably has a proportion by mass in the composition in the range from 10% by weight to 95% by weight, more preferably in the range from 15% by weight to 90% by weight, and very preferably in the range from 40% by weight to 70% by weight, based on the total mass of the composition.

It may further be the case that the compounds of one of the formulae (7), (8), (9), (10), (11), (12), (13), (14), (15), (16), (17) and (18) have a proportion by mass in the composition in the range from 5% by weight to 90% by weight, preferably in the range from 10% by weight to 85% by weight, more preferably in the range from 20% by weight to 85% by weight, even more preferably in the range from 30% by weight to 80% by weight, very particularly preferably in the range from 20% by weight to 60% by weight and most preferably in the range from 30% by weight to 50% by weight, based on the overall composition.

It may additionally be the case that the further matrix material is a hole-transporting matrix material of at least one of the formulae (7), (8), (9) and (10), and the hole-transporting matrix material has a proportion by mass in the composition in the range from 10% by weight to 95% by weight, preferably in the range from 15% by weight to 90% by weight, more preferably in the range from 15% by weight to 80% by weight, even more preferably in the range from 20% by weight to 70% by weight, very particularly preferably in the range from 40% by weight to 80% by weight and most preferably in the range from 50% by weight to 70% by weight, based on the overall composition.

It may additionally be the case that the further matrix material is an electron-transporting matrix material of at least one of the formulae (11), (12), (13), (14), (15), (16), (17) and (18), and the electron-transporting matrix material has a proportion by mass in the composition in the range from 10% by weight to 95% by weight, preferably in the range from 15% by weight to 90% by weight, more preferably in the range from 15% by weight to 80% by weight, even more preferably in the range from 20% by weight to 70% by weight, very particularly preferably in the range from 40% by weight to 80% by weight and most preferably in the range from 50% by weight to 70% by weight, based on the overall composition.

It may additionally be the case that the composition consists exclusively of the formula (1) or the preferred embodiments thereof that are set out above and one of the further matrix materials mentioned, preferably compounds of at least one of the formulae (7), (8), (9) and (10).

It may further be the case that the composition consists exclusively of the formula (1) or the preferred embodiments thereof that are set out above and one of the further matrix materials mentioned, preferably compounds of at least one of the formulae (11), (12), (13), (14), (15), (16), (17) and (18).

It may also be the case that the composition consists exclusively of the formula (1) or the preferred embodiments thereof that are set out above and one of the further matrix materials mentioned, preferably compounds of at least one of the formulae (7), (8), (9), (10), (11), (12), (13), (14), (15), (16), (17) and (18).

Suitable phosphorescent compounds (=triplet emitters) are especially compounds which, when suitably excited, emit light, preferably in the visible region, and also contain at least one atom of atomic number greater than 20, preferably greater than 38 and less than 84, more preferably greater than 56 and less than 80, especially a metal having this atomic number. Preferred phosphorescence emitters used are compounds containing copper, molybdenum, tungsten, rhenium, ruthenium, osmium, rhodium, iridium, palladium, platinum, silver, gold or europium, especially compounds containing iridium or platinum.

Examples of the above-described emitters can be found in applications WO 00/70655, WO 2001/41512, WO 2002/02714, WO 2002/15645, EP 1191613, EP 1191612, EP 1191614, WO 05/033244, WO 05/019373, US 2005/0258742, WO 2009/146770, WO 2010/015307, WO 2010/031485, WO 2010/054731, WO 2010/054728, WO 2010/086089, WO 2010/099852, WO 2010/102709, WO 2011/032626, WO 2011/066898, WO 2011/157339, WO 2012/007086, WO 2014/008982, WO 2014/023377, WO 2014/094961, WO 2014/094960, WO 2015/036074, WO 2015/104045, WO 2015/117718, WO 2016/015815, WO 2016/124304, WO 2017/032439 and WO 2018/011186. In general, all phosphorescent complexes as used for phosphorescent electroluminescent devices according to the prior art and as known to those skilled in the art in the field of organic electroluminescence are suitable, and the person skilled in the art will be able to use further phosphorescent complexes without exercising inventive skill.

Examples of phosphorescent dopants are listed in the following table:

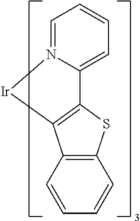

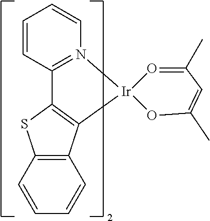

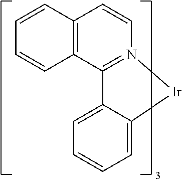

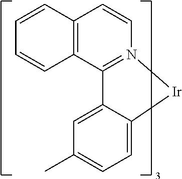

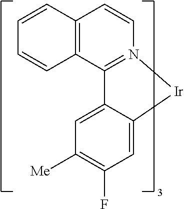

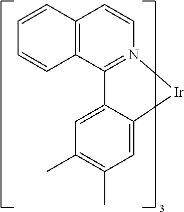

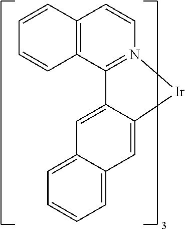

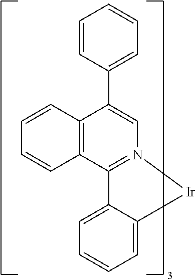

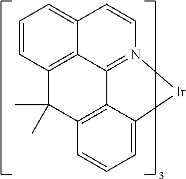

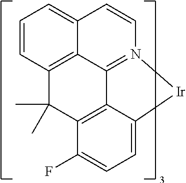

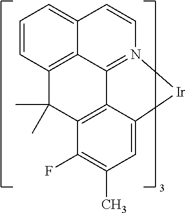

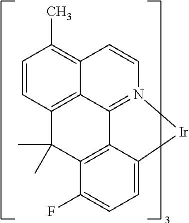

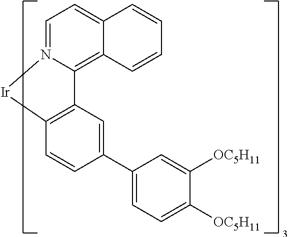

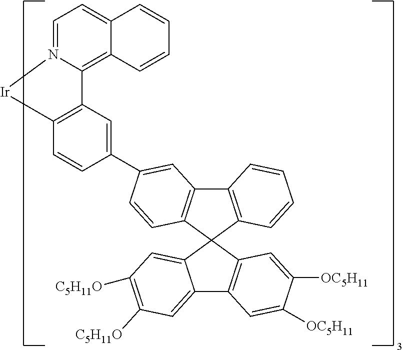

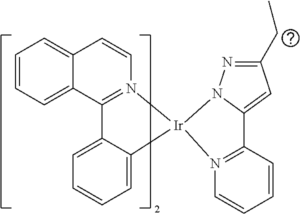

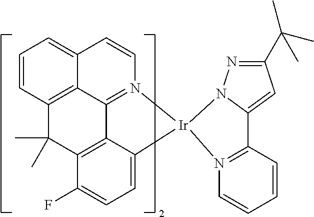

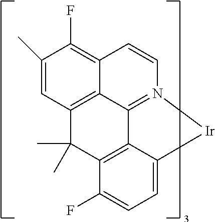

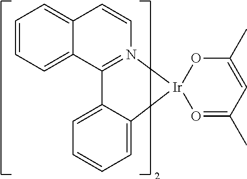

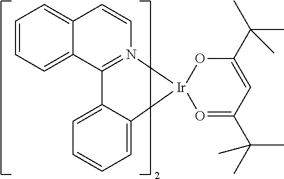

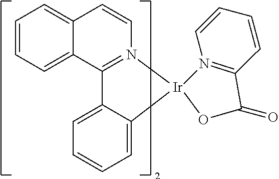

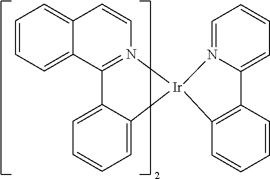

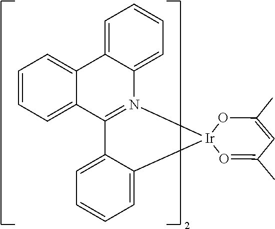

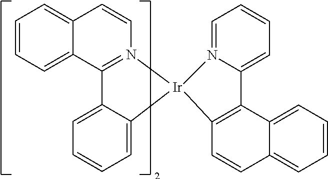

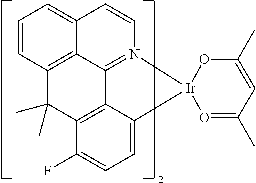

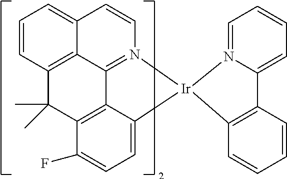

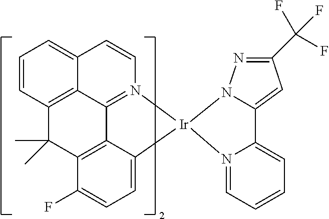

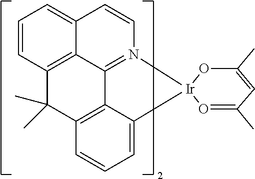

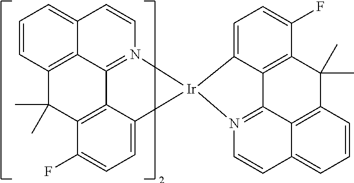

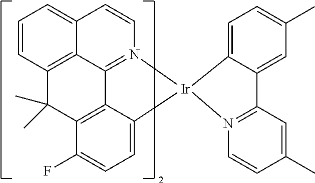

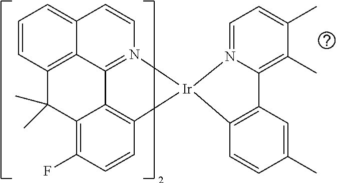

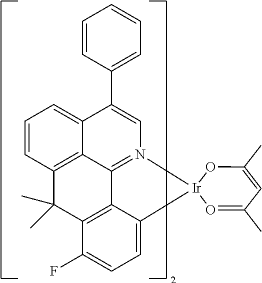

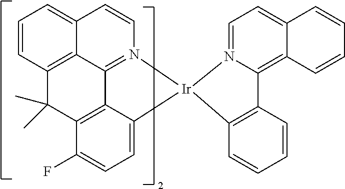

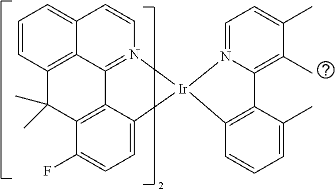

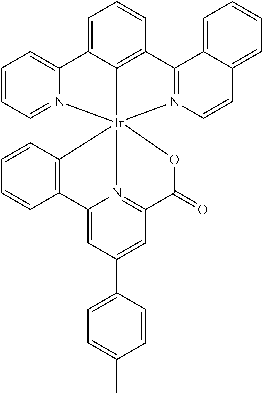

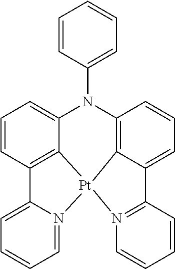

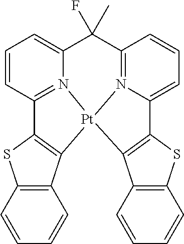

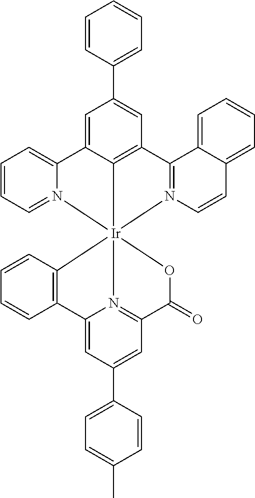

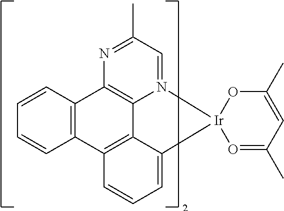

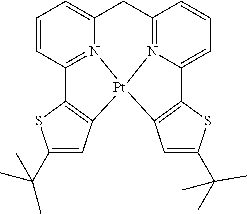

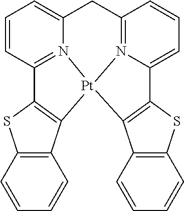

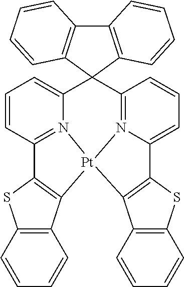

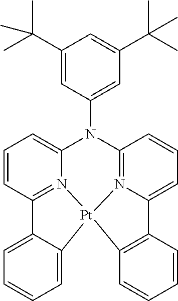

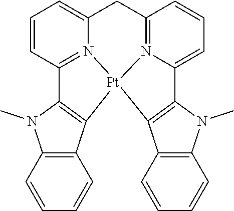

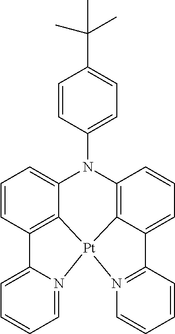

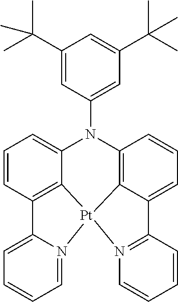

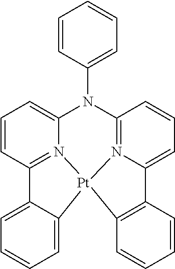

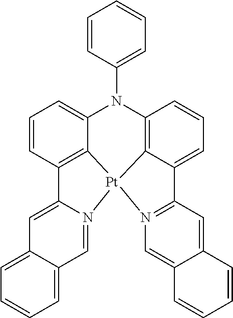

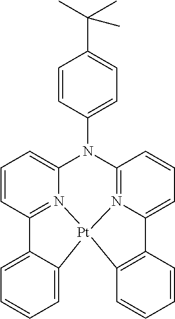

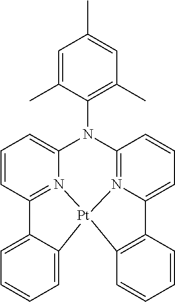

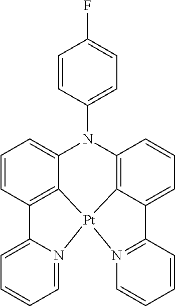

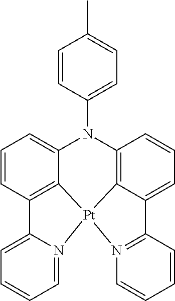

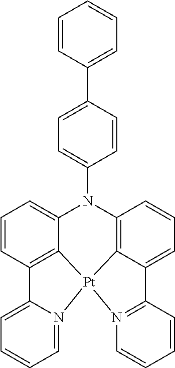

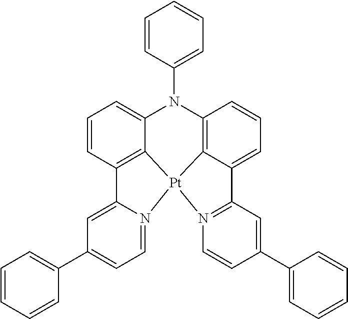

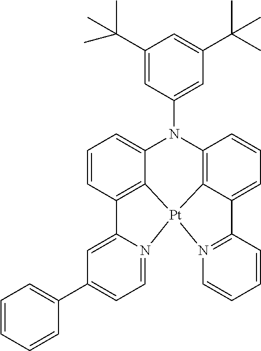

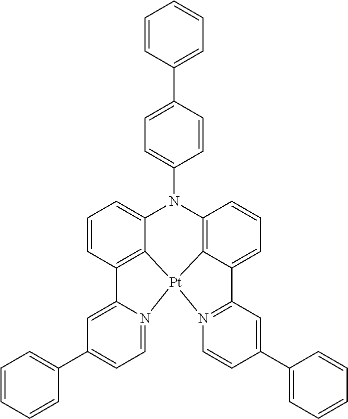

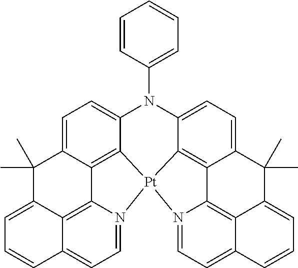

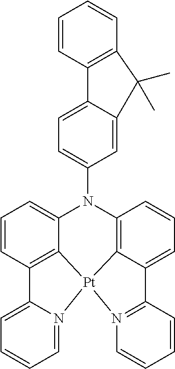

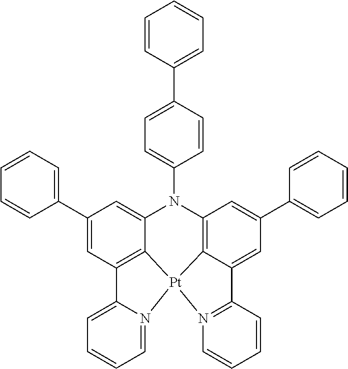

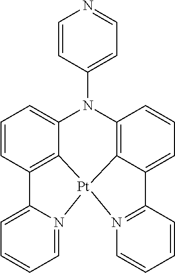

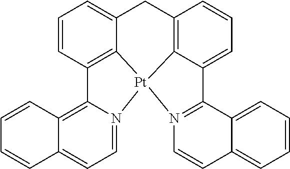

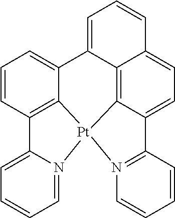

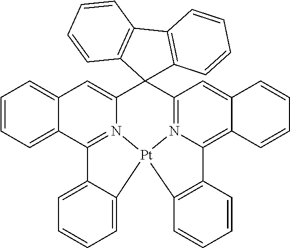

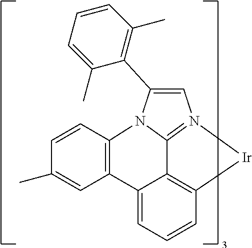

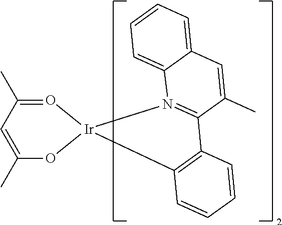

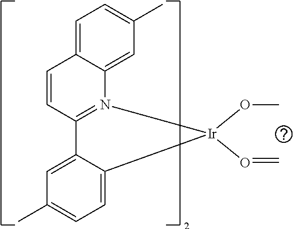

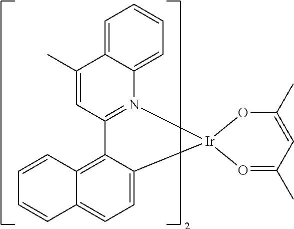

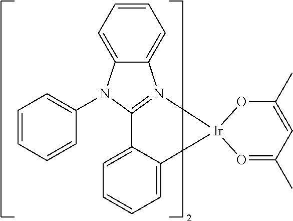

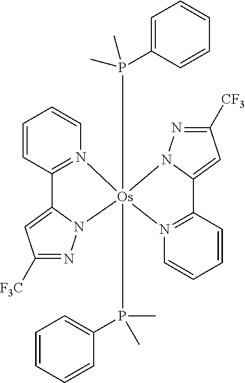

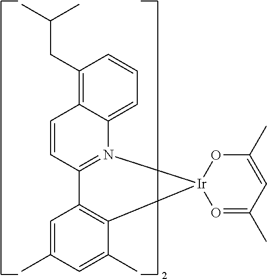

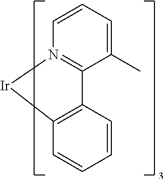

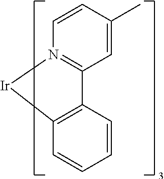

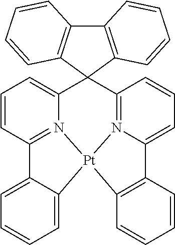

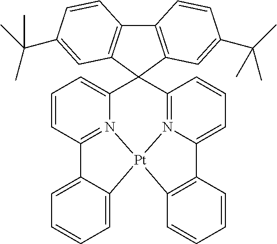

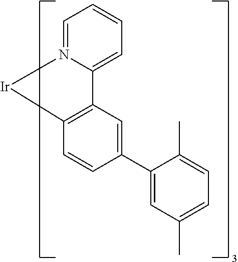

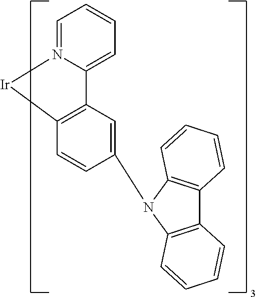

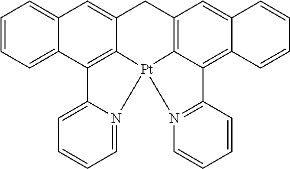

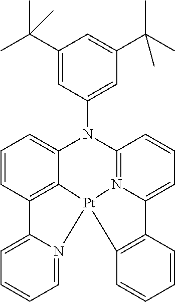

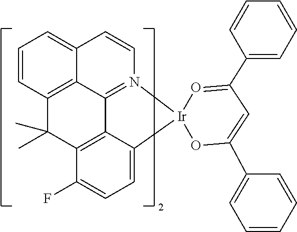

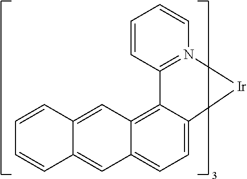

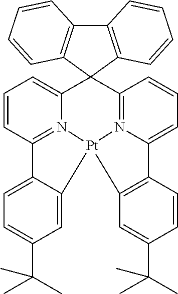

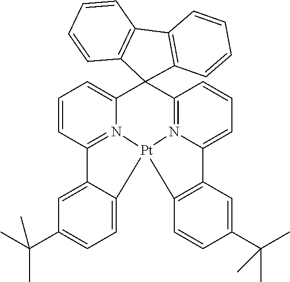

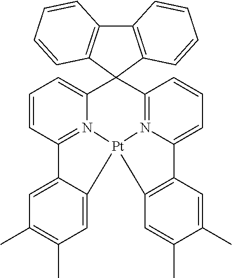

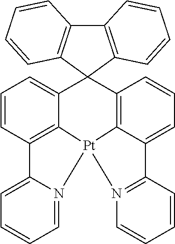

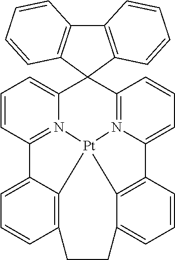

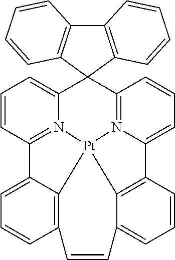

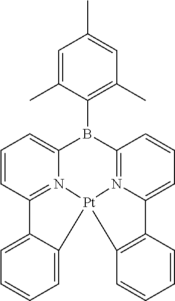

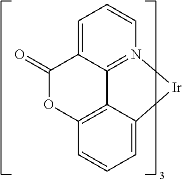

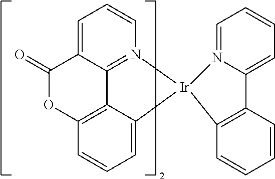

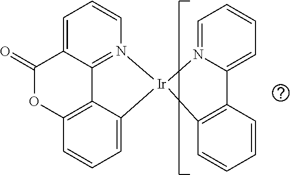

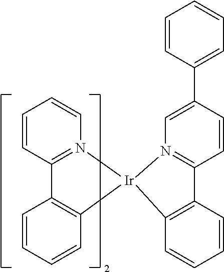

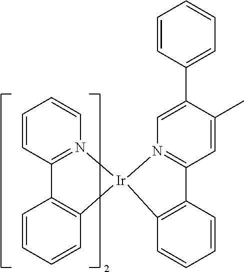

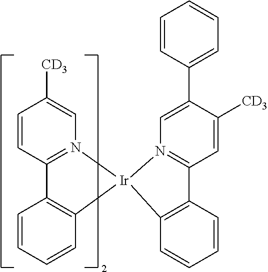

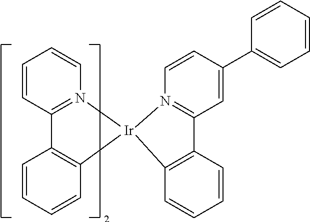

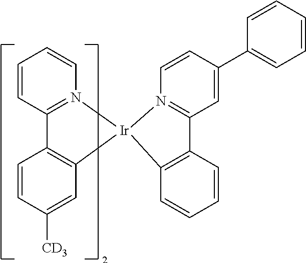

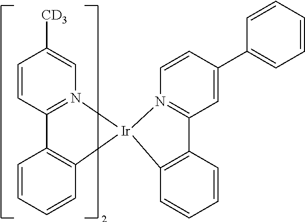

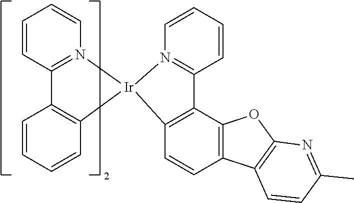

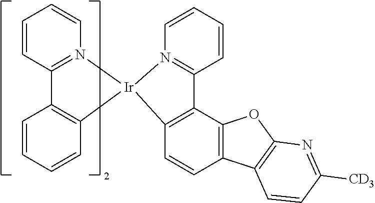

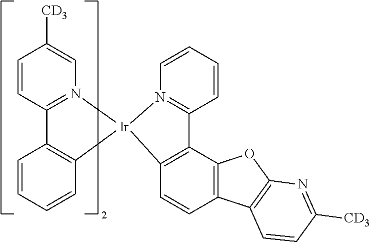

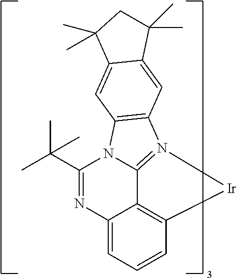

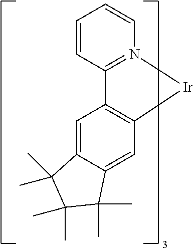

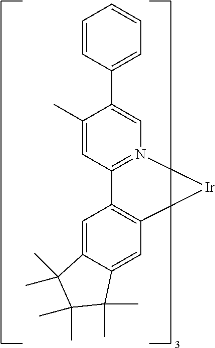

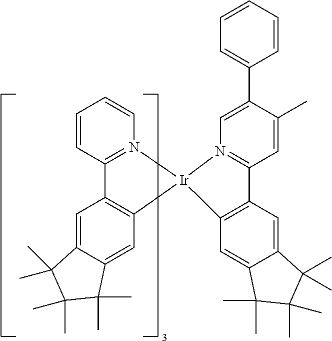

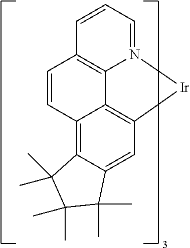

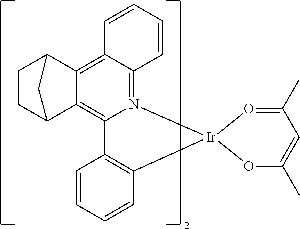

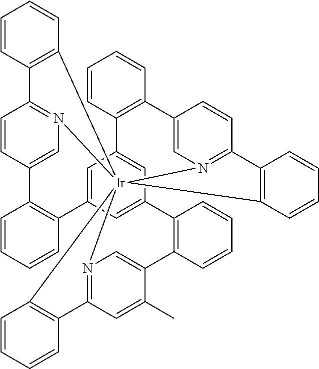

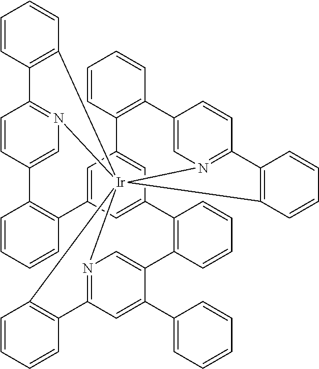

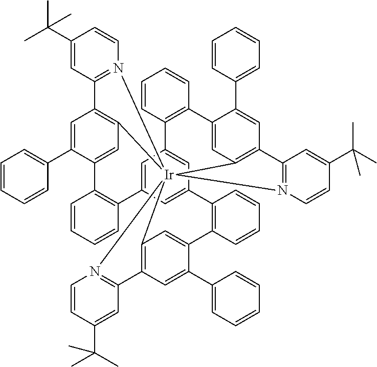

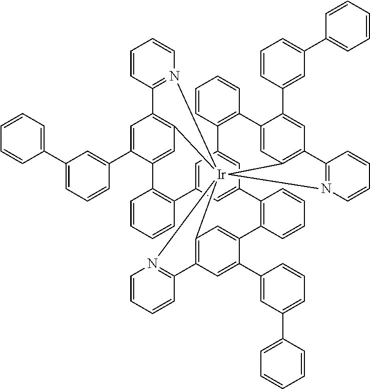

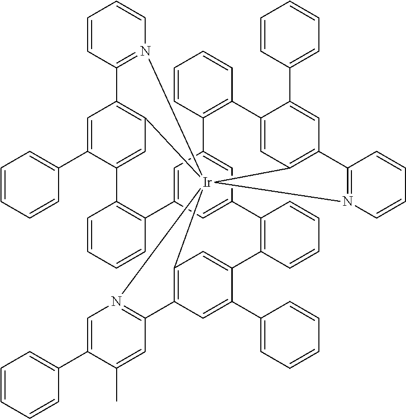

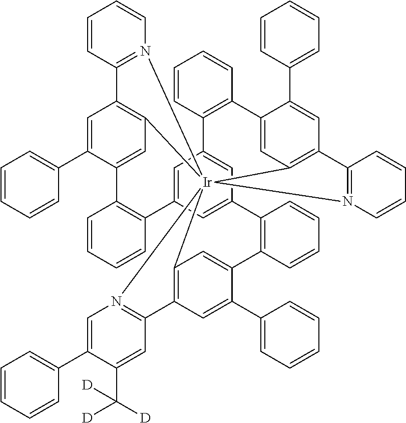

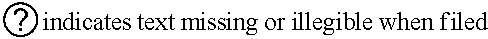

The compounds of the invention are especially also suitable as matrix materials for phosphorescent emitters in organic electroluminescent devices, as described, for example, in WO 98/24271, US 2011/0248247 and US 2012/0223633. In these multicolor display components, an additional blue emission layer is applied by vapor deposition over the full area to all pixels, including those having a color other than blue.

In a further embodiment of the invention, the organic electroluminescent device of the invention does not contain any separate hole injection layer and/or hole transport layer and/or hole blocker layer and/or electron transport layer, meaning that the emitting layer directly adjoins the hole injection layer or the anode, and/or the emitting layer directly adjoins the electron transport layer or the electron injection layer or the cathode, as described, for example, in WO 2005/053051. It is additionally possible to use a metal complex identical or similar to the metal complex in the emitting layer as hole transport or hole injection material directly adjoining the emitting layer, as described, for example, in WO 2009/030981.

In the further layers of the organic electroluminescent device of the invention, it is possible to use any materials as typically used according to the prior art. The person skilled in the art will therefore be able, without exercising inventive skill, to use any materials known for organic electroluminescent devices in combination with the inventive compounds of formula (1) or the above-recited preferred embodiments.

Additionally preferred is an organic electroluminescent device, characterized in that one or more layers are coated by a sublimation process. In this case, the materials are applied by vapor deposition in vacuum sublimation systems at an initial pressure of less than 10⁻⁵ mbar, preferably less than 10⁻⁶ mbar. However, it is also possible that the initial pressure is even lower, for example less than 10⁻⁷ mbar.

Preference is likewise given to an organic electroluminescent device, characterized in that one or more layers are coated by the OVPD (organic vapor phase deposition) method or with the aid of a carrier gas sublimation. In this case, the materials are applied at a pressure between 10⁻⁵ mbar and 1 bar. A special case of this method is the OVJP (organic vapor jet printing) method, in which the materials are applied directly by a nozzle and thus structured.

Preference is additionally given to an organic electroluminescent device, characterized in that one or more layers are produced from solution, for example by spin-coating, or by any printing method, for example screen printing, flexographic printing, offset printing, LITI (light-induced thermal imaging, thermal transfer printing), inkjet printing or nozzle printing. For this purpose, soluble compounds are needed, which are obtained, for example, through suitable substitution.

Formulations for application of a compound of formula (1) or the preferred embodiments thereof that are set out above are novel. The present invention therefore further provides a formulation comprising at least one solvent and a compound of formula (1) or the preferred embodiments thereof that are set out above. The present invention further provides a formulation comprising at least one solvent and a compound of formula (1) or the preferred embodiments thereof that are set out above, and a compound of at least one of the formulae (7), (8), (9) and (10). The present invention further provides a formulation comprising at least one solvent and a compound of formula (1) or the preferred embodiments thereof that are set out above, and a compound of at least one of the formulae (11), (12), (13), (14), (15), (16), (17) and (18). It may further be the case that the formulations contain at least one solvent and a compound of formula (1) or the preferred embodiments set out above, and a compound according to at least one of the formulae (7), (8), (9), (10), (11), (12), (13), (14), (15), (16), (17) and (18).

In addition, hybrid methods are possible, in which, for example, one or more layers are applied from solution and one or more further layers are applied by vapor deposition.

Those skilled in the art are generally aware of these methods and are able to apply them without exercising inventive skill to organic electroluminescent devices comprising the compounds of the invention.

The compounds of the invention and the organic electroluminescent devices of the invention have the particular feature of an improved lifetime over the prior art. At the same time, the further electronic properties of the electroluminescent devices, such as efficiency or operating voltage, remain at least equally good. In a further variant, the compounds of the invention and the organic electroluminescent devices of the invention especially feature improved efficiency and/or operating voltage and higher lifetime compared to the prior art.

The electronic devices of the invention, especially organic electroluminescent devices, are notable for one or more of the following surprising advantages over the prior art:
1. Electronic devices, especially organic electroluminescent devices, comprising compounds of formula (1) or the preferred embodiments recited above and hereinafter, especially as matrix material or as hole-conducting materials, have a very good lifetime. In this context, these compounds especially bring about low roll-off, i.e. a small drop in power efficiency of the device at high luminances.
2. Electronic devices, especially organic electroluminescent devices, comprising compounds of formula (1) or the preferred embodiments recited above and hereinafter, have excellent efficiency as hole-conducting materials and/or matrix materials. In this context, compounds of the invention having structures of formula (1) or the preferred embodiments recited above and hereinafter bring about a low operating voltage when used in electronic devices.
3. The inventive compounds of formula (1) or the preferred embodiments recited above and hereinafter exhibit very high stability and lifetime.
4. With compounds of formula (1) or the preferred embodiments recited above and hereinafter, it is possible to avoid the formation of optical loss channels in electronic devices, especially organic electroluminescent devices. As a result, these devices feature a high PL efficiency and hence high EL efficiency of emitters, and excellent energy transmission of the matrices to dopants.
5. Compounds of formula (1) or the preferred embodiments recited above and hereinafter have excellent glass film formation.
6. Compounds of formula (1) or the preferred embodiments recited above and hereinafter form very good films from solutions.
7. The compounds of formula (1) or the preferred embodiments recited above and hereinafter have a low triplet level T₁ which may, for example, be in the range of −2.22 eV to −2.9 eV.

These abovementioned advantages are not accompanied by an inordinately high deterioration in the further electronic properties.

It should be pointed out that variations of the embodiments described in the present invention are covered by the scope of this invention. Any feature disclosed in the present invention may, unless this is explicitly ruled out, be exchanged for alternative features which serve the same purpose or an equivalent or similar purpose. Thus, any feature disclosed in the present invention, unless stated otherwise, should be considered as an example of a generic series or as an equivalent or similar feature.

All features of the present invention may be combined with one another in any manner, unless particular features and/or steps are mutually exclusive. This is especially true of preferred features of the present invention. Equally, features of non-essential combinations may be used separately (and not in combination).

It should also be pointed out that many of the features, and especially those of the preferred embodiments of the present invention, should themselves be regarded as inventive and not merely as some of the embodiments of the present invention. For these features, independent protection may be sought in addition to or as an alternative to any currently claimed invention.

The technical teaching disclosed with the present invention may be abstracted and combined with other examples.

The invention is illustrated in detail by the examples which follow, without any intention of restricting it thereby. The person skilled in the art will be able to use the information given to execute the invention over the entire scope disclosed and to prepare further compounds of the invention without exercising inventive skill and to use them in electronic devices or to employ the process of the invention.

### EXAMPLES

The syntheses which follow, unless stated otherwise, are conducted under a protective gas atmosphere in dried solvents. The solvents and reagents can be purchased, for example, from Sigma-ALDRICH or ABCR. For the compounds known from the literature, the corresponding CAS numbers are also reported in each case.

### Synthesis Examples

a) Bromination

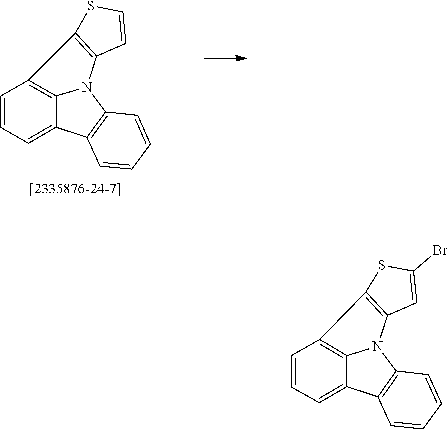 [SMILES: c1ccc2c(c1)c1cccc3c4sccc4n2c13 ; Brc1cc2c(s1)c1cccc3c4ccccc4n2c31]

To a solution of 37 g (150 mmol) of thieno[2′,3′:4,5]pyrrolo[3,2,1-jk]carbazole in chloroform (900 ml) is added N-bromosuccinimide (26.6 g, 150 mmol) in portions at −10° C. with exclusion of light, and the mixture is stirred at this temperature for 2 h. The reaction is ended by addition of sodium sulfite solution and the mixture is stirred at room temperature for a further 30 min. After phase separation, the organic phase is washed with water and the aqueous phase is extracted with dichloromethane. The combined organic phases are dried over sodium sulfate and concentrated under reduced pressure. The residue is dissolved in toluene and filtered through silica gel. Subsequently, the crude product is recrystallized from toluene/heptane.

Yield: 34 g (106 mmol), 70% of theory, colorless solid.

The following compounds are prepared in an analogous manner:

| | Reactant 1 | Product | Yield |
|---|---|---|---|
| 1a | 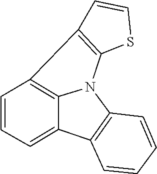 [SMILES: c1ccc2c(c1)c1cccc3c4ccsc4n2c13] | 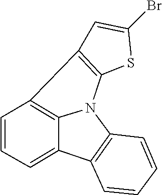 [SMILES: Brc1cc2c3cccc4c5ccccc5n(c2s1)c43] | 75% |
| | [2201847-06-3] | | |
| 2a | 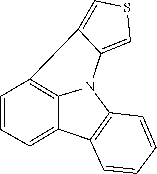 [SMILES: c1ccc2c(c1)c1cccc3c4cscc4n2c13] | 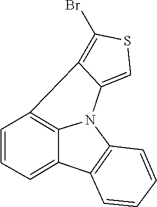 [SMILES: Brc1scc2c1c1cccc3c4ccccc4n2c31] | 48% |
| | [2335876-25-8] | | |
| 3a | 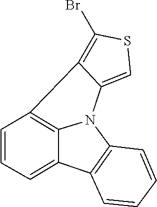 [SMILES: Brc1scc2c1c1cccc3c4ccccc4n2c31] | 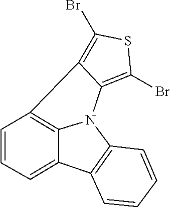 [SMILES: Brc1sc(Br)c2c1c1cccc3c4ccccc4n2c31] | 72% |

b) Suzuki Reaction

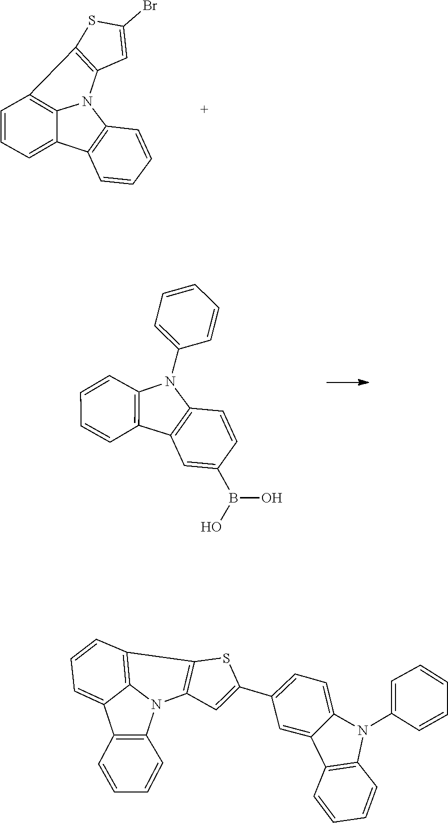 [SMILES: Brc1cc2c(s1)c1cccc3c4ccccc4n2c31 ; OB(O)c1ccc2c(c1)c1ccccc1n2-c1ccccc1 ; c1ccc(-n2c3ccccc3c3cc(-c4cc5c(s4)c4cccc6c7ccccc7n5c64)ccc32)cc1]

51.4 g (150 mmol) of compound a, 50 g (160 mmol) of N-phenylcarbazole-3-boronic acid and 36 g (340 mmol) of sodium carbonate are suspended in 1000 ml of ethylene glycol dimethyl ether and 280 ml of water. 1.8 g (1.5 mmol) of tetrakis(triphenylphosphine)palladium(0) are added to this suspension, and the reaction mixture is heated under reflux for 16 h. After cooling, the organic phase is removed, filtered through silica gel, washed three times with 200 ml of water and then concentrated to dryness. The residue is subjected to hot extraction with toluene and recrystallized from toluene/n-heptane and finally sublimed under high vacuum.

The yield is 51 g (104 mmol), corresponding to 67% of theory.

The following compounds are prepared in an analogous manner:

| | Reactant 1 | Reactant 2 | Product | Yield |
|---|---|---|---|---|
| 1b | 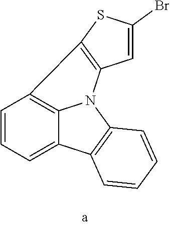 [SMILES: Brc1cc2c(s1)c1cccc3c4ccccc4n2c31] | 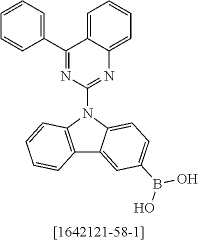 [SMILES: OB(O)c1ccc2c(c1)c1ccccc1n2-c1nc(-c2ccccc2)c2ccccc2n1] | 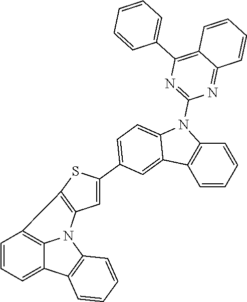 [SMILES: c1ccc(-c2nc(-n3c4ccccc4c4cc(-c5cc6c(s5)c5cccc7c8ccccc8n6c75)ccc43)nc3ccccc23)cc1] | 67% |
| 2b | 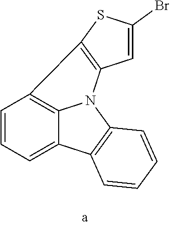 [SMILES: Brc1cc2c(s1)c1cccc3c4ccccc4n2c31] | 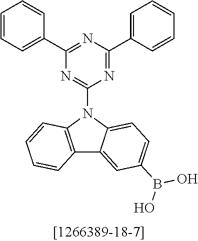 [SMILES: OB(O)c1ccc2c(c1)c1ccccc1n2-c1nc(-c2ccccc2)nc(-c2ccccc2)n1] | 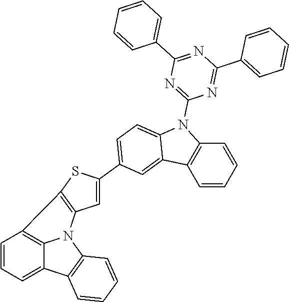 [SMILES: c1ccc(-c2nc(-c3ccccc3)nc(-n3c4ccccc4c4cc(-c5cc6c(s5)c5cccc7c8ccccc8n6c75)ccc43)n2)cc1] | 63% |
| 3b | 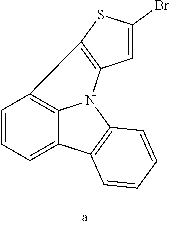 [SMILES: Brc1cc2c(s1)c1cccc3c4ccccc4n2c31] | 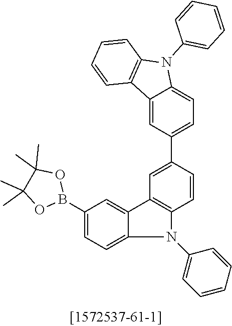 [SMILES: CC1(C)OB(c2ccc3c(c2)c2cc(-c4ccc5c(c4)c4ccccc4n5-c4ccccc4)ccc2n3-c2ccccc2)OC1(C)C] | 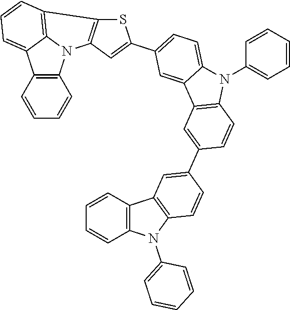 [SMILES: c1ccc(-n2c3ccccc3c3cc(-c4ccc5c(c4)c4cc(-c6cc7c(s6)c6cccc8c9ccccc9n7c86)ccc4n5-c4ccccc4)ccc32)cc1] | 63% |
| 4b | 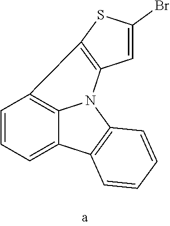 [SMILES: Brc1cc2c(s1)c1cccc3c4ccccc4n2c31] | 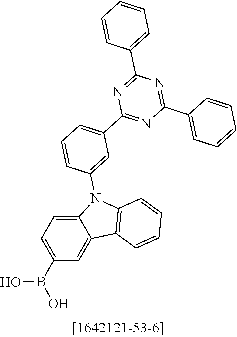 [SMILES: OB(O)c1ccc2c(c1)c1ccccc1n2-c1cccc(-c2nc(-c3ccccc3)nc(-c3ccccc3)n2)c1] | 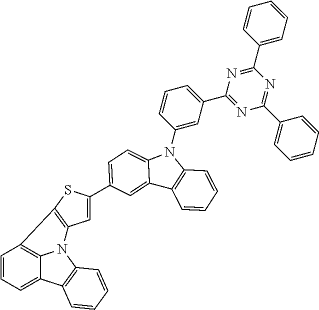 [SMILES: c1ccc(-c2nc(-c3ccccc3)nc(-c3cccc(-n4c5ccccc5c5cc(-c6cc7c(s6)c6cccc8c9ccccc9n7c86)ccc54)c3)n2)cc1] | 67% |
| 5b | 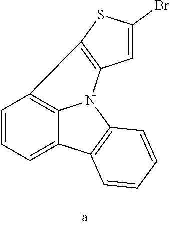 [SMILES: Brc1cc2c(s1)c1cccc3c4ccccc4n2c31] | 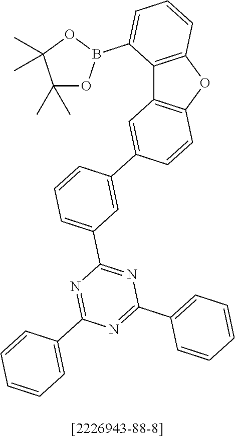 [SMILES: CC1(C)OB(c2cccc3oc4ccc(-c5cccc(-c6nc(-c7ccccc7)nc(-c7ccccc7)n6)c5)cc4c23)OC1(C)C] | 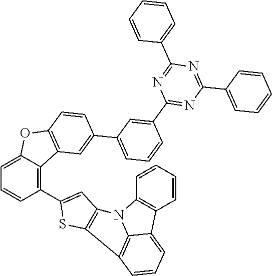 [SMILES: c1ccc(-c2nc(-c3ccccc3)nc(-c3cccc(-c4ccc5oc6cccc(-c7cc8c(s7)c7cccc9c%10ccccc%10n8c97)c6c5c4)c3)n2)cc1] | 69% |
| 6b | 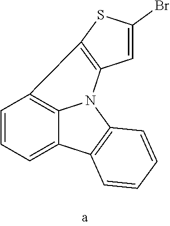 [SMILES: Brc1cc2c(s1)c1cccc3c4ccccc4n2c31] | 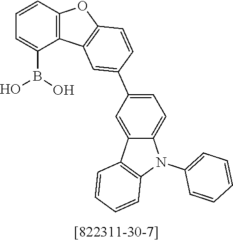 [SMILES: OB(O)c1cccc2oc3ccc(-c4ccc5c(c4)c4ccccc4n5-c4ccccc4)cc3c12] | 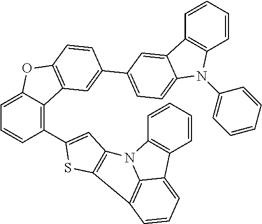 [SMILES: c1ccc(-n2c3ccccc3c3cc(-c4ccc5oc6cccc(-c7cc8c(s7)c7cccc9c%10ccccc%10n8c97)c6c5c4)ccc32)cc1] | 66% |
| 7b | 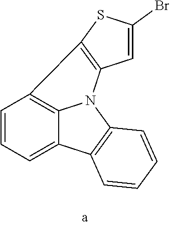 [SMILES: Brc1cc2c(s1)c1cccc3c4ccccc4n2c31] | 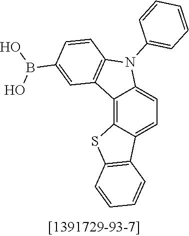 [SMILES: OB(O)c1ccc2c(c1)c1c3sc4ccccc4c3ccc1n2-c1ccccc1] | 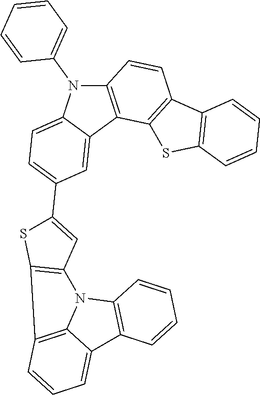 [SMILES: c1ccc(-n2c3ccc(-c4cc5c(s4)c4cccc6c7ccccc7n5c64)cc3c3c4sc5ccccc5c4ccc32)cc1] | 64% |
| 8b | 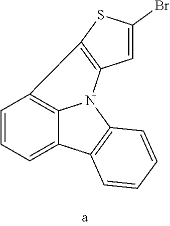 [SMILES: Brc1cc2c(s1)c1cccc3c4ccccc4n2c31] | 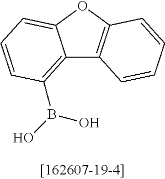 [SMILES: OB(O)c1cccc2oc3ccccc3c12] | 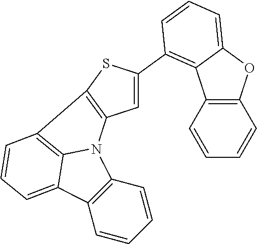 [SMILES: c1ccc2c(c1)oc1cccc(-c3cc4c(s3)c3cccc5c6ccccc6n4c53)c12] | 70% |
| 9b | 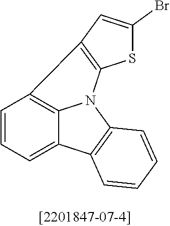 [SMILES: Brc1cc2c3cccc4c5ccccc5n(c2s1)c43] | 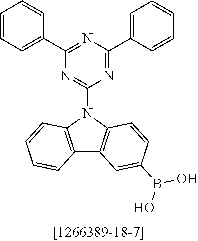 [SMILES: OB(O)c1ccc2c(c1)c1ccccc1n2-c1nc(-c2ccccc2)nc(-c2ccccc2)n1] | 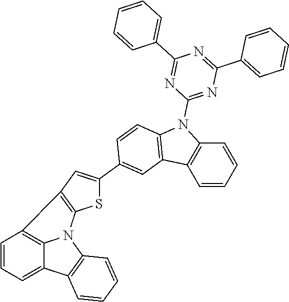 [SMILES: c1ccc(-c2nc(-c3ccccc3)nc(-n3c4ccccc4c4cc(-c5cc6c7cccc8c9ccccc9n(c6s5)c87)ccc43)n2)cc1] | 73% |
| 10b | 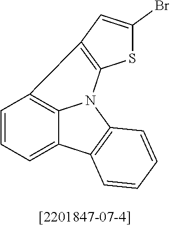 [SMILES: Brc1cc2c3cccc4c5ccccc5n(c2s1)c43] | 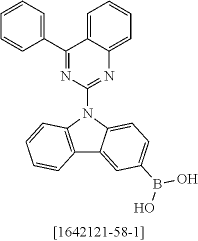 [SMILES: OB(O)c1ccc2c(c1)c1ccccc1n2-c1nc(-c2ccccc2)c2ccccc2n1] | 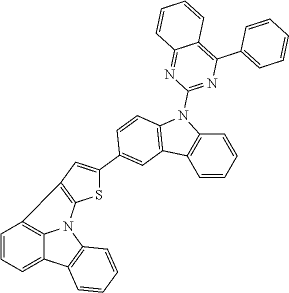 [SMILES: c1ccc(-c2nc(-n3c4ccccc4c4cc(-c5cc6c7cccc8c9ccccc9n(c6s5)c87)ccc43)nc3ccccc23)cc1] | 61% |
| 11b | 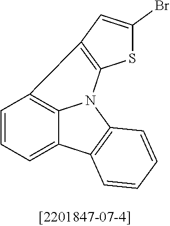 [SMILES: Brc1cc2c3cccc4c5ccccc5n(c2s1)c43] | 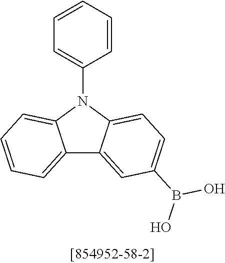 [SMILES: OB(O)c1ccc2c(c1)c1ccccc1n2-c1ccccc1] | 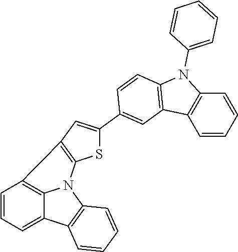 [SMILES: c1ccc(-n2c3ccccc3c3cc(-c4cc5c6cccc7c8ccccc8n(c5s4)c76)ccc32)cc1] | 64% |
| 12b | 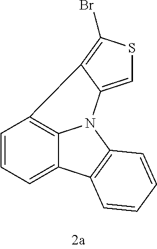 [SMILES: Brc1scc2c1c1cccc3c4ccccc4n2c31] | 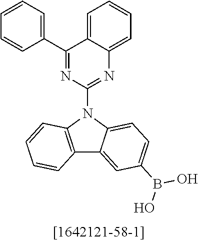 [SMILES: OB(O)c1ccc2c(c1)c1ccccc1n2-c1nc(-c2ccccc2)c2ccccc2n1] | 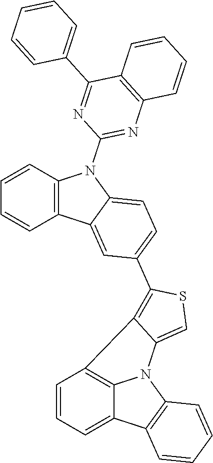 [SMILES: c1ccc(-c2nc(-n3c4ccccc4c4cc(-c5scc6c5c5cccc7c8ccccc8n6c75)ccc43)nc3ccccc23)cc1] | 66% |
| 13b | 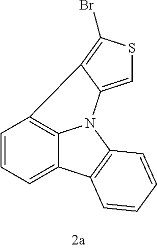 [SMILES: Brc1scc2c1c1cccc3c4ccccc4n2c31] | 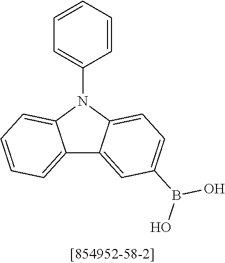 [SMILES: OB(O)c1ccc2c(c1)c1ccccc1n2-c1ccccc1] | 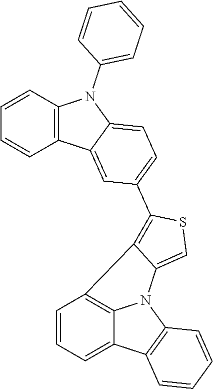 [SMILES: c1ccc(-n2c3ccccc3c3cc(-c4scc5c4c4cccc6c7ccccc7n5c64)ccc32)cc1] | 68% |
| 14b | 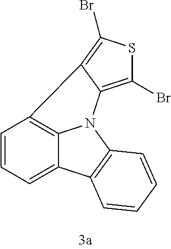 [SMILES: Brc1sc(Br)c2c1c1cccc3c4ccccc4n2c31] | 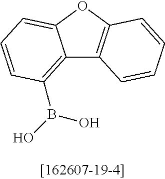 [SMILES: OB(O)c1cccc2oc3ccccc3c12] | 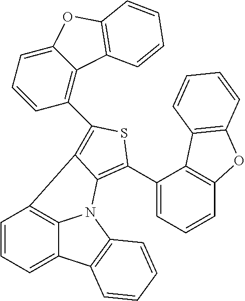 [SMILES: c1ccc2c(c1)oc1cccc(-c3sc(-c4cccc5oc6ccccc6c45)c4c3c3cccc5c6ccccc6n4c53)c12] | 70% |
| 15b | 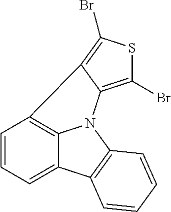 [SMILES: Brc1sc(Br)c2c1c1cccc3c4ccccc4n2c31] | 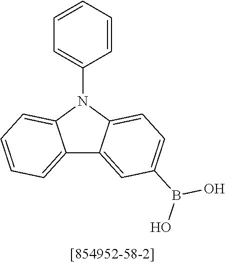 [SMILES: OB(O)c1ccc2c(c1)c1ccccc1n2-c1ccccc1] | 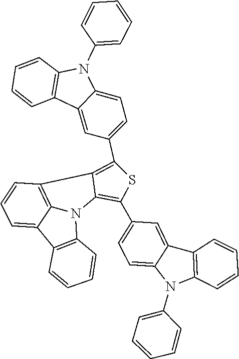 [SMILES: c1ccc(-n2c3ccccc3c3cc(-c4sc(-c5ccc6c(c5)c5ccccc5n6-c5ccccc5)c5c4c4cccc6c7ccccc7n5c64)ccc32)cc1] | 78% |
| 16b | 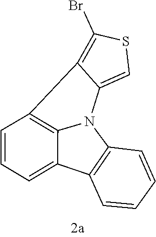 [SMILES: Brc1scc2c1c1cccc3c4ccccc4n2c31] | 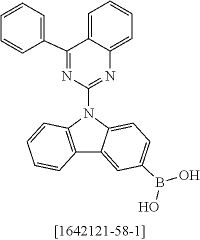 [SMILES: OB(O)c1ccc2c(c1)c1ccccc1n2-c1nc(-c2ccccc2)c2ccccc2n1] | 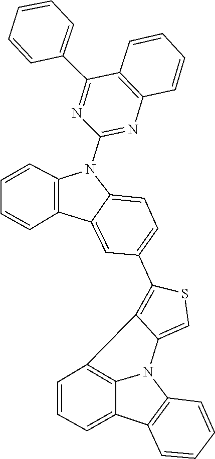 [SMILES: c1ccc(-c2nc(-n3c4ccccc4c4cc(-c5scc6c5c5cccc7c8ccccc8n6c75)ccc43)nc3ccccc23)cc1] | 66% |
| 17b | 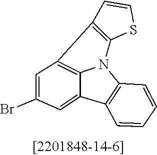 [SMILES: Brc1cc2c3ccccc3n3c4sccc4c(c1)c23] | 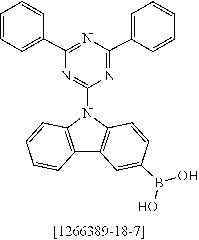 [SMILES: OB(O)c1ccc2c(c1)c1ccccc1n2-c1nc(-c2ccccc2)nc(-c2ccccc2)n1] | 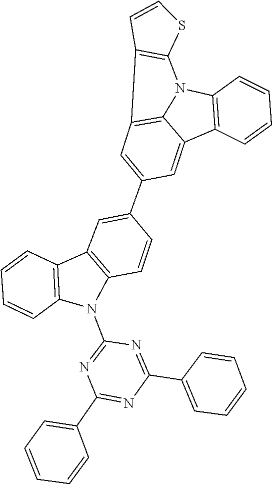 [SMILES: c1ccc(-c2nc(-c3ccccc3)nc(-n3c4ccccc4c4cc(-c5cc6c7ccccc7n7c8sccc8c(c5)c67)ccc43)n2)cc1] | 69% |
| 18b | 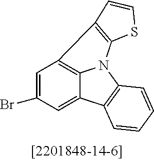 [SMILES: Brc1cc2c3ccccc3n3c4sccc4c(c1)c23] | 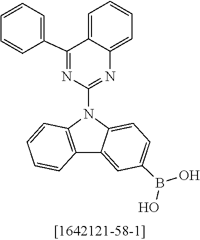 [SMILES: OB(O)c1ccc2c(c1)c1ccccc1n2-c1nc(-c2ccccc2)c2ccccc2n1] | 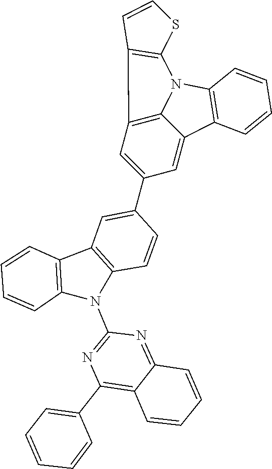 [SMILES: c1ccc(-c2nc(-n3c4ccccc4c4cc(-c5cc6c7ccccc7n7c8sccc8c(c5)c67)ccc43)nc3ccccc23)cc1] | 62% |
| 19b | 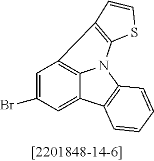 [SMILES: Brc1cc2c3ccccc3n3c4sccc4c(c1)c23] | 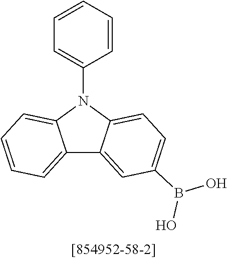 [SMILES: OB(O)c1ccc2c(c1)c1ccccc1n2-c1ccccc1] | 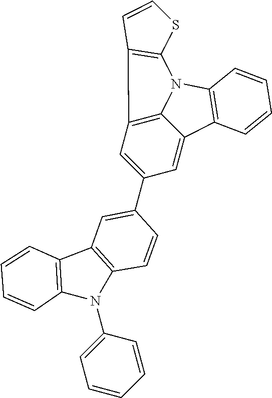 [SMILES: c1ccc(-n2c3ccccc3c3cc(-c4cc5c6ccccc6n6c7sccc7c(c4)c56)ccc32)cc1] | 71% |
| 20b | 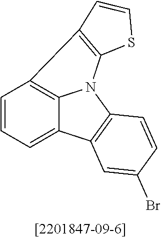 [SMILES: Brc1ccc2c(c1)c1cccc3c4ccsc4n2c13] | 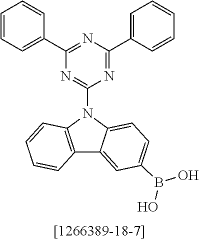 [SMILES: OB(O)c1ccc2c(c1)c1ccccc1n2-c1nc(-c2ccccc2)nc(-c2ccccc2)n1] | 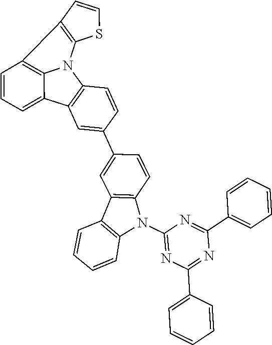 [SMILES: c1ccc(-c2nc(-c3ccccc3)nc(-n3c4ccccc4c4cc(-c5ccc6c(c5)c5cccc7c8ccsc8n6c57)ccc43)n2)cc1] | 70% |
| 21b | 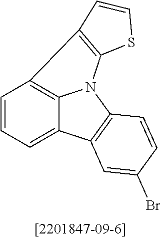 [SMILES: Brc1ccc2c(c1)c1cccc3c4ccsc4n2c13] | 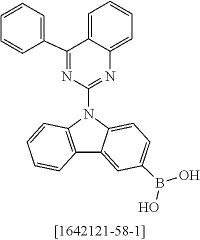 [SMILES: OB(O)c1ccc2c(c1)c1ccccc1n2-c1nc(-c2ccccc2)c2ccccc2n1] | 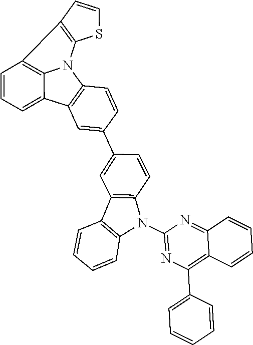 [SMILES: c1ccc(-c2nc(-n3c4ccccc4c4cc(-c5ccc6c(c5)c5cccc7c8ccsc8n6c57)ccc43)nc3ccccc23)cc1] | 68% |
| 22b | 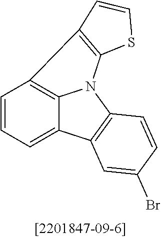 [SMILES: Brc1ccc2c(c1)c1cccc3c4ccsc4n2c13] | 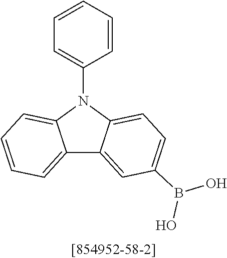 [SMILES: OB(O)c1ccc2c(c1)c1ccccc1n2-c1ccccc1] | 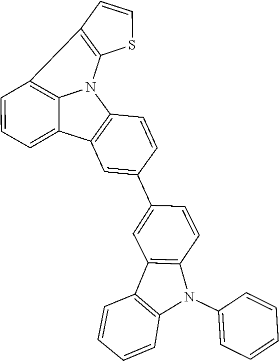 [SMILES: c1ccc(-n2c3ccccc3c3cc(-c4ccc5c(c4)c4cccc6c7ccsc7n5c46)ccc32)cc1] | 73% |
| 23b | 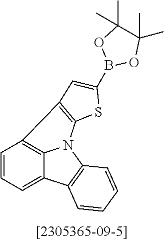 [SMILES: CC1(C)OB(c2cc3c4cccc5c6ccccc6n(c3s2)c54)OC1(C)C] | 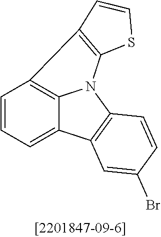 [SMILES: Brc1ccc2c(c1)c1cccc3c4ccsc4n2c13] | 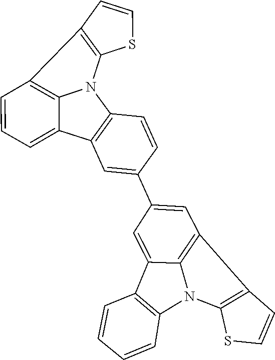 [SMILES: c1ccc2c(c1)c1cc(-c3ccc4c(c3)c3cccc5c6ccsc6n4c35)cc3c4ccsc4n2c13] | 65% |
| 24b | 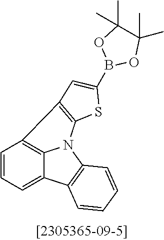 [SMILES: CC1(C)OB(c2cc3c4cccc5c6ccccc6n(c3s2)c54)OC1(C)C] | 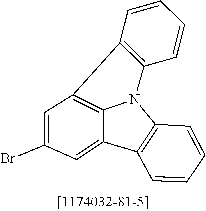 [SMILES: Brc1cc2c3ccccc3n3c4ccccc4c(c1)c23] | 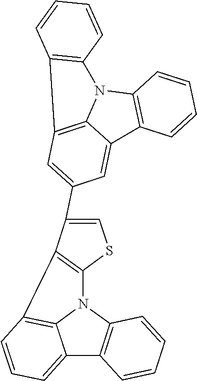 [SMILES: c1ccc2c(c1)c1cc(-c3csc4c3c3cccc5c6ccccc6n4c53)cc3c4ccccc4n2c13] | 71% |
| 25b | 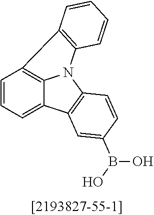 [SMILES: OB(O)c1ccc2c(c1)c1cccc3c4ccccc4n2c31] | 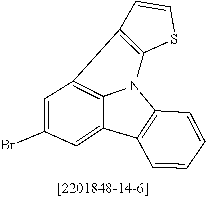 [SMILES: Brc1cc2c3ccccc3n3c4sccc4c(c1)c23] | 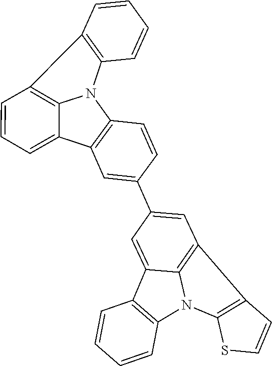 [SMILES: c1ccc2c(c1)c1cccc3c4cc(-c5cc6c7ccccc7n7c8sccc8c(c5)c67)ccc4n2c13] | 66% |
| 26b | 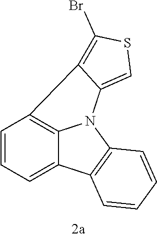 [SMILES: Brc1scc2c1c1cccc3c4ccccc4n2c31] | 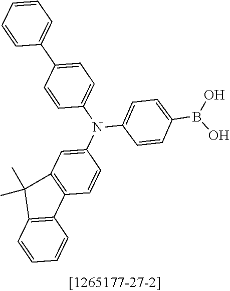 [SMILES: CC1(C)c2ccccc2-c2ccc(N(c3ccc(B(O)O)cc3)c3ccc(-c4ccccc4)cc3)cc21] | 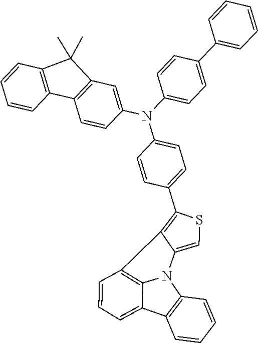 [SMILES: CC1(C)c2ccccc2-c2ccc(N(c3ccc(-c4ccccc4)cc3)c3ccc(-c4scc5c4c4cccc6c7ccccc7n5c64)cc3)cc21] | 70% |
| 27b | 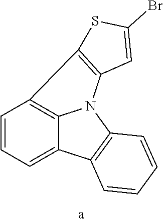 [SMILES: Brc1cc2c(s1)c1cccc3c4ccccc4n2c31] | 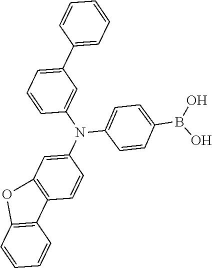 [SMILES: OB(O)c1ccc(N(c2cccc(-c3ccccc3)c2)c2ccc3c(c2)oc2ccccc23)cc1] | 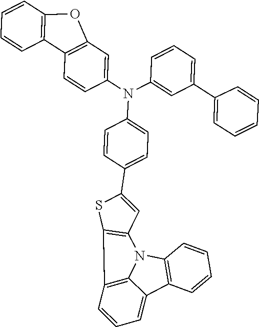 [SMILES: c1ccc(-c2cccc(N(c3ccc(-c4cc5c(s4)c4cccc6c7ccccc7n5c64)cc3)c3ccc4c(c3)oc3ccccc34)c2)cc1] | 75% |
| 28b | 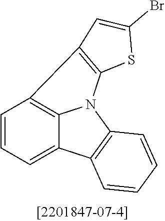 [SMILES: Brc1cc2c3cccc4c5ccccc5n(c2s1)c43] | 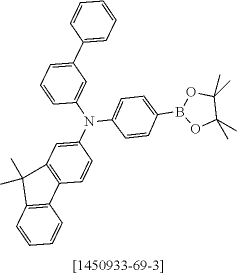 | 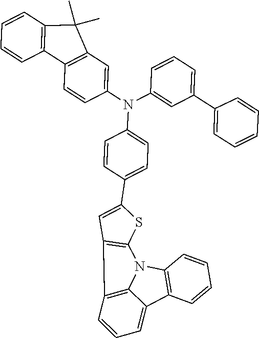 [SMILES: CC1(C)c2ccccc2-c2ccc(N(c3ccc(-c4cc5c6cccc7c8ccccc8n(c5s4)c76)cc3)c3cccc(-c4ccccc4)c3)cc21] | 72% |

c) Copper-Catalyzed Condensation

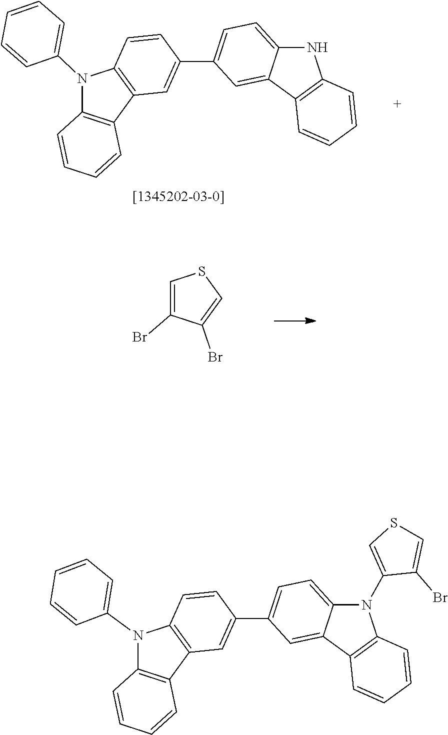 [SMILES: c1ccc(-n2c3ccccc3c3cc(-c4ccc5[nH]c6ccccc6c5c4)ccc32)cc1 ; Brc1cscc1Br ; Brc1cscc1-n1c2ccccc2c2cc(-c3ccc4c(c3)c3ccccc3n4-c3ccccc3)ccc21]

Under protective gas and without solvent, 10.6 g (44 mmol) of 3,4-dibromothiophene, 18 g (40 mmol) of 9-phenyl-3,3′-bi-9H-carbazole, 6 g (44 mmol) of K₂CO₃ and 500 mg (2 mmol) of CuSO₄-5H₂O are stirred in a reaction vessel equipped with a stirrer bar and purged three times with argon. The reaction mixture is stirred under reflux (250° C.) for 24 h. After cooling, the mixture is admixed with 100 ml of dichloromethane and 100 ml of water, the organic phase is separated, dried over MgSO4 and filtered, and the solvent is removed under reduced pressure. The residue is purified by column chromatography using silica gel (eluent: DCM/heptane (1:3)).

The yield is 7 g (12.5 mmol), corresponding to 48% of theory.

The following compounds can be prepared analogously:

| | Reactant 1 | Reactant 2 | Product | Yield |
|---|---|---|---|---|
| 1c | 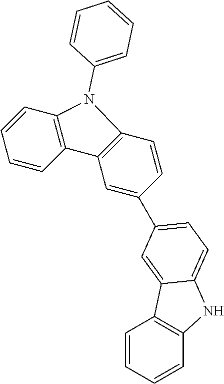 [SMILES: c1ccc(-n2c3ccccc3c3cc(-c4ccc5[nH]c6ccccc6c5c4)ccc32)cc1] | 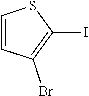 | 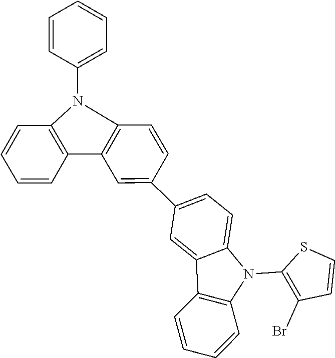 [SMILES: Brc1ccsc1-n1c2ccccc2c2cc(-c3ccc4c(c3)c3ccccc3n4-c3ccccc3)ccc21] | 38% |
| | [1345202-03-0] | | | |
| 2c | 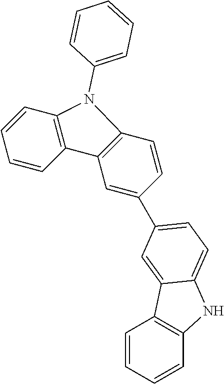 [SMILES: c1ccc(-n2c3ccccc3c3cc(-c4ccc5[nH]c6ccccc6c5c4)ccc32)cc1] | 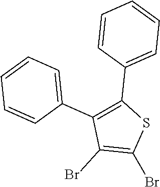 [SMILES: Brc1sc(-c2ccccc2)c(-c2ccccc2)c1Br] | 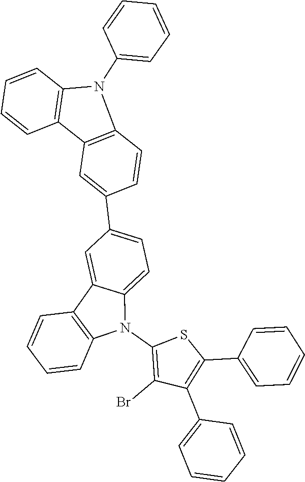 [SMILES: Brc1c(-n2c3ccccc3c3cc(-c4ccc5c(c4)c4ccccc4n5-c4ccccc4)ccc32)sc(-c2ccccc2)c1-c1ccccc1] | 34% |
| | [1345202-03-0] | [859490-18-9] | | |
| 3c | 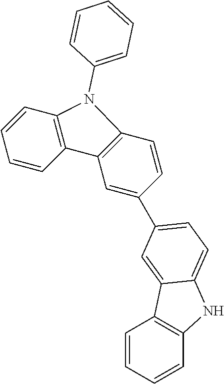 [SMILES: c1ccc(-n2c3ccccc3c3cc(-c4ccc5[nH]c6ccccc6c5c4)ccc32)cc1] | 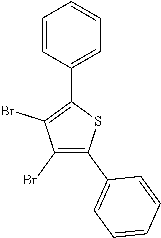 [SMILES: Brc1c(-c2ccccc2)sc(-c2ccccc2)c1Br] | 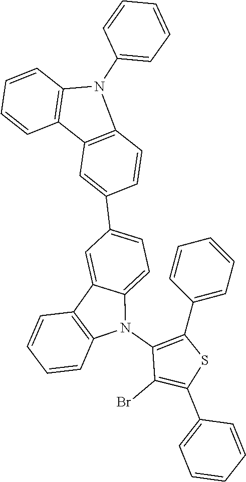 [SMILES: Brc1c(-c2ccccc2)sc(-c2ccccc2)c1-n1c2ccccc2c2cc(-c3ccc4c(c3)c3ccccc3n4-c3ccccc3)ccc21] | 38% |
| | [1345202-03-0] | [874511-95-2] | | |
| 4c | 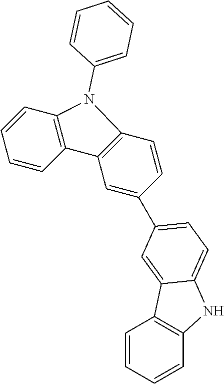 [SMILES: c1ccc(-n2c3ccccc3c3cc(-c4ccc5[nH]c6ccccc6c5c4)ccc32)cc1] | 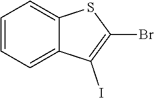 [SMILES: Brc1sc2ccccc2c1I] | 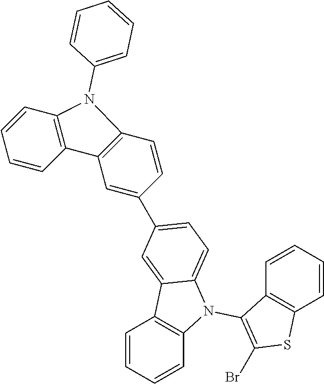 [SMILES: Brc1sc2ccccc2c1-n1c2ccccc2c2cc(-c3ccc4c(c3)c3ccccc3n4-c3ccccc3)ccc21] | 41% |
| | [1345202-03-0] | [2226857-79-4] | | |
| 5c | 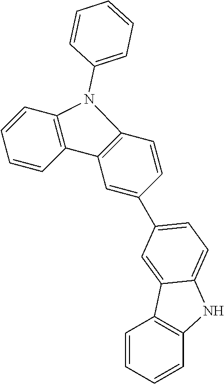 [SMILES: c1ccc(-n2c3ccccc3c3cc(-c4ccc5[nH]c6ccccc6c5c4)ccc32)cc1] | 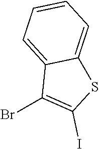 [SMILES: Brc1c(I)sc2ccccc12] | 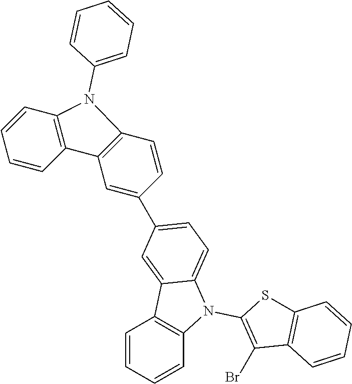 [SMILES: Brc1c(-n2c3ccccc3c3cc(-c4ccc5c(c4)c4ccccc4n5-c4ccccc4)ccc32)sc2ccccc12] | 37% |
| | [1345202-03-0] | [140898-76-6] | 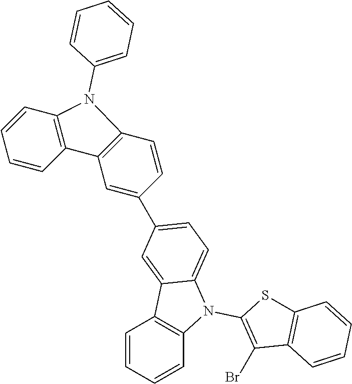 [SMILES: Brc1c(-n2c3ccccc3c3cc(-c4ccc5c(c4)c4ccccc4n5-c4ccccc4)ccc32)sc2ccccc12] | 37% |
| 6c | 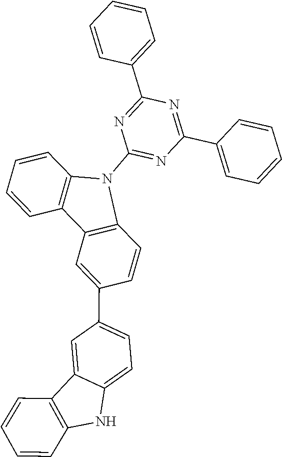 [SMILES: c1ccc(-c2nc(-c3ccccc3)nc(-n3c4ccccc4c4cc(-c5ccc6[nH]c7ccccc7c6c5)ccc43)n2)cc1] | 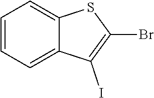 [SMILES: Brc1sc2ccccc2c1I] | 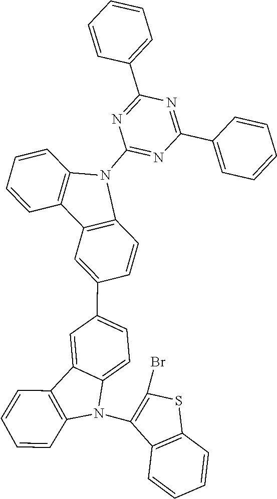 [SMILES: Brc1sc2ccccc2c1-n1c2ccccc2c2cc(-c3ccc4c(c3)c3ccccc3n4-c3nc(-c4ccccc4)nc(-c4ccccc4)n3)ccc21] | 31% |
| | [1346669-47-3] | [2226857-79-4] | | |

d) C—H-Activated Cyclization

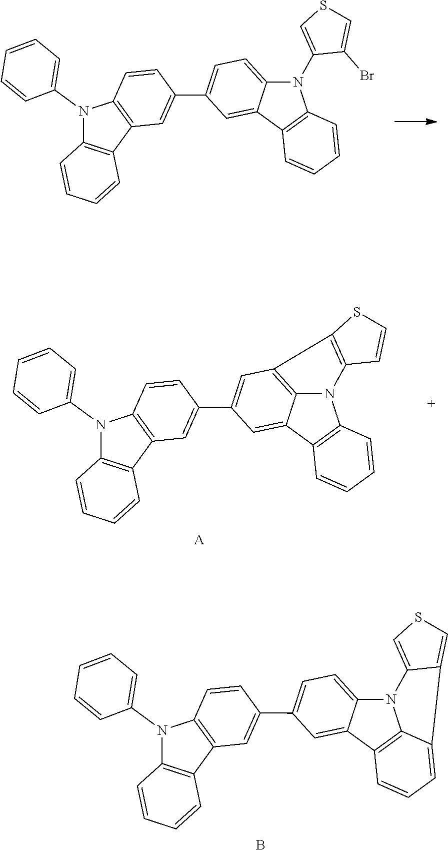

A round-bottom flask is charged with 20.8 g (63.7 mmol) of compound b, 17.3 g (126 mmol) of K₂CO₃, 1.8 g (3.21 mmol) of (NHC)Pd(allyl)Cl, and then degassed and filled with argon. Under an argon atmosphere, 200 ml of degassed dimethylacetamide with a water content of less than 1000 ppm is added. The mixture is heated to 130° C. for 24 hours under an argon counterflow and stirred until completion. After cooling, 100 ml of dichloromethane and 100 ml of water are added to the mixture, the organic phase is separated, dried over MgSO4 and filtered, and the solvent is removed under reduced pressure. The residue is purified by column chromatography using silica gel (eluent: DCM/heptane (1:3)). The residue is subjected to hot extraction with toluene and recrystallized from toluene/n-heptane and finally sublimed under high vacuum. The yield is 10.5 (21.9 mmol) of the mixture of A+B, corresponding to 60% of theory. After column chromatography separation and subsequent workup, 22% A and 38% B are obtained.

The following compounds can be prepared analogously:

| | Reactant 1 | Product A | Product B | Yield |
|---|---|---|---|---|
| 1d | 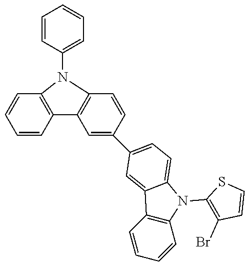 [SMILES: Brc1ccsc1-n1c2ccccc2c2cc(-c3ccc4c(c3)c3ccccc3n4-c3ccccc3)ccc21] | 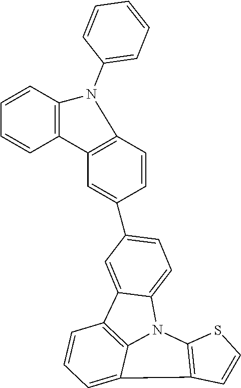 [SMILES: c1ccc(-n2c3ccccc3c3cc(-c4ccc5c(c4)c4cccc6c7ccsc7n5c46)ccc32)cc1] | 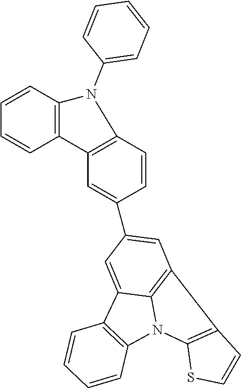 [SMILES: c1ccc(-n2c3ccccc3c3cc(-c4cc5c6ccccc6n6c7sccc7c(c4)c56)ccc32)cc1] | 35% A 21% B |
| 2d | 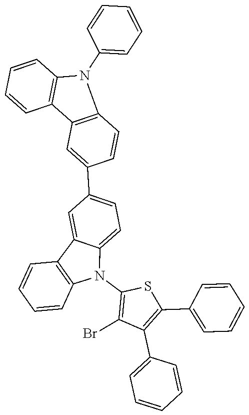 [SMILES: Brc1c(-n2c3ccccc3c3cc(-c4ccc5c(c4)c4ccccc4n5-c4ccccc4)ccc32)sc(-c2ccccc2)c1-c1ccccc1] | 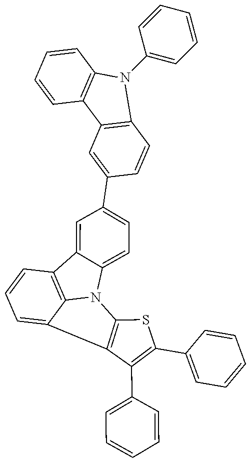 [SMILES: c1ccc(-c2sc3c(c2-c2ccccc2)c2cccc4c5cc(-c6ccc7c(c6)c6ccccc6n7-c6ccccc6)ccc5n3c42)cc1] | 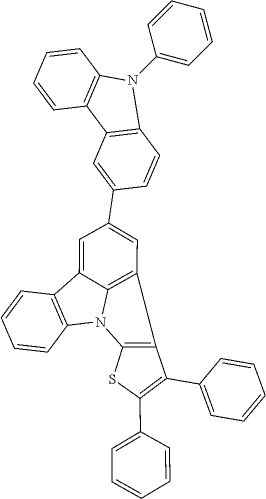 [SMILES: c1ccc(-c2sc3c(c2-c2ccccc2)c2cc(-c4ccc5c(c4)c4ccccc4n5-c4ccccc4)cc4c5ccccc5n3c42)cc1] | 32% A 18% B |
| 3d | 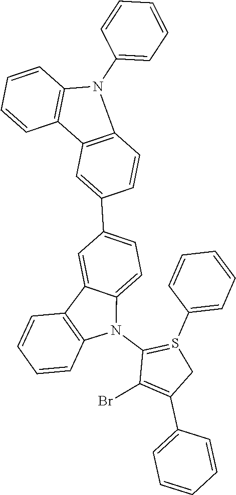 [SMILES: BrC1=C(c2ccccc2)CS(c2ccccc2)=C1n1c2ccccc2c2cc(-c3ccc4c(c3)c3ccccc3n4-c3ccccc3)ccc21] | 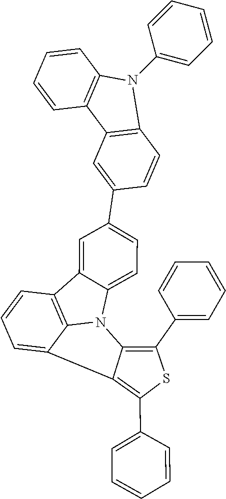 [SMILES: c1ccc(-c2sc(-c3ccccc3)c3c2c2cccc4c5cc(-c6ccc7c(c6)c6ccccc6n7-c6ccccc6)ccc5n3c42)cc1] | 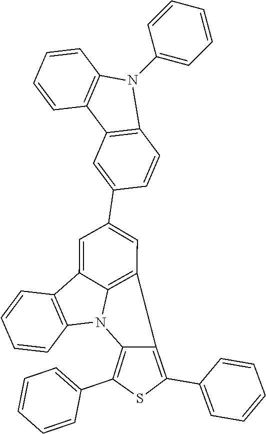 [SMILES: c1ccc(-c2sc(-c3ccccc3)c3c2c2cc(-c4ccc5c(c4)c4ccccc4n5-c4ccccc4)cc4c5ccccc5n3c42)cc1] | 32% A 16% B |
| 4d | 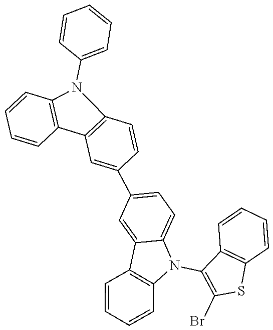 [SMILES: Brc1sc2ccccc2c1-n1c2ccccc2c2cc(-c3ccc4c(c3)c3ccccc3n4-c3ccccc3)ccc21] | 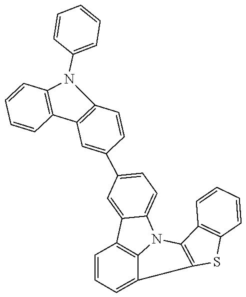 [SMILES: c1ccc(-n2c3ccccc3c3cc(-c4ccc5c(c4)c4cccc6c7sc8ccccc8c7n5c46)ccc32)cc1] | 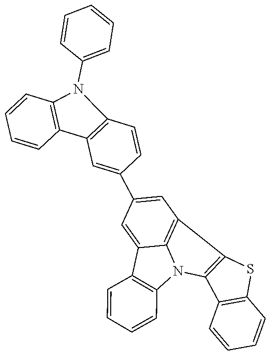 [SMILES: c1ccc(-n2c3ccccc3c3cc(-c4cc5c6ccccc6n6c5c(c4)c4sc5ccccc5c46)ccc32)cc1] | 30% A 15% B |

Production of the Electroluminescent Devices

Examples E1 to E20 which follow (see table 1) present the use of the materials of the invention in electroluminescent devices.

Pretreatment for examples E1-E20: Glass plates coated with structured ITO (indium tin oxide) of thickness 50 nm are treated prior to coating, first with an oxygen plasma, followed by an argon plasma. These plasma-treated glass plates form the substrates to which the OLEDs are applied.

The electroluminescent devices basically have the following layer structure: substrate/hole injection layer (HIL)/hole transport layer (HTL)/electron blocker layer (EBL)/emission layer (EML)/optional hole blocker layer (HBL)/electron transport layer (ETL)/optional electron injection layer (EIL) and finally a cathode. The cathode is formed by an aluminum layer of thickness 100 nm. The exact structure of the OLEDs can be found in table 1. The materials required for production of the electroluminescent devices are shown in table 2. The data of the electroluminescent devices are listed in table 3.

All materials are applied by thermal vapor deposition in a vacuum chamber. In this case, the emission layer always consists of at least one matrix material (host material), for the purposes of the invention at least two matrix materials, and an emitting dopant (emitter) which is added to the matrix material(s) in a particular proportion by volume by co-evaporation. Details given in such a form as 2b:BisC1:TEG1 (45%:45%:10%) mean here that the material 2b is present in the layer in a proportion by volume of 45%, BisC1 in a proportion of 45% and TEG1 in a proportion of 10%. Analogously, the electron transport layer may also consist of a mixture of two materials.

The electroluminescent devices are characterized in a standard manner. For this purpose, the electroluminescence spectra, the current efficiency (CE, measured in cd/A) and the external quantum efficiency (EQE, measured in %) are determined as a function of luminance, calculated from current-voltage-luminance characteristics assuming Lambertian emission characteristics, as is the lifetime. Electroluminescence spectra are determined at a luminance of 1000 cd/m², and these are used to calculate the CIE 1931 x and y color coordinates. The parameter U1000 in table 18 refers to the voltage which is required for a luminance of 1000 cd/m². CE1000 and EQE1000 respectively denote the current efficiency and external quantum efficiency that are attained at 1000 cd/m².

Use of Mixtures of the Invention in Electroluminescent Devices

The material combinations of the invention can be used in the emission layer in phosphorescent OLEDs. The inventive compounds 2b and 9b are used in examples E1 to E2 as green matrix material in the emission layer, and 10b and 18b are used in examples E16 to E17 as red matrix material in the emission layer.

The inventive combination of compound BisC1 with corresponding compounds b, 2b and 9b are used in examples E3 to E5 as matrix material in the emission layer. Further inventive combinations of compounds 24a and 2 dB with compounds Tz1 to Tz8 are used in examples E6 to E15 as matrix material in the emission layer.

A further inventive combination of compounds 18b with compound BisC2 is used in example E18 as red matrix material in the emission layer.

In example E19, inventive compound 9b is used as electron blocker material in the EBL.

In example E20, inventive compound 28b is used as hole transport material.

TABLE 1
Structure of the electroluminescent devices

| | HIL | HTL | EBL | EML | HBL | ETL | EIL |
|---|---|---|---|---|---|---|---|
| Ex. | thickness | thickness | thickness | thickness | thickness | thickness | thickness |
| E1 | HATCN | SpMA1 | SpMA2 | 2b: TEG1 | ST2 | ST2:LiQ | LiQ |
| | 5 nm | 125 nm | 10 nm | (97%:3%) | 10 nm | (50%:50%) | 1 nm |
| | | | | 35 nm | | 30 nm | |
| E2 | HATCN | SpMA1 | SpMA2 | 9b: TEG1 | ST2 | ST2:LiQ | LiQ |
| | 5 nm | 125 nm | 10 nm | (97%:3%) | 10 nm | (50%:50%) | 1 nm |
| | | | | 35 nm | | 30 nm | |
| E3 | HATCN | SpMA1 | SpMA2 | 2b:BisC1: | ST2 | ST2:LiQ | LiQ |
| | 5 nm | 125 nm | 10 nm | TEG1 | 10 nm | (50%:50%) | 1 nm |
| | | | | ((46%:47%: | | 30 nm | |
| | | | | 7%) 30 nm | | | |
| E4 | HATCN | SpMA1 | SpMA2 | 9b:BisC1: | ST2 | ST2:LiQ | LiQ |
| | 5 nm | 125 nm | 10 nm | TEG1 | 10 nm | (50%:50%) | 1 nm |
| | | | | ((46%:47%: | | 30 nm | |
| | | | | 7%) 30 nm | | | |
| E5 | HATCN | SpMA1 | SpMA2 | b:BisC1: | ST2 | ST2:LiQ | LiQ |
| | 5 nm | 125 nm | 10 nm | TEG1 | 10 nm | (50%:50%) | 1 nm |
| | | | | ((46%:47%: | | 30 nm | |
| | | | | 7%) 30 nm | | | |
| E6 | HATCN | SpMA1 | SpMA2 | 24b:TZ1: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E7 | HATCN | SpMA1 | SpMA2 | 24b:TZ2: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E8 | HATCN | SpMA1 | SpMA2 | 24b:Tz3: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E9 | HATCN | SpMA1 | SpMA2 | 24b:TZ4. | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E10 | HATCN | SpMA1 | SpMA2 | 24b:Tz5: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E11 | HATCN | SpMA1 | SpMA2 | 24b:Tz6: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E12 | HATCN | SpMA1 | SpMA2 | 24bTz7: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E13 | HATCN | SpMA1 | SpMA2 | 24b:Tz8: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E14 | HATCN | SpMA1 | SpMA2 | 2dB:Tz8: | ST2 | ST2:LiQ | LiQ |
| | 5 nm | 230 nm | 20 nm | TEG1 | 10 nm | (50%:50%) | 1 nm |
| | | | | (46%:47%: | | 30 nm | |
| | | | | 7%) 30 nm | | | |
| E15 | HATCN | SpMA1 | SpMA2 | 4dB:Tz8: | ST2 | ST2:LiQ | LiQ |
| | 5 nm | 230 nm | 20 nm | TEG1 | 10nm | (50%:50%) | 1 nm |
| | | | | (46%:47%: | | 30 nm | |
| | | | | 7%) 30 nm | | | |
| E16 | HATCN | SpMA1 | SpMA3 | 10b:-TER1 | 50 n | ST2:LiQ | LiQ |
| | 5 nm | 125 nm | 10 nm | (97%:3%) | 10 nm | (50%:50%) | 1 nm |
| | | | | 35 nm | | 30 nm | |
| E17 | HATCN | SpMA1 | SpMA3 | 18b:TER1 | 50 n | ST2:n:LiQ | LiQ |
| | 5 nm | 125 nm | 10 nm | (97%:3%) | 10 nm | (50%:50%) | 1 nm |
| | | | | 35 nm | | 30 nm | |
| E18 | HATCN | SpMA1 | SpMA3 | 18b: BisC2: | 50 n | ST2 :: LiQ | LiQ |
| | 5 nm | 125 nm | 10 nm | TER1 | 10 nm | (50%:50%) | 1 nm |
| | | | | (57%:40%: | | 30 nm | |
| | | | | 3%) | | | |
| | | | | 35 nm | | | |
| E19 | HATCN | SpMA1 | 9b | Tz4:BisC1: | ST2 | ST2:LiQ | LiQ |
| | 5 nm | 125 nm | 10 nm | TEG1 | 10 nm | (50%:50%) | 1 nm |
| | | | | (46%:47%: | | 30 nm | |
| | | | | 7%) 30 nm | | | |
| E20 | HATCN | 28b | SpMA3 | TZ5: BisC1: | 50 n | ST2::LiQ | LiQ |
| | 5 nm | 125 nm | 10 nm | TEG1 | 10 nm | (50%:50%) | 1 nm |
| | | | | (57%:40%: | | 30 nm | |
| | | | | 3%) | | | |
| | | | | 35 nm | | | |

TABLE 2
Structural formulae of the materials for the electroluminescent devices

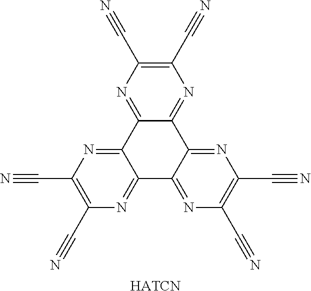

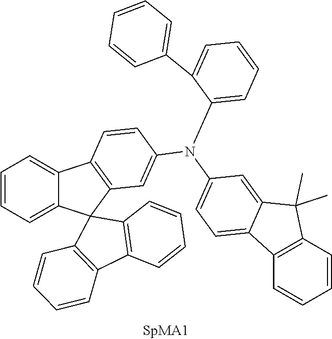 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

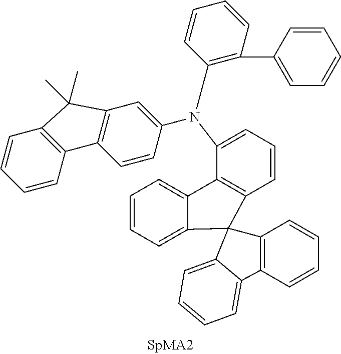 [SMILES: CC1(C)c2ccccc2-c2ccc(N(c3ccccc3-c3ccccc3)c3cccc4c3-c3ccccc3C43c4ccccc4-c4ccccc43)cc21]

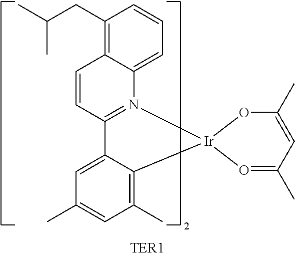

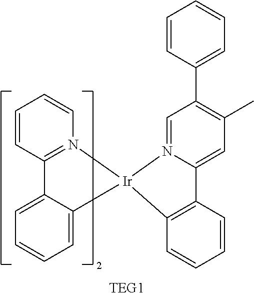

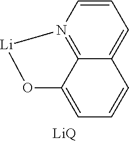

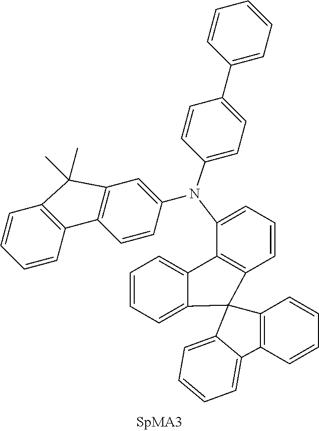 [SMILES: CC1(C)c2ccccc2-c2ccc(N(c3ccc(-c4ccccc4)cc3)c3cccc4c3-c3ccccc3C43c4ccccc4-c4ccccc43)cc21]

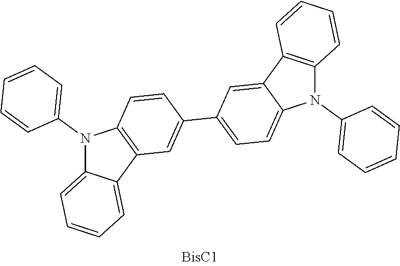 [SMILES: c1ccc(-n2c3ccccc3c3cc(-c4ccc5c(c4)c4ccccc4n5-c4ccccc4)ccc32)cc1]

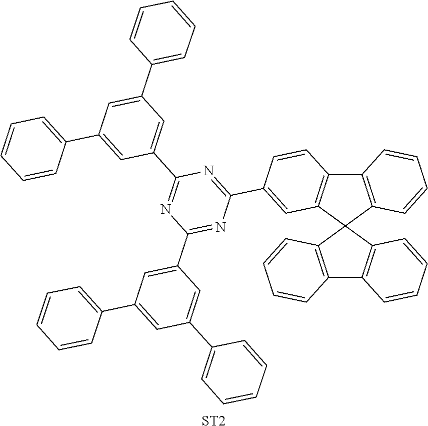

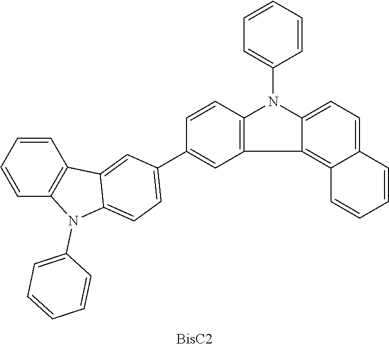 [SMILES: c1ccc(-n2c3ccccc3c3cc(-c4ccc5c(c4)c4c6ccccc6ccc4n5-c4ccccc4)ccc32)cc1]

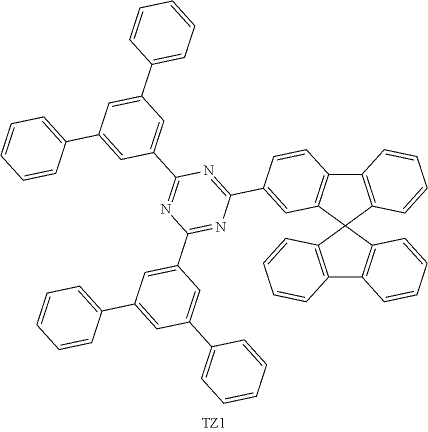 [SMILES: c1ccc(-c2cc(-c3ccccc3)cc(-c3nc(-c4cc(-c5ccccc5)cc(-c5ccccc5)c4)nc(-c4ccc5c(c4)C4(c6ccccc6-c6ccccc64)c4ccccc4-5)n3)c2)cc1]

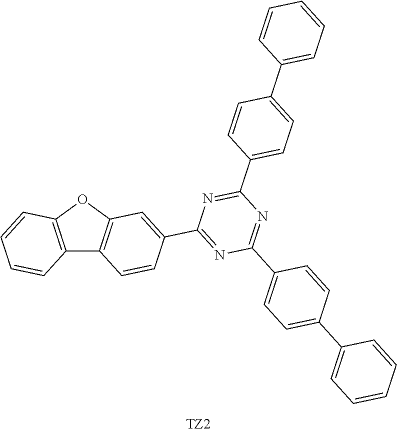 [SMILES: c1ccc(-c2ccc(-c3nc(-c4ccc(-c5ccccc5)cc4)nc(-c4ccc5c(c4)oc4ccccc45)n3)cc2)cc1]

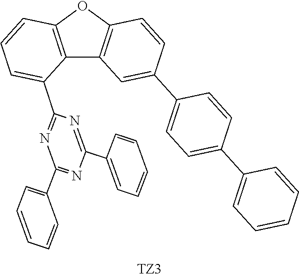 [SMILES: c1ccc(-c2ccc(-c3ccc4oc5cccc(-c6nc(-c7ccccc7)nc(-c7ccccc7)n6)c5c4c3)cc2)cc1]

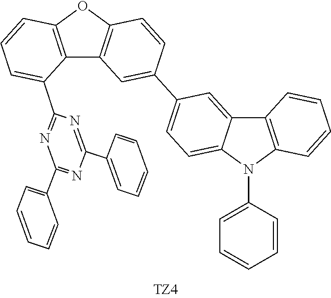 [SMILES: c1ccc(-c2nc(-c3ccccc3)nc(-c3cccc4oc5ccc(-c6ccc7c(c6)c6ccccc6n7-c6ccccc6)cc5c34)n2)cc1]

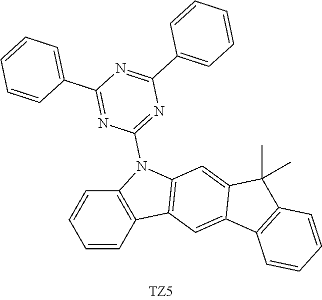 [SMILES: CC1(C)c2ccccc2-c2cc3c4ccccc4n(-c4nc(-c5ccccc5)nc(-c5ccccc5)n4)c3cc21]

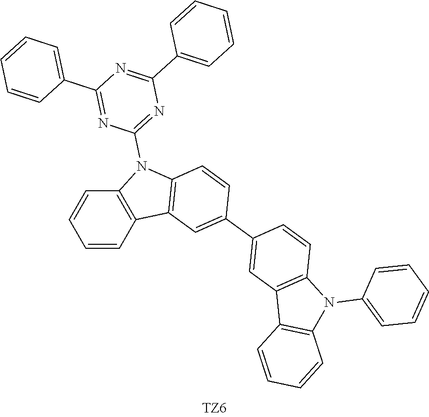 [SMILES: c1ccc(-c2nc(-c3ccccc3)nc(-n3c4ccccc4c4cc(-c5ccc6c(c5)c5ccccc5n6-c5ccccc5)ccc43)n2)cc1]

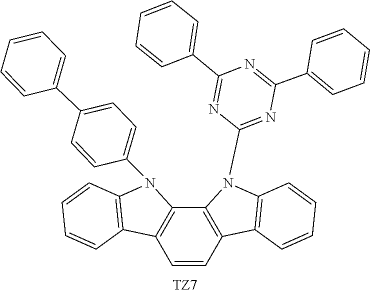 [SMILES: c1ccc(-c2ccc(-n3c4ccccc4c4ccc5c6ccccc6n(-c6nc(-c7ccccc7)nc(-c7ccccc7)n6)c5c43)cc2)cc1]

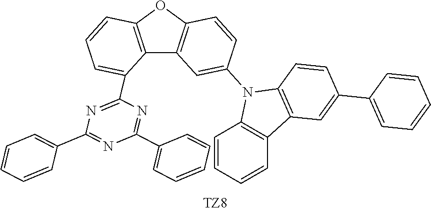 [SMILES: c1ccc(-c2ccc3c(c2)c2ccccc2n3-c2ccc3oc4cccc(-c5nc(-c6ccccc6)nc(-c6ccccc6)n5)c4c3c2)cc1]

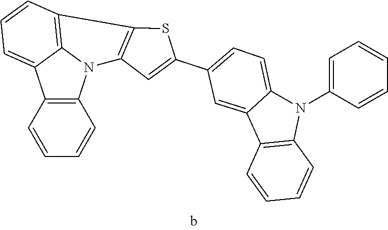 [SMILES: c1ccc(-n2c3ccccc3c3cc(-c4cc5c(s4)c4cccc6c7ccccc7n5c64)ccc32)cc1]

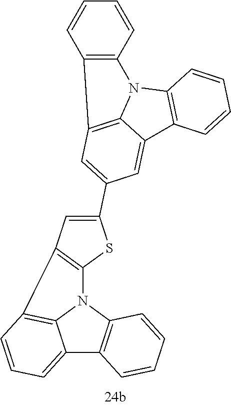 [SMILES: c1ccc2c(c1)c1cc(-c3cc4c5cccc6c7ccccc7n(c4s3)c65)cc3c4ccccc4n2c13]

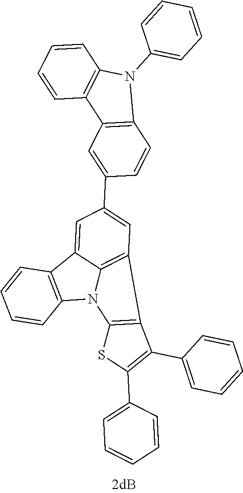 [SMILES: c1ccc(-c2sc3c(c2-c2ccccc2)c2cc(-c4ccc5c(c4)c4ccccc4n5-c4ccccc4)cc4c5ccccc5n3c42)cc1]

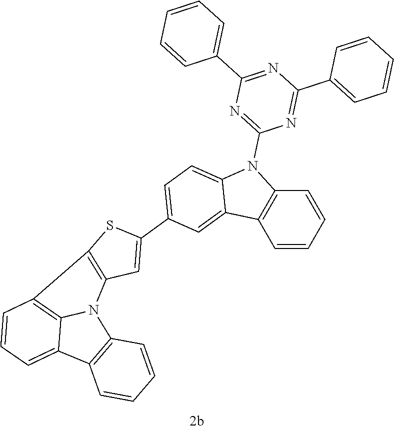 [SMILES: c1ccc(-c2nc(-c3ccccc3)nc(-n3c4ccccc4c4cc(-c5cc6c(s5)c5cccc7c8ccccc8n6c75)ccc43)n2)cc1]

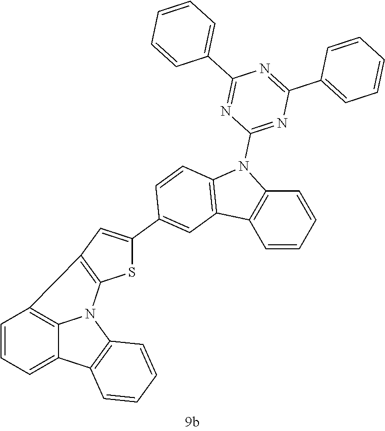 [SMILES: c1ccc(-c2nc(-c3ccccc3)nc(-n3c4ccccc4c4cc(-c5cc6c7cccc8c9ccccc9n(c6s5)c87)ccc43)n2)cc1]

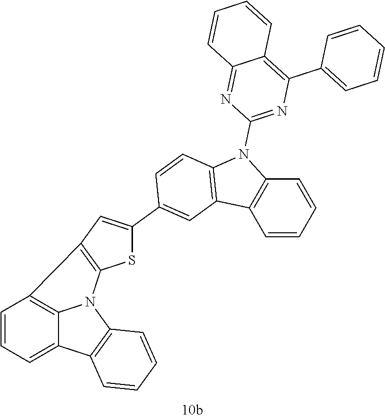 [SMILES: c1ccc(-c2nc(-n3c4ccccc4c4cc(-c5cc6c7cccc8c9ccccc9n(c6s5)c87)ccc43)nc3ccccc23)cc1]

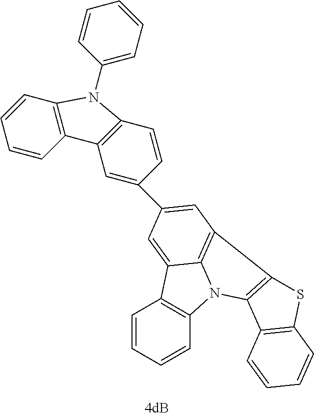 [SMILES: c1ccc(-n2c3ccccc3c3cc(-c4cc5c6ccccc6n6c5c(c4)c4sc5ccccc5c46)ccc32)cc1]

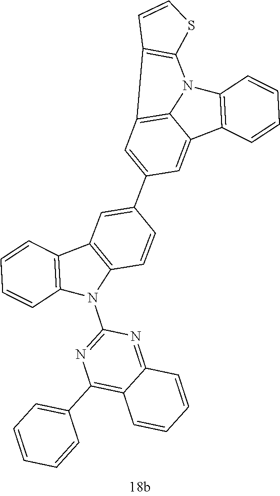 [SMILES: c1ccc(-c2nc(-n3c4ccccc4c4cc(-c5cc6c7ccccc7n7c8sccc8c(c5)c67)ccc43)nc3ccccc23)cc1]

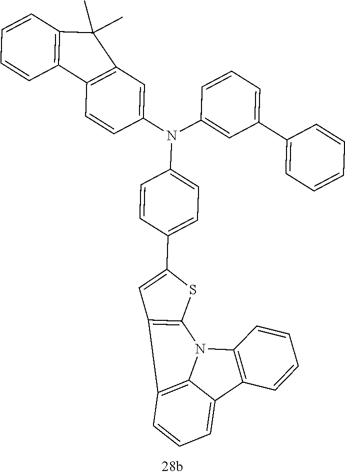 [SMILES: CC1(C)c2ccccc2-c2ccc(N(c3ccc(-c4cc5c6cccc7c8ccccc8n(c5s4)c76)cc3)c3cccc(-c4ccccc4)c3)cc21]

TABLE 3
Performance date of the electroluminescent devices

| | U1000 | CE1000 | EQE 1000 | CIE x/y at |
|---|---|---|---|---|
| Ex. | (V) | (cd/A) | (%) | 1000 cd/m² |
| E1 | 4.2 | 67 | 15.2 | 0.33/0.64 |
| E2 | 4.0 | 69 | 16.0 | 0.32/0.63 |
| E3 | 3.3 | 65 | 17.5 | 0.33/0.63 |
| E4 | 3.2 | 71 | 17.9 | 0.31/0.64 |
| E5 | 3.4 | 67 | 18 | 0.32/0.63 |
| E6 | 3.5 | 74 | 18.0 | 0.33/0.63 |
| E7 | 3.2 | 76 | 18.3 | 0.33/0.63 |
| E8 | 3.3 | 69 | 18.5 | 0.31/0.64 |
| E9 | 3.2 | 73 | 19.0 | 0.31/0.64 |
| E10 | 3.4 | 75 | 17.4 | 0.32/0.64 |
| E11 | 3.2 | 74 | 18.9 | 0.32/0.64 |
| E12 | 3.2 | 78 | 18.0 | 0.31/0.63 |
| E13 | 3.4 | 72 | 19.1 | 0.31/0.64 |
| E14 | 3.5 | 66 | 17.3 | 0.32/0.64 |
| E15 | 3.6 | 62 | 15.5 | 0.32/0.64 |
| E16 | 3.9 | 28 | 20.6 | 0.67/0.34 |
| E17 | 3.8 | 27 | 22.0 | 0.66/0.33 |
| E18 | 3.4 | 29 | 22.9 | 0.67/0.33 |
| E19 | 3.2 | 62 | 18 | 0.33/0.64 |
| E20 | 3.2 | 72 | 19 | 0.33/0.63 |

1.-20. (canceled)

21. A compound comprising at least one structure of the formula (1): 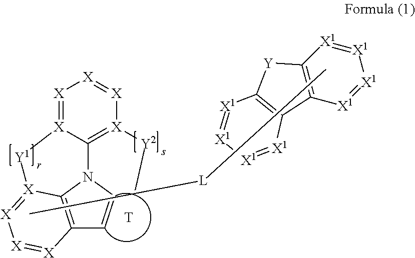
where the symbols and indices used are as follows:
T is a heteroaromatic 5-membered ring which has a sulfur atom and is fused to the azole ring via two adjacent and mutually bonded carbon atoms and which may be substituted by one or more R³ groups;
L is a connecting group which is selected from a bond or an aromatic or heteroaromatic ring system which has 5 to 40 aromatic ring atoms and may be substituted by one or more R² radicals;
X is N, CR, or C if an L, Y¹ or Y² group binds thereto, with the proviso that not more than two of the X groups in one cycle are N;
X¹ is N, CR¹, or C if the L group binds thereto, with the proviso that not more than two of the X groups in one cycle are N;
Y is NAr, NL, O, S, C(R²)₂, C(L)(R²), where NL means that the L group binds to the nitrogen atom of the NL group, and C(L)(R²) means that the L group binds to the carbon atom of the C(L)(R²) group;
Y¹ is a bond, NL, NR², NAr′, O, S, C(R²)₂, where NL means that the L group binds to the nitrogen atom of the NL group;
r is 0 or 1, where r=0 means that the Y¹ group is absent;
Y² is a bond, NL, NR², NAr′, O, S, C(R²)₂, where NL means that the L group binds to the nitrogen atom of the NL group;
s is 0 or 1, where s=0 means that the Y² group is absent;
Ar is the same or different at each instance and is an aromatic or heteroaromatic ring system which has 5 to 40 aromatic ring atoms and may be substituted by one or more R² radicals;
R is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals and where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R radicals together, or one R radical together with an R², R³ radical, may also form an aliphatic or heteroaliphatic ring system;
R¹ is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals and where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R¹ radicals together or one R¹ radical together with one R² radical may also form an aromatic, heteroaromatic, aliphatic or heteroaliphatic ring system;
R² is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals and where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R² radicals together or one R² radical together with one R, R¹, R³ radical may also form an aromatic, heteroaromatic, aliphatic or heteroaliphatic ring system;
R³ is the same or different at each instance and is H, D, F, Cl, Br, I, N(R⁴)₂, N(Ar′)₂, CN, NO₂, OR⁴, SR⁴, COOR⁴, C(═O)N(R⁴)₂, Si(R⁴)₃, B(OR⁴)₂, C(═O)R⁴, P(═O)(R⁴)₂, S(═O)R⁴, S(═O)₂R⁴, OSO₂R⁴, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals and where one or more nonadjacent CH₂ groups may be replaced by Si(R⁴)₂, C═O, NR⁴, O, S or CONR⁴, or an aromatic or heteroaromatic ring system which has 5 to 60 aromatic ring atoms, and may be substituted in each case by one or more R⁴ radicals; at the same time, two R³ radicals together or one R³ radical together with one R, R² radical may also form an aromatic, heteroaromatic, aliphatic or heteroaliphatic ring system;
Ar′ is the same or different at each instance and is an aromatic or heteroaromatic ring system which has 5 to 40 aromatic ring atoms and may be substituted by one or more R⁴ radicals;
R⁴ is the same or different at each instance and is H, D, F, Cl, Br, I, N(R⁵)₂, CN, NO₂, OR S, SR S, Si(R⁵)₃, B(OR⁵)₂, C(═O)R⁵, P(═O)(R⁵)₂, S(═O)R⁵, S(═O)₂R⁵, OSO₂R⁵, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁵ radicals and where one or more nonadjacent CH₂ groups may be replaced by Si(R⁵)₂, C═O, NR⁵, O, S or CONR⁵, or an aromatic or heteroaromatic ring system which has 5 to 40 aromatic ring atoms and may be substituted in each case by one or more R² radicals; at the same time, two or more R⁴ radicals together may form an aromatic, heteroaromatic, aliphatic or heteroaliphatic ring system;
R⁵ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
where the sum of r and s is 1 or 2.

22. The compound as claimed in claim 21, wherein the compound comprises at least one structure of the formulae (1a), (1b), (1c), (1d), (1e), (1f), (1g), (1h), (1i), (1j), (1k), (1l), (1m), (1n), (1o), (1p), (1q), (1r), (1s), (1t), (1u), (1v), (1w), (1x), (1y), (1z) and (1za): 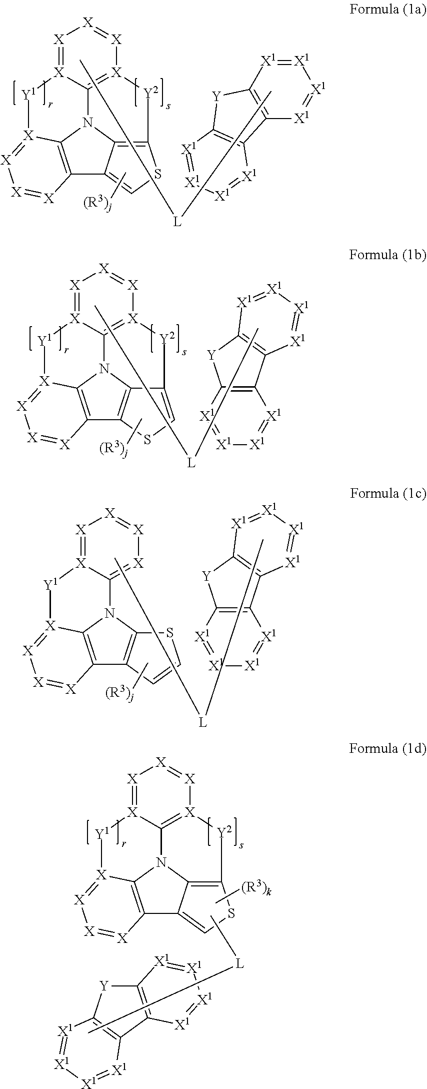 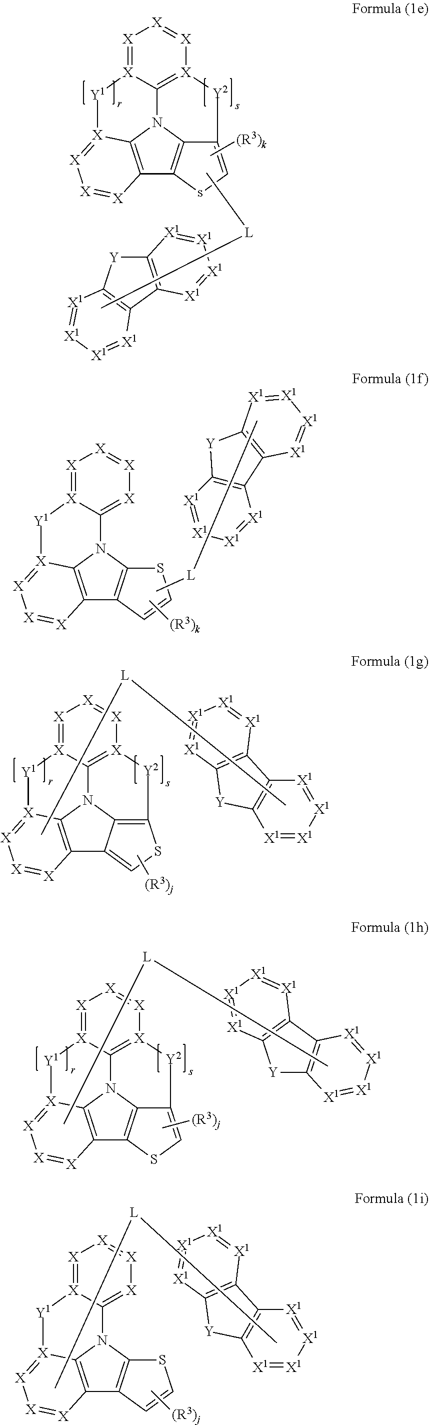 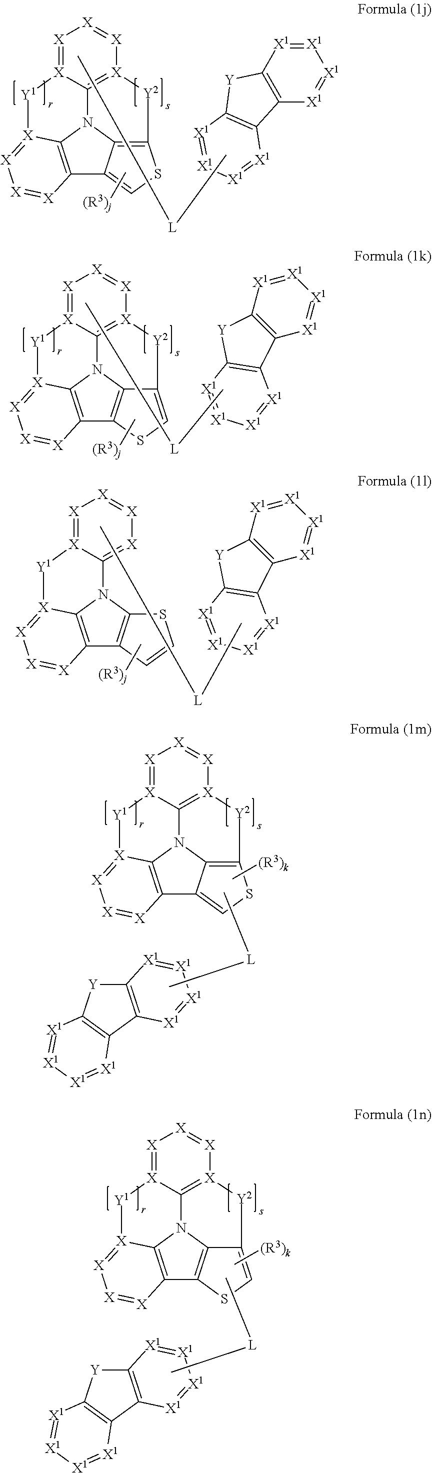 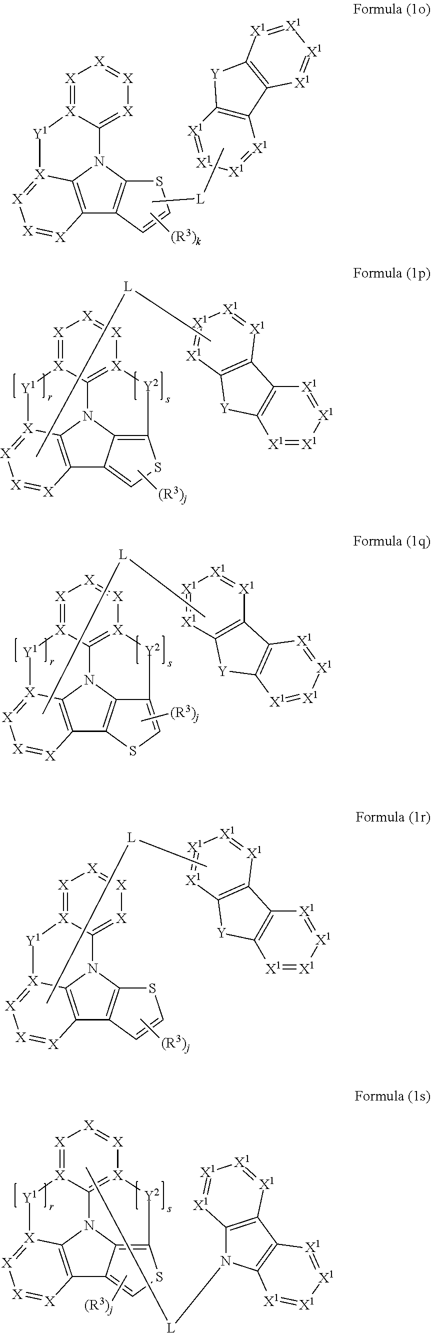 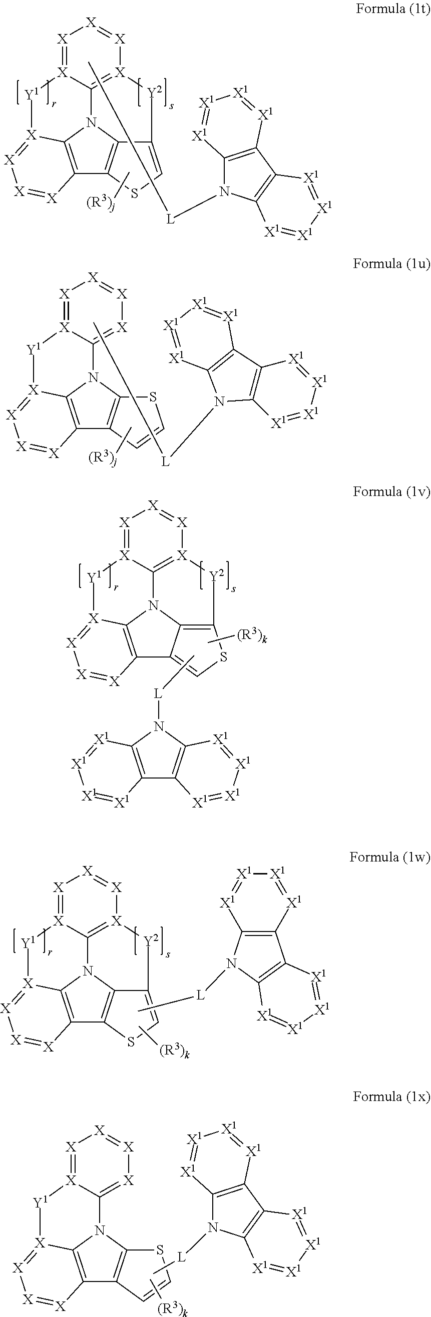 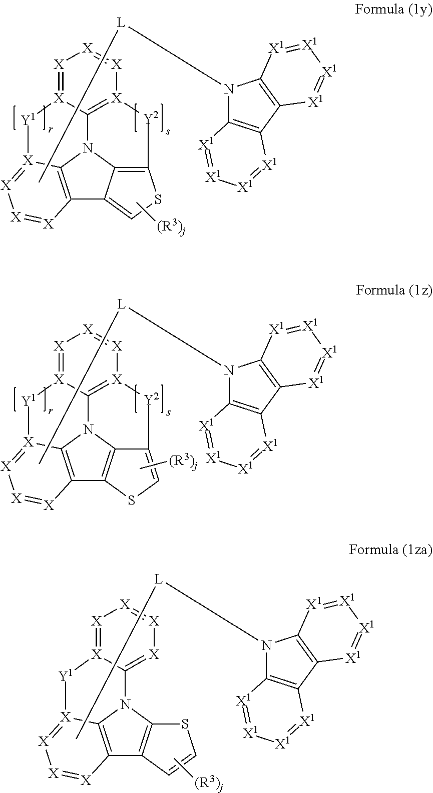
where Y, Y¹, Y², X, X¹, s, and R³ have the definitions given in claim 21, j is 0, 1 or 2, and k is 0 or 1, where s+k=0 or 1 and j+s=0, 1 or 2.

23. The compound as claimed in claim 21, wherein the compound comprises at least one structure of the formulae (2a), (2b), (2c), (2d), (2e), (2f), (2g), (2h), (2i), (2j), (2k), (2l), (2m), (2n), (2o), (2p), (2q), (2r), (2s), (2t), (2u), (2v), (2w), (2x), (2y), (2z) and (2za): 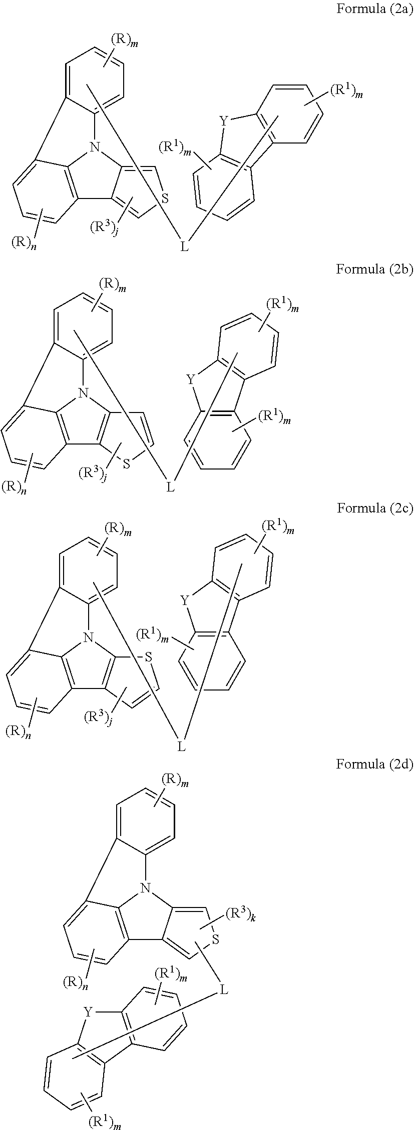 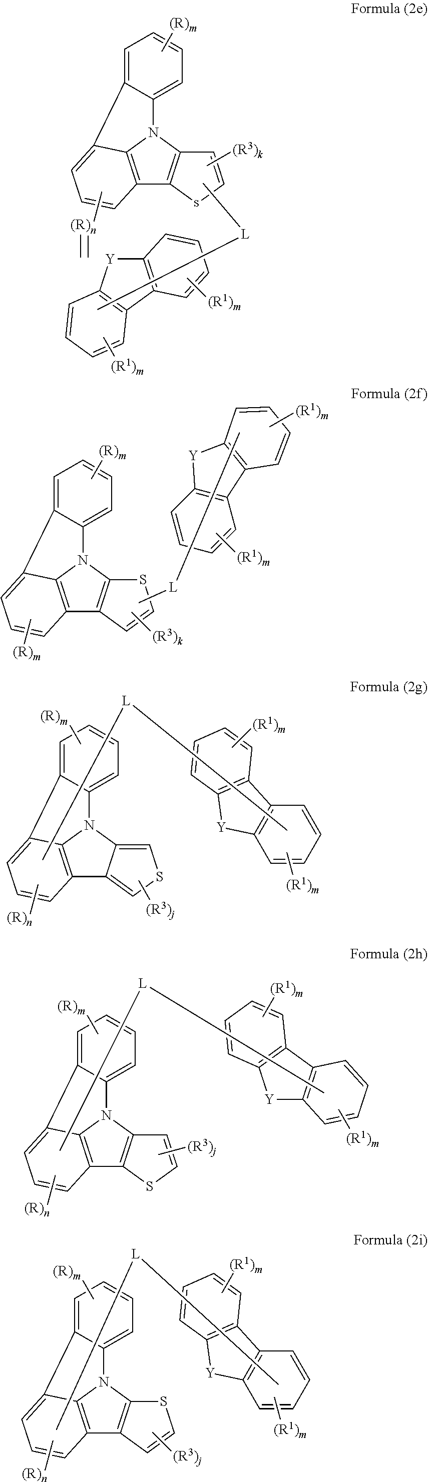 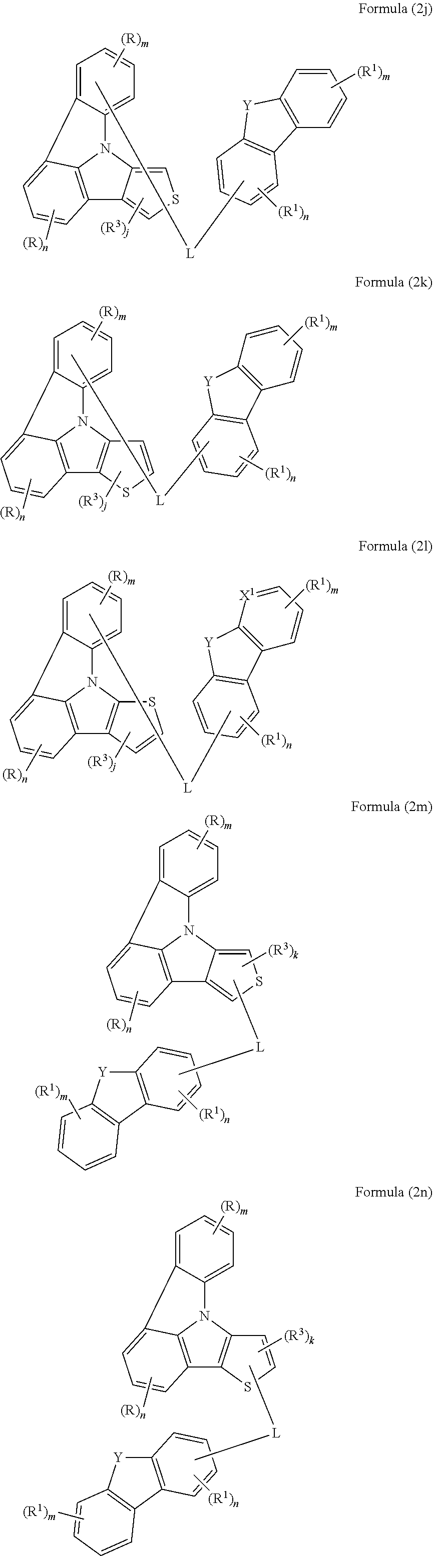 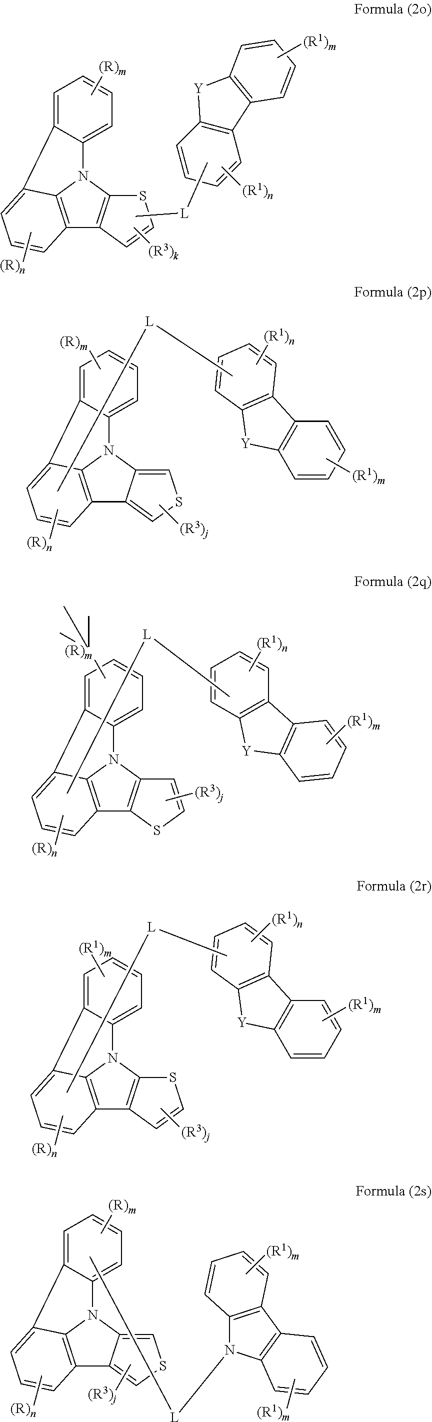 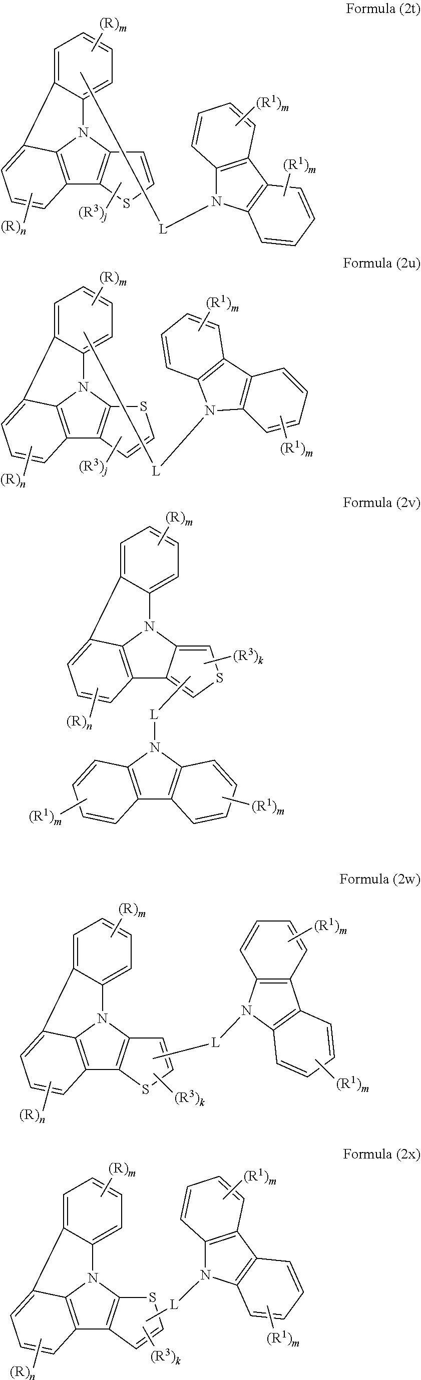 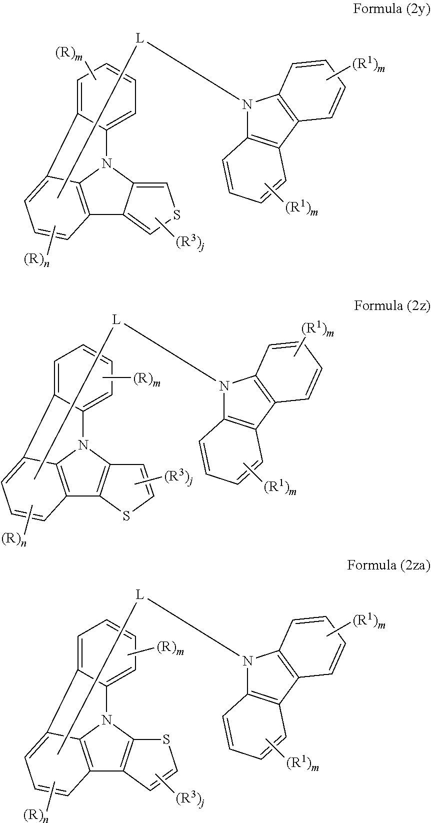
where L, Y, R, R¹ and R³ have the definitions given in claim 21, the index k is 0 or 1, the index j is 0, 1 or 2, the index n is 0, 1, 2 or 3, and the index m is 0, 1, 2, 3 or 4.

24. The compound as claimed in claim 21, wherein the compound comprises at least one structure of the formulae (3a), (3b), (3c), (3d), (3e), (3f), (3g), (3h), (3i), (3j), (3k), (3l), (3m), (3n), (3o), (3p), (3q) and (3r): 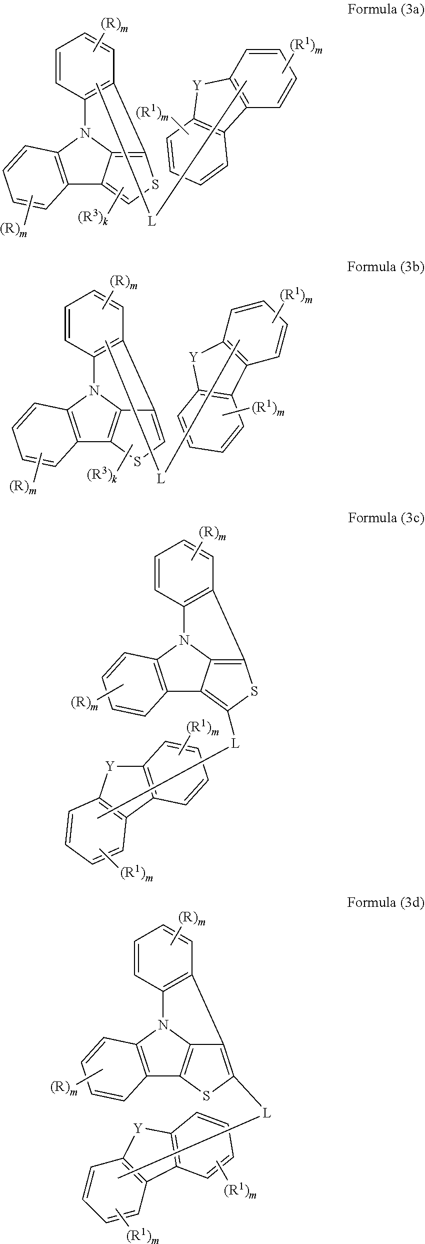 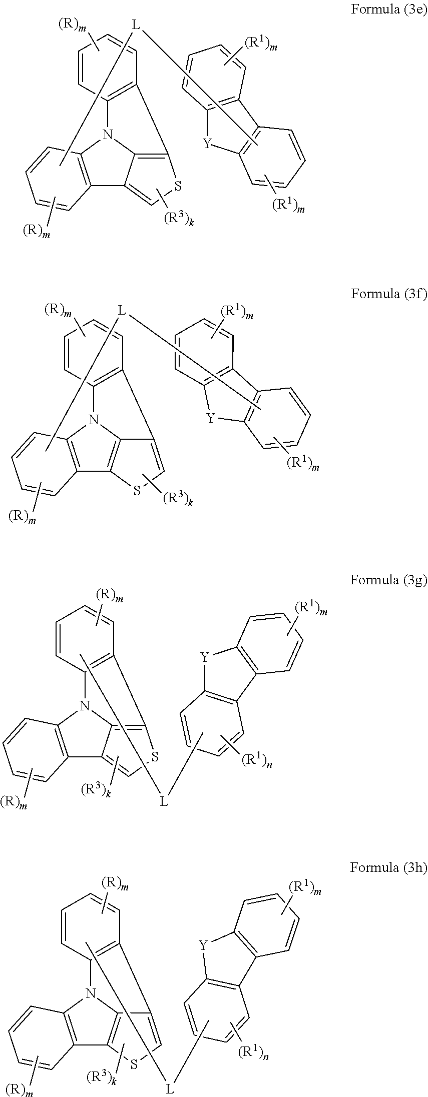 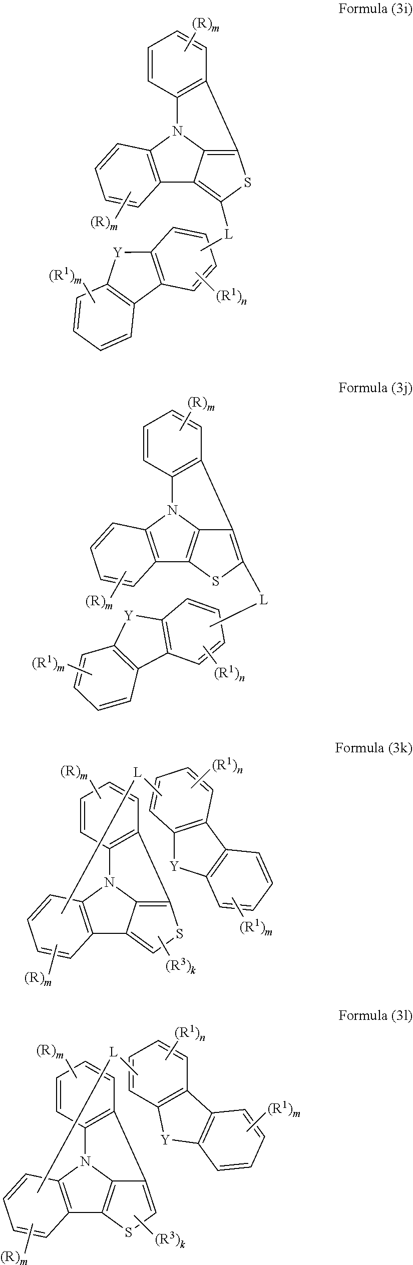 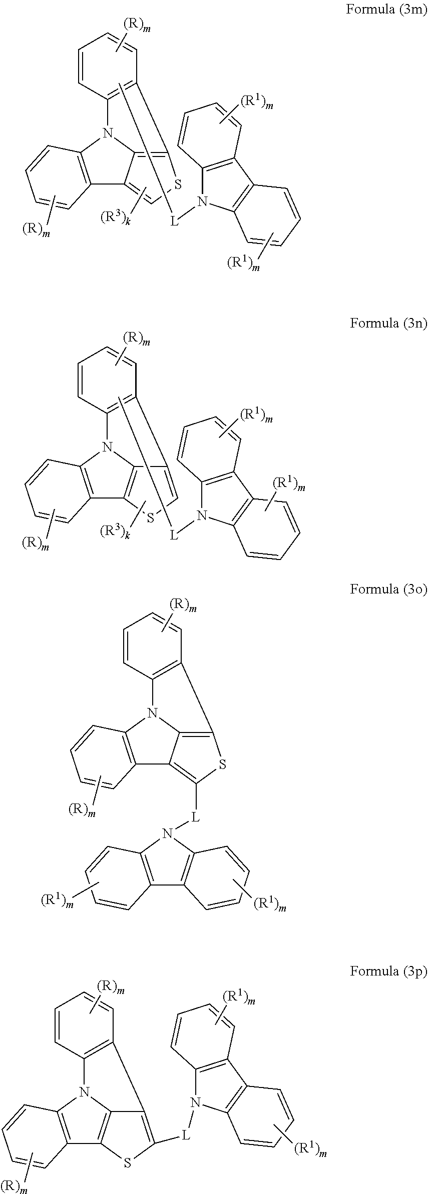 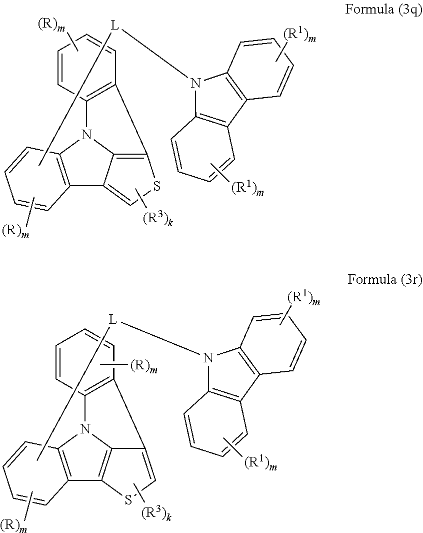
where L, Y, R, R¹ and R³ have the definitions given in claim 21, the index k is 0 or 1, the index n is 0, 1, 2 or 3 and the index m is 0, 1, 2, 3 or 4.

25. The compound as claimed in claim 21, wherein the compound comprises at least one structure of the formulae (4a), (4b), (4c), (4d), (4e), (4f), (4g), (4h), (4i), (4j), (4k), (4l), (4m), (4n), (4o), (4p), (4q) and (4r): 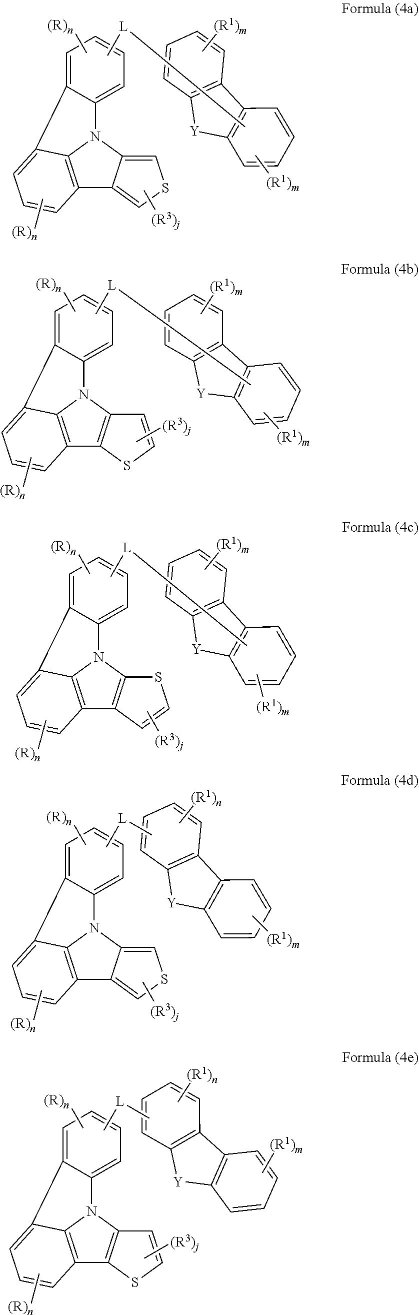 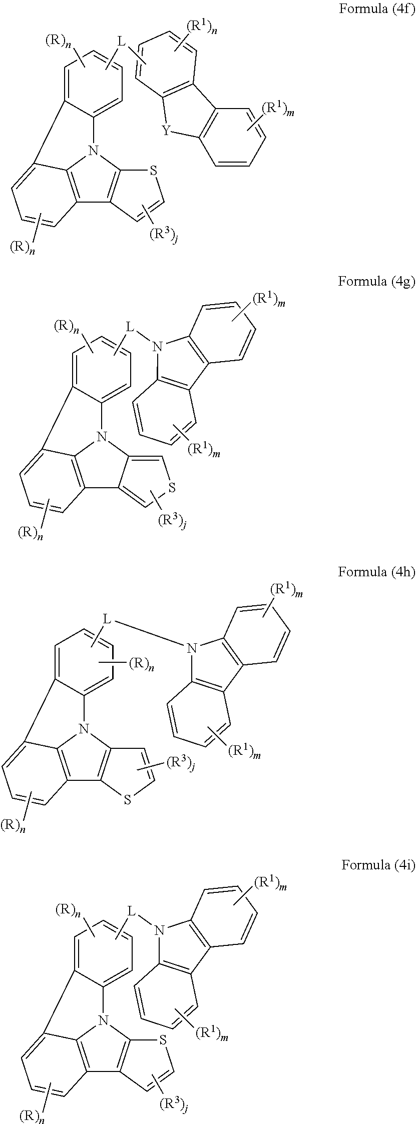 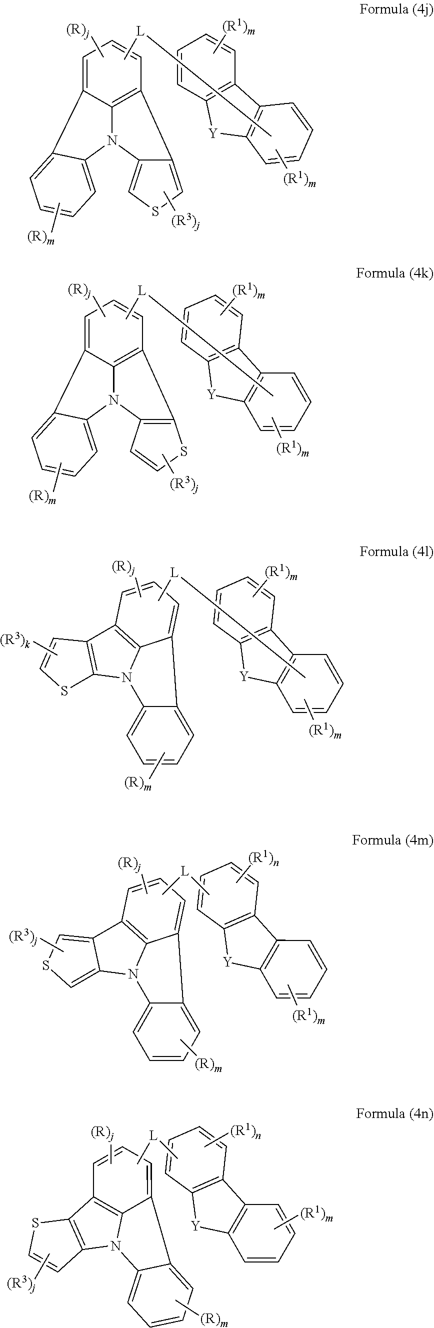 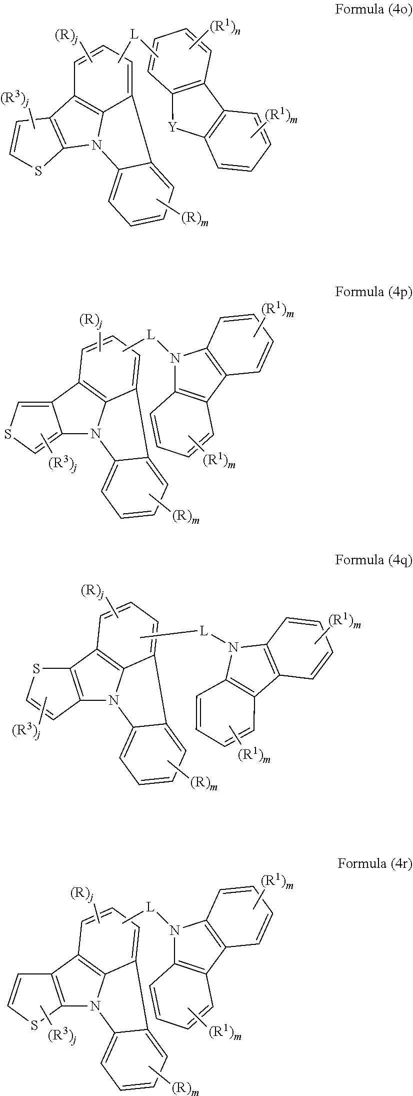
where L, Y, R, R¹ and R³ have the definitions given in claim 21, the index k is 0 or 1, the index j is 0, 1 or 2, the index n is 0, 1, 2 or 3, and the index m is 0, 1, 2, 3 or 4.

26. The compound as claimed in claim 21, wherein the compound comprises at least one structure of the formulae (5a), (5b), (5c), (5d), (5e), (5f), (5g), (5h) and (5i): 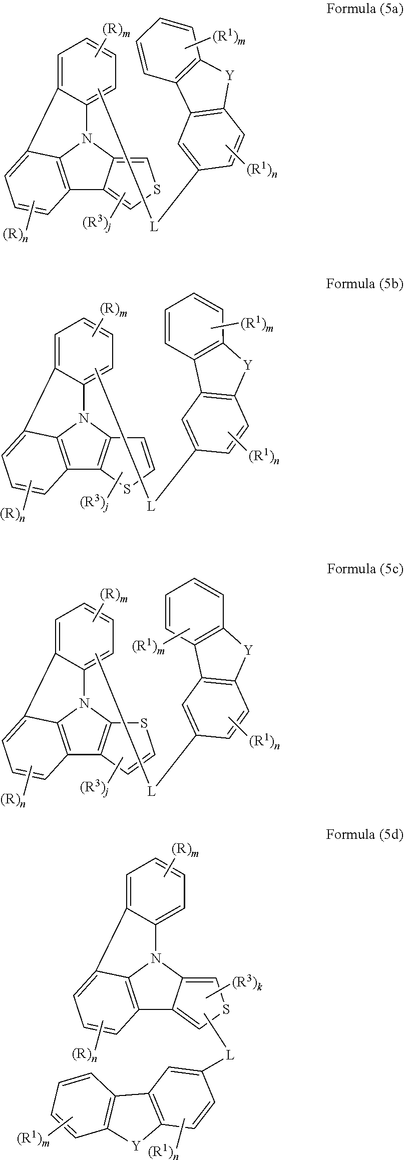 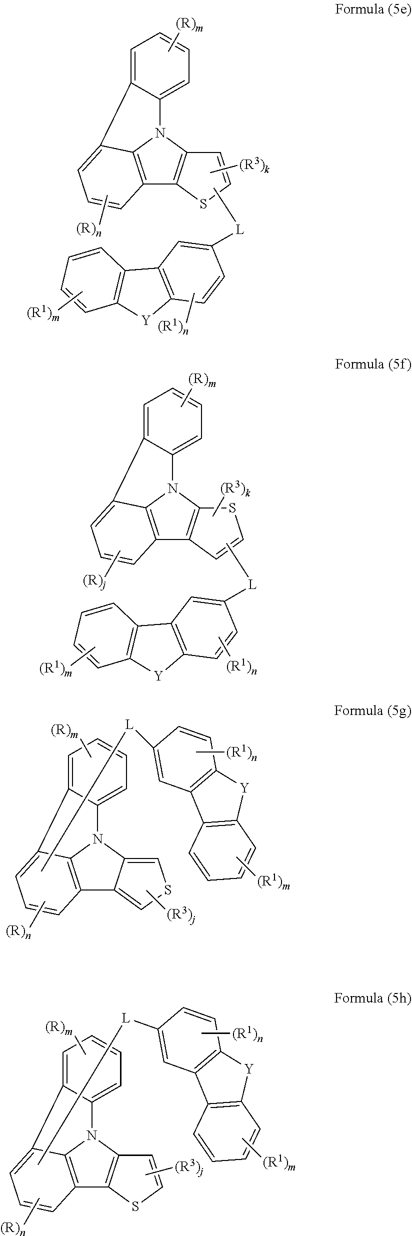 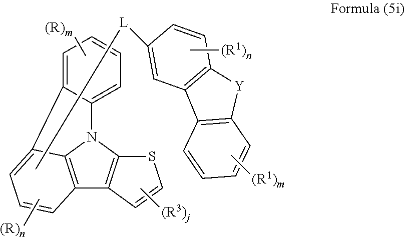
where L, Y, R, R¹ and R³ have the definitions given in claim 21, the index k is 0 or 1, the index j is 0, 1 or 2, the index n is 0, 1, 2 or 3, and the index m is 0, 1, 2, 3 or 4,

27. The compound as claimed in claim 21, wherein the compound comprises at least one structure of the formulae (6a), (6b), (6c), (6d), (6e), (6f), (6g) and (6h): 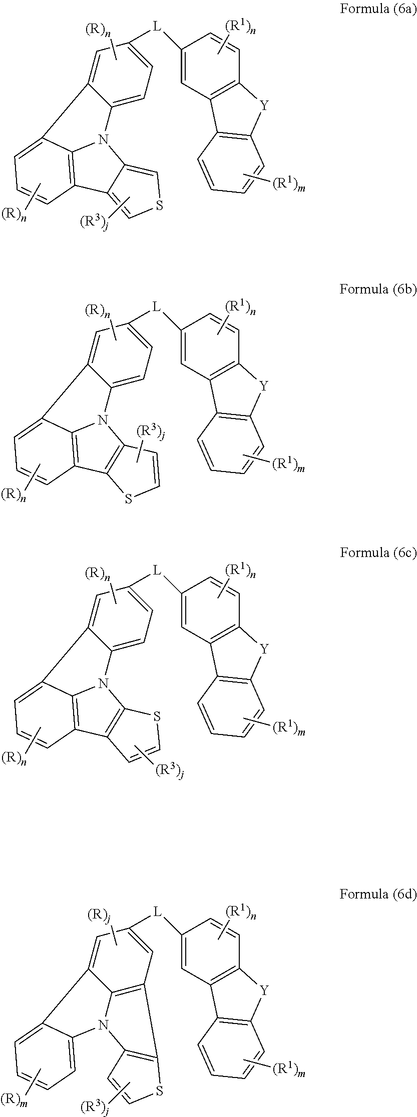 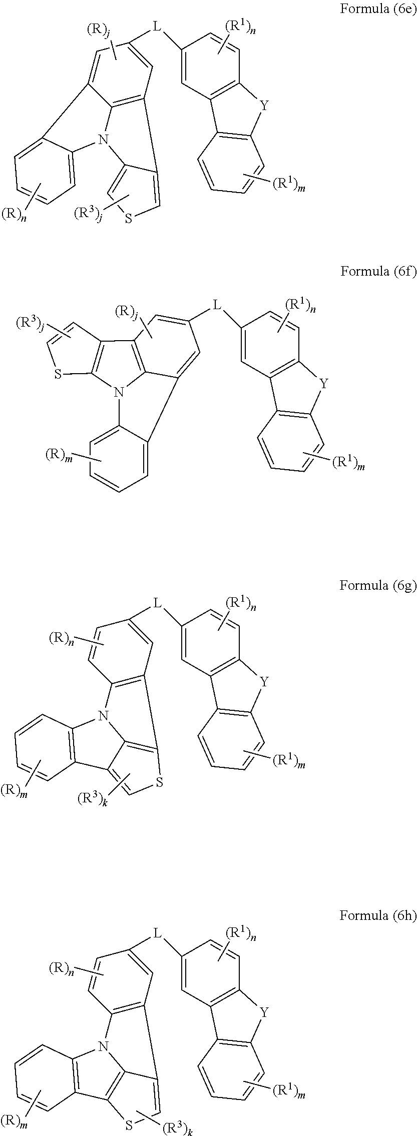
where L, Y, R, R¹ and R³ have the definitions given in claim 21,
the index k is 0 or 1;
the index j is 0, 1 or 2;
the index n is 0, 1, 2 or 3; and
the index m is 0, 1, 2, 3 or 4.

28. The compound as claimed in claim 21, wherein L is a bond or is a group selected from the formulae (L¹-1) to (L¹-16): 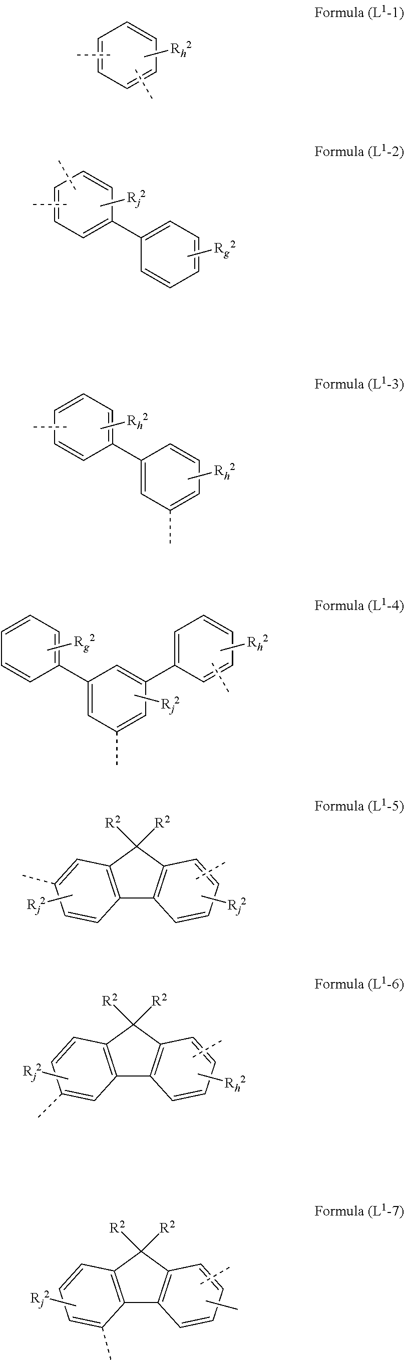 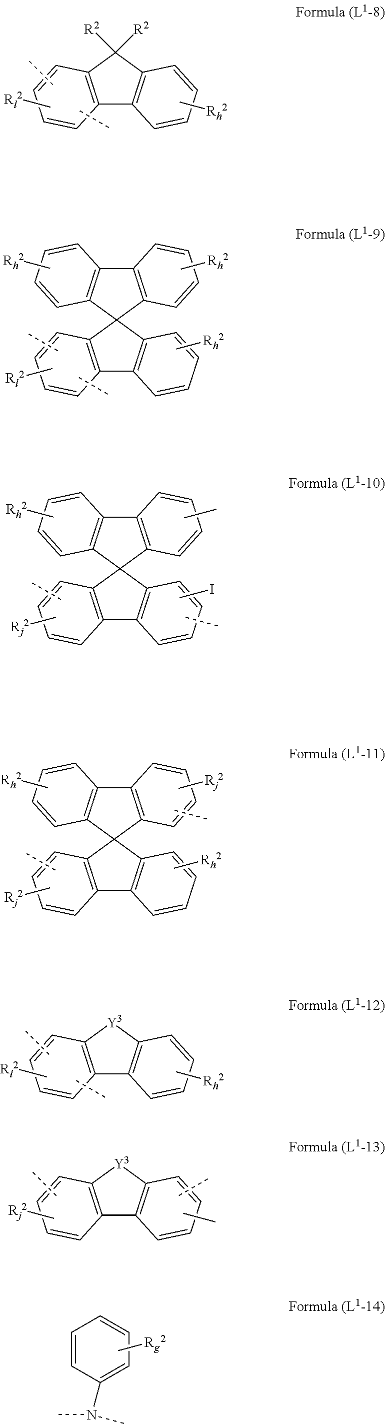 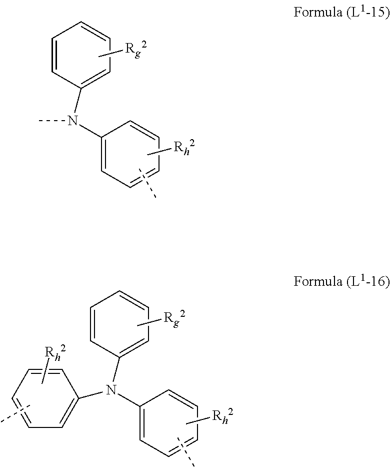
where the dotted bonds each mark the positions of attachment;
Y³ is the same or different at each instance and is O, S, NAr′, NR²;
the index k is 0 or 1;
the index l is 0, 1 or 2;
the index j is independently at each instance 0, 1, 2 or 3;
the index h is independently at each instance 0, 1, 2, 3 or 4;
the index g is 0, 1, 2, 3, 4 or 5; and
the symbol R² has the definition given in claim 21, where L is a bond or is an aromatic ring system which has 5 to 40 aromatic ring atoms and does not comprise any heteroatoms.

29. The compound as claimed in claim 21, wherein L is a bond and Y is NAr, O or S.

30. The compound as claimed in claim 21, wherein R, R¹, R² and/or R³ are the same or different at each instance and are selected from the group consisting of H, D or an aromatic or heteroaromatic ring system selected from the groups of the following formulae Ar-1 to Ar-75, and/or the Ar group is the same or different at each instance and is selected from the groups of the following formulae Ar-1 to Ar-75: 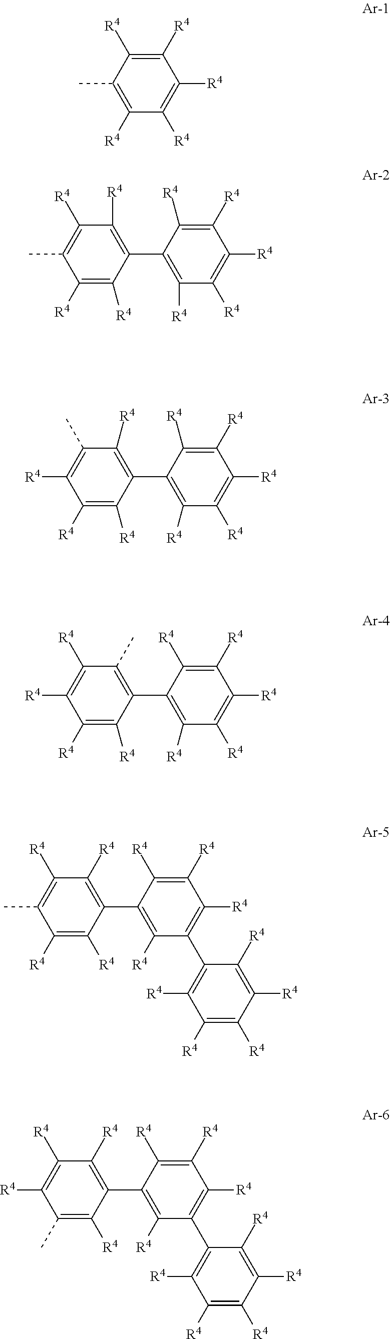 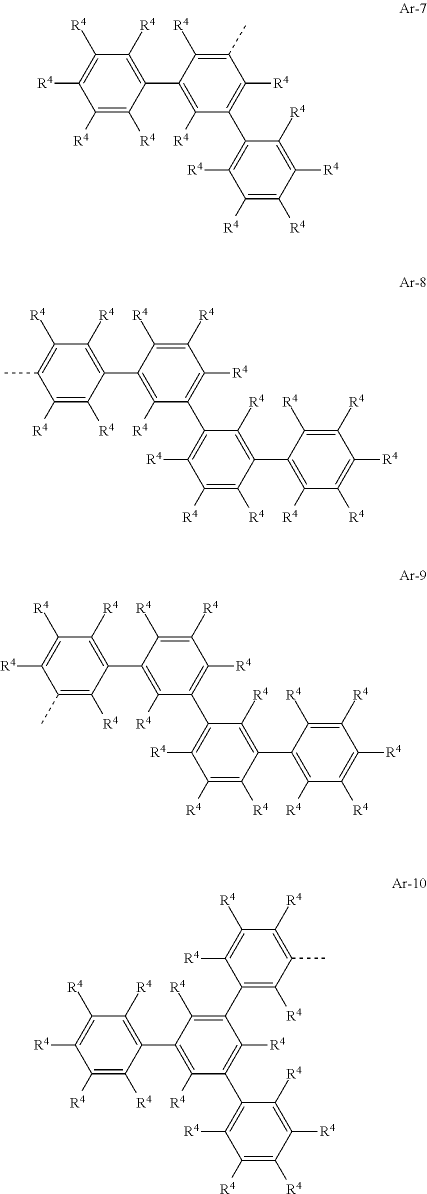 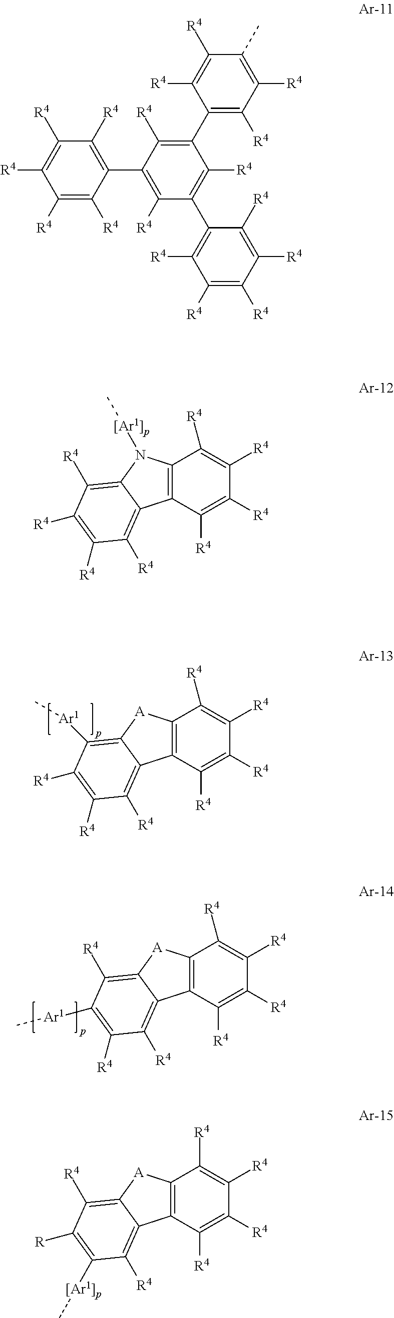 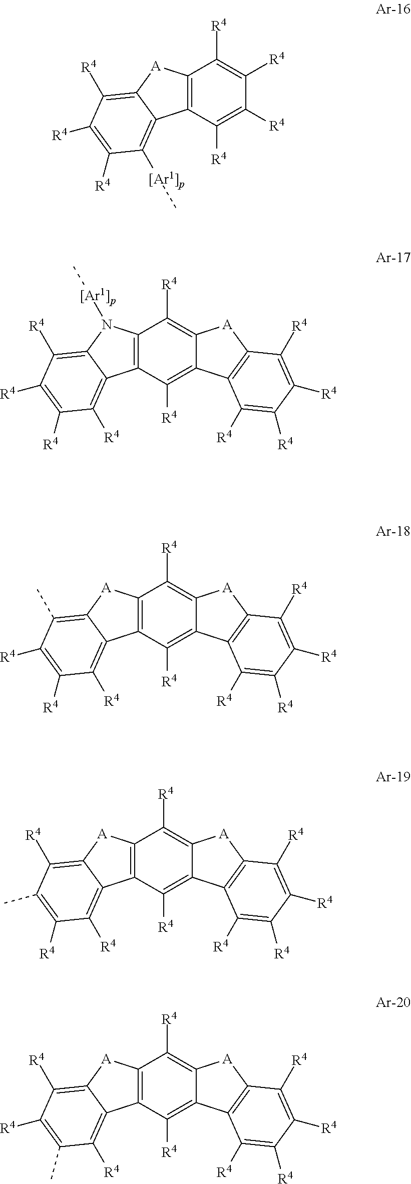 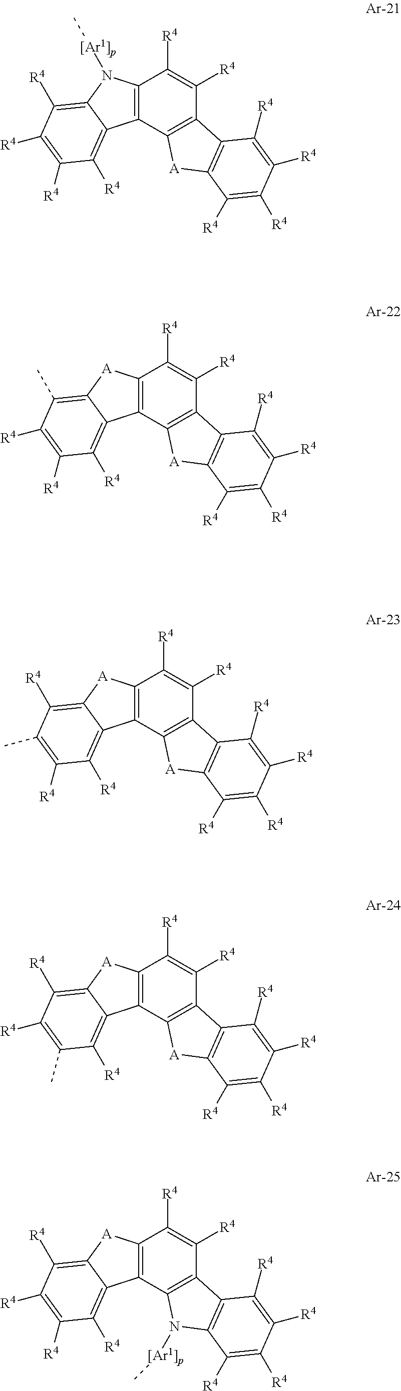 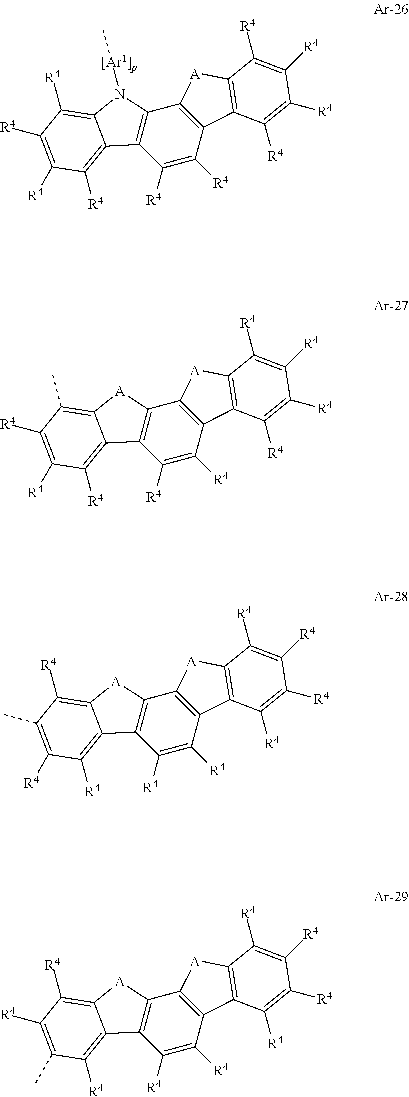 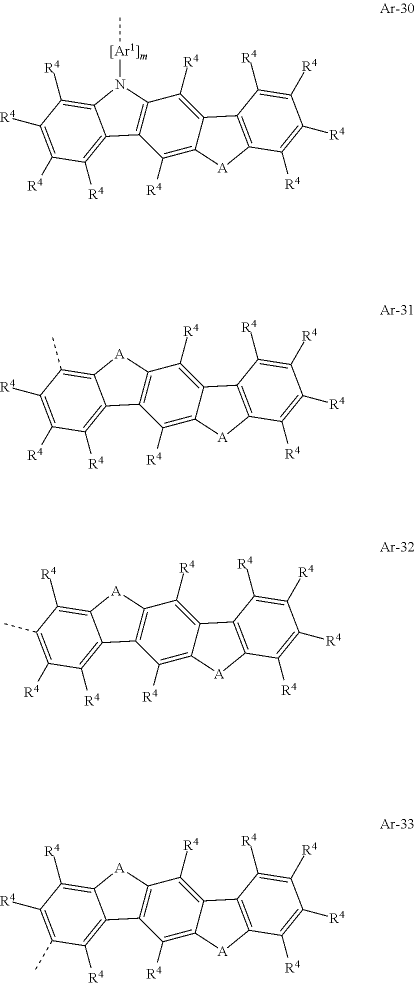 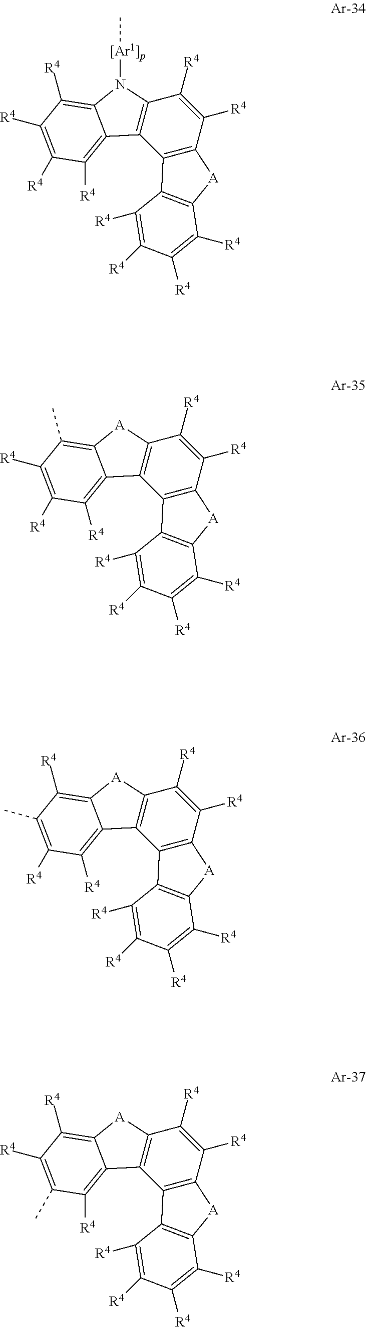 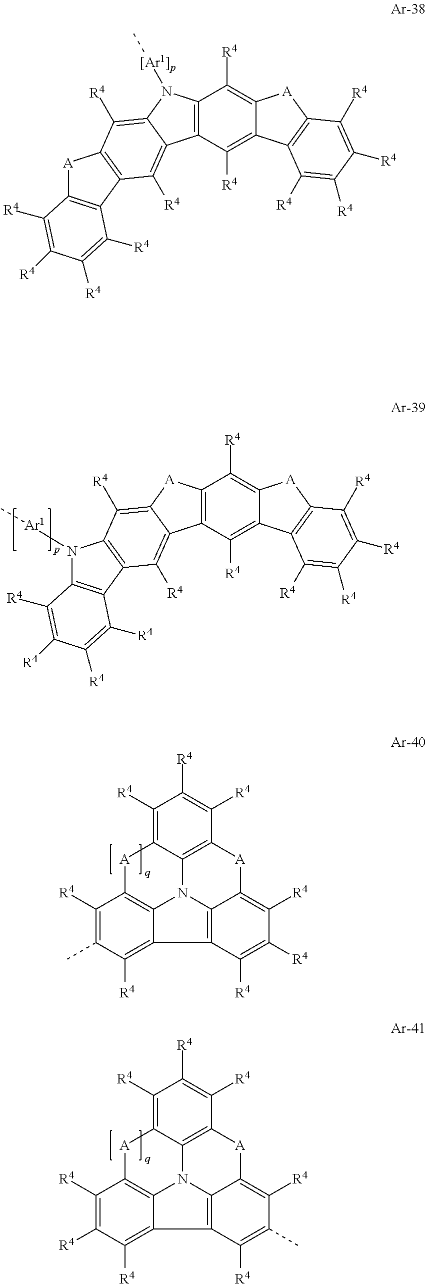 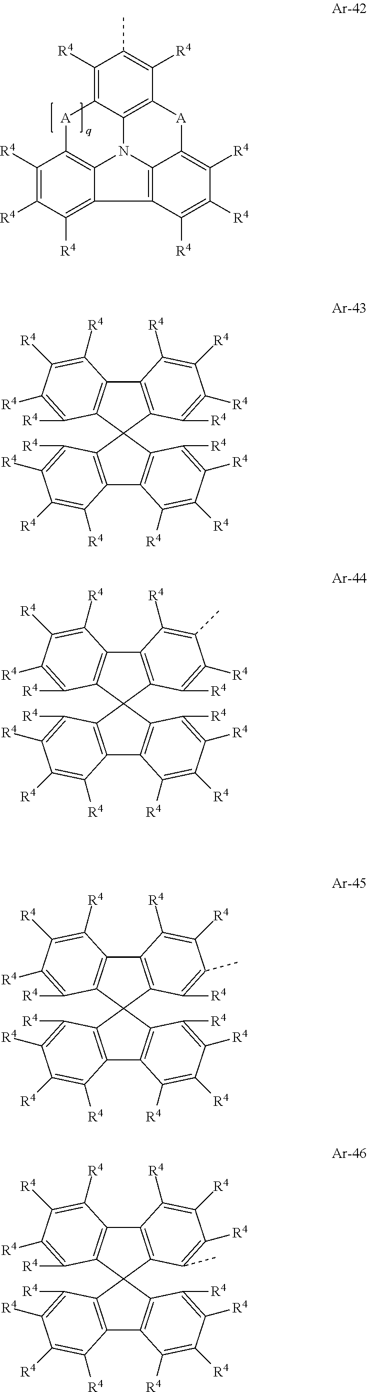 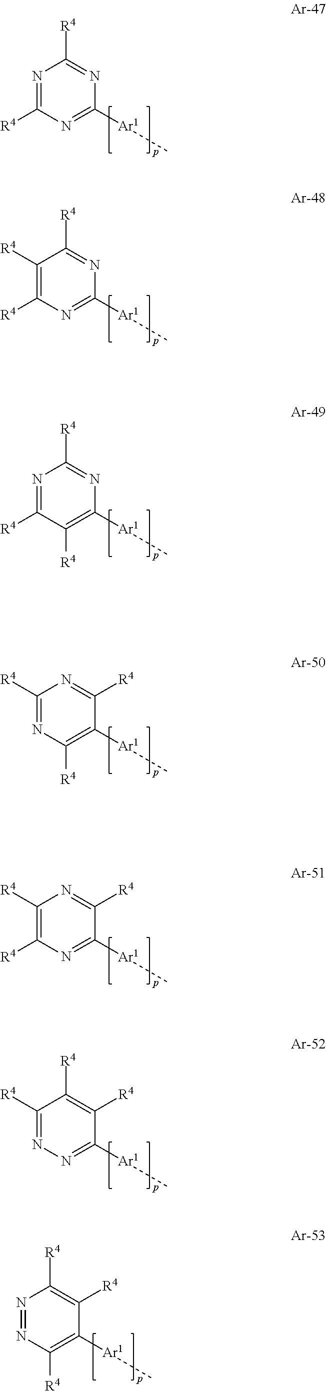 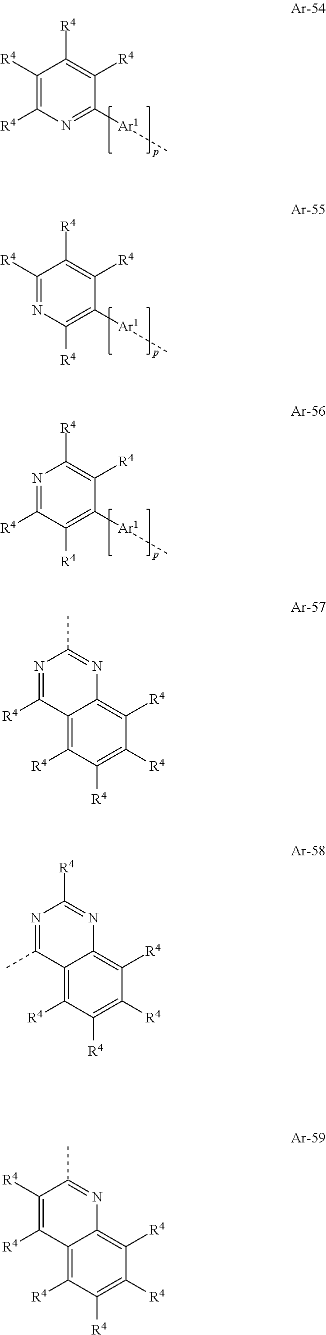 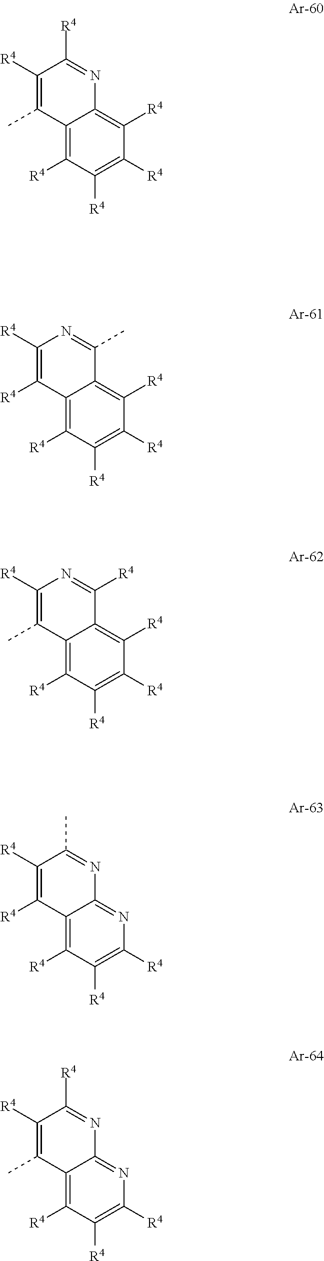 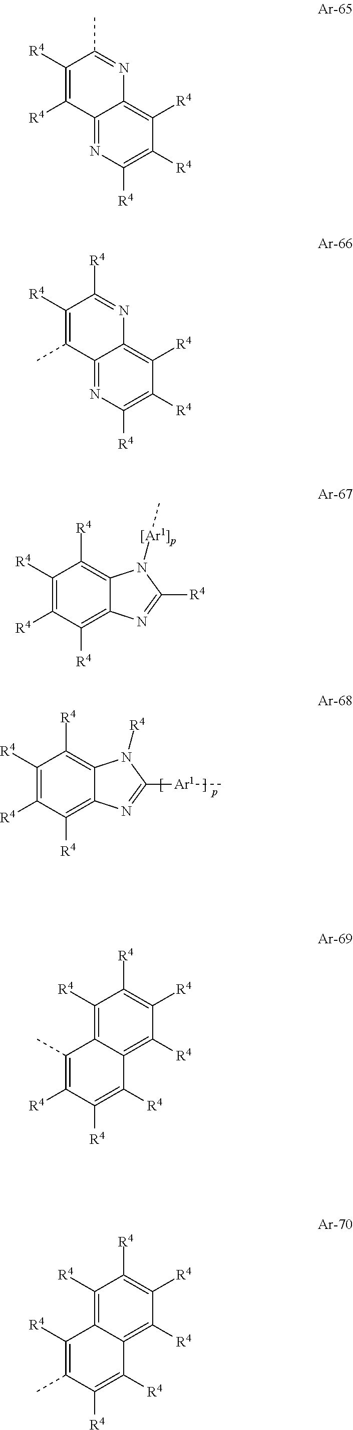 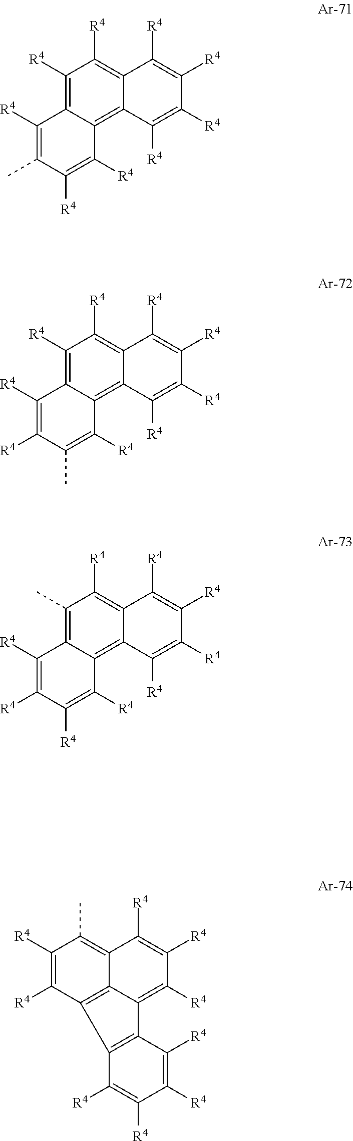 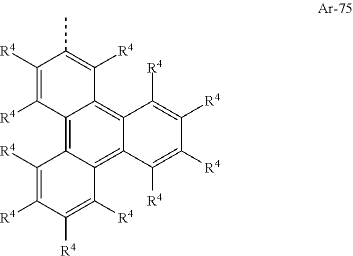
where R⁴ has the definitions given above, the dotted bond represents the bond of the corresponding group and in addition:
Ar¹ is the same or different at each instance and is a bivalent aromatic or heteroaromatic ring system which has 6 to 18 aromatic ring atoms and may be substituted in each case by one or more R⁴ radicals;
A is the same or different at each instance and is C(R⁴)₂, NR⁴, O or S;
p is 0 or 1, where p=0 means that the Ar¹ group is absent and that the corresponding aromatic or heteroaromatic group is bonded directly to the corresponding radical;
q is 0 or 1, where q=0 means that no A group is bonded at this position and R⁴ radicals are bonded to the corresponding carbon atoms instead.

31. A process for preparing the compound as claimed in claim 21, which comprises reacting a thiofuran compound with an aromatic or heteroaromatic nitrogen compound by a coupling reaction.

32. A composition comprising at least one compound as claimed in claim 21 and at least one further matrix material, wherein the further matrix material is selected from compounds of one of the formulae (7), (8), (9) and (10): 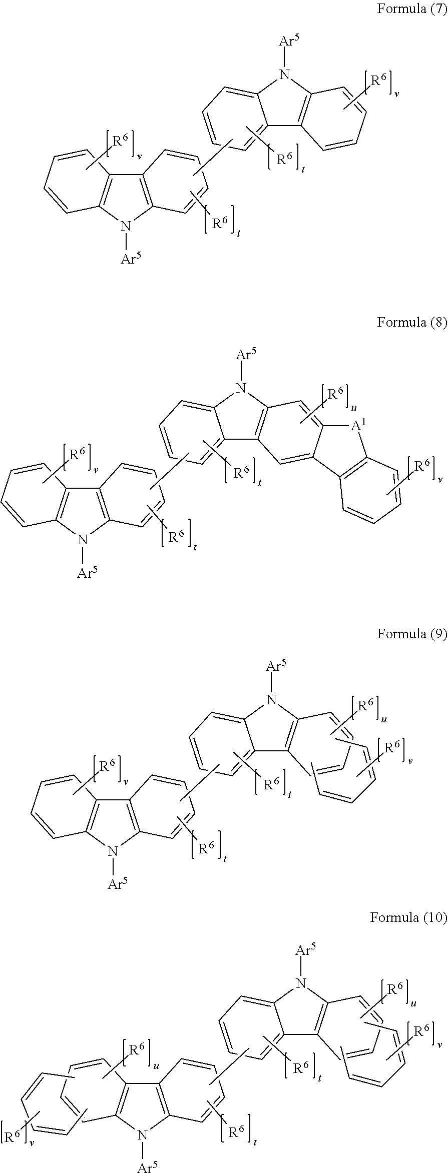
where the symbols and indices used are as follows:
R⁶ is the same or different at each instance and is H, D, F, Cl, Br, I, N(R⁷)₂, N(Ar′)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals, where one or more nonadjacent CH₂ groups may be replaced by Si(R⁷)₂, C═O, NR⁷, O, S or CONIC, or an aromatic or heteroaromatic ring system which has 5 to 60 aromatic ring atoms, and may be substituted in each case by one or more R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S;
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
s is the same or different at each instance and is 0, 1, 2, 3 or 4;
t is the same or different at each instance and is 0, 1, 2 or 3; and
u is the same or different at each instance and is 0, 1 or 2.

33. A composition comprising at least one compound as claimed in claim 21 and at least one further matrix material, wherein the further matrix material is selected from compounds of one of the formulae (11), (12), (13), (14), (15), (16), (17) and (18): 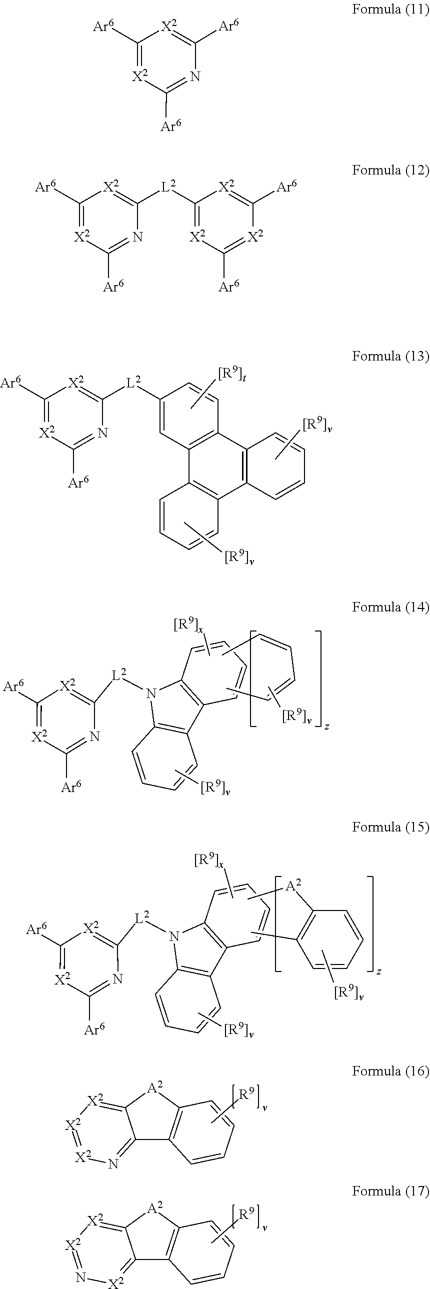 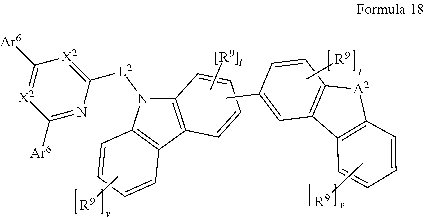
where the symbols and indices used are as follows:
X² is N or CR⁹, with the proviso that not more than two of the X² groups in one cycle are N;
L² is a connecting group which is selected from a bond or an aromatic or heteroaromatic ring system which has 5 to 40 aromatic ring atoms and may be substituted by one or more R⁹ radicals;
Ar⁶ is the same or different at each instance and is an aromatic or heteroaromatic ring system which has 5 to 40 aromatic ring atoms and may be substituted by one or more R¹⁰ radicals;
R⁹ is the same or different at each instance and is H, D, F, Cl, Br, I, N(R¹⁰)₂, N(Ar′″)₂, CN, NO₂, OR¹⁰, SR¹⁰, COOR¹⁰, C(═O)N(R¹⁰)₂, Si(R¹⁰)₃, B(OR¹⁰)₂, C(═O)R¹⁰, P(═O)(R¹⁰)₂, S(═O)R¹⁰, S(═O)₂R¹⁰, OSO₂R¹⁰, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals and where one or more nonadjacent CH₂ groups may be replaced by Si(R¹⁰)₂, C═O, NR¹⁰, O, S or CONR¹⁰, or an aromatic or heteroaromatic ring system which has 5 to 60 aromatic ring atoms, and may be substituted in each case by one or more R¹⁰ radicals; at the same time, two R⁹ radicals together may also form an aromatic, heteroaromatic, aliphatic or heteroaliphatic ring system;
Ar″′ is the same or different at each instance and is an aromatic or heteroaromatic ring system which has 5 to 40 aromatic ring atoms and may be substituted by one or more R¹⁰ radicals;
A² is C(R¹⁰)₂, NR¹⁰, O or S;
R¹⁰ is the same or different at each instance and is H, D, F, Cl, Br, I, N(R¹¹)₂, CN, NO₂, OR¹¹, SR¹¹, Si(R¹¹)₃, B(OR¹¹)₂, C(═O)R¹¹, P(═O)(R¹¹)₂, S(═O)R¹¹, S(═O)₂R¹¹, OSO₂R¹¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹¹ radicals and where one or more nonadjacent CH₂ groups may be replaced by Si(R¹¹)₂, C═O, NR¹¹, O, S or CONR¹¹, or an aromatic or heteroaromatic ring system which has 5 to 40 aromatic ring atoms and may be substituted in each case by one or more R¹¹ radicals; at the same time, two or more R¹⁰ radicals together may form an aromatic, heteroaromatic, aliphatic or heteroaliphatic ring system;
R¹¹ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4
t is the same or different at each instance and is 0, 1, 2 or 3;
x is the same or different at each instance and is 0, 1, 2, 3 or 4;
z is the same or different at each instance and is 0, 1 or 2,
where the sum total of x and 2z is not more than 4.

34. The composition as claimed in claim 32, wherein the compound has a proportion by mass in the composition in the range from 10% by weight to 95% by weight, based on the total mass of the composition.

35. The composition as claimed in claim 33, wherein the compounds of one of the formulae (7), (8), (9), (10), (11), (12), (13), (14), (15), (16), (17) and (18) have a proportion by mass in the composition in the range from 5% by weight to 90% by weight, based on the overall composition 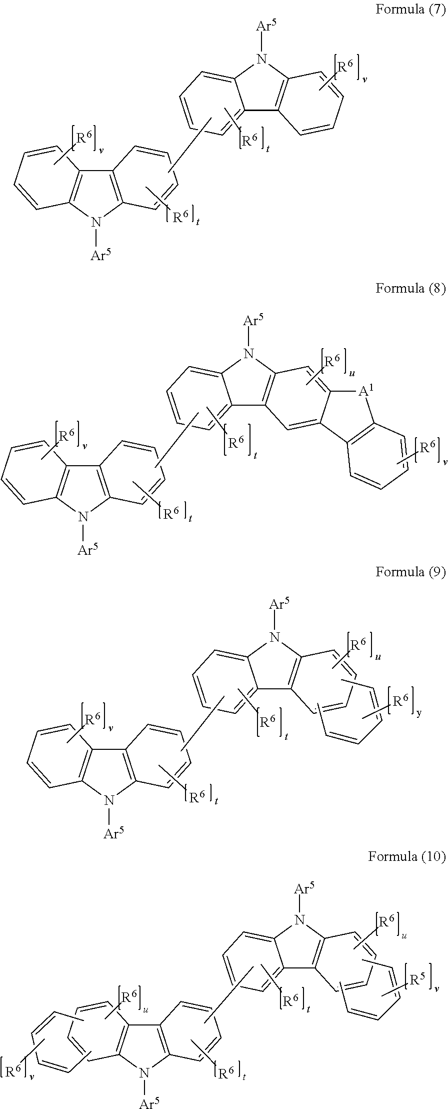
where the symbols and indices used are as follows:
R⁶ is the same or different at each instance and is H, D, F, Cl, Br, I, N(R⁷)₂, N(Ar′)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals, where one or more nonadjacent CH₂ groups may be replaced by Si(R⁷)₂, C═O, NR⁷, O, S or CONR⁷, or an aromatic or heteroaromatic ring system which has 5 to 60 aromatic ring atoms, and may be substituted in each case by one or more R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S;
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, OR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
s is the same or different at each instance and is 0, 1, 2, 3 or 4;
t is the same or different at each instance and is 0, 1, 2 or 3; and
u is the same or different at each instance and is 0, 1 or 2.

36. A formulation comprising at least one compound as claimed in claim 21 and at least one further compound.

37. A formulation comprising at least one composition as claimed in claim 32 and at least one further compound.

38. The formulation as claimed in claim 36, wherein the at least one further compound is a solvent.

39. An electronic device comprising at least one compound as claimed in claim 21.

40. An electronic device comprising the composition as claimed in claim 32.

41. The electronic device as claimed in claim 39, wherein the electronic device is an electroluminescent device.

42. An organic electroluminescent device, which comprises the compound as claimed in claim 21 is used as matrix material in an emitting layer and/or in an electron transport layer and/or in a hole blocker layer and/or in a hole transport layer and/or in an electron blocker layer.

43. An electronic device which comprises the compound as claimed in claim 21 is used as matrix material for phosphorescent emitters in combination with a further matrix material, where the further matrix material is selected from compounds of one of the formulae (7), (8), (9) and (10): 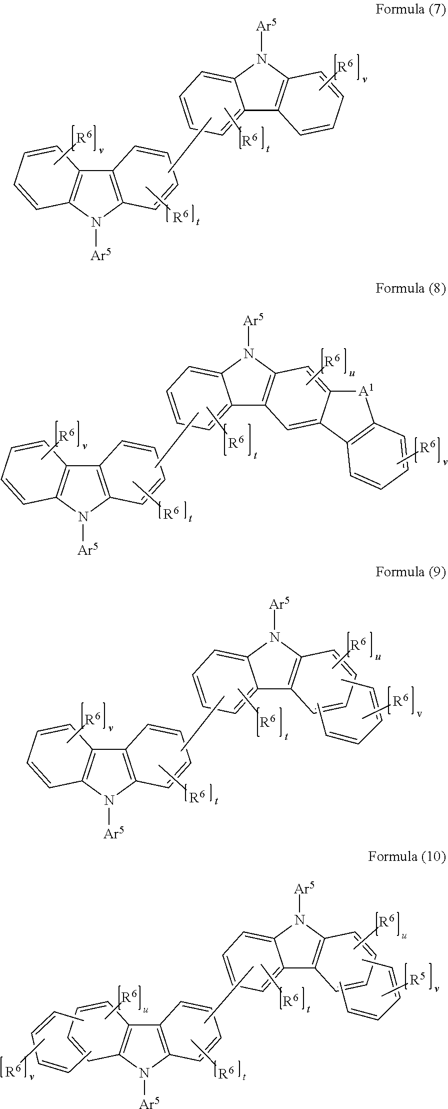
wherein
R⁶ is the same or different at each instance and is H, D, F, Cl, Br, I, N(R⁷)₂, N(Ar′)₂, CN, NO₂, OR⁷, SR⁷, COOR⁷, C(═O)N(R⁷)₂, Si(R⁷)₃, B(OR⁷)₂, C(═O)R⁷, P(═O)(R⁷)₂, S(═O)R⁷, S(═O)₂R⁷, OSO₂R⁷, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁷ radicals, where one or more nonadjacent CH₂ groups may be replaced by Si(R⁷)₂, C═O, NR⁷, O, S or CONIC, or an aromatic or heteroaromatic ring system which has 5 to 60 aromatic ring atoms, and may be substituted in each case by one or more R⁷ radicals; at the same time, two R⁶ radicals together may also form an aromatic, heteroaromatic, aliphatic or heteroaliphatic ring system;
Ar″ is the same or different at each instance and is an aromatic or heteroaromatic ring system which has 5 to 40 aromatic ring atoms and may be substituted by one or more R⁷ radicals;
A¹ is C(R⁷)₂, NR⁷, O or S;
Ar⁵ is the same or different at each instance and is an aromatic or heteroaromatic ring system which has 5 to 40 aromatic ring atoms and may be substituted by one or more R⁷ radicals;
R⁷ is the same or different at each instance and is H, D, F, Cl, Br, I, N(R⁸)₂, CN, NO₂, OR⁸, SR⁸, Si(R⁸)₃, B(OR⁸)₂, C(═O)R⁸, P(═O)(R⁸)₂, S(═O)R⁸, S(═O)₂R⁸, OSO₂R⁸, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁸ radicals and where one or more nonadjacent CH₂ groups may be replaced by Si(R⁸)₂, C═O, NR⁸, O, S or CONR⁸, or an aromatic or heteroaromatic ring system which has 5 to 40 aromatic ring atoms and may be substituted in each case by one or more R⁸ radicals; at the same time, two or more R⁷ radicals together may form an aromatic, heteroaromatic, aliphatic or heteroaliphatic ring system;
R⁸ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
s is the same or different at each instance and is 0, 1, 2, 3 or 4;
t is the same or different at each instance and is 0, 1, 2 or 3;
u is the same or different at each instance and is 0, 1 or 2;
and/or the further matrix material is selected from compounds of one of the formulae (11), (12), (13), (14), (15), (16), (17) and (18), 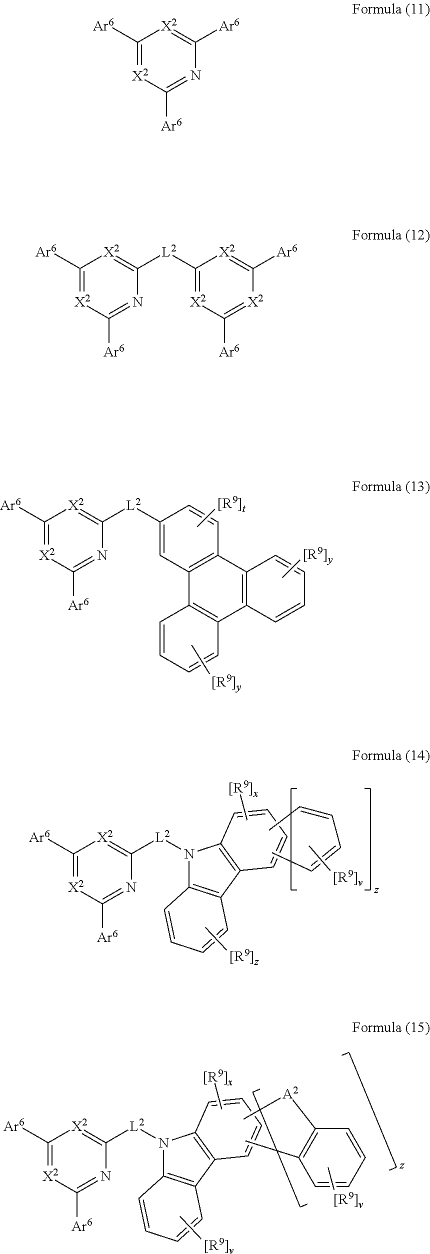 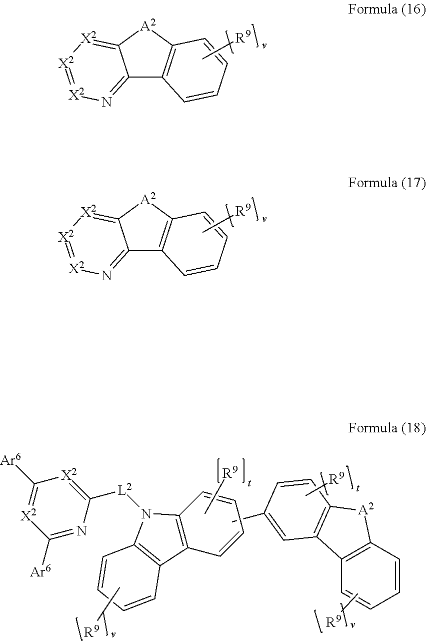
wherein
X² is N or CR⁹, with the proviso that not more than two of the X² groups in one cycle are N;
L² is a connecting group which is selected from a bond or an aromatic or heteroaromatic ring system which has 5 to 40 aromatic ring atoms and may be substituted by one or more R⁹ radicals;
Ar⁶ is the same or different at each instance and is an aromatic or heteroaromatic ring system which has 5 to 40 aromatic ring atoms and may be substituted by one or more R¹⁰ radicals;
R⁹ is the same or different at each instance and is H, D, F, Cl, Br, I, N(R¹⁰)₂, N(Ar′″)₂, CN, NO₂, OR¹⁰, SR¹⁰, COOR¹⁰, C(═O)N(R¹⁰)₂, Si(R¹⁰)₃, B(OR¹⁰)², C(═O)R¹⁰, P(═O)(R¹⁰)₂, S(═O)R¹⁰, S(═O)₂R¹⁰, OSO₂R¹⁰, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R⁴ radicals and where one or more nonadjacent CH₂ groups may be replaced by Si(R¹⁰)₂, C═O, NR¹⁰, O, S or CONR¹⁰, or an aromatic or heteroaromatic ring system which has 5 to 60 aromatic ring atoms, and may be substituted in each case by one or more R¹⁰ radicals; at the same time, two R⁹ radicals together may also form an aromatic, heteroaromatic, aliphatic or heteroaliphatic ring system;
Ar″′ is the same or different at each instance and is an aromatic or heteroaromatic ring system which has 5 to 40 aromatic ring atoms and may be substituted by one or more R¹⁰ radicals;
A² is C(R¹⁰)₂, NR¹⁰, O or S;
R¹⁰ is the same or different at each instance and is H, D, F, Cl, Br, I, N(R¹¹)₂, CN, NO₂, OR¹¹, SR¹¹, Si(R¹¹)₃, B(OR¹¹)₂, C(═O)R¹¹, P(═O)(R¹¹)₂, S(═O)R¹¹, S(═O)₂R¹¹, OSO₂R¹¹, a straight-chain alkyl group having 1 to 20 carbon atoms or an alkenyl or alkynyl group having 2 to 20 carbon atoms or a branched or cyclic alkyl group having 3 to 20 carbon atoms, where the alkyl, alkenyl or alkynyl group may in each case be substituted by one or more R¹¹ radicals and where one or more nonadjacent CH₂ groups may be replaced by Si(R¹¹)₂, C═O, NR¹¹, O, S or CONR¹¹, or an aromatic or heteroaromatic ring system which has 5 to 40 aromatic ring atoms and may be substituted in each case by one or more R¹¹ radicals; at the same time, two or more R¹⁰ radicals together may form an aromatic, heteroaromatic, aliphatic or heteroaliphatic ring system;
R¹¹ is the same or different at each instance and is H, D, F or an aliphatic, aromatic or heteroaromatic organic radical, especially a hydrocarbyl radical, having 1 to 20 carbon atoms, in which one or more hydrogen atoms may also be replaced by F;
v is the same or different at each instance and is 0, 1, 2, 3 or 4
t is the same or different at each instance and is 0, 1, 2 or 3;
x is the same or different at each instance and is 0, 1, 2, 3 or 4;
z is the same or different at each instance and is 0, 1 or 2,
where the sum total of x and 2z is not more than 4.
